(19) United States
(12) Patent Application Publication
(10) Pub. No.: US 2011/0091524 A1
(43) Pub. Date: Apr. 21, 2011

(54) BIOMARKERS FOR SENSITIVITY TO ANTI-IGF1R THERAPY

(76) Inventors: Yan Wang, Warren, NJ (US); Yaolin Wang, Short Hills, NJ (US); Diene Levitan, Tenafly, NJ (US); Cynthia Seidel-Dugan, Mountainside, NJ (US); Ming Liu, Fanwood, NJ (US); Wei Ding, Scotch Plains, NJ (US)

(21) Appl. No.: 12/808,270

(22) PCT Filed: Dec. 17, 2008
(86) PCT No.: PCT/US2008/087240

§ 371 Date: Sep. 7, 2010

Related U.S. Application Data
(60) Provisional application No. 61/014,556, filed on Dec. 18, 2007.
(60) Provisional application No. 61/015,938, filed on Dec. 21, 2007.
(60) Provisional application No. 61/022,909, filed on Jan. 23, 2008.

(51) Int. Cl. A61K 39/395; C12Q 1/68; A61K 38/20; A61K 38/21; A61K 9/127; A61P 35/00; A61P 35/02; G06Q 90/00

(52) U.S. Cl. 424/450; 435/6; 424/172.1; 424/175.1; 424/158.1; 424/142.1; 424/133.1; 424/85.2; 424/85.7; 705/500

(57) ABSTRACT

The present invention provides, for example, methods for conveniently determining if a cancerous condition in a subject will be responsive to an IGF1R inhibitor. The invention includes patient selection methods and methods of treatment.

This application claims the benefit of U.S. provisional patent application no. 61/014,556, filed Dec. 18, 2007; U.S. provisional patent application No. 61/015,938, filed Dec. 21, 2007; and U.S. provisional patent application No. 61/022,909, filed Jan. 23, 2008; each of which is herein incorporated by referenced in its entirety.

### FIELD OF THE INVENTION

The present invention relates, in general, to methods for determining if a malignant or neoplastic cell in a subject or any medical condition in a subject mediated by IGF1R is sensitive to an IGF1R inhibitor.

### BACKGROUND OF THE INVENTION

The insulin-like growth factors, also known as somatomedins, include insulin-like growth factor-I (IGF-I) and insulin-like growth factor-II (IGF-II) (Klapper, et al., (1983) Endocrinol. 112:2215 and Rinderknecht, et al., (1978) Febs. Lett. 89:283). These growth factors exert mitogenic activity on various cell types, including tumor cells (Macaulay, (1992) Br. J. Cancer 65:311), by binding to a common receptor named the insulin-like growth factor receptor-1 (IGF1R) (Sepp-Lorenzino, (1998) Breast Cancer Research and Treatment 47:235). There are several available anti-cancer therapies which target IGF1R; however, due to factors including, e.g., individual genetic variability which can render a particular patient non-responsive to a given therapy some patients are not fully responsive to the therapy. The use of biomarkers for responsiveness to a given therapy is, thus, a useful tool for quickly and conveniently determining the responsiveness of a patient before a course of treatment is initiated. Biomarkers include, for example, the expression of a given gene or post-translational modification of a protein (e.g., phosphorylation) in a patient (e.g., in the cells of a cancer patient's tumor), e.g., at a level greater or less than that of a known responder or known non-responder.

Often, early, successful treatment of a given cancer is critical to the patient's clinical outcome. The use of biomarkers can aid in this process by quickly helping to identify treatments likely to be effective in a given patient and/or helping to eliminate treatments likely to be ineffective in a given patient.

Another benefit of the use of biomarkers relates to patient compliance. Patients assured that a given IGF1R inhibitor therapy will likely be effective against their specific tumor will exhibit an enhanced likelihood of continuing with the prescribed IGF1R inhibitor-based regimen over time.

### SUMMARY OF THE INVENTION

The present invention provides a method for evaluating sensitivity of malignant or neoplastic cells (e.g., from an in vitro or in vivo source) to an IGF1R inhibitor (e.g., with about 70% certainty, e.g., about 72.5% or 75.7%) comprising determining if said cells exhibit high expression of one or more genes set forth in table 1 or low expression of one or more genes set forth in table 3 relative to that of a cell resistant to said inhibitor; wherein said cells are determined to be sensitive if said high expression or said low expression is observed. In an embodiment of the invention, the method comprises (a) obtaining a sample of one or more malignant or neoplastic cells from the body of a subject; (b) evaluating expression of one or more genes set forth in table 1 (e.g., ELLS1 and/or AUTS2 and/or TCF4 and/or TLE; e.g., all 4, 3, 2 or 1 in any combination whatsoever) or table 3 in the malignant or neoplastic cells; and (c) comparing said expression level to that of cells resistant to said IGF1R inhibitor; wherein the cells are determined to be sensitive to the inhibitor if expression of one or more genes in table 1 is higher than that of a cell resistant to said inhibitor or if expression of one or more genes in table 3 is lower than that of a cell resistant to said inhibitor. In an embodiment of the invention, the method further comprising administering a therapeutically effective dose of said inhibitor, optionally in association with a further therapeutic agent, to the body of a subject comprising said malignant or neoplastic cells if the cells are determined to be sensitive.

The present invention also provides a method for selecting a subject with malignant or neoplastic cells for treatment with an IGF1R inhibitor comprising evaluating sensitivity of the malignant or neoplastic cells to said inhibitor by the method for evaluating sensitivity discussed above; wherein said subject is selected if said cells are determined to be sensitive.

The present invention further provides a method for treating a tumor or cancerous condition with an IGF1R inhibitor comprising evaluating sensitivity of malignant or neoplastic cells, which are in said tumor or which mediate said cancerous condition, to said inhibitor by the method for evaluating sensitivity discussed above and, if said cells are determined to be sensitive, continuing or commencing treatment by administering, to the subject, a therapeutically effective dose of the inhibitor.

The present invention also provides a method for selecting a therapy for a subject with one or more malignant or neoplastic cells comprising evaluating sensitivity of the cells to an IGF1R inhibitor by the method for evaluating sensitivity discussed above; wherein said inhibitor is selected as the therapy if said cells are determined to be sensitive to the inhibitor.

The present invention further provides a method of advertising an IGF1R inhibitor or a pharmaceutically acceptable composition thereof or a therapeutic regimen comprising administration of said inhibitor or composition comprising promoting, to a target audience, the use of the inhibitor or composition for treating a patient or patient population whose tumors or cancerous conditions are mediated by malignant or neoplastic cells that exhibit increased expression of one or more genes set forth in table 1 (e.g., ELLS1 and/or AUTS2 and/or TCF4 and/or TLE; e.g., all 4, 3, 2 or 1 in any combination whatsoever), relative to cells resistant to said inhibitor; or that exhibit decreased expression of one or more genes set forth in table 1, relative to cells resistant to said inhibitor.

The scope of the present invention further includes an article of manufacture comprising, packaged together, an IGF1R inhibitor or a pharmaceutical composition thereof comprising a pharmaceutically acceptable carrier; and a label stating that the agent or pharmaceutical composition is indicated for treating patients having a tumor comprising malignant or neoplastic cells or a cancerous condition mediated by malignant or neoplastic cells that exhibit increased expression of one or more genes set forth in table 1 (e.g., ELLS1 and/or AUTS2 and/or TCF4 and/or TLE; e.g., all 4, 3, 2 or 1 in any combination whatsoever), relative to cells resistant to said inhibitor; or that exhibit decreased expression of one or more genes set forth in table 3, relative to cells resistant to said inhibitor.

Also provided by the present invention is a method for manufacturing an IGF1R inhibitor or a pharmaceutical composition thereof comprising a pharmaceutically acceptable carrier said method comprising combining, in a package, the inhibitor or composition; and a label conveying that the inhibitor or composition is indicated for treating patients having a tumor comprising malignant or neoplastic cells or a cancerous condition mediated by malignant or neoplastic cells that exhibit increased expression of one or more genes set forth in table 1 (e.g., ELLS1 and/or AUTS2 and/or TCF4 and/or TLE; e.g., all 4, 3, 2 or 1 in any combination whatsoever), relative to cells resistant to said inhibitor; or that exhibit decreased expression of one or more genes set forth in table 3, relative to cells resistant to said inhibitor.

In an embodiment of any of the inventions discussed herein an IGF1R inhibitor is administered in association with a further chemotherapeutic agent. For example, a further therapeutic agent is, in an embodiment of the invention, any member selected from the group consisting of everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR, KRX-0402, lucanthone, LY 317615, neuradiab, vitespan, Rta 744, Sdx 102, talampanel, atrasentan, Xr 311, romidepsin, ADS-100380,

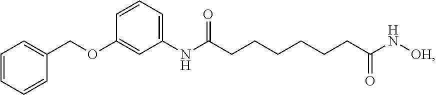

### CG-781, CG-1521,

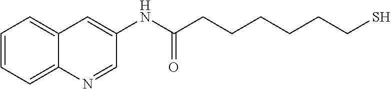

SB-556629, chlamydocin, JNJ-16241199,

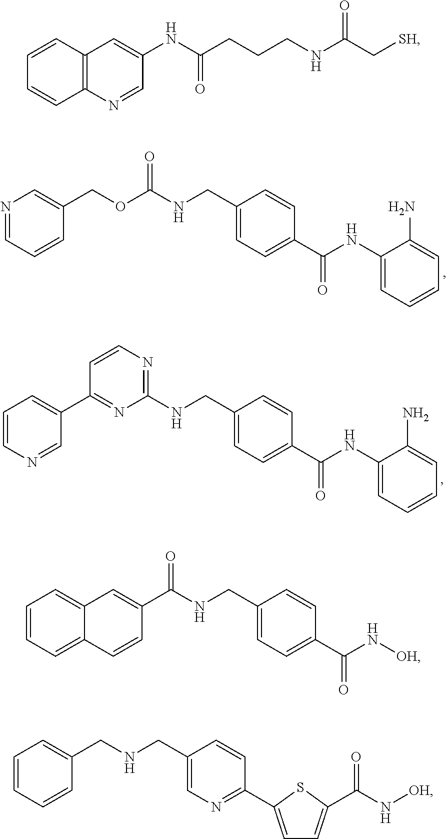

vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, irinotecan; a combination of irinotecan, 5-fluorouracil and leucovorin; PEG-labeled irinotecan, FOLFOX regimen, tamoxifen, toremifene citrate, anastrazole, exemestane, letrozole, DES (diethylstilbestrol), estradiol, estrogen, conjugated estrogen, bevacizumab, IMC-1C11, CHIR-258,

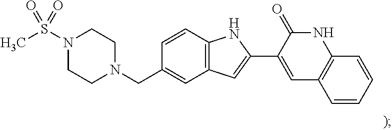

3-[5-(methylsulfonylpiperadinemethyl)-indolyl]-quinolone, vatalanib, AG-013736, AVE-0005, the acetate salt of [D-Ser(But) 6, Azgly 10] (pyro-Glu-His-Trp-Ser-Tyr-D-Ser(But)-Leu-Arg-Pro-Azgly-NH₂ acetate [C₅₉H₈₄N₁₈O₁₄.(C₂H₄O₂)ₓ where x=1 to 2.4], goserelin acetate, leuprolide acetate, triptorelin pamoate, sunitinib, sunitinib malate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, lonafarnib,

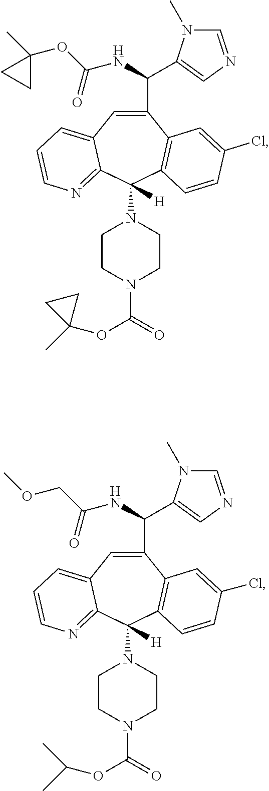

BMS-214662, tipifarnib; amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyclophosphamide, cyproterone, cytarabine, dacarbazine, dactinomycin, daunorubicin, diethylstilbestrol, epirubicin, fludarabine, fludrocortisone, fluoxymesterone, flutamide, hydroxyurea, idarubicin, ifosfamide, imatinib, leucovorin, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mercaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, 5-fluorouracil, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa and darbepoetin alfa.

Also, in an embodiment of any of the inventions set forth herein, an IGF1R inhibitor is any member selected from the group consisting of any antibody or antigen-binding fragment thereof which binds specifically to IGF1R,

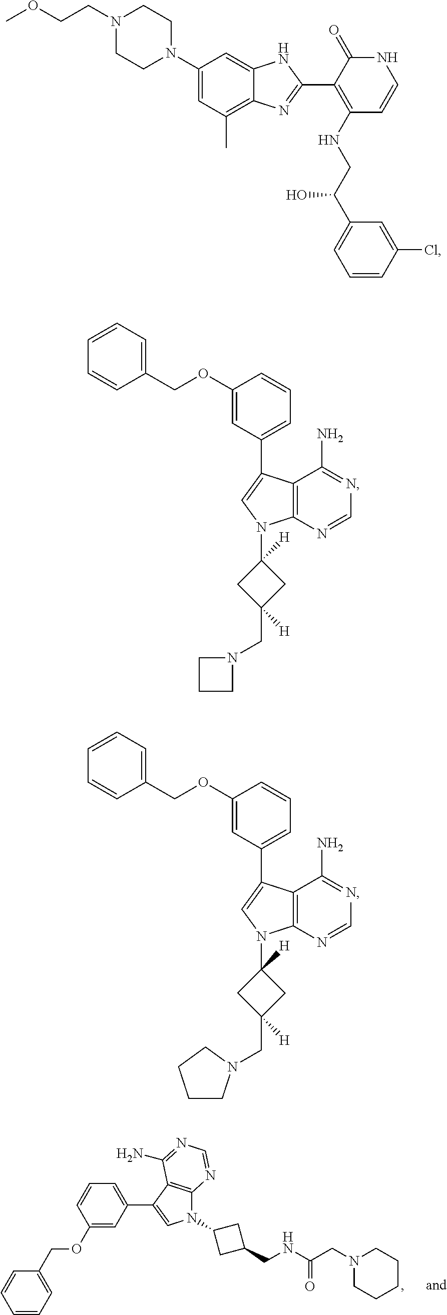

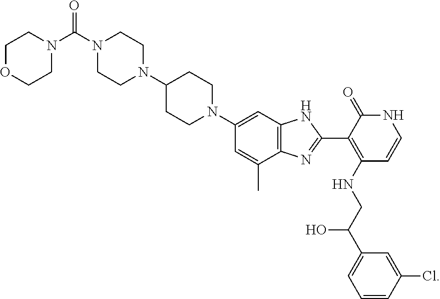

For example, such an antibody or fragment can, in an embodiment of the invention, comprise one or more complementarity determining regions (CDRs) selected from the group consisting of:

### RASQSIGSSLH (SEQ ID NO: 99),

### YASQSLS (SEQ ID NO: 100),

### HQSSRLPHT (SEQ ID NO: 101),

e.g., a light chain immunoglobulin comprising CDRs comprising the amino acid sequences of SEQ ID NOs: 99-101;

### SFAMH (SEQ ID NO:102),

### GFTFSSFAMH (SEQ ID NO: 107),

### VIDTRGATYYADSVKG (SEQ ID NO: 103), and

LGNFYYGMDV (SEQ ID NO: 104); e.g., a heavy chain immunoglobulin comprising a CDR comprising the amino acid sequence of SEQ ID NO: 102 or 107, a CDR comprising the amino acid sequence of SEQ ID NO: 103 and a CDR comprising the amino acid sequence of SEQ ID NO: 104;
or a mature fragment of a light chain immunoglobulin which comprises the amino acid sequence of SEQ ID NO: 2, 4, 6 or 8; and/or a mature fragment of a heavy chain immunoglobulin which comprises the amino acid sequence of SEQ ID NO: 10 or 12.

Embodiments of the present invention includes those wherein the malignant or neoplastic cells are in a tumor or mediate a cancerous condition which tumor or condition is selected from the group consisting of osteosarcoma, rhabdomyosarcoma, neuroblastoma, any pediatric cancer, kidney cancer, leukemia, renal transitional cell cancer, Werner-Morrison syndrome, bladder cancer, Wilm's cancer, ovarian cancer, pancreatic cancer, breast cancer, prostate cancer, benign prostatic hyperplasia, bone cancer, lung cancer, gastric cancer, colorectal cancer, cervical cancer, synovial sarcoma, diarrhea associated with metastatic carcinoid, vasoactive intestinal peptide secreting tumors, tumor angiogenesis, head and neck cancer, squamous cell carcinoma, multiple myeloma, solitary plasmacytoma, renal cell cancer, retinoblastoma, germ cell tumors, hepatoblastoma, hepatocellular carcinoma, melanoma, rhabdoid tumor of the kidney, Ewing Sarcoma, chondrosarcoma, haemotological malignancy, chronic lymphoblastic leukemia, chronic myelomonocytic leukemia, acute lymphoblastic leukemia, acute lymphocytic leukemia, acute myelogenous leukemia, acute myeloblastic leukemia, chronic myeloblastic leukemia, Hodgekin's disease, non-Hodgekin's lymphoma, chronic lymphocytic leukemia, chronic myelogenous leukemia, myelodysplastic syndrome, hairy cell leukemia, mast cell leukemia, mast cell neoplasm, follicular lymphoma, diffuse large cell lymphoma, mantle cell lymphoma, Burkitt Lymphoma, mycosis fungoides, seary syndrome, cutaneous T-cell lymphoma, chronic myeloproliferative disorders, a central nervous system tumor, brain cancer, glioblastoma, non-glioblastoma brain cancer, meningioma, pituitary adenoma, vestibular schwannoma, a primitive neuroectodermal tumor, medulloblastoma, astrocytoma, anaplastic astrocytoma, oligodendroglioma, ependymoma, choroid plexus papilloma, a myeloproliferative disorder, polycythemia vera, thrombocythemia, idiopathic myelfibrosis, soft tissue sarcoma, thyroid cancer, endometrial cancer, carcinoid cancer, germ cell tumors and liver cancer.

Embodiments of the present invention also includes those wherein expression of one or more of said genes is identified by Northern blot analysis.

### DETAILED DESCRIPTION OF THE INVENTION

The present invention relates e.g., to methods for selecting patients for treatment with an IGF1R inhibitor. Such patients comprise one or more malignant or neoplastic cells and, in an embodiment of the invention, suffer from a disease or medical condition which is mediated by such a malignant or neoplastic cell. Malignant or neoplastic cells include, for example, cancerous cells. Malignant cells include, for example, cells exhibiting anaplasia, metastasis, invasiveness, tendency to form a tumor and/or a tendency to lead to death (e.g., due to cancer caused by a tumor including such malignant cells). Neoplastic cells include, for example, cells which abnormally divide at a supra-normal level (e.g., high numbers of lifetime divisions or division at a high rate) and/or to exhibit low mortality or immortality. In an embodiment of the invention, said malignant and/or neoplastic properties are mediated by IGF1R activity or expression in the cell.

The term “subject” or “patient” includes any animal including, e.g., a mammal such as a human.

The term IGF1R inhibitor resistant cell or the like includes any cell that is resistant to an IGF1R inhibitor, e.g., with respect to its growth and/or proliferation and/or survival. For example, in an embodiment of the invention, an IGF1R inhibitor sensitive cell or cell line exhibits 50% or more tumor growth inhibition (e.g., reduction in tumor volume and/or tumor mass) in a mouse xenograft system (wherein the tested cells form the tumor) wherein, when the inhibitor is an anti-IGF1R antibody or antigen-binding fragment thereof, the mouse is administered 0.5 mg of antibody or fragment twice a week for about 3 weeks. In an embodiment of the invention, the cell is resistant if less than 50% in vivo tumor growth inhibition is exhibited. In an embodiment of the invention, a cell or cell line is sensitive to an IGF1R inhibitor if, in vitro, the cell or cell line exhibits 30% or more growth inhibition, wherein, when the inhibitor is an anti-IGF1R antibody or antigen-binding fragment thereof, the cell or cell line is exposed to about 20 nM to about 100 nm of the antibody or fragment, e.g., by a luminescent cell viability assay such as a CellTiter Glo assay. In an embodiment of the invention, the cell or cell line is resistant when it exhibits less than 30% in vitro growth inhibition.

### IGF1R Inhibitors

The terms “IGF1R inhibitor” or “IGF1R antagonist” or the like include any substance that decreases the expression, ligand binding (e.g., binding to IGF-1 and/or IGF-2), kinase activity (e.g., autophosphorylation activity) or any other biological activity of IGF1R (e.g., mediation of anchorage-independent cellular growth) e.g., that will elicit a biological or medical response of a tissue, system, subject or patient that is being sought by the administrator (such as a researcher, doctor or veterinarian) which includes any measurable alleviation of the signs, symptoms and/or clinical indicia of cancer (e.g., tumor growth) and/or the prevention, slowing or halting of progression or metastasis of cancer to any degree.

In an embodiment of the invention, the IGF1R inhibitor is any isolated antibody or antigen-binding fragment thereof that binds specifically to insulin-like growth factor-1 receptor (e.g., human IGF1R) or any soluble fragment thereof (e.g., monoclonal antibodies (e.g., fully human monoclonal antibodies), polyclonal antibodies, bispecific antibodies, Fab antibody fragments, F(ab)₂ antibody fragments, Fv antibody fragments (e.g., VH or VL), single chain Fv antibody fragments, dsFv antibody fragments, humanized antibodies or chimeric antibodies) such as any of those disclosed in any of Burtrum et. al Cancer Research 63:8912-8921 (2003); in French Patent Applications FR2834990, FR2834991 and FR2834900 and in PCT Application Publication Nos. WO 03/100008; WO 03/59951; WO 04/71529; WO 03/106621; WO 04/83248; WO 04/87756, WO 05/16970; and WO 02/53596.

In an embodiment of the invention, an IGF1R inhibitor is an isolated anti-insulin-like growth factor-1 receptor (IGF1R) antibody comprising a mature 19D12/15H12 Light Chain (LC)-C, D, E or F and a mature 19D12/15H12 heavy chain (HC)-A or B (e.g., mature LCB/mature HCB, mature LCC/mature HCB or mature LCF/mature HCA). In an embodiment of the invention, an IGF1R inhibitor that is administered to a patient in a method according to the invention is an isolated antibody that specifically binds to IGF1R that comprises one or more complementarity determining regions (CDRs) of 19D12/15H12 Light Chain-C, D, E or F and/or 19D12/15H12 heavy chain-A or B (e.g., all 3 light chain CDRs and/or all 3 heavy chain CDRs).

The amino acid and nucleotide sequences of the some antibody chains of the invention are shown below. Dotted, underscored type indicates the signal peptide. Solid underscored type indicates the CDRs. Plain type indicates the framework regions. Mature fragments lack the signal peptide.

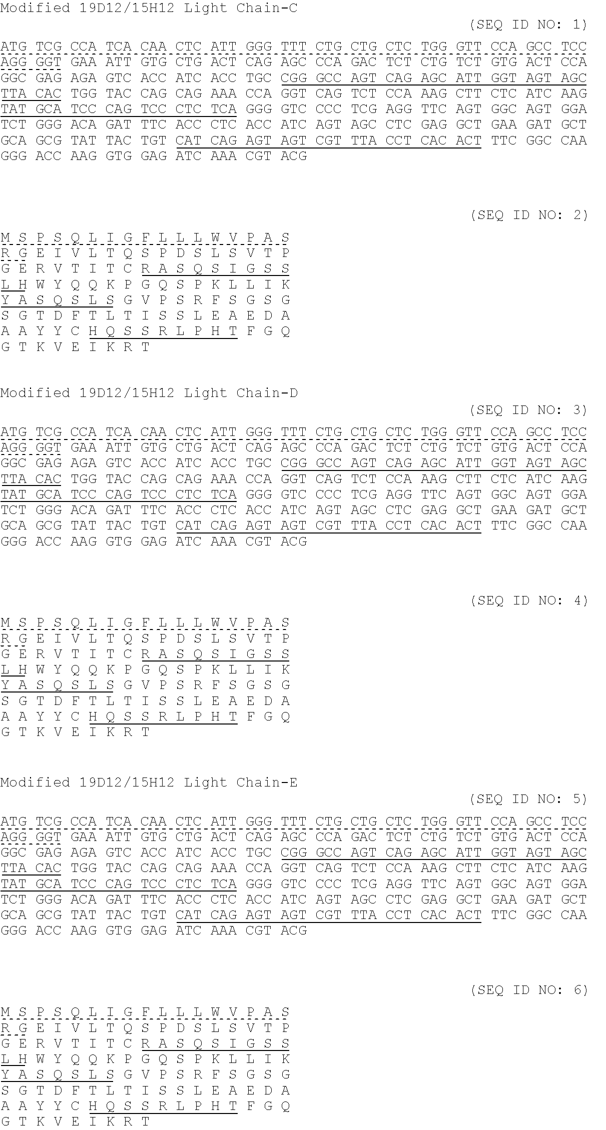

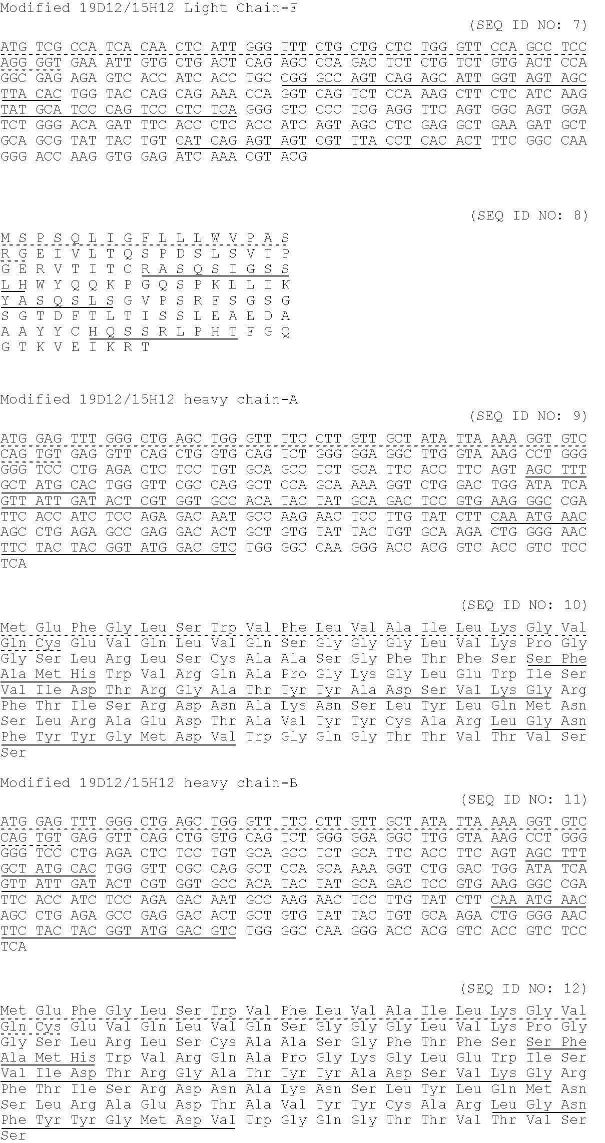

Plasmids comprising a CMV promoter operably linked to the 15H12/19D12 light chains and heavy chains have been deposited at the American Type Culture Collection (ATCC); 10801 University Boulevard; Manassas, Va. 20110-2209 on May 21, 2003. The deposit name and the ATCC accession numbers for the cell lines are set forth below:

CMV promoter-15H12/19D12 LCC (κ)—

Deposit name: “15H12/19D12 LCC (κ)”;

ATCC accession No.: PTA-5217

CMV promoter-15H12/19D12 LCD (κ)—

Deposit name: “15H12/19D12 LCD (κ)”;

ATCC accession No.: PTA-5218

CMV promoter-15H12/19D12 LCE (κ)—

Deposit name: “15H12/19D12 LCE (κ)”;

ATCC accession No.: PTA-5219

CMV promoter-15H12/19D12 LCF (κ)—

Deposit name: “15H12/19D12 LCF (κ)”;

ATCC accession No.: PTA-5220

CMV promoter-15H12/19D12 HCA (γ4)—

Deposit name: “15H12/19D12 HCA (γ4)”

ATCC accession No.: PTA-5214

CMV promoter-15H12/19D12 HCB (γ4)—

Deposit name: “15H12/19D12 HCB (γ4)”

ATCC accession No.: PTA-5215

CMV promoter-15H12/19D12 HCA (γ1)—

Deposit name: “15H12/19D12 HCA (γ1)”;

ATCC accession No.: PTA-5216

The present invention includes methods and compositions (e.g., any disclosed herein) comprising anti-IGF1R antibodies and antigen-binding fragments thereof comprising any of the light and/or heavy immunoglobulin chains or mature fragments thereof located in any of the foregoing plasmids deposited at the ATCC.

In an embodiment of the invention, the IGF1R inhibitor is an isolated antibody or antigen-binding fragment thereof comprising one or more (e.g., 3) of the following CDR sequences:

| | |
|---|---|
| RASQSIGSSLH; | (SEQ ID NO: 157) |
| YASQSLS; | (SEQ ID NO: 158) |
| HQSSRLPHT; | (SEQ ID NO: 159) |
| SFAMH; | (SEQ ID NO: 160) |
| VIDTRGATYYADSVKG; | (SEQ ID NO: 161) |
| LGNFYYGMDV. | (SEQ ID NO: 162) |

For example, in an embodiment of the invention, a light chain immunoglobulin comprises 3 CDRs and/or a heavy chain immunoglobulin comprises 3 CDRs.

In an embodiment, an antibody that binds “specifically” to human IGF1R binds with a Kd of about 10⁻⁸ M or 10⁻⁷ M or a lower number; or, in an embodiment of the invention, with a Kd of about 1.28×10⁻¹⁰ M or a lower number by Biacore measurement or with a Kd of about 2.05×10⁻¹² or a lower number by KinExA measurement. In another embodiment, an antibody that binds “specifically” to human IGF1R binds exclusively to human IGF1R and to no other protein at significant or at detectable levels.

In an embodiment of the invention, the IGF1R inhibitor comprises any light chain immunoglobulin and/or a heavy chain immunoglobulin as set forth in Published International Application No. WO 2002/53596 which is herein incorporated by reference in its entirety. For example, in an embodiment, the antibody comprises a light chain variable region comprising an amino acid sequence selected from the group consisting of SEQ ID NOs: 2, 6, 10, 14, 18, 22, 47 and 51 as set forth in WO 2002/53596 and/or a heavy chain variable region comprising an amino acid sequence selected from the group consisting of SEQ ID NOs: 4, 8, 12, 16, 20, 24, 45 and 49 as set forth in WO 2002/53596. In an embodiment, the antibody comprises a heavy and/or light chain selected from that of antibody 2.12.1; 2.13.2; 2.14.3; 3.1.1; 4.9.2; and 4.17.3 in WO 2002/53596.

In an embodiment of the invention, the IGF1R inhibitor comprises any light chain immunoglobulin and/or a heavy chain immunoglobulin as set forth in Published International Application No. WO 2003/59951 which is herein incorporated by reference in its entirety. For example, in an embodiment, the antibody comprises a light chain variable region comprising an amino acid sequence selected from the group consisting of SEQ ID NOs: 54, 61 and 65 as set forth in WO 2003/59951 and/or a heavy chain variable region comprising an amino acids sequence selected from the group consisting of SEQ ID NOs: 69, 75, 79 and 83 as set forth in WO 2003/59951.

In an embodiment of the invention, the IGF1R inhibitor comprises any light chain immunoglobulin and/or a heavy chain immunoglobulin as set forth in Published International Application No. WO 2004/83248 which is herein incorporated by reference in its entirety. For example, in an embodiment, the antibody comprises a light chain variable region comprising an amino acid sequence selected from the group consisting of SEQ ID NOs: 109, 111, 113, 115, 117, 119, 121, 123, 125, 127, 129, 131, 133, 135, 137, 139, 141 and 143 as set forth in WO 2004/83248 and/or a heavy chain variable region comprising an amino acids sequence selected from the group consisting of SEQ ID NOs: 108, 110, 112, 114, 116, 118, 120, 122, 124, 126, 128, 130, 132, 134, 136, 138, 140 and 142 as set forth in WO 2004/83248. In an embodiment, the antibody comprises a light and/or heavy chain selected from that of PINT-6A1; PINT-7A2; PINT-7A4; PINT-7A5; PINT-7A6; PINT-8A1; PINT-9A2; PINT-11A1; PINT-11A2; PINT-11A3; PINT-11A4; PINT-11A5; PINT-11A7; PINT-12A1; PINT-12A2; PINT-12A3; PINT-12A4 and PINT-12A5 in WO 2004/83248.

In an embodiment of the invention, the IGF1R inhibitor comprises any light chain immunoglobulin and/or a heavy chain immunoglobulin as set forth in Published International Application No. WO 2003/106621 which is herein incorporated by reference in its entirety. For example, in an embodiment, the antibody comprises a light chain variable region comprising an amino acid sequence selected from the group consisting of SEQ ID NOs: 8-12, 58-69, 82-86, 90, 94, 96, 98, as set forth in WO 2003/106621 and/or a heavy chain variable region comprising an amino acids sequence selected from the group consisting of SEQ ID NOs: 7, 13, 70-81, 87, 88, 92 as set forth in WO 2003/106621.

In an embodiment of the invention, the IGF1R inhibitor comprises any light chain immunoglobulin and/or a heavy chain immunoglobulin as set forth in Published International Application No. WO 2004/87756 which is herein incorporated by reference in its entirety. For example, in an embodiment, the antibody comprises a light chain variable region comprising an amino acid sequence of SEQ ID NO: 2 as set forth in WO 2004/87756 and/or a heavy chain variable region comprising an amino acid sequence of SEQ ID NO: 1 as set forth in WO 2004/87756.

In an embodiment of the invention, the IGF1R inhibitor comprises any light chain immunoglobulin and/or a heavy chain immunoglobulin as set forth in Published International Application No. WO 2005/16970 which is herein incorporated by reference in its entirety. For example, in an embodiment, the antibody comprises a light chain variable region comprising an amino acid sequence of SEQ ID NO: 6 or 10 as set forth in WO 2005/16970 and/or a heavy chain variable region comprising an amino acid sequence of SEQ ID NO: 2 as set forth in WO 2005/16970.

In an embodiment of the invention, an anti-IGF1R antibody or antigen-binding fragment thereof comprises an immunoglobulin heavy chain variable region comprising an amino acid sequence selected from the group consisting of:

| (SEQ ID NO: 13) | |
|---|---|
| 1 | griggawrsl rlscaasgft fsdyymswir qapgkglewv syisssgstr |
| 51 | dyadsvkgrf tisrdnakns lylqmnslra edtavyycvr dgvettfyyy |
| 101 | yygmdvwgqg ttvtvssast kgpsvfplap csrstsesta algclvkdyf |
| 151 | pepvtvswns galtsgvhtf psca |
| (SEQ ID NO: 14) | |
| 1 | vqllesgggl vqpggslrls ctasgftfss yamnwvrqap gkglewvsai |
| 51 | sgsggttfya dsvkgrftis rdnsrttlyl qmnslraedt avyycakdlg |
| 101 | wsdsyyyyyg mdvwgqgttv tvss |
| (SEQ ID NO: 15) | |
| 1 | gpglvkpset lsltctvsgg sisnyywswi rqpagkglew igriytsgsp |
| 51 | nynpslksrv tmsvdtsknq fslklnsvta adtavyycav tifgvviifd |
| 101 | ywgqgtivtv ss |
| (SEQ ID NO: 16) | |
| 1 | evqllesggg lvqpggslrl scaasgftfs syamswvrqa pgkglewvsa |
| 51 | isgsggityy adsvkgrfti srdnskntly lqmnslraed tavyycakdl |
| 101 | gygdfyyyyy gmdvwgqgtt vtvss |
| (SEQ ID NO: 17) | |
| 1 | pglvkpsetl sltctvsggs issyywswir qppgkglewi gyiyysgstn |
| 51 | ynpslksrvt isvdtsknqf slklssvtaa dtavyycart ysssfyyygm |
| 101 | dvwgqgttvt vss |
| (SEQ ID NO: 18) | |
| 1 | evqllesggg lvqpggslrl scaasgftfs syamswvrqa pgkglewvsg |
| 51 | itgsggstyy adsvkgrfti srdnskntly lqmnslraed tavyycakdp |
| 101 | gttvimswfd pwgqgtlvtv ss |

In an embodiment of the invention, an anti-IGF1R antibody or antigen-binding fragment thereof comprises an immunoglobulin light chain variable region comprising an amino acid sequence selected from the group consisting of:

| (SEQ ID NO: 19) | |
|---|---|
| 1 | asvgdrvtft crasqdirrd lgwyqqkpgk apkrliyaas rlqsgvpsrf |
| 51 | sgsgsgteft ltisslqped fatyyclqhn nyprtfgqgt eveiirtvaa |
| 101 | psvfifppsd eqlksgtasv vcllnnfypr eakvqw |
| (SEQ ID NO: 20) | |
| 1 | diqmtqfpss lsasvgdrvt itcrasqgir ndlgwyqqkp gkapkrliya |
| 51 | asrlhrgvps rfsgsgsgte ftltisslqp edfatyyclq hnsypcsfgq |
| 101 | gtkleik |
| (SEQ ID NO: 21) | |
| 1 | sslsasvgdr vtftcrasqd irrdlgwygq kpgkapkrli yaasrlqsgv |
| 51 | psrfsgsgsg teftltissl qpedfatyyc lqhnnyprtf gqgteveiir |
| (SEQ ID NO: 22) | |
| 1 | diqmtqspss lsasvgdrvt itcrasqgir sdlgwfqqkp gkapkrliya |
| 51 | asklhrgvps rfsgsgsgte ftltisrlqp edfatyyclq hnsypltfgg |
| 101 | gtkveik |
| (SEQ ID NO: 23) | |
| 1 | gdrvtitcra sqsistflnw yqqkpgkapk llihvasslq ggvpsrfsgs |
| 51 | gsgtdftlti sslqpedfat yycqqsynap ltfgggtkve ik |
| (SEQ ID NO: 24) | |
| 1 | ratlscrasq svrgrylawy qqkpgqaprl liygassrat gipdrfsgsg |
| 51 | sgtdftltis rlepedfavf ycqqygsspr tfgqgtkvei k |

In an embodiment of the invention, the anti-IGF1R antibody comprises a light chain immunoglobulin, or a mature fragment thereof (i.e., lacking signal sequence), or variable region thereof, comprising the amino acid sequence of:

| (SEQ ID NO: 25) | |
|---|---|
| 1 | mdmrvpaqll gllllwfpga rcdiqmtqsp sslsasvgdr vtitc [symbol] |
| 51 | [symbol] wyqq kpgkapkrli y [symbol] gv psrfsgsgsg teftltissl |
| 101 | qpedfatyyc [symbol] f gqgtkveikr tvaapsvfif ppsdeqlksg |
| 151 | tasvvcllnn fypreakvqw kvdnalqsgn sqesvtegds kdstyslsst |
| 201 | ltlskadyek hkvyacevth qglsspvtks fnrgec; |
| (SEQ ID NO: 26) | |
| 1 | mdmrvpaqll gllllwfpga rcdiqmtqsp sslsasvgdr vtftc [symbol] |
| 51 | [symbol] wyqq kpgkapkrli y [symbol] gv psrfsgsgsg teftltissl |
| 101 | qpedfatyyc [symbol] f gqgteveiir tvaapsvfif ppsdeqlksg |
| 151 | tasvvcllnn fypreakvqw kvdnalqsgn sqesvtegds kdstyslsst |
| 201 | ltlskadyek hkvyacevth qglsspvtks fnrgec; |
| (SEQ ID NO: 27) | |
| 1 | mdmrvpaqll gllllwfpga rcdiqmtqsp sslsasvgdr vtitc [symbol] |
| 51 | [symbol] wyqq kpgkapkrli y [symbol] gv psrfsgsgsg teftltissl |
| 101 | qpedfatyyc [symbol] f gqgtkleikr tvaapsvfif ppsdeqlksg |
| 151 | tasvvcllnn fypreakvqw kvdnalqsgn sqesvteqds kdstyslsst |
| 201 | ltlskadyek hkvyacevth qglsspvtks fnrgec; |
| or | |
| (SEQ ID NO: 28) | |
| 1 | mdmrvpaqll gllllwfpga rcdiqmtqfp sslsasvgdr vtitc [symbol] |
| 51 | [symbol] wyqq kpgkapkrli y [symbol] gv psrfsgsgsg teftltissl |
| 101 | qpedfatyyc [symbol] f gqgtkleikr tvaapsvfif ppsdeqlksg |
| 151 | tasvvcllnn fypreakvqw kvdnalqsgn sqesvteqds kdstyslsst |
| 201 | ltlskadyek hkvyacevth qglsspvtks fnrgec. |

In an embodiment of the invention, the signal sequence is amino acids 1-22 of SEQ ID NOs: 25-28. In an embodiment of the invention, the mature variable region is underscored. In an embodiment of the invention, the CDRs are in bold/italicized font. In an embodiment of the invention, the anti-IGF1R antibody or antigen-binding fragment thereof of the invention comprises one or more CDRs (e.g., 3 light chain CDRS) as set forth above.

In an embodiment of the invention, the anti-IGF1R antibody comprises a heavy chain immunoglobulin or a mature fragment thereof (i.e., lacking signal sequence), or a variable region thereof, comprising the amino acid sequence of:

| (SEQ ID NO: 29) | |
|---|---|
| 1 | mefglswvfl vaiikgvqcq vqlvesgggl vkpggslrls caas [symbol] |
| 51 | [symbol] wirqap gkglewvs [symbol] [symbol] [symbol] rftis rdnaknslyl |
| 101 | qmnslraedt avyycar [symbol] [symbol] [symbol] wgqg ttvtvssast |
| 151 | kgpsvfplap csrstsesta algclvkdyf pepvtvswns galtsgvhtf |
| 201 | pavlqssgly slssvvtvps snfgtqtytc nvdhkpsntk vdktverkcc |
| 251 | vecppcpapp vagpsvflfp pkpkdtlmis rtpevtcvvv dvshedpevq |
| 301 | fnwyvdgvev hnaktkpree qfnstfrvvs vltvvhqdwl ngkeykckvs |
| 351 | nkglpapiek tisktkgqpr epqvytlpps reemtknqvs ltclvkgfyp |
| 401 | sdiavewesn gqpennyktt ppmldsdgsf flyskltvdk srwqqgnvfs |
| 451 | csvmhealhn hytqkslsls pgk; |
| (SEQ ID NO: 30) | |
| 1 | mefglswvfl vaiikgvqcq aqlvesgggl vkpggslrls caas [symbol] |
| 51 | [symbol] wirqap gkglewvs [symbol] [symbol] [symbol] rftis rdnaknslyl |
| 101 | qmnslraedt avyycvr [symbol] [symbol] [symbol] wgqgttv tvssastkgp |
| 151 | svfplapcsr stsestaalg clvkdyfpep vtvswnsgal tsgvhtfpav |
| 201 | lqssglysls svvtvpssnf gtqtytcnvd hkpsntkvdk tverkccvec |
| 251 | ppcpappvag psvflfppkp kdtlmisrtp evtcvvvdvs hedpevqfnw |
| 301 | yvdgvevhna ktkpreeqfn stfrvvsvlt vvhqdwlngk eykckvsnkg |
| 351 | lpapiektis ktkgqprepq vytlppsree mtknqvsltc lvkgfypsdi |
| 401 | avewesngqp ennykttppm ldsdgsffly skltvdksrw qqgnvfscsv |
| 451 | mhealhnhyt qkslslspgk; |
| (SEQ ID NO: 31) | |
| 1 | mefglswlfl vailkgvqce vqllesgggl vqpggslrls caas [symbol] |
| 51 | [symbol] wvrqap gkglewvs [symbol] [symbol] [symbol] rftis rdnskntlyl |
| 101 | qmnslraedt avyycak [symbol] [symbol] [symbol] wgqgttv tvssastkgp |
| 151 | svfplapcsr stsestaalg clvkdyfpep vtvswnsgal tsgvhtfpav |
| 201 | lqssglysls svvtvpssnf gtqtytcnvd hkpsntkvdk tverkccvec |
| 251 | ppcpappvag psvflfppkp kdtlmisrtp evtcvvvdvs hedpevqfnw |
| 301 | yvdgvevhna ktkpreeqfn stfrvvsvlt vvhqdwlngk eykckvsnkg |
| 351 | lpapiektis ktkgqprepq vytlppsree mtknqvsltc lvkgfypsdi |
| 401 | avewesngqp ennykttppm ldsdgsffly skltvdksrw qqgnvfscsv |
| 451 | mhealhnhyt qkslslspgk; |
| or | |
| (SEQ ID NO: 32) | |
| 1 | mefglswlfl vailkgvqce vqllesgggl vqpggslrls ctas [symbol] |
| 51 | [symbol] wvrqap gkglewvs [symbol] [symbol] [symbol] rftis rdnsrttlyl |
| 101 | qmnslraedt avyycak [symbol] [symbol] [symbol] wgqgttv tvssastkgp |
| 151 | svfplapcsr stsestaalg clvkdyfpep vtvswnsgal tsgvhtfpav |
| 201 | lqssglysls svvtvpssnf gtqtytcnvd hkpsntkvdk tverkccvec |
| 251 | ppcpappvag psvflfppkp kdtlmisrtp evtcvvvdvs hedpevqfnw |
| 301 | yvdgvevhna ktkpreeqfn stfrvvsvlt vvhqdwlngk eykckvsnkg |
| 351 | lpapiektis ktkgqprepq vytlppsree mtknqvsltc lvkgfypsdi |
| 401 | avewesngqp ennykttppm ldsdgsffly skltvdksrw qqgnvfscsv |
| 451 | mhealhnhyt qkslslspgk. |

In an embodiment of the invention, the signal sequence is amino acids 1-19 of SEQ ID NOs: 29-32. In an embodiment of the invention, the mature variable region is underscored. In an embodiment of the invention, the anti-IGF1R antibody or antigen-binding fragment thereof of the invention comprises one or more CDRs (e.g., 3 light chain CDRS) as set forth above.

In an embodiment of the invention, the anti-IGF1R antibody comprises a light chain variable region comprising the amino acid sequence of any of SEQ ID NOs: 19-24 paired with a heavy chain variable region comprising an amino acid sequence of any of SEQ ID NOs: 13-18, respectively. In an embodiment of the invention, the anti-IGF1R antibody comprises a mature light chain variable region comprising an amino acid sequence of any of SEQ ID NOs: 25 or 26 paired with a heavy chain variable region comprising an amino acid sequence of any of SEQ ID NOs: 29 or 30. In an embodiment of the invention, the anti-IGF1R antibody comprises a mature light chain variable region comprising an amino acid sequence of any of SEQ ID NOs: 27 or 28 paired with a heavy chain variable region comprising an amino acid sequence of any of SEQ ID NOs: 31 or 32.

In an embodiment of the invention, an anti-IGF1R antibody or antigen-binding fragment thereof comprises an immunoglobulin heavy chain or mature fragment or variable region of 2.12.1 fx (SEQ ID NO: 33) (in an embodiment of the invention, the leader sequence is underscored; in an embodiment of the invention, the CDRs are in bold/italicized font):

| | |
|---|---|
| 1 | mefglswvfl vaiikgvqcq vqlvesgggl vkpggslrls caas [symbol] |
| 51 | [symbol] wirqap gkglewvs [symbol] [symbol] [symbol] rftis rdnaknslyl |
| 101 | qmnslraedt avyycar [symbol] [symbol] [symbol] wgqgttv tvssastkgp |
| 151 | svfplapcsr stsestaalg clvkdyfpep vtvswnsgal tsgvhtfpav |
| 201 | lqssglysls svvtvpssnf gtqtytcnvd hkpsntkvdk tverkccvec |
| 251 | ppcpappvag psvflfppkp kdtlmisrtp evtcvvvdvs hedpevqfnw |
| 301 | yvdgvevhna ktkpreeqfn stfrvvsvlt vvhqdwlngk eykckvsnkg |
| 351 | lpapiektis ktkgqprepq vytlppsree mtknqvsltc lvkgfypsdi |
| 401 | avewesngqp ennykttppm ldsdgsffly skltvdksrw qqgnvfscsv |
| 451 | mhealhnhyt qkslslspgk |

In an embodiment of the invention, the anti-IGF1R antibody or antigen-binding fragment thereof comprises amino acids 20-470 of 2.12.1 fx (SEQ ID NO: 33).

In an embodiment of the invention, an anti-IGF1R antibody or antigen-binding fragment thereof comprises mature immunoglobulin heavy chain variable region 2.12.1 fx (amino acids 20-144 or SEQ ID NO: 33; SEQ ID NO: 34):

q vqlvesgggl vkpggslrls caasgftfsd yymswirqap
gkglewvsyi sssgstrdya dsvkgrftis rdnaknslyl
qmnslraedt avyycardgv ettfyyyyyg mdvwgqgttv tvss

In an embodiment of the invention, an anti-IGF1R antibody or antigen-binding fragment thereof comprises an immunoglobulin light chain or mature fragment or variable region 2.12.1 fx (SEQ ID NO: 35) (in an embodiment of the invention, the leader sequence is underscored; in an embodiment of the invention, the CDRs are in bold/italicized font):

| | |
|---|---|
| 1 | mdmrvpaqll gllllwfpga rcdiqmtqsp sslsasvgdr vtitc [symbol] |
| 51 | [symbol] wyqq kpgkapkrli y [symbol] gv psrfsgsgsg teftltissl |
| 101 | qpedfatyyc [symbol] f gqgtkveikr tvaapsvfif ppsdeqlksg |
| 151 | tasvvcllnn fypreakvqw kvdnalqsgn sqesvteqds kdstyslsst |
| 201 | ltlskadyek hkvyacevth qglsspvtks fnrgec |

In an embodiment of the invention, an anti-IGF1R antibody or antigen-binding fragment thereof comprises amino acids 23-236 of 2.12.1 fx (SEQ ID NO: 35).

In an embodiment of the invention, an anti-IGF1R antibody or antigen-binding fragment thereof comprises mature immunoglobulin light chain variable region 2.12.1 fx (amino acids 23-130 of SEQ ID NO: 35; SEQ ID NO: 36):

diqmtqsp sslsasvgdr vtitcrasqd irrdlgwyqq
kpgkapkrli yaasrlqsgv psrfsgsgsg teftltissl
qpedfatyyc lqhnnyprtf gqgtkveikr

In an embodiment of the invention, an anti-IGF1R antibody or antigen-binding fragment thereof comprises or consists of a light chain immunoglobulin chain comprising or consisting of amino acids 23-236 of 2.12.1 fx (SEQ ID NO: 35) and a heavy chain immunoglobulin chain comprising or consisting of amino acids 20-470 of 2.12.1 fx (SEQ ID NO: 33).

In an embodiment of the invention, the anti-IGF1R antibody or antigen-binding fragment thereof comprises one or more 2.12.1 fx CDRs (e.g., 3 light chain CDRs and/or 3 heavy chain CDRs) as set forth above.

In an embodiment of the invention, an anti-IGF1R antibody or antigen-binding fragment thereof or antigen-binding fragment thereof comprises a humanized 7C10 immunoglobulin light chain variable region; version 1 (SEQ ID NO: 37):

| | |
|---|---|
| 1 | dvvmtqspls lpvtpgepas iscrssqsiv hsngntylqw ylqkpgqspq |
| 51 | lliykvsnrl ygvpdrfsgs gsgtdftlki srveaedvgv yycfqgshvp |
| 101 | wtfgqgtkve ik |

In an embodiment of the invention, an anti-IGF1R antibody or antigen-binding fragment thereof comprises humanized 7C10 immunoglobulin light chain variable region; version 2 (SEQ ID NO: 38):

| | |
|---|---|
| 1 | divmtgspls lpvtpgepas iscrssqsiv hsngntylqw ylqkpgqspq |
| 51 | lliykvsnrl ygvpdrfsgs gsgtdftlki srveaedvgv yycfqgshvp |
| 101 | wtfgqgtkve ik |

In an embodiment of the invention, an anti-IGF1R antibody or antigen-binding fragment thereof comprises a humanized 7C10 immunoglobulin heavy chain variable region; version 1 (SEQ ID NO: 39):

| | |
|---|---|
| 1 | qvqlqesgpg lvkpsetlsl tctvsgysit ggylwnwirq ppgkglewmg |
| 51 | yisydgtnny kpslkdriti srdtsknqfs lklssvtaad tavyycaryg |
| 101 | rvffdywgqg tlvtvss |

In an embodiment of the invention, an anti-IGF1R antibody or antigen-binding fragment thereof comprises the humanized 7C10 immunoglobulin heavy chain variable region; version 2 (SEQ ID NO: 40):

| | |
|---|---|
| 1 | qvqlqesgpg lvkpsetlsl tctvsgysit ggylwnwirq ppgkglewig |
| 51 | yisydgtnny kpslkdrvti srdtsknqfs lklssvtaad tavyycaryg |
| 101 | rvffdywgqg tlvtvss |

In an embodiment of the invention, an anti-IGF1R antibody or antigen-binding fragment thereof comprises the humanized 7C10 immunoglobulin heavy chain variable region; version 3 (SEQ D NO: 41):

| | |
|---|---|
| 1 | qvqlqesgpg lvkpsetlsl tctvsgysis ggylwnwirq ppgkglewig |
| 51 | yisydgtnny kpslkdrvti svdtsknqfs lklssvtaad tavyycaryg |
| 101 | rvffdywgqg tlvtvss |

In an embodiment of the invention, an anti-IGF1R antibody or antigen-binding fragment thereof comprises A12 immunoglobulin heavy chain variable region (SEQ ID NO: 42):

| | |
|---|---|
| 1 | evqlvqsgae vkkpgssvkv sckasggtfs syaiswvrqa pgqglewmgg |
| 51 | iipifgtany aqkfqgrvti tadkststay melsslrsed tavyycarap |
| 101 | lrflewstqd hyyyyymdvw gkgttvtvss |

In an embodiment of the invention, an anti-IGF1R antibody or antigen-binding fragment thereof comprises A12 immunoglobulin light chain variable region (SEQ ID NO: 43):

| | |
|---|---|
| 1 | sseltqdpav svalgqtvri tcqgdslrsy yaswyqqkpg qapvlviygk |
| 51 | nnrpsgipdr fsgsssgnta sltitgaqae deadyycnsr dnsdnrlifg |
| 101 | ggtkltvls |

or

### (SEQ ID NO: 106):

| | |
|---|---|
| 1 | sseltqdpav svalgqtvri tcqgdslrsy yatwyqqkpg qapilviyge |
| 51 | nkrpsgipdr fsgsssgnta sltitgaqae deadyycksr dgsgqhlvfg |
| 101 | ggtkltvlg |

In an embodiment of the invention, an anti-IGF1R antibody or antigen-binding fragment thereof comprises 1A immunoglobulin heavy chain variable region (SEQ ID NO: 44):

| | |
|---|---|
| 1 | evqlvqsggg lvhpggslrl scagsgftfr nyamywvrqa pgkglewvsa |
| 51 | igsgggtyya dsvkgrftis rdnaknslyl qmnslraedm avyycarapn |
| 101 | wgsdafdiwg qgtmvtvss |

; optionally including one or more of the following mutations: R30, S30, N31, S31, Y94, H94, D104, E104.

In an embodiment of the invention, an anti-IGF1R antibody or antigen-binding fragment thereof comprises 1A immunoglobulin light chain variable region (SEQ ID NO: 45):

| | |
|---|---|
| 1 | digmtqspss lsasvgdrvt itcrasqgis swlawyqqkp ekapksliya |
| 51 | asslqsgvps rfsgsgsgtd ftltisslqp edfatyycqq ynsypptfgp |
| 101 | gtkvdik |

; optionally including one or more of the following mutations: P96, I96, P100, Q100, R103, K103, V104, L104, D105, E105

In an embodiment of the invention, an anti-IGF1R antibody or antigen-binding fragment thereof comprises single chain antibody (fv) 8A1 (SEQ ID NO: 46):

| | |
|---|---|
| 1 | evqlvqsgae vkkpgeslti sckgpgynff nywigwvrqm pgkglewmgi |
| 51 | iyptdsdtry spsfqgqvti svdksistay lqwsslkasd tamyycarsi |
| 101 | rycpggrcys gyygmdvwgq gtmvtvssgg ggsggggsgg ggsseltqdp |
| 151 | aysvalgqtv ritcqgdslr syyaswyqqk pgqapvlviy gknnrpsgip |
| 201 | drfsgsssgn tasltitgaq aedeadyycn srdssgnhvv fgggtkltvl |
| 251 | g |

In an embodiment of the invention, an anti-IGF1R antibody or antigen-binding fragment thereof comprises single chain antibody (fv) 9A2 (SEQ ID NO: 47):

| | |
|---|---|
| 1 | qvqlvqsgae vrkpgasvkv scktsgytfr nydinwvrqa pgqglewmgr |
| 51 | isghygntdh aqkfqgrftm tkdtststay melrsltfdd tavyycarsq |
| 101 | wnvdywgrgt lvtvssgggg sggggsgggg salnfmltqp hsysespgkt |
| 151 | vtisctrssg siasnyvqwy qqrpgssptt vifednrrps gvpdrfsgsi |
| 201 | dtssnsaslt isglktedea dyycqsfdst nlvvfgggtk vtvlg |

In an embodiment of the invention, an anti-IGF1R antibody or antigen-binding fragment thereof comprises single chain antibody (fv) 11A4 (SEQ ID NO: 48):

| | |
|---|---|
| 1 | evqllesggg lvqpggslrl scaasgftfs syamswvrqa pgkglewvsa |
| 51 | isgsggstyy adsvkgrfti srdnskntly lqmnslraed tavyycassp |
| 101 | yssrwysfdp wgqgtmvtvs sggggsgggg sggggsalsy eltqppsvsv |
| 151 | spgqtatitc sgddlgnkyv swyqqkpgqs pvlviyqdtk rpsgiperfs |
| 201 | gsnsgniatl tisgtqavde adyycqvwdt gtvvfgggtk ltvlg |

In an embodiment of the invention, an anti-IGF1R antibody or antigen-binding fragment thereof comprises single chain antibody (fv) 7A4 (SEQ ID NO: 49):

| | |
|---|---|
| 1 | evqlvqsgae vkkpgeslti sckgsgynff nywigwvrqm pgkdlewmgi |
| 51 | iyptdsdtry spsfqgqvti svdksistay lqwsslkasd tamyycarsi |
| 101 | rycpggrcys gyygmdvwgq gtmvtvssgg gssggggsgg ggsseltqdp |
| 151 | aysvalgqtv ritcrgdslr nyyaswyqqk pgqapvlviy gknnrpsgip |
| 201 | drfsgsssgn tasltitgaq aedeadyycn srdssgnhmv fgggtkltvl |
| 251 | g |

In an embodiment of the invention, an anti-IGF1R antibody or antigen-binding fragment thereof comprises single chain antibody (fv) 11A1 (SEQ ID NO: 50):

| | |
|---|---|
| 1 | evqlvesggg vvqpgrslrl scaasgftfs dfamhwvrqi pgkglewlsg |
| 51 | lrhdgstayy agsvkgrfti srdnsrntvy lqmnslraed tatyycvtgs |
| 101 | gssgphafpv wgkgtlvtvs sggggsgggg sggggsalsy vltqppsasg |
| 151 | tpgqrvtisc sgsnsnigty tvnwfqqlpg tapklliysn nqrpsgvpdr |
| 201 | fsgsksgtsa slaisglqse deadyycaaw ddslngpvfg ggtkvtvlg |

In an embodiment of the invention, an anti-IGF1R antibody or antigen-binding fragment thereof comprises single chain antibody (fv) 7A6 (SEQ ID NO: 51)

| | |
|---|---|
| 1 | evqlvqsgae ykkpgeslti sckgsgynff nywigwvrqm pgkglewmgi |
| 51 | iyptdsdtry spsfqgqvti svdksistay lqwsslkasd tamyycarsi |
| 101 | rycpggrcys gyygmdvwgq gtlvtvssgg ggsggggsgg ggsseltqdp |
| 151 | aysvalgqtv ritcggdslr syytnwfqqk pgqapllvvy aknkrpsgip |
| 201 | drfsgsssgn tasltitgaq aedeadyycn srdssgnhvv fgggtkltvl |
| 251 | g |

In an embodiment of the invention, an anti-IGF1R antibody or an antigen-binding fragment thereof (e.g., a heavy chain or light chain immunoglobulin) comprises one or more complementarity determining regions (CDR) selected from the group consisting of:

| | |
|---|---|
| sywmh; | (SEQ ID NO: 52) |
| einpsngrtnynekfkr; | (SEQ ID NO: 53) |
| grpdyygsskwyfdv; | (SEQ ID NO: 54) |
| rssgsivhsnvntyle; | (SEQ ID NO: 55) |
| kvsnrfs; | (SEQ ID NO: 56) |
| and | |
| fggshvppt. | (SEQ ID NO: 57) |

In an embodiment of the invention, an anti-IGF1R antibody or an antigen-binding fragment thereof comprises a heavy chain immunoglobulin variable region selected from the group consisting of:

| (SEQ ID NO: 58) | |
|---|---|
| 1 | qvglvqsgae vvkpgasvkl sckasgytft sywmhwvkqr pgqglewige |
| 51 | inpsngrtny nqkfqgkatl tvdkssstay mqlssltsed savyyfargr |
| 101 | pdyygsskwy fdvwgqgttv tvs; |
| (SEQ ID NO: 59) | |
| 1 | qvqfqqsgae lvkpgasvkl sckasgytft sylmhwikqr pgrglewigr |
| 51 | idpnnvvtkf nekfkskatl tvdkpsstay melssltsed savyycarya |
| 101 | ycrpmdywgq gttvtvss; |
| (SEQ ID NO: 60) | |
| 1 | qvqlqqsgae lvkpgasvkl sckasgytft sywmhwvkqr pgqglewige |
| 51 | inpsngrtny nekfkrkatl tvdkssstay mqlssltsed savyyfargr |
| 101 | pdyygsskwy fdvwgagttv tvs; |
| (SEQ ID NO: 61) | |
| 1 | qvqlqqsgae lmkpgasvki sckatgytfs sfwiewvkqr pghglewige |
| 51 | ilpgsggthy nekfkgkatf tadkssntay mqlssltsed savyycargh |
| 101 | syyfydgdyw gqgtsvtvss; |
| (SEQ ID NO: 62) | |
| 1 | qvglqqpgsv lvrpgasvkl sckasgytft sswihwakqr pgqglewige |
| 51 | ihpnsgntny nekfkgkatl tvdtssstay vdlssltsed savyycarwr |
| 101 | ygspyyfdyw gqgttltvss; |
| (SEQ ID NO: 63) | |
| 1 | qvqlqqpgae lvkpgasvkl sckasgytft sywmhwvkqr pgrglewigr |
| 51 | idpnsggtky nekfkskatl tvdkpsstay mqlssltsed savyycaryd |
| 101 | yygssyfdyw gqgttltvss; |
| (SEQ ID NO: 64) | |
| 1 | qvqlvqsgae vvkpgasvkl sckasgytft sywmhwvkqr pgqglewige |
| 51 | inpsngrtny nqkfqgkatl tvdkssstay mqlssltsed savyyfargr |
| 101 | pdyygsskwy fdvwgqgttv tvs; |
| (SEQ ID NO: 65) | |
| 1 | qvqlqqsgae lvkpgasvkl sckasgytft sywmhwvkqr pgqglewige |
| 51 | inpsngrtny nekfkrkatl tvdkssstay mqlssltsed savyyfargr |
| 101 | pdyygsskwy fdvwgagttv tvss; |
| (SEQ ID NO: 66) | |
| 1 | qvqlvqsgae vvkpgasvkl sckasgytft sywmhwvkqr pgqglewige |
| 51 | inpsngrtny nqkfqgkatl tvdkssstay mqlssltsed savyyfargr |
| 101 | pdyygsskwy fdvwgqgttv tvss; |
| (SEQ ID NO: 67) | |
| 1 | qvqlqqsgae lvkpgasvkl sckasgytft sywmhwvkqr pgrglewigr |
| 51 | idpnsggtky nekfkskatl tvdkpsstay mqlssltsed savyycaryd |
| 101 | yygssyfdyw gqgttvtvss; |
| (SEQ ID NO: 68) | |
| 1 | qiqlqqsgpe lvrpgasvki sckasgytft dyyihwvkqr pgeglewigw |
| 51 | iypgsgntky nekfkgkatl tvdtssstay mqlssltsed savyfcargg |
| 101 | kfamdywgqg tsvtvss; |
| (SEQ ID NO: 69) | |
| 1 | qvqlqqsgae lvkpgasvkl sckasgytft sywmhwvkqr pgqglewige |
| 51 | inpsngrtny nekfkrkatl tvdkssstay mqlssltsed savyyfargr |
| 101 | pdyygsskwy fdvwgagttv tvss; |
| (SEQ ID NO: 70) | |
| 1 | qiqlqqsgpe lvkpgasvki sckasgytft dyyinwmkqk pgqglewigw |
| 51 | idpgsgntky nekfkgkatl tvdtssstay mqlssltsed tavyfcarek |
| 101 | ttyyyamdyw gqgtsvtvsa; |
| (SEQ ID NO: 71) | |
| 1 | vqlqqsgael mkpgasvkis ckasgytfsd ywiewvkqrp ghglewigei |
| 51 | 1pgsgstnyh erfkgkatft adtssstaym qlnsltseds gvyyclhgny |
| 101 | dfdgwgqgtt ltvss; |
| and | |
| (SEQ ID NO: 72) | |
| 1 | qvqllesgae lmkpgasvki sckatgytfs sfwiewvkqr pghglewige |
| 51 | ilpgsggthy nekfkgkatf tadkssntay mqlssltsed savyycargh |
| 101 | syyfydgdyw gqgtsvtvss; |
| and/or | |
| a light chain immunoglobulin variable region selected | |
| from the group consisting of: | |
| (SEQ ID NO: 73) | |
| 1 | dvlmtqipvs lpvslgdqas iscrssqiiv hnngntylew ylqkpgqspq |
| 51 | lliykvsnrf sgvpdrfsgs gsgtdftlki srveaedlgv yycfqgshvp |
| 101 | ftfgsgtkle ikr; |
| (SEQ ID NO: 74) | |
| 1 | dvlmtqtpls lpvslgdpas iscrssqsiv hsnvntylew ylqkpgqspk |
| 51 | lliykvsnrf sgvpdrfsgs gagtdftlri srveaedlgi yycfqgshvp |
| 101 | ptfgggtkle ikr; |
| (SEQ ID NO: 75) | |
| 1 | dvlmtqtpls lpvslgdpas iscrssqsiv hsnvntylew ylqkpgqspr |
| 51 | lliykvsnrf sgvpdrfsgs gagtdftlri srveaedlgi yycfqgshvp |
| 101 | ptfgggtkle ikr; |
| (SEQ ID NO: 76) | |
| 1 | dvlmtqtpls lpvslgdpas iscrssgsiv hsnvntylew ylqkpgqspk |
| 51 | lliykvsnrf sgvpdrfsgs gagtdftlri srveaedlgi yycfqgshvp |
| 101 | ptfgggtkle ikr; |
| (SEQ ID NO: 77) | |
| 1 | dvlmtqtpls lpvslgdpas iscrssgsiv hsnvntylew ylqkpgqspr |
| 51 | lliykvsnrf sgvpdrfsgs gagtdftlri srveaedlgi yycfggshvp |
| 101 | ptfgggtkle ikr; |
| (SEQ ID NO: 78) | |
| 1 | dvlmtqtpls lpvslgdqas iscrssqxiv hsngntylew ylqkpgqspk |
| 51 | lliykvsnrf sgvpdrfsgs gsgtdftlki srveaedlgv yycfqgshvp |
| 101 | xtfgggtkle ikr; |
| (SEQ ID NO: 79) | |
| 1 | dvvmtqtpls lpvslgdpas iscrssqsiv hsnvntylew ylqkpgqspk |
| 51 | lliykvsnrf sgvpdrfsgs gagtdftlri srveaedlgi yycfqgshvp |
| 101 | ptfgggtkle ikr; |
| (SEQ ID NO: 80) | |
| 1 | dvvmtqtpls lpvslgdpas iscrssqsiv hsnvntylew ylqkpgqspr |
| 51 | lliykvsnrf sgvpdrfsgs gagtdftlri srveaedlgi yycfggshvp |
| 101 | ptfgggtkle ikr; |
| (SEQ ID NO: 81) | |
| 1 | dvlmtqtpls lpvslgdpas iscrssqsiv hsnvntylew ylqkpgqspr |
| 51 | lliykvsnrf sgvpdrfsgs gagtdftlri srveaedlgi yycfqgshvp |
| 101 | ptfgggtkle ikr; |
| (SEQ ID NO: 82) | |
| 1 | dvlmtqipvs lpvslgdqas iscrssqiiv hnngntylew ylqkpgqspq |
| 51 | lliykvsnrf sgvpdrfsgs gsgtdftlki srveaedlgv yycfqgshvp |
| 101 | ftfgsgtkle ikr; |
| (SEQ ID NO: 83) | |
| 1 | dvlmtqtpls lpvslgdqas iscrfsqsiv hsngntylew ylqksgqspk |
| 51 | lliykvsnrf sgvpdrfsgs gsgtdftlki srveaedlgv yycfqgshvp |
| 101 | rtfgggtkle ikr; |
| (SEQ ID NO: 84) | |
| 1 | dvlmtqtpls lpvslgdqas iscrssqsiv hsnvntylew ylqkpgqspk |
| 51 | lliykvsnrf sgvpdrfsgs gsgtdftlri srveaedlgi yycfqgshvp |
| 101 | ptfgggtkle ikr; |
| (SEQ ID NO: 85) | |
| 1 | dvvmtqtpls lpvslgdpas iscrssqsiv hsnvntylew ylqkpgqspk |
| 51 | lliykvsnrf sgvpdrfsgs gagtdftlri srveaedlgi yycfqgshvp |
| 101 | ptfgggtkle ikr; |
| (SEQ ID NO: 86) | |
| 1 | elvmtqtpls lpvslgdqas iscrssqtiv hsngdtyldw flqkpgqspk |
| 51 | lliykvsnrf sgvpdrfsgs gsgtdftlki srveaedlgv yycfqgshvp |
| 101 | ptfgggtkle ikr; |
| (SEQ ID NO: 87) | |
| 1 | dvlmtqtpls lpvslgdpas iscrssqsiv hsnvntylew ylqkpgqspk |
| 51 | lliykvsnrf sgvpdrfsgs gagtdftlri srveaedlgi yycfqgshvp |
| 101 | ptfgggtkle ikr; |
| (SEQ ID NO: 88) | |
| 1 | dvvmtqtpls lpvslgdpas iscrssqsiv hsnvntylew ylqkpgqspr |
| 51 | lliykvsnrf sgvpdrfsgs gagtdftlri srveaedlgi yycfqgshvp |
| 101 | ptfgggtkle ikr; |
| (SEQ ID NO: 89) | |
| 1 | dvlmtqtpvs lsyslgdqas iscrssqsiv hstgntylew ylqkpgqspk |
| 51 | lliykisnrf sgvpdrfsgs gsgtdftlki srveaedlgv yycfqashap |
| 101 | rtfgggtkle ikr; |
| (SEQ ID NO: 90) | |
| 1 | dvlmtqtpls lpvslgdqas isckssgsiv hssgntyfew ylqkpgqspk |
| 51 | lliykvsnrf sgvpdrfsgs gsgtdftlki srveaedlgv yycfqgship |
| 101 | ftfgsgtkle ikr; |
| (SEQ ID NO: 91) | |
| 1 | dieltqtpls lpvslgdqas iscrssqsiv hsngntylew ylqkpgqspk |
| 51 | lliykvsnrf sgvpdrfsgs gsgtdftlki srveaedlgv yycfqgshvp |
| 101 | ytfgggtkle ikr; |
| (SEQ ID NO: 92) | |
| 1 | dvlmtqtpls lpvslgdqas iscrssqsiv hsnvntylew ylqkpgqspk |
| 51 | lliykvsnrf sgvpdrfsgs gsgtdftlri srveaedlgi yycfqgshvp |
| 101 | ptfgggtkle ikr; |
| (SEQ ID NO: 93) | |
| 1 | dvvmtqtpls lpvslgdpas iscrssqsiv hsnvntylew ylqkpgqspr |
| 51 | lliykvsnrf sgvpdrfsgs gagtdftlri srveaedlgi yycfqgshvp |
| 101 | ptfgggtkle ikr; |
| (SEQ ID NO: 94) | |
| 1 | dvlmtqtpls lpvslgdqas iscrssqsiv hsnvntylew ylqkpgqspk |
| 51 | lliykvsnrf sgvpdrfsgs gsgtdftlri srveaedlgi yycfqgshvp |
| 101 | ptfgggtkle ikr; |
| (SEQ ID NO: 95) | |
| 1 | dvvmtqtpls lpvslgdpas iscrssqsiv hsnvntylew ylqkpgqspk |
| 51 | lliykvsnrf sgvpdrfsgs gagtdftlri srveaedlgi yycfqgshvp |
| 101 | ptfgggtkle ikr; |
| (SEQ ID NO: 96) | |
| 1 | dvlmtqtpls lpvslgdqas iscrsnqtil lsdgdtylew ylqkpgqspk |
| 51 | lliykvsnrf sgvpdrfsgs gsgtdftlki srveaedlgv yycfqgshvp |
| 101 | ptfgggtkle ikr; |
| (SEQ ID NO: 97) | |
| 1 | dvlmtqtpls lpvslgdqas iscrssqtiv hsngntylew ylqkpgqspk |
| 51 | lliykvtnrf sgvpdrfsgs gsgtdftlki srveaedlgv yycfqgthap |
| 101 | ytfgggtkle ikr; |
| and | |
| (SEQ ID NO: 98) | |
| 1 | dvlmtqtpls lpvslgdqas iscrssqsiv hsngntylew ylqkpgqspk |
| 51 | lliysissrf sgvpdrfsgs gsgtdftlki srvqaedlgv yycfqgshvp |
| 101 | ytfgggtkle ikr. |

The scope of the present invention includes embodiments wherein the variable region of an anti-IGF1R antibody is linked to any immunoglobulin constant region. In an embodiment, the light chain variable region is linked to a κ chain constant region. In an embodiment, the heavy chain variable region is linked to a γ1, γ2, γ3 or γ4 chain constant region. Any of the immunoglobulin variable regions set forth herein, in embodiments of the invention, can be linked to any of the foregoing constant regions.

Furthermore, the scope of the present invention comprises any antibody or antibody fragment comprising one or more CDRs (3 light chain CDRs and/or 3 heavy chain CDRs) and/or framework regions of any of the light chain immunoglobulin or heavy chain immunoglobulins set forth herein as identified by any of the methods set forth in Chothia et al., J. Mol. Biol. 186:651-663 (1985); Novotny and Haber, Proc. Natl. Acad. Sci. USA 82:4592-4596 (1985) or Kabat, E. A. et al., Sequences of Proteins of Immunological Interest, National Institutes of Health, Bethesda, Md., (1987)).

In an embodiment of the invention, the term “monoclonal antibody,” as used herein, includes an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Monoclonal antibodies are advantageous in that they may be synthesized by a hybridoma culture, essentially uncontaminated by other immunoglobulins. The modifier “monoclonal” indicates the character of the antibody as being amongst a substantially homogeneous population of antibodies, and is not to be construed as requiring production of the antibody by any particular method. As mentioned above, the monoclonal antibodies to be used in accordance with the present invention may be made by the hybridoma method described by Kohler, et al., (1975) Nature 256: 495.

In an embodiment of the invention, a polyclonal antibody is an antibody which was produced among or in the presence of one or more other, non-identical antibodies. In general, polyclonal antibodies are produced from a B-lymphocyte in the presence of several other B-lymphocytes which produced non-identical antibodies. Usually, polyclonal antibodies are obtained directly from an immunized animal.

In an embodiment of the invention, a bispecific or bifunctional antibody is an artificial hybrid antibody having two different heavy/light chain pairs and two different binding sites. Bispecific antibodies can be produced by a variety of methods including fusion of hybridomas or linking of Fab′ fragments. See, e.g., Songsivilai, et al., (1990) Clin. Exp. Immunol. 79: 315-321, Kostelny, et al., (1992) J. Immunol. 148:1547-1553. In addition, bispecific antibodies may be formed as “diabodies” (Holliger, et al., (1993) PNAS USA 90:6444-6448) or as “Janusins” (Traunecker, et al., (1991) EMBO J. 10:3655-3659 and Traunecker, et al., (1992) Int. J. Cancer Suppl. 7:51-52).

In an embodiment of the invention, the term “fully human antibody” refers to an antibody which comprises human immunoglobulin protein sequences only (lacking non-human sequences). A fully human antibody may contain murine carbohydrate chains if produced in a mouse, in a mouse cell or in a hybridoma derived from a mouse cell. Similarly, “mouse antibody” refers to an antibody which comprises mouse immunoglobulin protein sequences only.

The present invention includes “chimeric antibodies”; in an embodiment of the invention, an antibody which comprises a variable region of the present invention fused or chimerized with an antibody region (e.g., constant region) from another, human or non-human species (e.g., mouse, horse, rabbit, dog, cow, chicken). These antibodies may be used e.g., to modulate the expression or activity of IGF1R in a non-human species.

“Single-chain Fv” or “sFv” antibody fragments have, in an embodiment of the invention, the V_{H} and V_{L} domains of an antibody, wherein these domains are present in a single polypeptide chain. Generally, the sFv polypeptide further comprises a polypeptide linker between the V_{H} and V_{L} domains which enables the sFv to form the desired structure for antigen binding. Techniques described for the production of single chain antibodies (U.S. Pat. Nos. 5,476,786; 5,132,405 and 4,946,778) can be adapted to produce anti-IGF1R-specific single chain antibodies. For a review of sFv see Pluckthun in The Pharmacology of Monoclonal Antibodies, vol. 113, Rosenburg and Moore eds. Springer-Verlag, N.Y., pp. 269-315 (1994).

In an embodiment of the invention, “disulfide stabilized Fv fragments” and “dsFv” refer to immunoglobulins comprising a variable heavy chain (V_{H}) and a variable light chain (V_{L}) which are linked by a disulfide bridge.

Antigen-binding fragments of antibodies within the scope of the present invention also include F(ab)₂ fragments which may, in an embodiment of the invention, be produced by enzymatic cleavage of an IgG by, for example, pepsin. Fab fragments may be produced by, for example, reduction of F(ab)₂ with dithiothreitol or mercaptoethylamine. A Fab fragment is, in an embodiment of the invention, a V_{L}-C_{L} chain appended to a V_{H}-C_{H1} chain by a disulfide bridge. A F(ab)₂ fragment is, in an embodiment of the invention, two Fab fragments which, in turn, are appended by two disulfide bridges. The Fab portion of an F(ab)₂ molecule includes, in an embodiment of the invention, a portion of the F_{c} region between which disulfide bridges are located.

In an embodiment of the invention, an F_{V} fragment is a V_{L} or V_{H} region.

Depending on the amino acid sequences of the constant domain of their heavy chains, immunoglobulins can be assigned to different classes. There are at least five major classes of immunoglobulins: IgA, IgD, IgE, IgG and IgM, and several of these may be further divided into subclasses (isotypes), e.g. IgG-1, IgG-2, IgG-3 and IgG-4; IgA-1 and IgA-2. As discussed herein, any such antibody or antigen-binding fragment thereof is within the scope of the present invention.

The anti-IGF1R antibodies of the invention may, in an embodiment of the invention, be conjugated to a chemical moiety. The chemical moiety may be, inter alia, a polymer, a radionuclide or a cytotoxic factor. In an embodiment of the invention, the chemical moiety is a polymer which increases the half-life of the antibody or antigen-binding fragment thereof in the body of a subject. Polymers include, but are not limited to, polyethylene glycol (PEG) (e.g., PEG with a molecular weight of 2 kDa, 5 kDa, 10 kDa, 12 kDa, 20 kDa, 30 kDa or 40 kDa), dextran and monomethoxypolyethylene glycol (mPEG). Lee, et al., (1999) (Bioconj. Chem. 10:973-981) discloses PEG conjugated single-chain antibodies. Wen, et al., (2001) (Bioconj. Chem. 12:545-553) disclose conjugating antibodies with PEG which is attached to a radiometal chelator (diethylenetriaminpentaacetic acid (DTPA)).

The antibodies and antibody fragments may, in an embodiment of the invention, be conjugated with labels such as ⁹⁹Tc, ⁹⁰Y, ¹¹¹In, ³²P, ¹⁴C, ¹²⁵I, ³H, ¹³¹I, ¹¹C, ¹⁵O, ¹³N, ¹⁸F, ³⁵S, ⁵¹Cr, ⁵⁷To, ²²⁶Ra, ⁶⁰Co, ⁵⁹Fe, ⁵⁷Se, ¹⁵²Eu, ⁶⁷Cu, ²¹⁷Ci, ²¹¹At, ²¹²Pb, ⁴⁷Sc, ¹⁰⁹Pd, ²³⁴Th, and ⁴⁰K, ¹⁵⁷Gd, ⁵⁵Mn, ⁵²Tr, and ⁵⁶Fe.

The antibodies and antibody fragments may also be, in an embodiment of the invention, conjugated with fluorescent or chemilluminescent labels, including fluorophores such as rare earth chelates, fluorescein and its derivatives, rhodamine and its derivatives, isothiocyanate, phycoerythrin, phycocyanin, allophycocyanin, o-phthaladehyde, fluorescamine, ¹⁵²Eu, dansyl, umbelliferone, luciferin, luminal label, isoluminal label, an aromatic acridinium ester label, an imidazole label, an acridimium salt label, an oxalate ester label, an aequorin label, 2,3-dihydrophthalazinediones, biotin/avidin, spin labels and stable free radicals.

The antibodies and antibody fragments may also be, in an embodiment of the invention, conjugated to a cytotoxic factor such as diptheria toxin, Pseudomonas aeruginosa exotoxin A chain, ricin A chain, abrin A chain, modeccin A chain, alpha-sarcin, Aleurites fordii proteins and compounds (e.g., fatty acids), dianthin proteins, Phytoiacca americana proteins PAPI, PAPII, and PAP-S, momordica charantia inhibitor, curcin, crotin, saponaria officinalis inhibitor, mitogellin, restrictocin, phenomycin, and enomycin.

Any method known in the art for conjugating the antibodies or antigen-binding fragments thereof of the invention to the various moieties may be employed, including those methods described by Hunter, et al., (1962) Nature 144:945; David, et al., (1974) Biochemistry 13:1014; Pain, et al., (1981) J. Immunol. Meth. 40:219; and Nygren, J., (1982) Histochem. and Cytochem. 30:407. Methods for conjugating antibodies are conventional and very well known in the art.

In an embodiment of the invention, an IGF1R inhibitor is

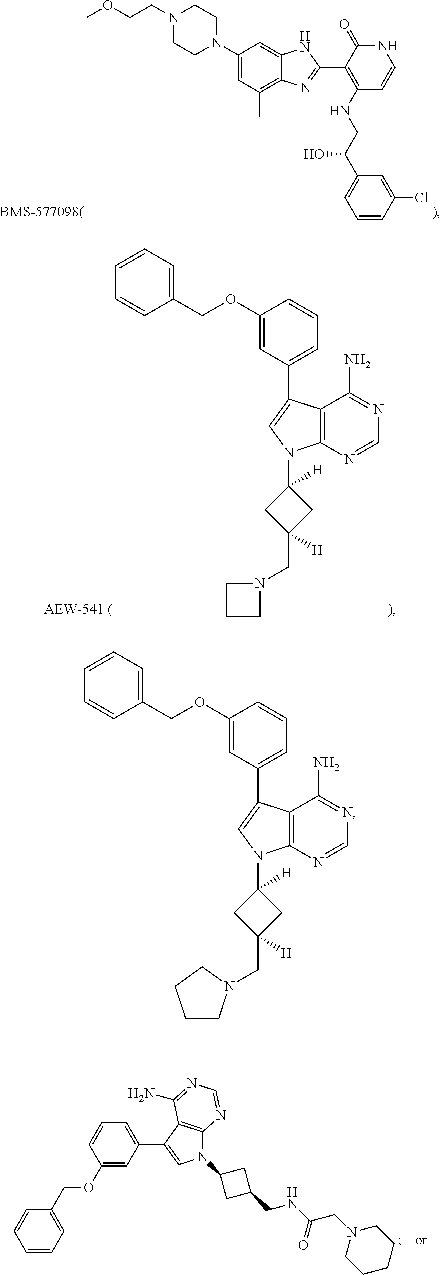

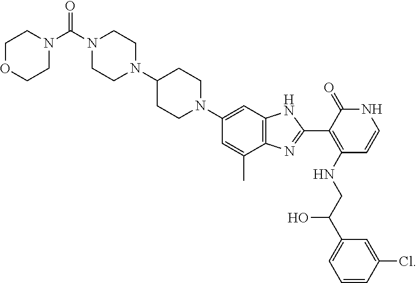

### Further Therapeutic Agents

In an embodiment of the invention, an IGF1R inhibitor is provided in association with erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR, KRX-0402, lucanthone, LY 317615, neuradiab, vitespan, Rta 744, Sdx 102, talampanel, atrasentan, Xr 311, everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763 or AT-9263.

Abraxane is an injectable suspension of paclitaxel protein-bound particles comprising an albumin-bound form of paclitaxel with a mean particle size of approximately 130 nanometers. Abraxane is supplied as a white to yellow, sterile, lyophilized powder for reconstitution with 20 mL of 0.9% Sodium Chloride Injection, USP prior to intravenous infusion. Each single-use vial contains 100 mg of paclitaxel and approximately 900 mg of human albumin. Each milliliter (mL) of reconstituted suspension contains 5 mg paclitaxel. Abraxane is free of solvents and is free of cremophor (polyoxyethylated castor oil).

In an embodiment of the invention, an IGF1R inhibitor is provided in association with romidepsin

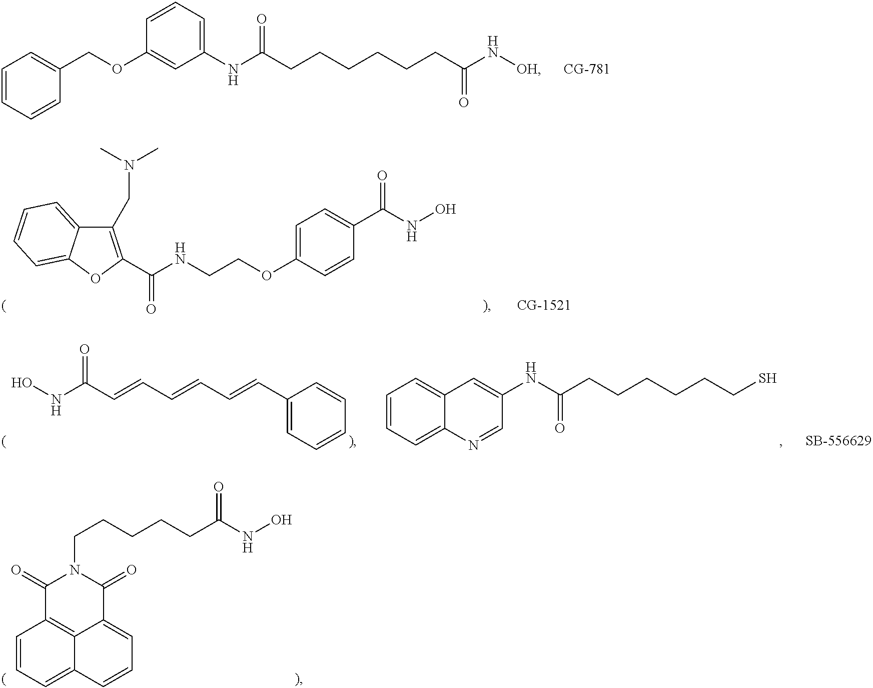

chlamydocin

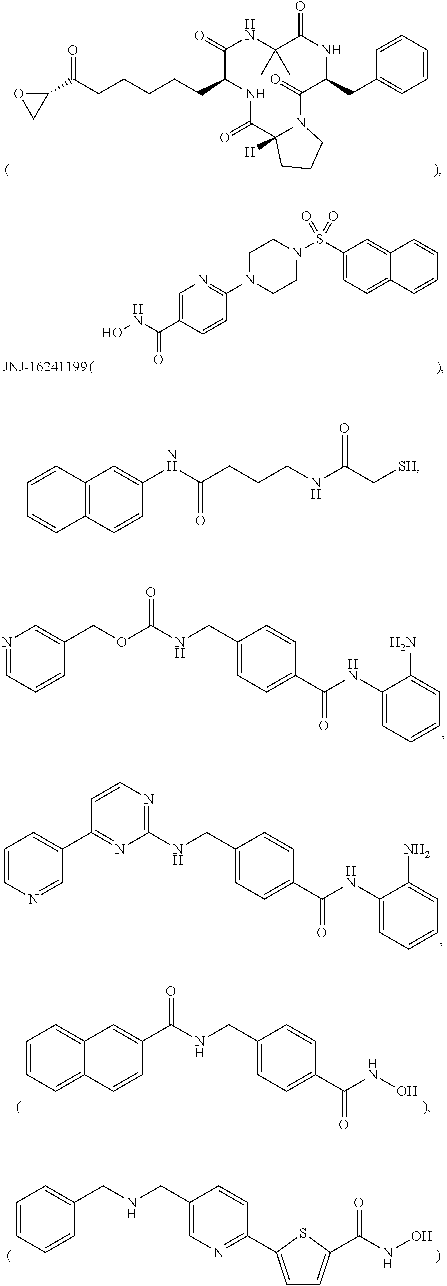

or vorinostat

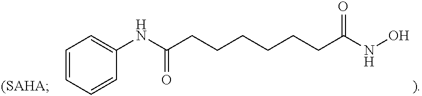

In an embodiment of the invention, an IGF1R inhibitor is provided in association with etoposide

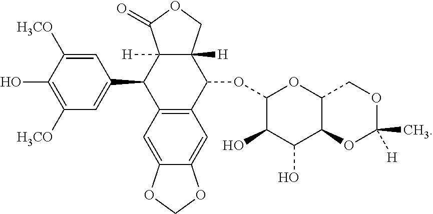

In an embodiment of the invention, an IGF1R inhibitor is provided in association with gemcitabine

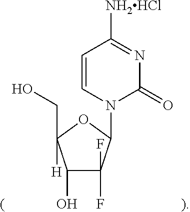

In an embodiment of the invention, an IGF1R inhibitor is provided in association with any compound disclosed in published U.S. patent application no. U.S. 2004/0209878A1 (e.g., comprising a core structure represented by

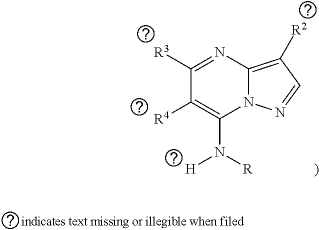

or doxorubicin

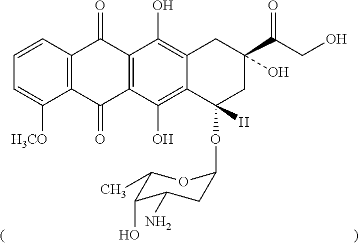

including Caelyx or Doxil® (doxorubicin HCl liposome injection; Ortho Biotech Products L.P; Raritan, N.J.). Doxil® comprises doxorubicin in STEALTH® liposome carriers which are composed of N-(carbonyl-methoxypolyethylene glycol 2000)-1,2-distearoyl-sn-glycero-3-phosphoethanolamine sodium salt (MPEG-DSPE); fully hydrogenated soy phosphatidylcholine (HSPC), and cholesterol.

In an embodiment of the invention, an IGF1R inhibitor is provided in association with 5′-deoxy-5-fluorouridine

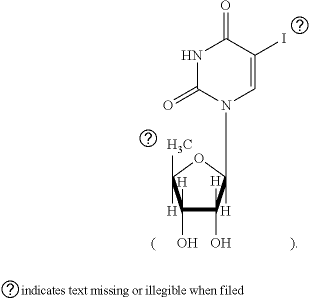

In an embodiment of the invention, an IGF1R inhibitor is provided in association with vincristine

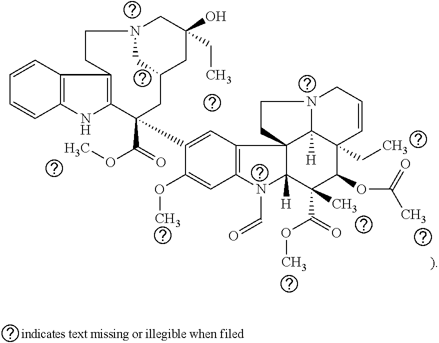

In an embodiment of the invention, an IGF1R inhibitor is provided in association with temozolomide

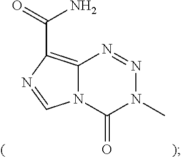

any CDK inhibitor such as ZK-304709, Seliciclib (R-roscovitine)

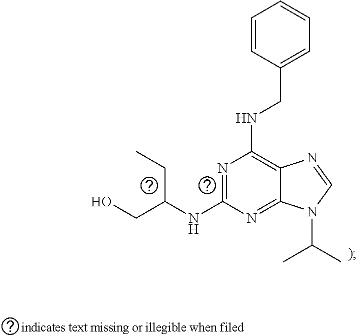

any MEK inhibitor such as PD0325901

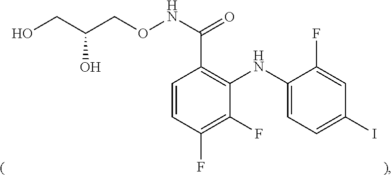

AZD-6244; capecitabine (5′-deoxy-5-fluoro-N-[(pentyloxy)carbonyl]-cytidine); or L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate

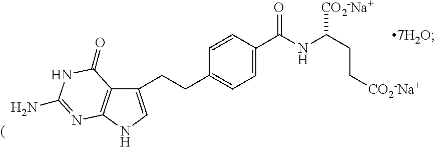

Pemetrexed disodium heptahydrate).

In an embodiment of the invention, an IGF1R inhibitor is provided in association with camptothecin

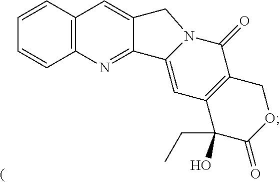

Stork et al., J. Am. Chem. Soc. 93(16): 4074-4075 (1971); Beisler et al., J. Med. Chem. 14(11): 1116-1117 (1962)), irinotecan

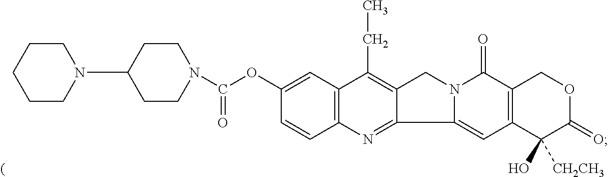

sold as Camptosar®; Pharmacia & Upjohn Co.; Kalamazoo, Mich.); a combination of irinotecan, 5-fluorouracil and leucovorin; or PEG-labeled irinotecan.

In an embodiment of the invention, an IGF1R inhibitor is provided in association with the FOLFOX regimen (oxaliplatin

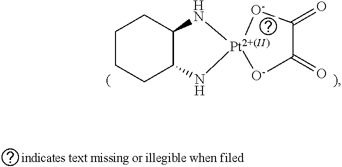

together with infusional fluorouracil

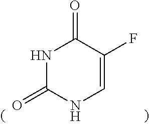

and folinic acid

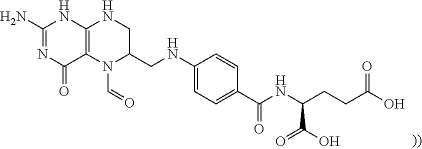

(Chaouche et al., Am. J. Clin. Oncol. 23(3):288-289 (2000); de Gramont et al., J. Clin. Oncol. 18(16):2938-2947 (2000)).

In an embodiment of the invention, an IGF1R inhibitor is provided in association with an antiestrogen such as

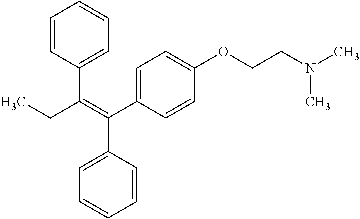

(tamoxifen; sold as Nolvadex® by AstraZeneca Pharmaceuticals LP; Wilmington, Del.) or

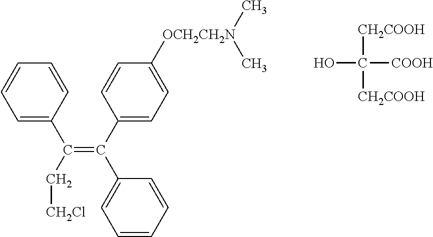

(toremifene citrate; sold as Fareston® by Shire US, Inc.; Florence, Ky.).

In an embodiment of the invention, an IGF1R inhibitor is provided in association with an aromatase inhibitor such as

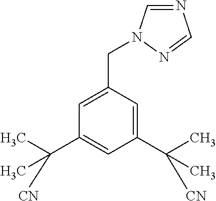

(anastrazole; sold as Arimidex® by AstraZeneca Pharmaceuticals LP; Wilmington, Del.),

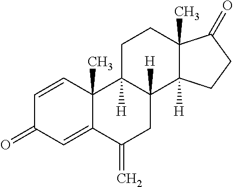

(exemestane; sold as Aromasin® by Pharmacia Corporation; Kalamazoo, Mich.) or

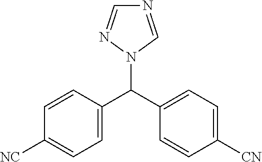

(letrozole; sold as Femara® by Novartis Pharmaceuticals Corporation; East Hanover, N.J.).

In an embodiment of the invention, an IGF1R inhibitor is provided in association with an estrogen such as DES (diethylstilbestrol),

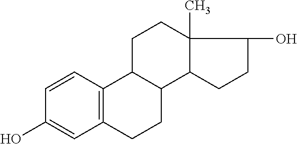

(estradiol; sold as Estrol® by Warner Chilcott, Inc.; Rockaway, N.J.) or conjugated estrogens (sold as Premarin® by Wyeth Pharmaceuticals Inc.; Philadelphia, Pa.).

In an embodiment of the invention, an IGF1R inhibitor is provided in association with anti-angiogenesis agents including bevacizumab (Avastin™; Genentech; San Francisco, Calif.), the anti-VEGFR-2 antibody IMC-1C11, other VEGFR inhibitors such as: CHIR-258

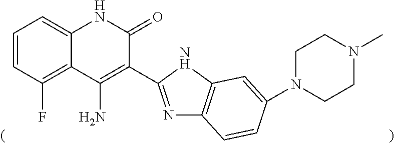

any of the inhibitors set forth in WO2004/13145 (e.g., comprising the core structural formula:

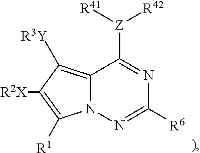

WO2004/09542 (e.g., comprising the core structural formula:

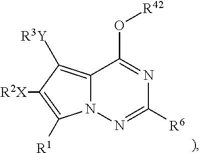

WO00/71129 (e.g., comprising the core structural formula:

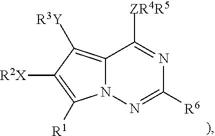

WO2004/09601 (e.g., comprising the core structural formula:

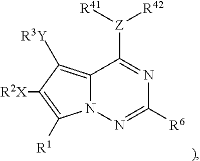

WO2004/01059 (e.g., comprising the core structural formula:

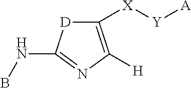

WO01/29025 (e.g., comprising the core structural formula:

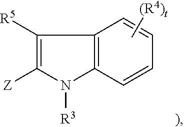

WO02/32861 (e.g., comprising the core structural formula:

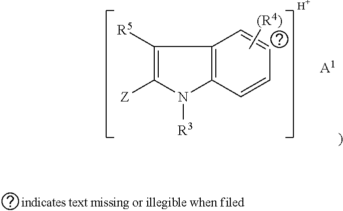

or set forth in WO03/88900 (e.g., comprising the core structural formula

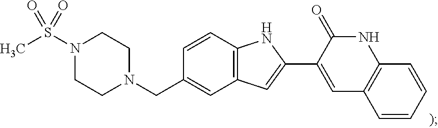

3-[5-(methylsulfonylpiperadinemethyl)-indolyl]-quinolone; Vatalanib

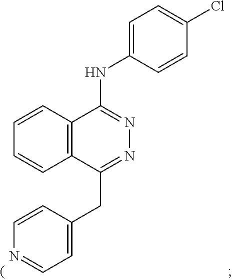

### PTK/ZK; CPG-79787; ZK-222584), AG-013736

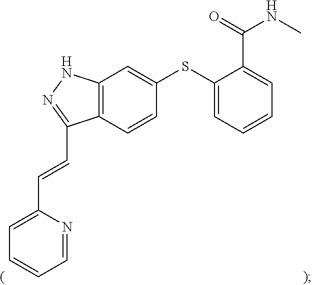

and the VEGF trap (AVE-0005), a soluble decoy receptor comprising portions of VEGF receptors 1 and 2.

In an embodiment of the invention, an IGF1R inhibitor is provided in association with a LHRH (Lutenizing hormone-releasing hormone) agonist such as the acetate salt of [D-Ser(But) 6, Azgly 10] (pyro-Glu-His-Trp-Ser-Tyr-D-Ser(But)-Leu-Arg-Pro-Azgly-NH₂ acetate [C₅₉H₈₄N₁₈O₁₄.(C₂H₄O₂)ₓ where x=1 to 2.4];

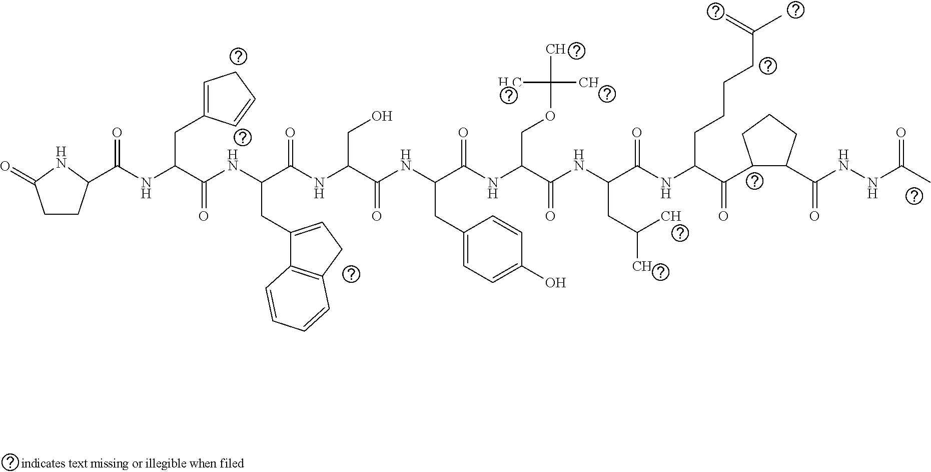

(goserelin acetate; sold as Zoladex® by AstraZeneca UK Limited; Macclesfield, England),

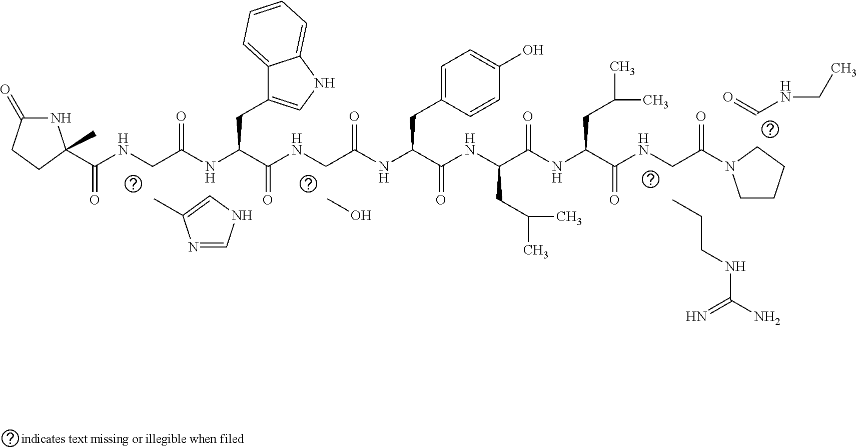

(leuprolide acetate; sold as Eligard® by Sanofi-Synthelabo Inc.; New York, N.Y.) or

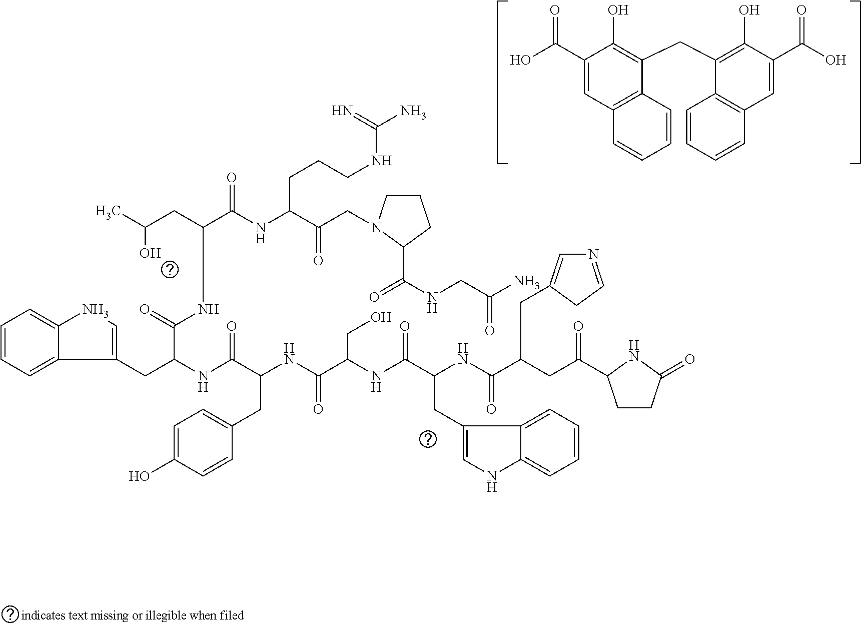

(triptorelin pamoate; sold as Trelstar® by Pharmacia Company, Kalamazoo, Mich.).

In an embodiment of the invention, an IGF1R inhibitor is provided in association with sunitinib or sunitinib malate

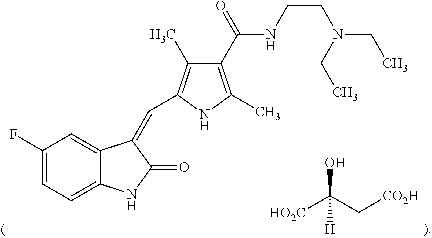

In an embodiment of the invention, an IGF1R inhibitor is provided in association with a progestational agent such as

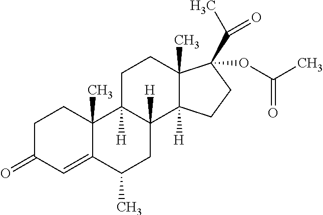

(medroxyprogesterone acetate; sold as Provera® by Pharmacia & Upjohn Co.; Kalamazoo, Mich.),

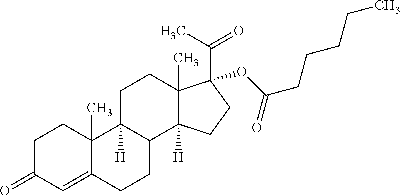

(hydroxyprogesterone caproate; 17-((1-Oxohexyl)oxy)pregn-4-ene-3,20-dione;), megestrol acetate or progestins.

In an embodiment of the invention, an IGF1R inhibitor is provided in association with selective estrogen receptor modulator (SERM) such as

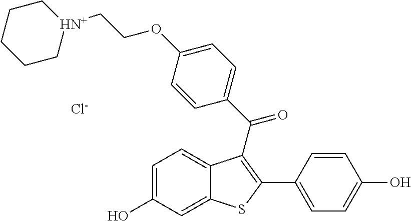

(raloxifene; sold as Evista® by Eli Lilly and Company; Indianapolis, Ind.).

In an embodiment of the invention, an IGF1R inhibitor is provided in association with an anti-androgen including, but not limited to:

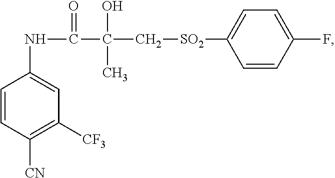

(bicalutamide; sold at CASODEX® by AstraZeneca Pharmaceuticals LP; Wilmington, Del.);

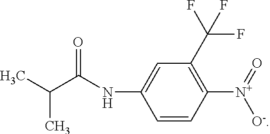

(flutamide; 2-methyl-N-[4-nitro-3 (trifluoromethyl)phenyl] propanamide; sold as Eulexin® by Schering Corporation; Kenilworth, N.J.);

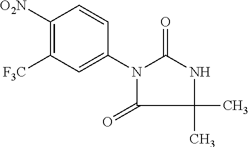

(nilutamide; sold as Nilandron® by Aventis Pharmaceuticals Inc.; Kansas City, Mo.) and

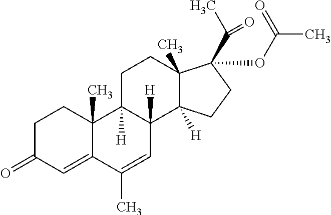

(Megestrol acetate; sold as Megace® by Bristol-Myers Squibb).

In an embodiment of the invention, an IGF1R inhibitor is provided in association with one or more inhibitors which antagonize the action of the EGF Receptor or HER2, including, but not limited to,

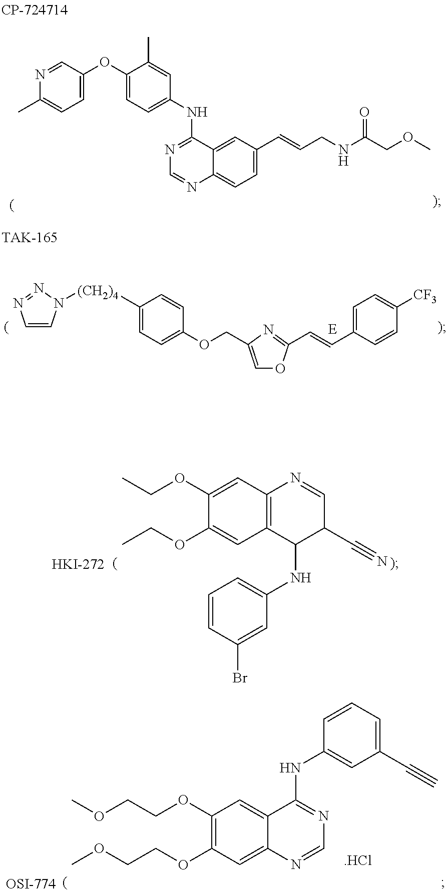

erlotinib, Hidalgo et al., J. Clin. Oncol. 19(13): 3267-3279 (2001)), Lapatanib

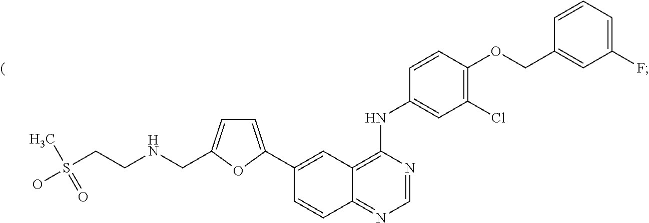

GW2016; Rusnak et al., Molecular Cancer Therapeutics 1:85-94 (2001); N-{3-Chloro-4-[(3-fluorobenzyl)oxy]phenyl}-6-[5-({[2-(methylsulfonypethyl]amino}methyl)-2-furyl]-4-quinazolinamine; PCT Application No. WO99/35146), Canertinib

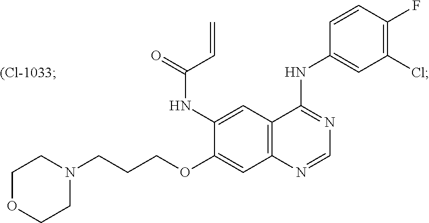

Erlichman et al., Cancer Res. 61(2):739-48 (2001); Smaill et al., J. Med. Chem. 43(7):1380-97 (2000)), ABX-EGF antibody (Abgenix, Inc.; Freemont, Calif.; Yang et al., Cancer Res. 59(6):1236-43 (1999); Yang et al., Crit. Rev Oncol Hematol. 38(1):17-23 (2001)), erbitux (U.S. Pat. No. 6,217,866; IMC-C225, cetuximab; Imclone; New York, N.Y.), EKB-569

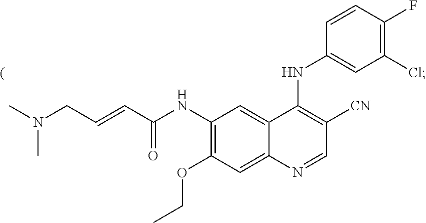

Wissner et al., J. Med. Chem. 46(1): 49-63 (2003)), PKI-166

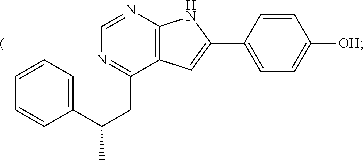

CGP-75166), GW-572016, any anti-EGFR antibody and any anti-HER2 antibody.

In an embodiment of the invention, an IGF1R inhibitor is provided in association with:

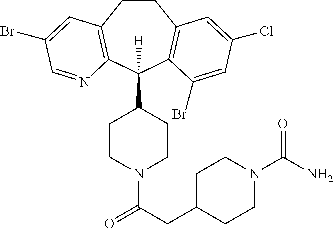

(Ionafarnib; Sarasar™; Schering-Plough; Kenilworth, N.J.). In another embodiment, one of the following FPT inhibitors is provided in association with an IGF1R inhibitor:

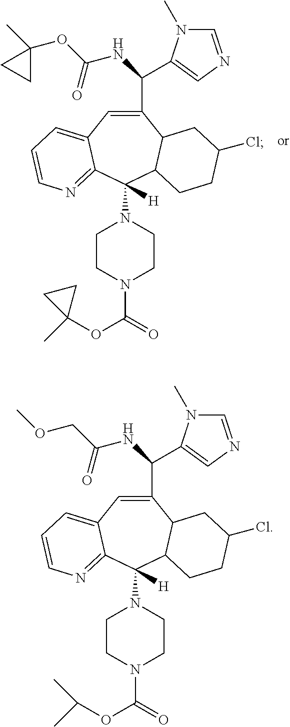

Other FPT inhibitors, that can be provided in association with an IGF1R inhibitor include BMS-214662

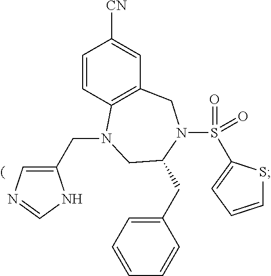

Hunt et al., J. Med. Chem. 43(20):3587-95 (2000); Dancey et al., Curr. Pharm. Des. 8:2259-2267 (2002); (R)-7-cyano-2,3,4,5-tetrahydro-1-(1H-imidazol-4-ylmethyl)-3-(phenylmethyl)-4-(2-thienylsulfonyl)-1H-1,4-benzodiazepine)) and R155777 (tipifarnib; Garner et al., Drug Metab. Dispos. 30(7):823-30 (2002); Dancey et al., Curr. Pharm. Des. 8:2259-2267 (2002); (B)-6-[amino(4-chlorophenyl)(1-methyl-1H-imidazol-5-yl)-methyl]-4-(3-chlorophenyl)-1-methyl-2(1H)-quinolinone];

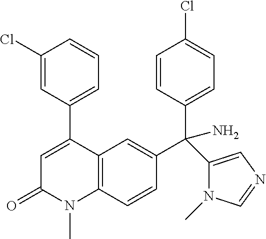

sold as Zarnestra™; Johnson & Johnson; New Brunswick, N.J.).

In an embodiment of the invention, an IGF1R inhibitor is provided in association with

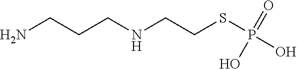

### (Amifostine);

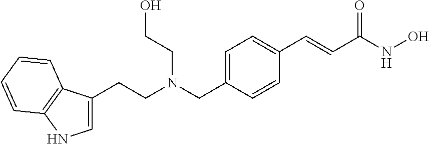

### (NVP-LAQ824; Atadja et al., Cancer Research 64: 689-695 (2004)),

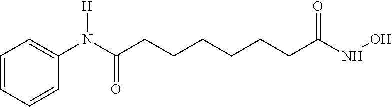

(suberoyl analide hydroxamic acid),

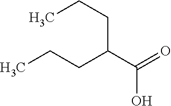

(Valproic acid; Michaelis et al., Mol. Pharmacol. 65:520-527 (2004)),

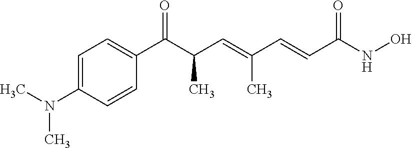

(trichostatin A),

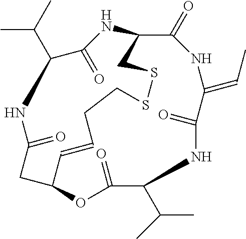

### (FK-228; Furumai et al., Cancer Research 62: 4916-4921 (2002)),

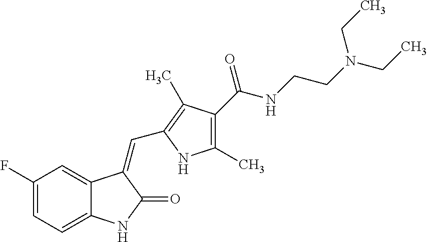

(SU11248; Mendel et al., Clin. Cancer Res. 9(1):327-37 (2003)),

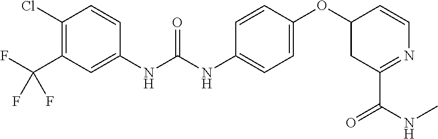

(BAY43-9006; sorafenib),

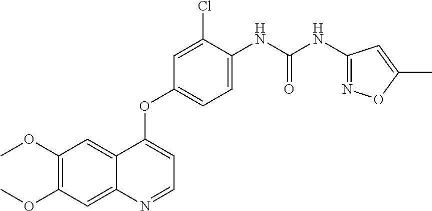

### (KRN951),

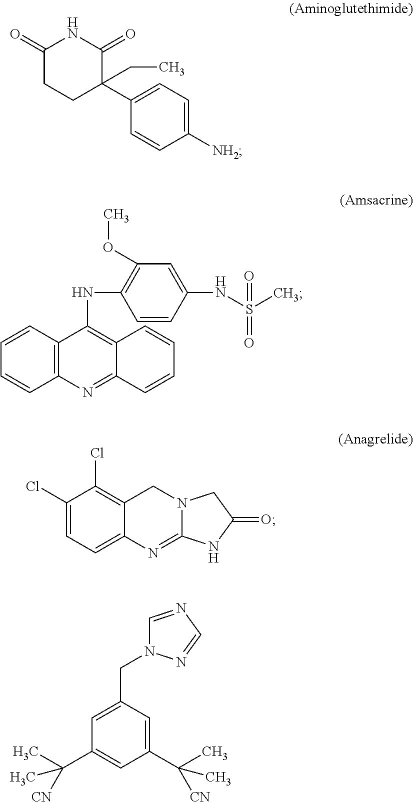

(Anastrozole; sold as Arimidex by AstraZeneca Pharmaceuticals LP; Wilmington, Del.); Asparaginase; Bacillus Calmette-Guerin (BCG) vaccine (Gamido et al., Cytobios. 90(360):47-65 (1997));

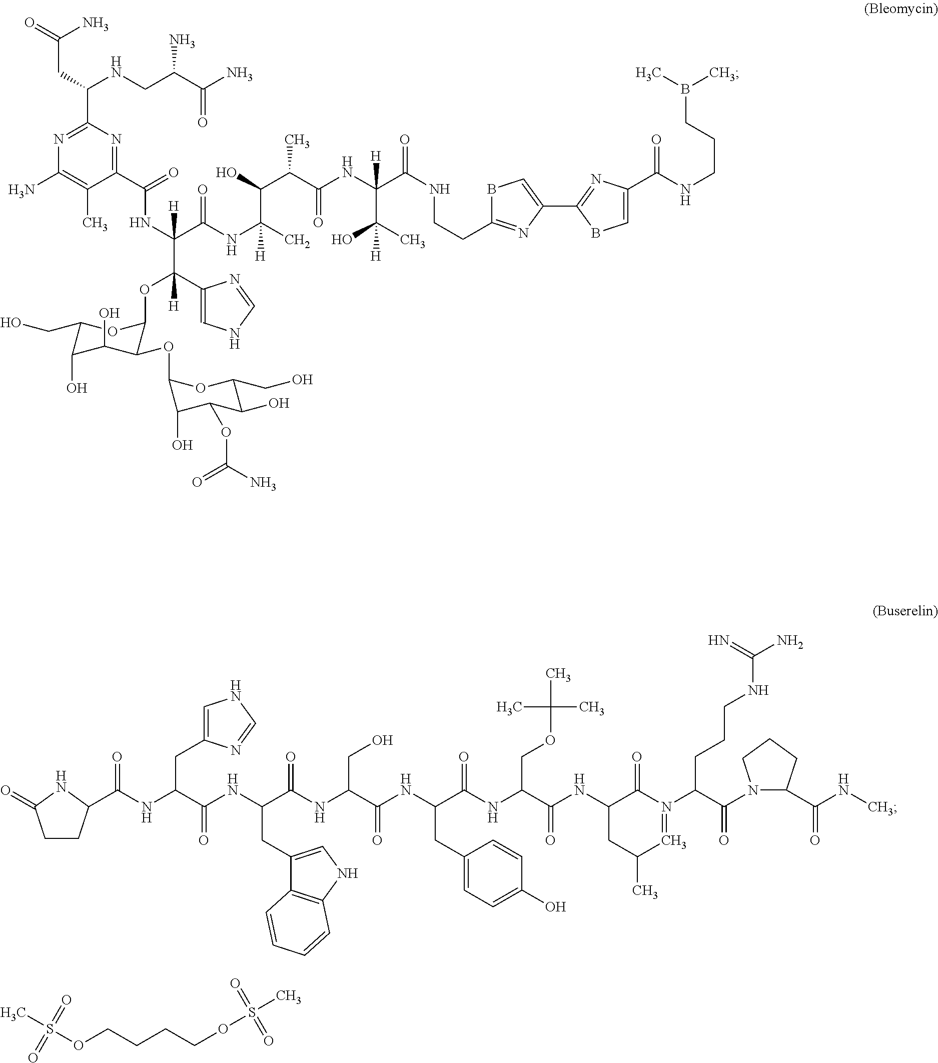

(Busulfan; 1,4-butanediol, dimethanesulfonate; sold as Busulfex® by ESP Pharma, Inc.; Edison, N.J.);

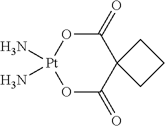

(Carboplatin; sold as Paraplatin® by Bristol-Myers Squibb; Princeton, N.J.);

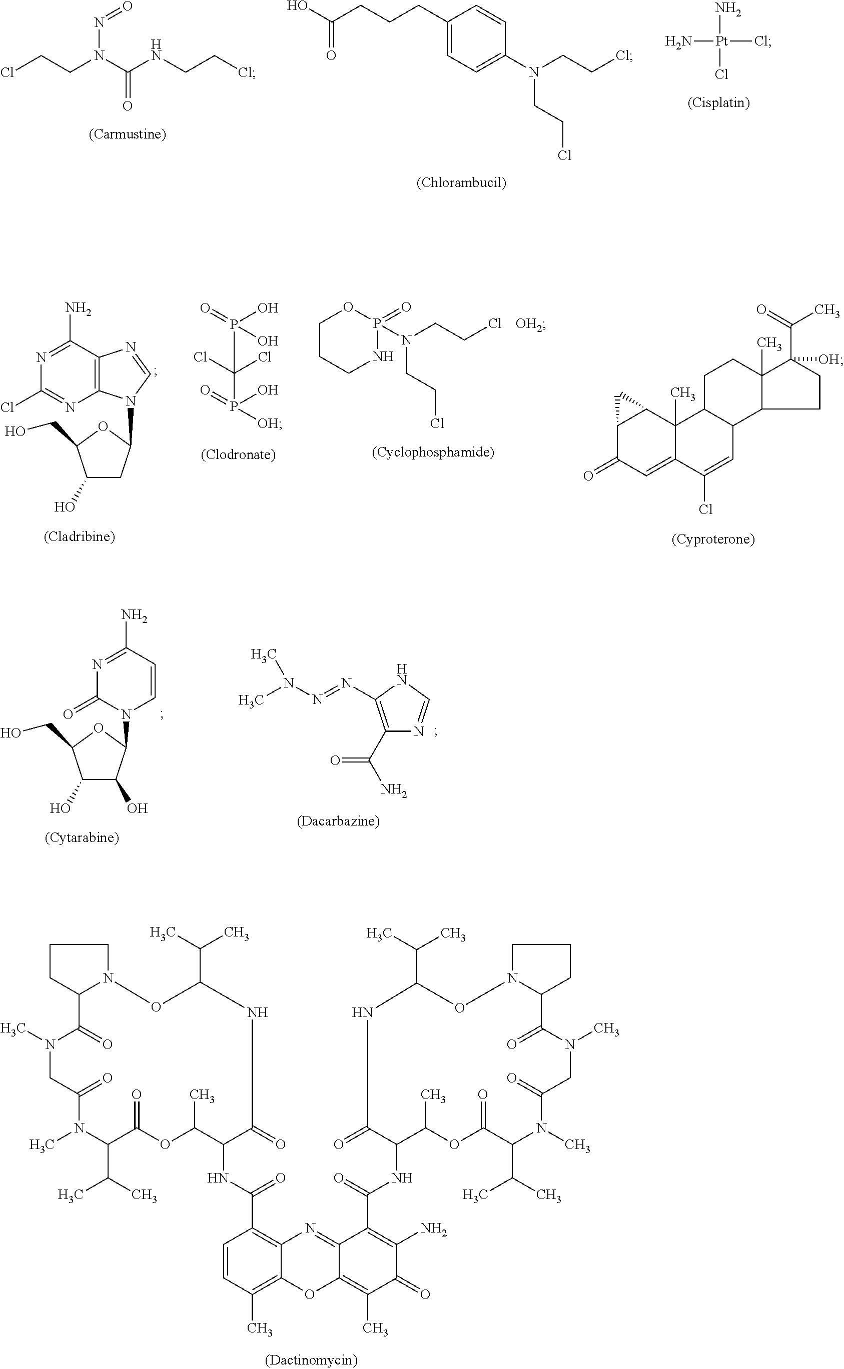

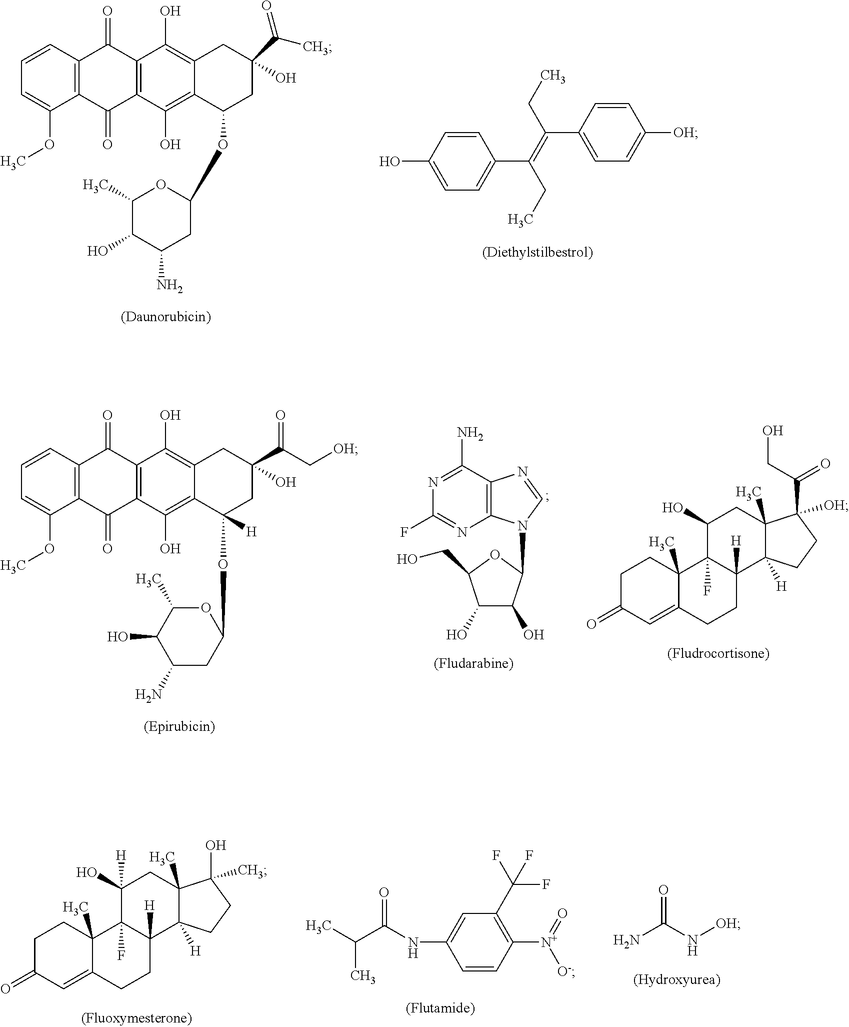

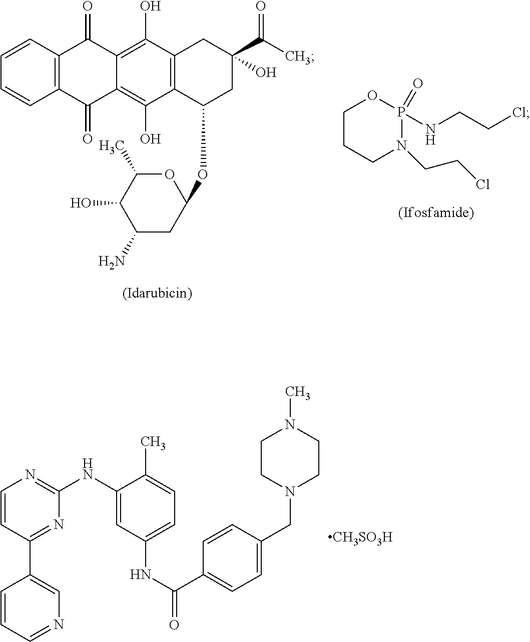

(Imatinib; sold as Gleevec® by Novartis Pharmaceuticals Corporation; East Hanover, N.J.);

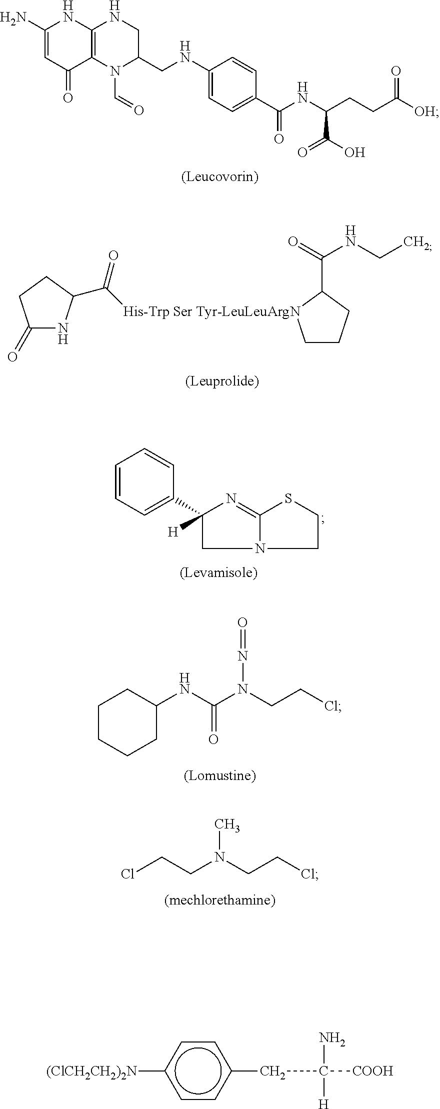

(Melphalan; sold as Alkeran® by Celgene Corporation; Warren, N.J.);

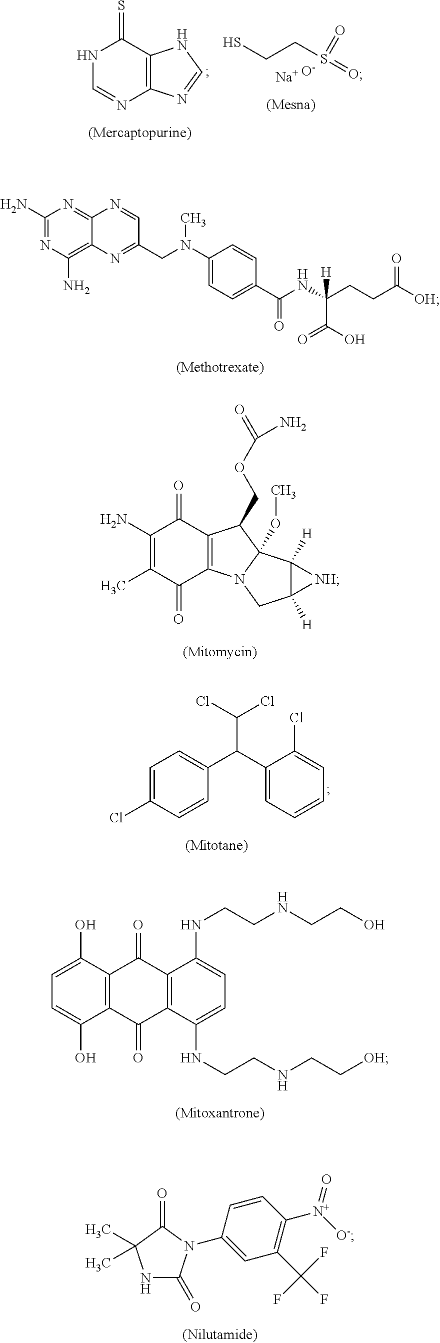

octreotide

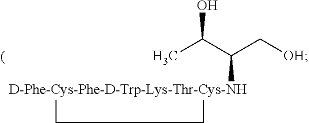

Katz et al., Clin Pharm. 8(4):255-73 (1989); sold as Sandostatin LAR® Depot; Novartis Pharm. Corp; E. Hanover, N.J.); edotreotide (yttrium-90 labeled or unlabeled); oxaliplatin

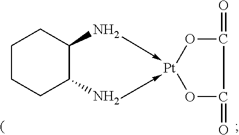

sold as Eloxatin™ by Sanofi-Synthelabo Inc.; New York, N.Y.);

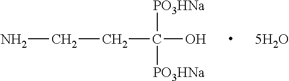

(Pamidronate; sold as Aredia® by Novartis Pharmaceuticals Corporation; East Hanover, N.J.);

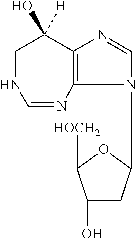

(Pentostatin; sold as Nipent® by Supergen; Dublin, Calif.);

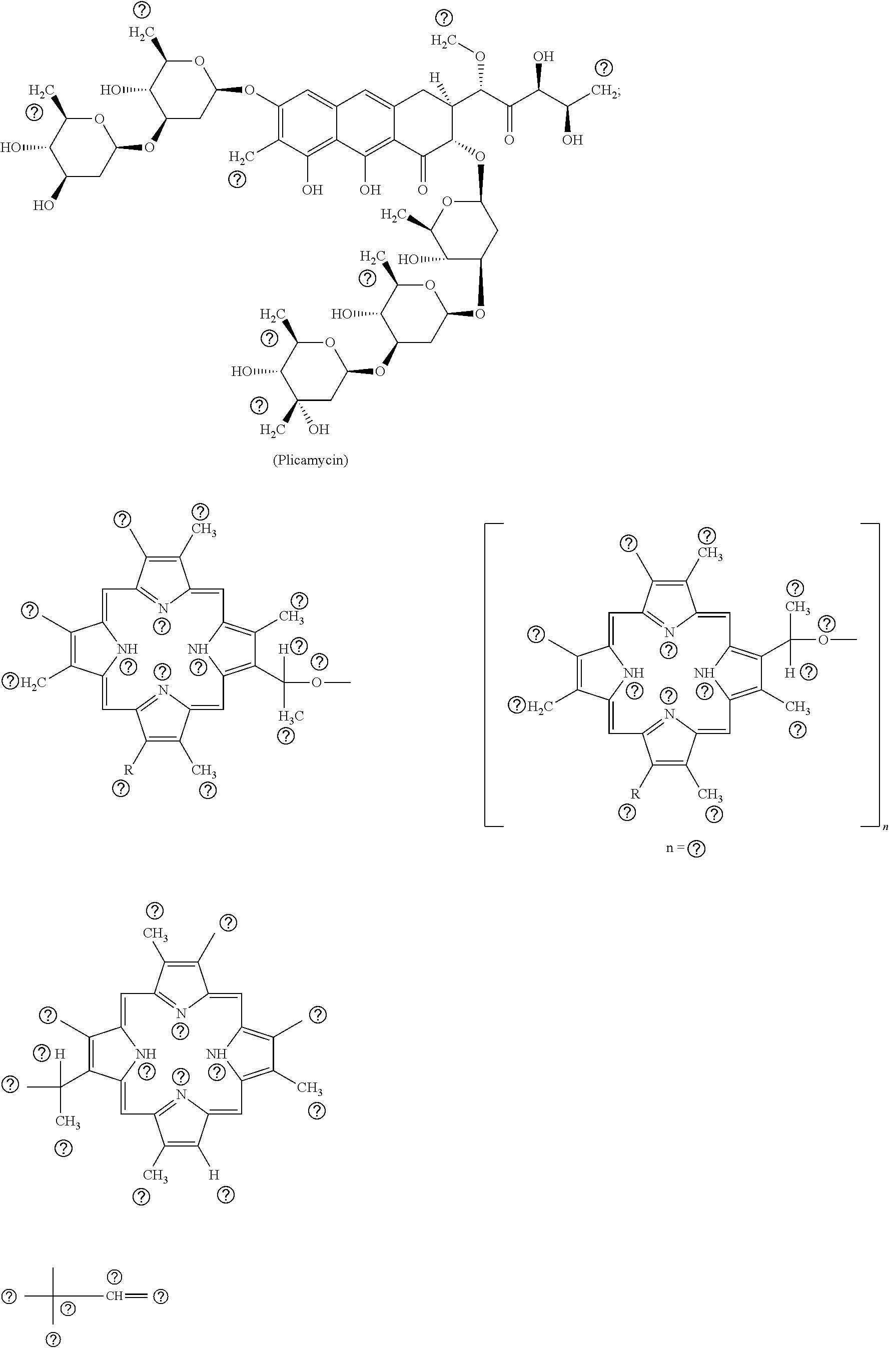

(Porfimer; sold as Photofrin® by Axcan Scandipharm Inc.; Birmingham, Ala.);

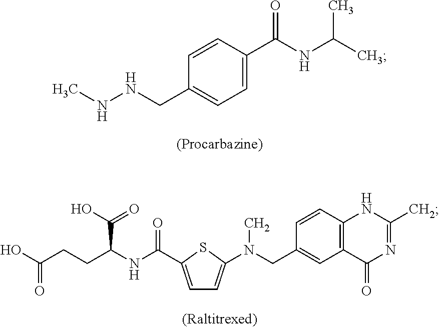

Rituximab (sold as Rituxan® by Genentech, Inc.; South San Francisco, Calif.);

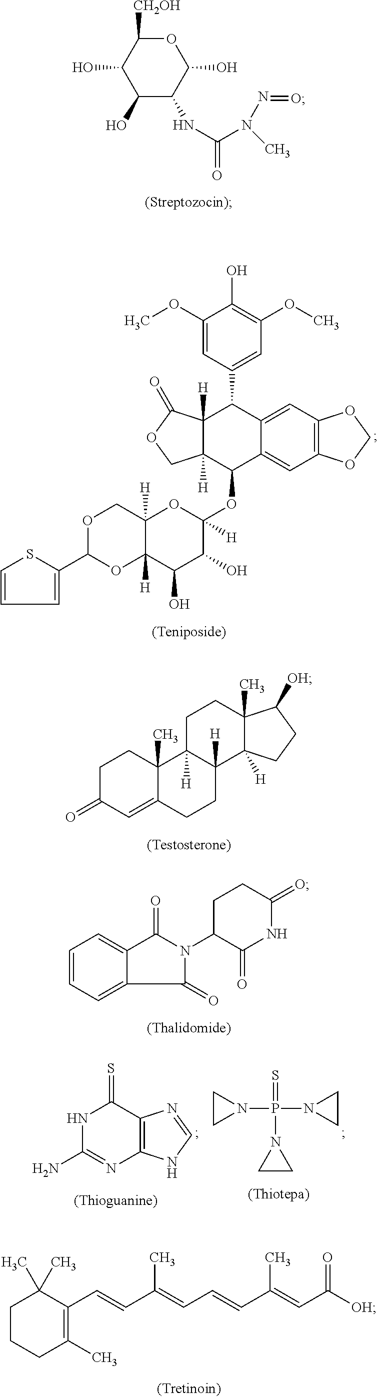

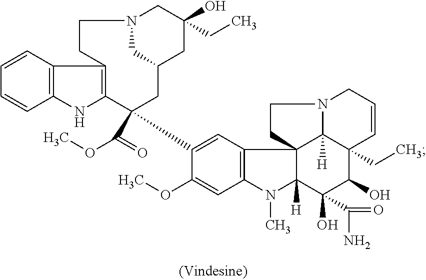

or 13-cis-retinoic acid

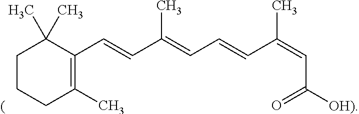

In an embodiment of the invention, an IGF1R inhibitor is provided in association with one or more of any of: phenylalanine mustard, uracil mustard, estramustine, altretamine, floxuridine, 5-deooxyuridine, cytosine arabinoside, 6-mercaptopurine, deoxycoformycin, calcitriol, vairubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin, diftitox, gefitinib, bortezimib, paclitaxel, docetaxel, epithilone B, BMS-247550 (see e.g., Lee et al., Clin. Cancer Res. 7:1429-1437 (2001)), BMS-310705, droloxifene (3-hydroxytamoxifen), 4-hydroxytamoxifen, pipendoxifene, ERA-923, arzoxifene, fulvestrant, acolbifene, lasofoxifene (CP-336156), idoxifene, TSE-424, HMR-3339, ZK186619, topotecan, PTK787/ZK 222584 (Thomas et al., Semin Oncol. 30(3 Suppl 6):32-8 (2003)), the humanized anti-VEGF antibody Bevacizumab, VX-745 (Haddad, Curr Opin. Investig. Drugs 2(8):1070-6 (2001)), PD 184352 (Sebolt-Leopold, et al. Nature Med. 5: 810-816 (1999)), any mTOR inhibitor, rapamycin

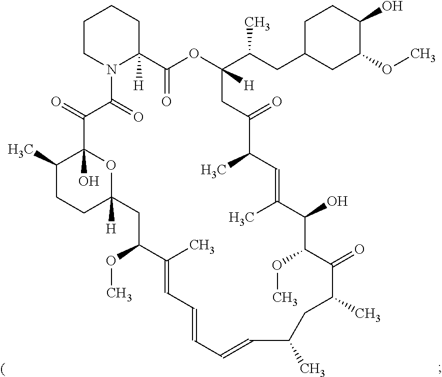

sirolimus), 40-O-(2-hydroxyethyl)-rapamycin, CCI-779

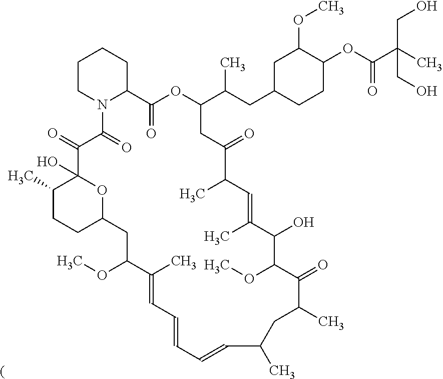

temsirolimus; Sehgal et al., Med. Res. Rev., 14:1-22 (1994); Elit, Curr. Opin. Investig. Drugs 3(8):1249-53 (2002)),

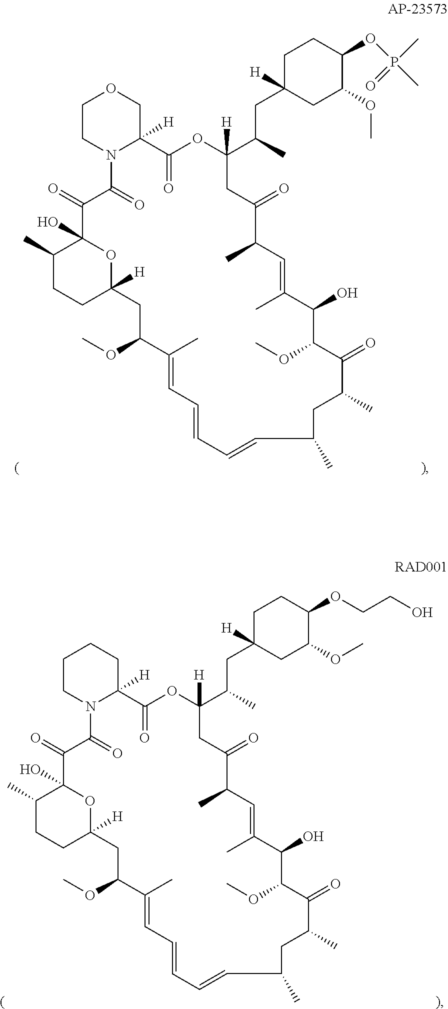

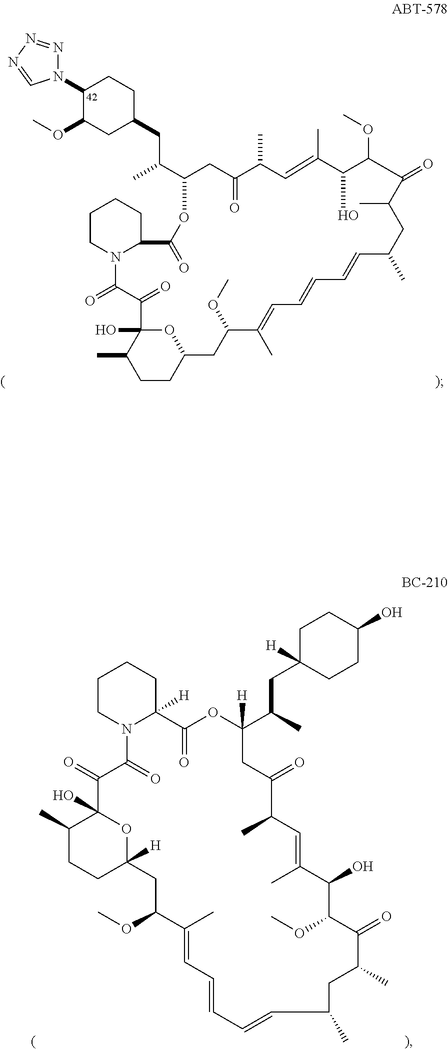

LY294002, LY292223, LY292696, LY293684, LY293646 (Vlahos et al., J. Biol. Chem. 269(7): 5241-5248 (1994)), wortmannin, BAY-43-9006, (Wilhelm et al., Curr. Pharm. Des. 8:2255-2257 (2002)), ZM336372, L-779,450, any Raf inhibitor disclosed in Lowinger et al., Curr. Pharm Des. 8:2269-2278 (2002); flavopiridol (L86-8275/HMR 1275; Senderowicz, Oncogene 19(56): 6600-6606 (2000)) or UCN-01 (7-hydroxy staurosporine; Senderowicz, Oncogene 19(56): 6600-6606 (2000)).

In an embodiment of the invention, an IGF1R inhibitor is provided in association with one or more of any of the compounds set forth in U.S. Pat. No. 5,656,655, which discloses styryl substituted heteroaryl EGFR inhibitors; in U.S. Pat. No. 5,646,153 which discloses bis mono and/or bicyclic aryl heteroaryl carbocyclic and heterocarbocyclic EGFR and PDGFR inhibitors; in U.S. Pat. No. 5,679,683 which discloses tricyclic pyrimidine compounds that inhibit the EGFR; in U.S. Pat. No. 5,616,582 which discloses quinazoline derivatives that have receptor tyrosine kinase inhibitory activity; in Fry et al., Science 265 1093-1095 (1994) which discloses a compound having a structure that inhibits EGFR (see FIG. 1 of Fry et al.); in U.S. Pat. No. 5,196,446 which discloses heteroarylethenediyl or heteroarylethenediylaryl compounds that inhibit EGFR; in Panek, et al., Journal of Pharmacology and Experimental Therapeutics 283: 1433-1444 (1997) which disclose a compound identified as PD166285 that inhibits the EGFR, PDGFR, and FGFR families of receptors-PD166285 is identified as 6-(2,6-dichlorophenyl)-2-(4-(2-diethylaminoethoxy)phenylamino)-8-methyl-8H-pyrido(2,3-d)pyrimidin-7-one.

In an embodiment of the invention, an IGF1R inhibitor is provided in association with one or more of any of: pegylated or unpegylated interferon alfa-2a, pegylated or unpegylated interferon alfa-2b, pegylated or unpegylated interferon alfa-2c, pegylated or unpegylated interferon alfa n−1, pegylated or unpegylated interferon alfa n−3 and pegylated, unpegylated consensus interferon or albumin-interferon-alpha.

The term “interferon alpha” as used herein means the family of highly homologous species-specific proteins that inhibit cellular proliferation and modulate immune response. Typical suitable interferon-alphas include, but are not limited to, recombinant interferon alpha-2b, recombinant interferon alpha-2a, recombinant interferon alpha-2c, alpha 2 interferon, interferon alpha-n1 (INS), a purified blend of natural alpha interferons, a consensus alpha interferon such as those described in U.S. Pat. Nos. 4,897,471 and 4,695,623 (especially Examples 7, 8 or 9 thereof), or interferon alpha-n3, a mixture of natural alpha interferons.

Interferon alfa-2a is sold as ROFERON-A® by Hoffmann-La Roche (Nutley, N.J.).

Interferon alfa-2b is sold as INTRON-A® by Schering Corporation (Kenilworth, N.J.). The manufacture of interferon alpha 2b is described, for example, in U.S. Pat. No. 4,530,901.

Interferon alfa-n3 is a mixture of natural interferons sold as ALFERON N INJECTION® by Hemispherx Biopharma, Inc. (Philadelphia, Pa.).

Interferon alfa-n1 (INS) is a mixture of natural interferons sold as WELLFERON® by Glaxo-Smith-Kline (Research Triangle Park, N.C.).

Consensus interferon is sold as INFERGEN® by Intermune, Inc. (Brisbane, Calif.).

Interferon alfa-2c is sold as BEROFOR® by Boehringer Ingelheim Pharmaceutical, Inc. (Ridgefield, Conn.).

A purified blend of natural interferons is sold as SUMIFERON® by Sumitomo; Tokyo, Japan.

The term “pegylated interferon alpha” as used herein means polyethylene glycol modified conjugates of interferon alpha, preferably interferon alpha-2a and alpha-2b. The preferred polyethylene-glycol-interferon alpha-2b conjugate is PEG 12000-interferon alpha-2b. The phrases “12,000 molecular weight polyethylene glycol conjugated interferon alpha” and “PEG 12000-IFN alpha” as used herein include conjugates such as are prepared according to the methods of International Application No. WO 95/13090 and EP1039922 and containing urethane linkages between the interferon alpha-2a or -2b amino groups and polyethylene glycol having an average molecular weight of 12000. The pegylated inteferon alpha, PEG 12000-IFN-alpha-2b is available from Schering-Plough, Kenilworth, N.J.

Pegylated interferon alfa-2b is sold as PEG-INTRON® by Schering Corporation (Kenilworth, N.J.).

Pegylated interferon-alfa-2a is sold as PEGASYS® by Hoffmann-La Roche (Nutley, N.J.).

Other interferon alpha conjugates can be prepared by coupling an interferon alpha to a water-soluble polymer. A non-limiting list of such polymers includes other polyalkylene oxide homopolymers such as polypropylene glycols, polyoxyethylenated polyols, copolymers thereof and block copolymers thereof. As an alternative to polyalkylene oxide-based polymers, effectively non-antigenic materials such as dextran, polyvinylpyrrolidones, polyacrylamides, polyvinyl alcohols, carbohydrate-based polymers and the like can be used. Such interferon alpha-polymer conjugates are described, for example, in U.S. Pat. No. 4,766,106, U.S. Pat. No. 4,917,888, European Patent Application No. 0 236 987 or 0 593 868 or International Publication No. WO 95/13090.

Pharmaceutical compositions of pegylated interferon alpha suitable for parenteral administration can be formulated with a suitable buffer, e.g., Tris-HCl, acetate or phosphate such as dibasic sodium phosphate/monobasic sodium phosphate buffer, and pharmaceutically acceptable excipients (e.g., sucrose), carriers (e.g. human plasma albumin), toxicity agents (e.g., NaCl), preservatives (e.g., thimerosol, cresol or benzyl alcohol), and surfactants (e.g., tween or polysorbates) in sterile water for injection. The pegylated interferon alpha can be stored as lyophilized powder under refrigeration at 2°-8° C. The reconstituted aqueous solutions are stable when stored between 2° and 8° C. and used within 24 hours of reconstitution. See for example U.S. Pat. Nos. 4,492,537; 5,762,923 and 5,766,582. The reconstituted aqueous solutions may also be stored in prefilled, multi-dose syringes such as those useful for delivery of drugs such as insulin. Typical, suitable syringes include systems comprising a prefilled vial attached to a pen-type syringe such as the NOVOLET® Novo Pen available from Novo Nordisk or the REDIPEN®, available from Schering Corporation, Kenilworth, N.J. Other syringe systems include a pen-type syringe comprising a glass cartridge containing a diluent and lyophilized pegylated interferon alpha powder in a separate compartment.

The scope of the present invention also includes compositions comprising an IGF1R inhibitor in association with one or more other anti-cancer chemotherapeutic agents (e.g., as described herein) in association with one or more antiemetics including, but not limited to, casopitant (GlaxoSmithKline), Netupitant (MGI-Helsinn) and other NK-1 receptor antagonists, palonosetron (sold as Aloxi by MGI Pharma), aprepitant (sold as Emend by Merck and Co.; Rahway, N.J.), diphenhydramine (sold as Benadryl® by Pfizer; New York, N.Y.), hydroxyzine (sold as Atarax® by Pfizer; New York, N.Y.), metoclopramide (sold as Reglan® by AH Robins Co,; Richmond, Va.), lorazepam (sold as Ativan® by Wyeth; Madison, N.J.), alprazolam (sold as Xanax® by Pfizer; New York, N.Y.), haloperidol (sold as Haldol® by Ortho-McNeil; Raritan, N.J.), droperidol (Inapsine®), dronabinol (sold as Marinol® by Solvay Pharmaceuticals, Inc.; Marietta, Ga.), dexamethasone (sold as Decadron® by Merck and Co.; Rahway, N.J.), methylprednisolone (sold as Medrol® by Pfizer; New York, N.Y.), prochlorperazine (sold as Compazine® by Glaxosmithkline; Research Triangle Park, N.C.), granisetron (sold as Kytril® by Hoffmann-La Roche Inc.; Nutley, N.J.), ondansetron (sold as Zofran® by Glaxosmithkline; Research Triangle Park, N.C.), dolasetron (sold as Anzemet® by Sanofi-Aventis; New York, N.Y.), tropisetron (sold as Navoban® by Novartis; East Hanover, N.J.).

Compositions comprising an antiemetic are useful for preventing or treating nausea; a common side effect of anti-cancer chemotherapy. Accordingly, the present invention also includes methods for treating or preventing cancer in a subject by administering an IGF1R inhibitor optionally in association with one or more other chemotherapeutic agents (e.g., as described herein) and/or optionally in association with one or more antiemetics.

Other side effects of cancer treatment include red and white blood cell deficiency. Accordingly, the present invention includes compositions comprising an IGF1R inhibitor optionally in association with an agent which treats or prevents such a deficiency, such as, e.g., pegfilgrastim, erythropoietin, epoetin alfa or darbepoetin alfa.

The present invention further comprises a method for treating or preventing any stage or type of any medical condition set forth herein by administering an IGF1R inhibitor in association with a therapeutic procedure such as surgical tumorectomy or anti-cancer radiation treatment; optionally in association with a further chemotherapeutic agent and/or antiemetic, for example, as set forth above.

The term “in association with” indicates that the components of a composition of the invention (e.g., anti-IGF1R antibody or antigen-binding fragment thereof along with imatinib) can be formulated into a single composition for simultaneous delivery or formulated separately into two or more compositions (e.g., a kit). Furthermore, each component can be administered to a subject at a different time than when the other component is administered; for example, each administration may be given non-simultaneously (e.g., separately or sequentially) at several intervals over a given period of time. Moreover, the separate components may be administered to a subject by the same or by a different route (e.g., wherein an anti-IGF1R antibody is administered parenterally and gosrelin acetate is administered orally).

### Therapeutic Methods, Dosage and Administration

The present invention provides methods for determining the expression levels of any of the genes set forth in table 1 or table 3 in a subject receiving IGF1R inhibitor therapy. In an embodiment of the invention, the subject suffers from a medical condition mediated by cellular IGF1R expression or activity or the expression or activity of any member of the IGF1R pathway (including e.g., IRS-1, PI3 kinase, ERK2 or AKT). In an embodiment of the invention, the medical condition is any of the following: osteosarcoma, rhabdomyosarcoma, neuroblastoma, any pediatric cancer, kidney cancer, leukemia, renal transitional cell cancer, Werner-Morrison syndrome, acromegaly, bladder cancer, Wilm's cancer, ovarian cancer, pancreatic cancer, benign prostatic hyperplasia, breast cancer, prostate cancer, bone cancer, lung cancer, gastric cancer, colorectal cancer, cervical cancer, synovial sarcoma, diarrhea associated with metastatic carcinoid, vasoactive intestinal peptide secreting tumors, gigantism, psoriasis, atherosclerosis, smooth muscle restenosis of blood vessels and inappropriate microvascular proliferation, head and neck cancer, squamous cell carcinoma, multiple myeloma, solitary plasmacytoma, renal cell cancer, retinoblastoma, germ cell tumors, hepatoblastoma, hepatocellular carcinoma, melanoma, rhabdoid tumor of the kidney, Ewing Sarcoma, chondrosarcoma, haemotological malignancy, chronic lymphoblastic leukemia, chronic myelomonocytic leukemia, acute lymphoblastic leukemia, acute lymphocytic leukemia, acute myelogenous leukemia, acute myeloblastic leukemia, chronic myeloblastic leukemia, Hodgekin's disease, non-Hodgekin's lymphoma, chronic lymphocytic leukemia, chronic myelogenous leukemia, myelodysplastic syndrome, hairy cell leukemia, mast cell leukemia, mast cell neoplasm, follicular lymphoma, diffuse large cell lymphoma, mantle cell lymphoma, Burkitt Lymphoma, mycosis fungoides, seary syndrome, cutaneous T-cell lymphoma, chronic myeloproliferative disorders, a cental nervous system tumor, brain cancer, glioblastoma, non-glioblastoma brain cancer, meningioma, pituitary adenoma, vestibular schwannoma, a primitive neuroectodermal tumor, medulloblastoma, astrocytoma, anaplastic astrocytoma, oligodendroglioma, ependymoma and choroid plexus papilloma, a myeloproliferative disorder, polycythemia vera, thrombocythemia, idiopathic myelfibrosis, soft tissue sarcoma, thyroid cancer, endometrial cancer, carcinoid cancer, germ cell tumors, liver cancer, gigantism, psoriasis, atherosclerosis, smooth muscle restenosis of blood vessels, inappropriate microvascular proliferation, acromegaly, gigantism, psoriasis, atherosclerosis, smooth muscle restenosis of blood vessels or inappropriate microvascular proliferation, Grave's disease, multiple sclerosis, systemic lupus erythematosus, Hashimoto's Thyroiditis, Myasthenia Gravis, auto-immune thyroiditis and Bechet's disease.

The IGF1R inhibitors discussed herein (e.g., anti-IGF1R antibodies and antigen-binding fragments thereof) and compositions thereof are, in an embodiment of the invention, administered at a therapeutically effective dosage. The term “therapeutically effective amount” or “therapeutically effective dosage” means that amount or dosage of an IGF1R inhibitor or composition thereof that will elicit a biological or medical response of a tissue, system, patient, subject or host that is being sought by the administrator (such as a researcher, doctor or veterinarian) which includes any measurable alleviation of the signs, symptoms and/or clinical indicia of a medical disorder, such as cancer (e.g., tumor growth and/or metastasis) including the prevention, slowing or halting of progression of the medical disorder to any degree whatsoever. For example, in one embodiment of the invention, a “therapeutically effective dosage” of any anti-IGF1R antibody or antigen-binding fragment thereof discussed herein (e.g., an anti-IGF1R antibody comprising mature LCC, LCD, LCE or LCF light chain and/or mature HCA or HCB heavy chain) is between about 0.3 and 20 mg/kg of body weight (e.g., about 0.3 mg/kg of body weight, about 0.6 mg/kg of body weight, about 0.9 mg/kg of body weight, about 1 mg/kg of body weight, about 2 mg/kg of body weight, about 3 mg/kg of body weight, about 4 mg/kg of body weight, about 5 mg/kg of body weight, about 6 mg/kg of body weight, about 7 mg/kg of body weight, about 8 mg/kg of body weight, about 9 mg/kg of body weight, about 10 mg/kg of body weight, about 11 mg/kg of body weight, about 12 mg/kg of body weight, about 13 mg/kg of body weight, about 14 mg/kg of body weight, about 15 mg/kg of body weight, about 16 mg/kg of body weight, about 17 mg/kg of body weight, about 18 mg/kg of body weight, about 19 mg/kg of body weight, about 20 mg/kg of body weight), about once per week to about once every 3 weeks (e.g., about once every 1 week or once every 2 weeks or once every 3 weeks). The therapeutically effective dosage of an IGF1R inhibitor or any further therapeutic agent is, when possible, as set forth in the Physicians' Desk Reference.

### Biomarkers for Sensitivity to IGF1R Inhibitors and Uses Thereof

Genes upregulated in sensitive cells relative to resistant cells are:

### TRE2;

### SMC4;

### TRIB2;

### TLE4;

### BMP7;

### PCDHGC3;

### AUTS2;

C14orf132;

### CERK;

### HDGFRP3;

### TCF4;

### MEIS2;

### EML4;

C7orf41;

### KIAA1450;

### ZNF136;

### D15F37

### CDK6

### TIMP

### Clu; and

### PRL1

Genes downregulated in sensitive cells relative to resistant cells are:

### ACAT1;

### ALDOC;

C6orf192;

### COL4A5;

### C1QBP;

### CRIP1;

### DEADC1;

### GSTK1

### GSTO2;

### PPAPDC1B;

### TMEM107;

### JOSD3;

### TMEM77;

### MST1;

### MT1E;

### PARVB;

### PRDX4;

### RASGEF1A;

### RPL14;

### IFI30;

### ATF1;

### ACADVL;

### FBXO6;

### NQO2;

### TMEM64;

### ZFAND1;

### TMED5;

### PDIA5;

### MYO1C;

### GNPTAB;

### LACTB2;

### RPL22;

### TSPAN4;

### RPL15;

### PCCB;

### CRYZ;

### DNAJC10;

C19orf54;

### HSPE1; and

hqp0376

Embodiments of the present include those in which any of the foregoing genes comprise any of the following nucleotide sequences or any allelic variant thereof (e.g., a sequence conserved variant or a functionally conserved variant thereof):

### Sequence Accession No.: NM_{—}004505

### Gene: TRE2

| (SEQ ID NO: 99) | |
|---|---|
| atggacatgg tagagaatgc agatagtttg caggcacagg agcggaagga catacttatg | 60 |
| aagtatgaca agggacaccg agctgggctg ccagaggaca aggggcctga gcccgctgga | 120 |
| atcaacagca gcattgatcg ctttggcatt ttgcatgaga cggagctgcc tcctgtgact | 180 |
| gcacgggagg cgaagaaaac tcggcgggag atgacacgaa cgagcaagtg gatggaaatg | 240 |
| ccgggagaac gggagacata taagcacagt agcaaactca tagaccgagt gtacaaggga | 300 |
| attcccatga acatccgggg cccggtgtgg tcagtcctcc tgaacattca ggaaaccaag | 360 |
| ttgaaaaacc ccggaagata ccagatcatg aaggagaggg gcaagaggtc atctgaacac | 420 |
| atccaccaca tcgacctgga cgtgaggacg actctccgga accatgtctt ctttagggat | 480 |
| cgacatggag ccaagcagag ggaactattc tacatcctcc tggcctattc ggagtataac | 540 |
| ccggaggtgg gctactgcag ggacctgagc cacatcaccg ccttgttcct ccttcatctg | 600 |
| cctgaggagg acgcattctg ggcactggtg cagctgctgg ccagtgagag gcactccctg | 660 |
| ccaggatccc acagcccaaa tggtgggaca gtccaggggc tccaagacca acaggagcat | 720 |
| gtggtaccca agtcacaacc caagaccatg tggcatcagg acaaggaagg tctatgcggg | 780 |
| cagtgtgcct cgttaggctg ccttctccgg aacctgattg acgggatctc tctcgggctc | 840 |
| accctgcgcc tgtgggacgt gtatttggtg gaaggagaac aggtgttgat gccaataacc | 900 |
| agcattgctc ttaaggttca gcagaagcgc ctcatgaaga catccaggtg tggcctgtgg | 960 |
| gcacgtctgc ggaaccaatt cttcgatacc tgggccatga acgatgacac cgtgctcaag | 1020 |
| catcttaggg cctctacgaa gaaactaaca aggaagcaag gggacctgcc acccccagcc | 1080 |
| aaacgcgagc aagggtcctt ggcacccagg cccgtgccgg cttcacgtgg tgggaagacc | 1140 |
| ctctgcaagg ggtataggca ggcccctcca ggcccaccag cccagttcca gcggcccatt | 1200 |
| tgctcagctt ccccgccatg ggcatctcgt ttttccacgc cctgtcctgg tggggctgtc | 1260 |
| cgggaagaca cgtaccctgt gggcactcag ggtgtgccca gcctggccct ggctcaggga | 1320 |
| ggacctcagg gttcctggag attcctggag tggaagtcaa tgccccggct cccaacggac | 1380 |
| ctggatatag ggggcccctg gttcccccat tatgattttg aatggagctg ctgggtccgt | 1440 |
| gccatatccc aggaggacca gctggccacc tgctggcagg ctgaacactg cggagaggtt | 1500 |
| cacaacaaag atatgagttg gcctgaggag atgtctttta cagcaaatag tagtaaaata | 1560 |
| gatagacaaa aggttcccac agaaaaggga gccacaggtc taagcaacct gggaaacaca | 1620 |
| tgcttcatga actcaagcat ccagtgcgtt agtaacacac agccactgac acagtatttt | 1680 |
| atctcaggga gacatcttta tgaactcaac aggacaaatc ccattggtat gaaggggcat | 1740 |
| atggctaaat gctatggtga tttagtgcag gaactctgga gtggaactca gaagagtgtt | 1800 |
| gccccattaa agcttcggcg gaccatagca aaatatgctc ccaagtttga tgggtttcag | 1860 |
| caacaagact cccaagaact tctggctttt ctcttggatg gtcttcatga agatctcaac | 1920 |
| cgagcccatg aaaagccata tgtggaactg aaggacagtg atggccgacc agactgggaa | 1980 |
| gtagctgcag aggcctggga caaccatcta agaagaaata gatcaattat tgtggatttg | 2040 |
| ttccatgggc agctaagatc tcaagtcaaa cgcaagacat gtgggcatat aagtgtccga | 2100 |
| tttgaccctt tcaatttttt gtctttgcca ctaccaatgg acagttacat ggacttagaa | 2160 |
| ataacagtga ttaagttaga tggtactacc cctgtacggt atggactaag actgaatatg | 2220 |
| gatgaaaagt acacaggttt aaaaaaacag ctgagggatc tctgtggact taattcagaa | 2280 |
| caaatcctac tagcagaagt acatgattcc aacataaaga actttcctca ggataaccaa | 2340 |
| aaagtacaac tctcagtgag cggatttttg tgtgcatttg aaattcctgt cccttcatct | 2400 |
| ccaatttcag cttctagtcc aacacaaata gatttctcct cttcaccatc tacaaatgga | 2460 |
| atgttcaccc taactaccaa tggggaccta cccaaaccaa tattcatccc caatagaatg | 2520 |
| ccaaacactg ttgtgccatg tggaactgag aagaacttca caaatggaat ggttaatggt | 2580 |
| cacatgccat ctcttcctga cagccccttt acaggttaca tcattgcagt ccaccgaaaa | 2640 |
| atgatgagga cagaactgta tttcctgtca cctcaggaga atcgccccag cctctttgga | 2700 |
| atgccattga ttgttccatg cactgtgcat acccggaaga aagacctata tgatgcggtt | 2760 |
| tggattcaag tatcctggtt agcaagacca ctcccacctc aggaagctag tattcatgcc | 2820 |
| caggatcgtg ataactgtat gggctatcaa tatccattca ctctacgagt tgtgcagaaa | 2880 |
| gatgggaact cctgtgcttg gtgcccacag tatagatttt gcagaggctg taaaattgat | 2940 |
| tgtggggaag acagagcttt cattggaaat gcctatattg ctgtggattg gcaccccaca | 3000 |
| gcccttcacc ttcgctatca aacatcccag gaaagggttg tagataagca tgagagtgtg | 3060 |
| gagcagagtc ggcgagcgca agccgagccc atcaacctgg acagctgtct ccgtgctttc | 3120 |
| accagtgagg aagagctagg ggaaagtgag atgtactact gttccaagtg taagacccac | 3180 |
| tgcttagcaa caaagaagct ggatctctgg aggcttccac ccttcctgat tattcacctt | 3240 |
| aagcgatttc aatttgtaaa tgatcagtgg ataaaatcac agaaaattgt cagatttctt | 3300 |
| cgggaaagtt ttgatccgag tgcttttttg gtaccacgag acccggccct ctgccagcat | 3360 |
| aaaccactca caccccaggg ggatgagctc tccaagccca ggattctggc aagagaggtg | 3420 |
| aagaaagtgg atgcgcagag ttcggctgga aaagaggaca tgctcctaag caaaagccca | 3480 |
| tcctcactca gcgctaacat cagcagcagc ccaaaaggtt ctccttcttc atcaagaaaa | 3540 |
| agtggaacca gctgtccctc cagcaaaaac agcagcccta atagcagccc acggactttg | 3600 |
| gggaggagca aagggaggct ccggctgccc cagattggca gcaaaaataa gccgtcaagt | 3660 |
| agtaagaaga acttggatgc cagcaaagag aatggggctg ggcagatctg tgagctggct | 3720 |
| gacgccttga gccgagggca tatgcggggg ggcagccaac cagagctggt cactcctcag | 3780 |
| gaccatgagg tagctttggc caatggattc ctttatgagc atgaagcatg tggcaatggc | 3840 |
| tgtggcgatg gctacagcaa tggtcagctt ggaaaccaca gtgaagaaga cagcactgat | 3900 |
| gaccaaagag aagacactca tattaagcct atttataatc tatatgcaat ttcatgccat | 3960 |
| tcaggaattc tgagtggggg ccattacatc acttatgcca aaaacccaaa ctgcaagtgg | 4020 |
| tactgttata atgacagcag ctgtgaggaa cttcaccctg atgaaattga caccgactct | 4080 |
| gcctacattc ttttctatga gcagcagggg atagactacg cacaatttct gccaaagatt | 4140 |
| gatggcaaaa agatggcaga cacaagcagt acggatgaag actctgagtc tgattacgaa | 4200 |
| aagtactcta tgttacagta a | 4221 |

### Sequence Accession No.: NM_{—}001002799

### Gene: SMC4

| (SEQ ID NO: 100) | |
|---|---|
| atgccccgta aaggcaccca gccctccact gcccggcgca gagaggaagg gccgccgccg | 60 |
| ccgtcccctg acggcgccag cagcgacgcg gagcctgagc cgccgtccgg ccgcacggag | 120 |
| agcccagcca ccgccgcagc aatgaccaat gaagctggag ctcctcggct tatgataact | 180 |
| catattgtaa accagaactt caaatcctat gctggggaga aaattctggg acctttccat | 240 |
| aagcgctttt cctgtattat cgggccaaat ggcagtggca aatccaatgt tattgattct | 300 |
| atgctttttg tgtttggcta tcgagcacaa aaaataagat ctaaaaaact ctcagtatta | 360 |
| atacataatt ctgatgaaca caaggacatt cagagttgta cagtagaagt tcattttcaa | 420 |
| aagataattg ataaggaagg ggatgattat gaagtcattc ctaacagtaa tttctatgta | 480 |
| tccagaacgg cctgcagaga taatacttct gtctatcaca taagtggaaa gaaaaagaca | 540 |
| tttaaggatg ttggaaatct tcttcgaagc catggaattg acttggacca taatagattt | 600 |
| ttaattttac agggtgaagt tgaacaaatt gctatgatga aaccaaaagg ccagactgaa | 660 |
| cacgatgagg gtatgcttga atatttagaa gatataattg gttgtggacg gctaaatgaa | 720 |
| cctattaaag tcttgtgtcg gagagttgaa atattaaatg aacacagagg agagaagtta | 780 |
| aacagggtaa agatggtgga aaaggaaaag gatgccttag aaggagagaa aaacatagct | 840 |
| atcgaatttc ttaccttgga aaatgaaata tttagaaaaa agaatcatgt ttgtcaatat | 900 |
| tatatttatg agttgcagaa acgaattgct gaaatggaaa ctcaaaagga aaaaattcat | 960 |
| gaagatacca aagaaattaa tgagaagagc aatatactat caaatgaaat gaaagctaag | 1020 |
| aataaagatg taaaagatac agaaaagaaa ctgaataaaa ttacaaaatt tattgaggag | 1080 |
| aataaagaaa aatttacaca gctagatttg gaagatgttc aagttagaga aaagttaaaa | 1140 |
| catgccacga gtaaagccaa aaaactggag aaacaacttc aaaaagataa agaaaaggtt | 1200 |
| gaagaattta aaagtatacc tgccaagagt aacaatatca ttaatgaaac aacaaccaga | 1260 |
| aacaatgccc tcgagaagga aaaagagaaa gaagaaaaaa aattaaagga agttatggat | 1320 |
| agccttaaac aggaaacaca agggcttcag aaagaaaaag aaagtcgaga gaaagaactt | 1380 |
| atgggtttca gcaaatcggt aaatgaagca cgttcaaaga tggatgtagc ccagtcagaa | 1440 |
| cttgatatct atctcagtcg tcataatact gcagtgtctc aattaactaa ggctaaggaa | 1500 |
| gctctaattg cagcttctga gactctcaaa gaaaggaaag ctgcaatcag agatatagaa | 1560 |
| ggaaaactcc ctcaaactga acaagaatta aaggagaaag aaaaagaact tcaaaaactt | 1620 |
| acacaagaag aaacaaactt taaaagtttg gttcatgatc tctttcaaaa agttgaagaa | 1680 |
| gcaaagagct cattagcaat gaatcgaagt agggggaaag tccttgatgc aataattcaa | 1740 |
| gaaaaaaaat ctggcaggat tccaggaata tatggaagat tgggggactt aggagccatt | 1800 |
| gatgaaaaat acgacgtggc tatatcatcc tgttgtcatg cactggacta cattgttgtt | 1860 |
| gattctattg atatagccca agaatgtgta aacttcctta aaagacaaaa tattggagtt | 1920 |
| gcaaccttta taggtttaga taagatggct gtatgggcga aaaagatgac cgaaattcaa | 1980 |
| actcctgaaa atactcctcg tttatttgat ttagtaaaag taaaagatga gaaaattcgc | 2040 |
| caagcttttt attttgcttt acgagatacc ttagtagctg acaacttgga tcaagccaca | 2100 |
| agagtagcat atcaaaaaga tagaagatgg agagtggtaa ctttacaggg acaaatcata | 2160 |
| gaacagtcag gtacaatgac tggtggtgga agcaaagtaa tgaaaggaag aatgggttcc | 2220 |
| tcacttgtta ttgaaatctc tgaagaagag gtaaacaaaa tggaatcaca gttgcaaaac | 2280 |
| gactctaaaa aagcaatgca aatccaagaa cagaaagtac aacttgaaga aagagtagtt | 2340 |
| aagttacggc atagtgaacg agaaatgagg aacacactag aaaaatttac tgcaagcatc | 2400 |
| cagcgtttaa tagagcaaga agaatatttg aatgtccaag ttaaggaact tgaagctaat | 2460 |
| gtacttgcta cagcccctga caaaaaaaag cagaaattgc tagaagaaaa cgttagtgct | 2520 |
| ttcaaaacag aatatgatgc tgtggctgag aaagctggta aagtagaagc tgaggttaaa | 2580 |
| cgcttacaca ataccatcgt agaaatcaat aatcataaac tcaaggccca acaagacaaa | 2640 |
| cttgataaaa taaataagca attagatgaa tgtgcttctg ctattactaa agcccaagta | 2700 |
| gcaatcaaga ctgctgacag accttcaaaa ggcacaagac tctgtcttgc gtacagagaa | 2760 |
| agaaataaaa gatactga | 2778 |

### Sequence Accession No.: NM_{—}021643

### Gene: TRIB2

| (SEQ ID NO: 101) | |
|---|---|
| atgaacatac acaggtctac ccccatcaca atagcgagat atgggagatc gcggaacaaa | 60 |
| acccaggatt tcgaagagtt gtcgtctata aggtccgcgg agcccagcca gagtttcagc | 120 |
| ccgaacctcg gctccccgag cccgcccgag actccgaact tgtcgcattg cgtttcttgt | 180 |
| atcgggaaat acttattgtt ggaacctctg gagggagacc acgtttttcg tgccgtgcat | 240 |
| ctgcacagcg gagaggagct ggtgtgcaag gtgtttgata tcagctgcta ccaggaatcc | 300 |
| ctggcaccgt gcttttgcct gtctgctcat agtaacatca accaaatcac tgaaattatc | 360 |
| ctgggtgaga ccaaagccta tgtgttcttt gagcgaagct atggggacat gcattccttc | 420 |
| gtccgcacct gcaagaagct gagagaggag gaggcagcca gactgttcta ccagattgcc | 480 |
| tcggcagtgg cccactgcca tgacgggggg ctggtgctgc gggacctcaa gctgcggaaa | 540 |
| ttcatcttta aggacgaaga gaggactcgg gtcaagctgg aaagcctgga agacgcctac | 600 |
| attctgcggg gagatgatga ttccctctcc gacaagcatg gctgcccggc ttacgtaagc | 660 |
| ccagagatct tgaacaccag tggcagctac tcgggcaaag cagccgacgt gtggagcctg | 720 |
| ggggtgatgc tgtacaccat gttggtgggg cggtaccctt tccatgacat tgaacccagc | 780 |
| tccctcttca gcaagatccg gcgtggccag ttcaacattc cagagactct gtcgcccaag | 840 |
| gccaagtgcc tcatccgaag cattctgcgt cgggagccct cagagcggct gacctcgcag | 900 |
| gaaattctgg accatccttg gttttctaca gattttagcg tctcgaattc agcatatggt | 960 |
| gctaaggaag tgtctgacca gctggtgccg gacgtcaaca tggaagagaa cttggaccct | 1020 |
| ttctttaact ga | 1032 |

### Sequence Accession No.: NM_{—}007005

Gene: Homo sapiens transducin-like enhancer of split 4 (E(sp1) homolog, Drosophila) (TLE4)

| (SEQ ID NO: 102) | |
|---|---|
| atgattcgcg acctgagcaa gatgtacccg cagaccagac acccggcacc gcatcagcct | 60 |
| gctcaaccct ttaaatttac aatttccgaa tcctgtgatc ggattaagga agagtttcag | 120 |
| tttttacagg ctcaatacca cagtctgaag ctggaatgtg agaaactcgc cagtgagaag | 180 |
| acagagatgc agcggcatta tgtcatgtat tatgaaatgt cctatgggtt gaatatagaa | 240 |
| atgcacaagc aggcagagat tgtcaagagg ctgaatgcta tctgtgcaca agtcatccct | 30O |
| ttcctgtccc aagagcacca gcaacaagtg gtgcaggctg tggaacgggc caagcaggtg | 360 |
| accatggcag aactgaacgc catcattggg caacaactcc aggcccagca tttatcacat | 420 |
| ggacatggtc tccccgtacc tctgactcca cacccttcag ggctccagcc ccctgccatt | 480 |
| ccacccatcg gtagcagtgc cgggcttctg gccctctcca gtgctctagg aggtcagtcc | 540 |
| catcttccaa ttaaagatga gaagaagcac catgacaatg atcaccaaag agacagagac | 600 |
| tccatcaaga gctcttcagt atccccatca gccagtttcc gaggtgctga gaagcacaga | 660 |
| aactccgcag actactcctc agagagcaaa aagcagaaaa ctgaagaaaa ggaaattgca | 720 |
| gctcgttatg acagcgatgg tgagaaaagt gatgacaact tggtggttga cgtttccaat | 780 |
| gaggacccat cttcccctcg agggagccca gcacattccc ccagagagaa tggcctagac | 840 |
| aagacacgcc tgctcaagaa agatgccccg attagtccag cctctattgc atcttccagc | 900 |
| agtactccct cctccaaatc caaagaactt agccttaatg aaaaatctac tactcccgtc | 960 |
| tcaaagtcca atacccctac tccacgaact gatgcgccca ccccaggcag taactctact | 1020 |
| cccggattga ggcctgtacc tggaaaacca ccaggagttg accctttggc ctcaagccta | 1080 |
| aggaccccaa tggcagtacc ttgtccatat ccaactccat ttgggattgt gccccatgct | 1140 |
| ggaatgaacg gagagctgac cagccccgga gcggcctacg ctgggctcca caacatctcc | 1200 |
| cctcagatga gcgcagctgc tgccgccgcc gctgctgctg ccgcctacgg gagatcacca | 1260 |
| gtggtgggat ttgatccaca ccatcacatg cgtgtgccag caatacctcc aaacctgaca | 1320 |
| ggcattccag gaggaaaacc agcatactcc ttccatgtta gcgcagatgg tcagatgcag | 1380 |
| cctgtccctt ttccacccga cgccctcatc ggacctggaa tcccccggca tgctcgccag | 1440 |
| atcaacaccc tcaaccacgg ggaggtggtg tgcgcggtga ccatcagcaa ccccacgaga | 1500 |
| cacgtgtaca cgggtgggaa gggctgcgtc aaggtctggg acatcagcca cccaggcaat | 1560 |
| aagagtcctg tctcccagct cgactgtccg aacagggata actacatccg ttcctgcaga | 1620 |
| ttgctccctg atggtcgcac cctaattgtt ggaggggaag ccagtacttt gtccatttgg | 1680 |
| gacctggcgg ctccaacccc acgcatcaag gcagagctga catcctcggc ccccgcctgc | 1740 |
| tatgccctgg ccatcagccc cgattccaag gtctgcttct catgctgcag cgacggcaac | 1800 |
| atcgctgtgt gggatctgca caaccagacc ttggtgaggc aattccaggg ccacacagat | 1860 |
| ggagccagct gtattgacat ttctaatgat ggcaccaagc tctggacagg tggtttggac | 1920 |
| aacacggtca ggtcctggga cctgcgcgag gggcggcagc tgcagcagca cgacttcacc | 1980 |
| tcccagatct tttctctggg ctactgccca actggagagt ggcttgcagt ggggatggag | 2040 |
| aacagcaatg tggaagtttt gcatgtcacc aagccagaca aataccaact acatctccat | 2100 |
| gagagctgtg tgctgtcgct caagtttgcc cattgtggca aatggtttgt aagcactgga | 2160 |
| aaggacaacc ttctgaatgc ctggagaaca ccttatgggg ccagtatatt ccagtccaaa | 2220 |
| gaatcctcat cggtgcctag ctgtgacatc tccgtggacg acaaatacat tgtcactggc | 2280 |
| tctggggata agaaggccac agtttatgaa gttatttatt aa | 2322 |

### Sequence Accession No.: NM_{—}001719

Gene: Homo sapiens bone morphogenetic protein 7 (osteogenic protein 1) (BMP7)

| (SEQ ID NO: 103) | |
|---|---|
| atgcacgtgc gctcactgcg agctgcggcg ccgcacagct tcgtggcgct ctgggcaccc | 60 |
| ctgttcctgc tgcgctccgc cctggccgac ttcagcctgg acaacgaggt gcactcgagc | 120 |
| ttcatccacc ggcgcctccg cagccaggag cggcgggaga tgcagcgcga gatcctctcc | 180 |
| attttgggct tgccccaccg cccgcgcccg cacctccagg gcaagcacaa ctcggcaccc | 240 |
| atgttcatgc tggacccgta caacgccatg gcggtggagg agggcggcgg gcccggcggc | 300 |
| cagggcttct cctaccccta caaggccgtc ttcagcaccc agggcccccc tctggccagc | 360 |
| ctgcaagata gccatttcct caccgacgcc gacatggtca tgagcttcgt caacctcgtg | 420 |
| gaacatgaca aggaattctt ccacccacgc taccaccatc gagagttccg gtttgatctt | 480 |
| tccaagatcc cagaagggga agctgtcacg gcagccgaat tccggatcta caaggactac | 540 |
| atccgggaac gcttcgacaa tgagacgttc cggatcagcg tttatcaggt gctccaggag | 600 |
| cacttgggca gggaatcgga tctcttcctg ctcgacagcc gtaccctctg ggcctcggag | 660 |
| gagggctggc tggtgtttga catcacagcc accagcaacc actgggtggt caatccgcgg | 720 |
| cacaacctgg gcctgcagct ctcggtggag acgctggatg ggcagagcat caaccccaag | 780 |
| ttggcgggcc tgattgggcg gcacgggccc cagaacaagc agcccttcat ggtggctttc | 840 |
| ttcaaggcca cggaggtcca cttccgcagc atccggtcca cggggagcaa acagcgcagc | 900 |
| cagaaccgct ccaagacgcc caagaaccag gaagccctgc ggatggccaa cgtggcagag | 960 |
| aacagcagca gcgaccagag gcaggcctgt aagaagcacg agctgtatgt cagcttccga | 1020 |
| gacctgggct ggcaggactg gatcatcgcg cctgaaggct acgccgccta ctactgtgag | 1080 |
| ggggagtgtg ccttccctct gaactcctac atgaacgcca ccaaccacgc catcgtgcag | 1140 |
| acgctggtcc acttcatcaa cccggaaacg gtgcccaagc cctgctgtgc gcccacgcag | 1200 |
| ctcaatgcca tctccgtcct ctacttcgat gacagctcca acgtcatcct gaagaaatac | 1260 |
| agaaacatgg tggtccgggc ctgtggctgc cactag | 1296 |

### Sequence Accession No.: NM_{—}002588

Gene: Homo sapiens protocadherin gamma subfamily C, 3 (PCDHGC3)

| (SEQ ID NO: 104) | |
|---|---|
| atggtcccag aggcctggag gagcggactg gtaagcaccg ggagggtagt gggagttttg | 60 |
| cttctgcttg gtgccttgaa caaggcttcc acggtcattc actatgagat cccggaggaa | 120 |
| agagagaagg gtttcgctgt gggcaacgtg gccgcgaacc tcggtttgga tctcggtagc | 180 |
| ctctcagccc gcaggttccg ggtggtgtct ggagctagcc gaagattctt tgaggtgaac | 240 |
| cgggagaccg gagagatgtt tgtgaacgac cgtctggatc gagaggagct gtgtgggaca | 300 |
| ctgccctctt gcactgtaac tctggagttg gtagtggaga acccgctgga gctgctcagc | 360 |
| gtggaagtgg tgatccagga catcaacgac aacaatcctg ctttccctac ccaggaaatg | 420 |
| aaattggaga ttagcgaggc cgtggctccg gggacgcgct ttccgctcga gagcgcgcac | 480 |
| gatcccgatg tgggaagcaa ctctttacaa acctatgagc tgagccgaaa cgaatacttt | 540 |
| gcgcctcgcg cgcagacgcg ggaggacagc accaagcacg cggagctggt gttggagcgc | 600 |
| gccctggacc gagaacggga gcctagtctc cagttagtgc tgacggcgtt ggacggaggg | 660 |
| accccagctc tctccgccag cctgcctatt cacatcaagg tgctggacgc gaatgacaat | 720 |
| gcgcctgtct tcaaccagtc cttgtaccgg gcgcgcgtcc tggaggatgc accctccggc | 780 |
| acgcgcgtgg tacaagtcct tgcaacggat ctggatgaag gccccaacgg tgaaattatt | 840 |
| tactccttcg gcagccacaa ccgcgccggc gtgcggcaac tattcgcctt agaccttgta | 900 |
| accgggatgc tgacaatcaa gggtcggctg gacttcgagg acaccaaact ccatgagatt | 960 |
| tacatccagg ccaaagacaa gggcgccaat cccgaaggag cacattgcaa agtgttggtg | 1020 |
| gaggttgtgg atgtgaatga caacgccccg gagatcacag tcacctccgt gtacagccca | 1080 |
| gtacccgagg atgcccctct ggggactgtc atcgctttgc tcagtgtgac tgacctggat | 1140 |
| gctggcgaga acgggctggt gacctgcgaa gttccaccgg gtctcccttt cagccttact | 1200 |
| tcttccctca agaattactt cactttgaaa accagtgcag acctggatcg ggagactgtg | 1260 |
| ccagaataca acctcagcat caccgcccga gacgccggaa ccccttccct ctcagccctt | 1320 |
| acaatagtgc gtgttcaagt gtccgacatc aatgacaacc ccccacaatc ttctcaatcc | 1380 |
| tcctacgacg tttacattga agaaaacaac ctccccgggg ctccaatact aaacctaagt | 1440 |
| gtctgggacc ccgacgcccc gcagaatgct cggctttctt tctttctctt ggagcaagga | 1500 |
| gctgaaaccg ggctagtggg tcgctatttc acaataaatc gtgacaatgg catagtgtca | 1560 |
| tccttagtgc ccctagacta tgaggatcgg cgggaatttg aattaacagc tcatatcagc | 1620 |
| gatgggggca ccccggtcct agccaccaac atcagcgtga acatatttgt cactgatcgc | 1680 |
| aatgacaatg ccccccaggt cctatatcct cggccaggtg ggagctcggt ggagatgctg | 1740 |
| cctcgaggta cctcagctgg ccacctagtg tcacgggtgg taggctggga cgcggatgca | 1800 |
| gggcacaatg cctggctctc ctacagtctc ttgggatccc ctaaccagag cctttttgcc | 1860 |
| atagggctgc acactggtca aatcagtact gcccgtccag tccaagacac agattcaccc | 1920 |
| aggcagactc tcacggtctt gatcaaagac aatggggagc cttcgctctc caccactgct | 1980 |
| accctcactg tgtcagtaac cgaggactct cctgaagccc gagccgagtt cccctctggc | 2040 |
| tctgcccccc gggagcagaa aaaaaatctc accttttacc tacttctttc tctaatcctg | 2100 |
| gtttctgtgg ggtttgtggt cacagtgttc ggagtaatca tattcaaagc ttacaagtgg | 2160 |
| aagcagccta gagacctata ccgagccccg gtgagctcac tgtaccgaac accagggccc | 2220 |
| tccttgcacg cggacgccgt gcggggaggc ctgatgtcgc cgcaccttta ccatcaggtg | 2280 |
| tatctcacca cggactcccg ccgcagcgac ccgctgctga agaaacctgg tgcagccagt | 2340 |
| ccactggcca gccgccagaa cacgctgcgg agctgtgatc cggtgttcta taggcaggtg | 2400 |
| ttgggtgcag agagcgcccc tcccggacag caagccccgc ccaacacgga ctggcgtttc | 2460 |
| tctcaggccc agagacccgg caccagcggc tcccaaaatg gcgatgacac cggcacctgg | 2520 |
| cccaacaacc agtttgacac agagatgctg caagccatga tcttggcgtc cgccagtgaa | 2580 |
| gctgctgatg ggagctccac cctgggaggg ggtgccggca ccatgggatt gagcgcccgc | 2640 |
| tacggacccc agttcaccct gcagcacgtg cccgactacc gccagaatgt ctacatccca | 2700 |
| ggcagcaatg ccacactgac caacgcagct ggcaagcggg atggcaaggc cccagcaggt | 2760 |
| ggcaatggca acaagaagaa gtcgggcaag aaggagaaga agtaa | 2805 |

### Sequence Accession No.: NM_{—}015570

Gene: Homo sapiens autism susceptibility candidate 2 (AUTS2)

| (SEQ ID NO: 105) | |
|---|---|
| atggatggcc cgacgcgggg ccatggactc cgcaaaaagc ggcggtcgcg gtcgcagcga | 60 |
| gaccgggaga ggcgctcccg gggcgggctg ggggccggcg cggccggcgg cggcggggct | 120 |
| ggccggaccc gggcgctctc actcgcctcg tcgtcgggct ccgacaagga agacaatggg | 180 |
| aagcccccgt cctccgcccc gtcccggccc agacccccgc ggaggaagcg gagagagtcc | 240 |
| acctcggcag aagaggacat cattgatgga tttgccatga ccagctttgt cacttttgaa | 300 |
| gcgctggaga aagatgtagc acttaagcct caggaacgtg tggagaaacg ccagacgccc | 360 |
| ctgaccaaga agaaacgaga agcacttacc aatggcttgt cctttcattc aaagaagagc | 420 |
| agactcagcc acccacacca ctacagctca gatcgagaaa atgaccgcaa tctctgccag | 480 |
| caccttggga agagaaagaa aatgccgaag gcactcagac agctcaagcc aggacagaac | 540 |
| agctgcaggg acagtgacag tgaaagtgcc agtggagaat ccaagggctt ccaccggagc | 600 |
| agctctcggg aaaggctcag tgatagttca gctccttcca gcttgggaac aggctacttc | 660 |
| tgtgacagtg acagtgacca ggaagagaag gcatcagatg ccagctctga aaaactcttc | 720 |
| aacactgtta ttgtaaacaa agatccggag ttaggtgttg gcacgctacc agaacatgac | 780 |
| agccaggatg cagggccgat tgtccccaag atatcgggtc tagagagaag ccaggagaag | 840 |
| agccaggact gttgcaaaga gccaatcttt gagcctgtgg tgcttaaaga cccctgccct | 900 |
| caggtcgcac agccaatacc ccagccgcag acggagcccc aactccgagc tccttctccg | 960 |
| gaccctgact tggtgcagcg cacagaggcc ccacctcaac ccccacctct gagtacacag | 1020 |
| ccaccacagg gccctcctga ggcccagctc cagcctgccc cgcagcctca ggtgcagagg | 1080 |
| ccacccaggc cacagtcccc cacccagctg ctccatcaga acctcccacc tgtgcaggcc | 1140 |
| cacccctctg ctcagagcct ctcccagcca ttgtcagcct acaacagcag tagcttaagc | 1200 |
| ctcaacagtt taagcagcag cagaagcagc actccagcga agactcagcc cgccccacct | 1260 |
| cacatctccc accacccctc cgcctccccg ttccccctct ccctgcccaa ccacagcccc | 1320 |
| ctgcacagct tcacacccac cctccagccc cccgcacact cacatcaccc caatatgttt | 1380 |
| gcccctccca ctgctctgcc tcctccacca ccactgacat caggaagtct gcaggtggcc | 1440 |
| ggacacccgg ccgggagcac ttactcagag caagacatct tgcgacagga actgaacact | 1500 |
| cgttttttgg cctctcagag tgctgaccgc ggggcttccc tgggccctcc gccctacctg | 1560 |
| cggaccgagt tccatcagca ccagcaccag caccagcaca cccaccagca cacgcaccag | 1620 |
| cacaccttca cgccgttccc ccacgccatc ccacccaccg ccatcatgcc gacgccagca | 1680 |
| cctcccatgt ctgacaaata ccctacaaaa gttgacccat tctaccggca cagtctcttc | 1740 |
| cattcctatc ctcctgcagt gtcgggcatc ccccctatga tcccacccac tggccctttt | 1800 |
| ggttcactac aaggagcatt tcagccgaag acatccaacc ctatcgatgt cgctgctcgg | 1860 |
| cctgggacag tcccacacac tttactccaa aaggacccga ggttgacaga tcctttcaga | 1920 |
| cctatgttaa ggaaaccagg gaagtggtgt gctatgcatg ttcacatcgc ctggcagatt | 1980 |
| taccaccacc aacagaaagt caagaaacag atgcagtcag acccacataa gctggacttt | 2040 |
| ggactgaaac ctgagttcct gagccgccct ccaggcccca gtctttttgg agccatccac | 2100 |
| cacccccatg acctggcacg gccttcaact ttgttctctg ccgctggtgc tgcacaccca | 2160 |
| actgggaccc cttttgggcc acctcctcat cacagcaact tcctcaaccc tgctgcccac | 2220 |
| ctagagcctt ttaatcggcc gtctacattc acaggcctag cagcagttgg tggcaatgcc | 2280 |
| ttcgggggac ttggaaatcc ttccgttaca cccaactcaa tgttcggcca caaggacggc | 2340 |
| cccagtgtgc agaactttag caaccctcac gaaccctgga accggctgca ccgaacgcct | 2400 |
| ccgtcgttcc cgacccctcc gccctggctg aagccagggg agctggagcg cagcgcgtcc | 2460 |
| gctgcagctc atgacagaga tagagatgta gataaacgag actcatctgt tagtaaagat | 2520 |
| gacaaagaaa gggaaagcgt cgagaagaga cactccagcc acccttcacc agcacctgtc | 2580 |
| ctcccggtga atgccctggg acatacccgc agctccactg aacagatccg ggctcacctg | 2640 |
| aacactgagg ctcgggagaa ggacaaaccc aaagagaggg agagagacca ctcggaatcc | 2700 |
| cgcaaggacc tggccgccga cgagcacaag gcgaaagagg gccacctgcc cgagaaggac | 2760 |
| gggcacggcc acgaggggcg cgccgcgggc gaagaggcca agcagctggc ccgggtgccg | 2820 |
| tctccctacg cgcggacccc ggtggtggag agtgccaggc ccaacagcac ctcgagccgg | 2880 |
| gaggccgagc cgcgcaaggg tgagccggcc tacgagaacc ccaagaagag ctccgaggtc | 2940 |
| aaggtgaagg aggagcggaa ggaagaccat gacctgcctc cagaggcccc gcagacccac | 3000 |
| cgggcctcgg agccgccgcc tcccaactcc tcgtccagcg tgcacccggg gcccctggcc | 3060 |
| tcgatgccca tgacggtggg ggtgacgggc attcacccca tgaacagcat cagcagcctg | 3120 |
| gacaggactc gcatgatgac ccccttcatg ggcatcagcc ccctcccggg cggagagcgc | 3180 |
| ttcccgtacc cttctttcca ctgggacccc atccgggacc ccttgaggga tccttaccga | 3240 |
| gaacttgaca ttcaccggag agacccgctg ggcagggact tcctgctaag gaacgacccg | 3300 |
| ctccaccggc tctcgactcc ccggctgtac gaagccgacc gctccttcag ggaccgggag | 3360 |
| cctcacgact acagccacca ccaccaccac caccaccacc cgctgtctgt ggaccctcgg | 3420 |
| cgggagcacg agcggggagg ccacctggac gagcgggagc gcttgcacat gctcagagaa | 3480 |
| gactacgagc acacgcggct ccactccgtg caccccgcct ccctcgacgg acacctcccc | 3540 |
| caccccagcc tcatcacccc gggactcccc agcatgcact atccccgcat cagccccacc | 3600 |
| gcgggcaacc agaacggact cctcaacaag acccctccga cagcagcgct gagcgcacct | 3660 |
| cccccgctca tctccacgct ggggggccgc ccggtctctc ccagaaggac gactcctctg | 3720 |
| tccgcagaga taagggagag gcccccttcc cacacgctga aggatatcga ggcccgataa | 3780 |

### Sequence Accession No.: NM_{—}020215

Gene: Homo sapiens chromosome 14 open reading frame 132 (C14orf132)

| (SEQ ID NO: 106) | |
|---|---|
| atgatggcgc agctcttcct actccaagcc aacgctgtcc ttcccctttc ccatgaaatc | 60 |
| aaggtcaaga ggcaaataag actccctgct ccactctacc ccccagagag aaatgattct | 120 |
| cgctcctttc agatccccca ggatctgagg gagaaaggat gggaggaggg gcagcagcat | 180 |
| ttcgctggaa aggcagcaga tgcttttcca gccccggttc agctggaagg cttggaggcc | 240 |
| ggccagacca ctctggcgtc tcctgaagtg ggtccctgga gaccgaagag gctcagtgga | 300 |
| gtctgtctgt tgtcagcact gctgcctgat ccctgcaaga caaatggcac tttccttctt | 360 |
| cagaagcatc atctgccttc attattagca gtaatattat tcccagttat tattcttacc | 420 |
| ggtgccagtt ttgcacatct ttttgttgct ctatttgtgt ctcatttact tctcaaattg | 480 |
| cccctggggg caggaatgag gatgcagaga gatgcacgtt aa | 522 |

### Sequence Accession No.: NM_{—}022766

Gene: Homo sapiens ceramide kinase (CERK)

| (SEQ ID NO: 107) | |
|---|---|
| atgggggcga cgggggcggc ggagccgctg caatccgtgc tgtgggtgaa gcagcagcgc | 60 |
| tgcgccgtga gcctggagcc cgcgcgggct ctgctgcgct ggtggcggag cccggggccc | 120 |
| ggagccggcg cccccggcgc ggatgcctgc tctgtgcctg tatctgagat catcgccgtt | 180 |
| gaggaaacag acgttcacgg gaaacatcaa ggcagtggaa aatggcagaa aatggaaaag | 240 |
| ccttacgctt ttacagttca ctgtgtaaag agagcacgac ggcaccgctg gaagtgggcg | 300 |
| caggtgactt tctggtgtcc agaggagcag ctgtgtcact tgtggctgca gaccctgcgg | 360 |
| gagatgctgg agaagctgac gtccagacca aagcatttac tggtatttat caacccgttt | 420 |
| ggaggaaaag gacaaggcaa gcggatatat gaaagaaaag tggcaccact gttcacctta | 480 |
| gcctccatca ccactgacat catcgttact gaacatgcta atcaggccaa ggagactctg | 540 |
| tatgagatta acatagacaa atacgacggc atcgtctgtg tcggcggaga tggtatgttc | 600 |
| agcgaggtgc tgcacggtct gattgggagg acgcagagga gcgccggggt cgaccagaac | 660 |
| cacccccggg ctgtgctggt ccccagtagc ctccggattg gaatcattcc cgcagggtca | 720 |
| acggactgcg tgtgttactc caccgtgggc accagcgacg cagaaacctc ggcgctgcat | 780 |
| atcgttgttg gggactcgct ggccatggat gtgtcctcag tccaccacaa cagcacactc | 840 |
| cttcgctact ccgtgtccct gctgggctac ggcttctacg gggacatcat caaggacagt | 900 |
| gagaagaaac ggtggttggg tcttgccaga tacgactttt caggtttaaa gaccttcctc | 960 |
| tcccaccact gctatgaagg gacagtgtcc ttcctccctg cacaacacac ggtgggatct | 1020 |
| ccaagggata ggaagccctg ccgggcagga tgctttgttt gcaggcaaag caagcagcag | 1080 |
| ctggaggagg agcagaagaa agcactgtat ggtttggaag ctgcggagga cgtggaggag | 1140 |
| tggcaagtcg tctgtgggaa gtttctggcc atcaatgcca caaacatgtc ctgtgcttgt | 1200 |
| cgccggagcc ccaggggcct ctccccggct gcccacttgg gagacgggtc tcctgacctc | 1260 |
| atcctcatcc ggaaatgctc caggttcaat tttctgagat ttctcatcag gcacaccaac | 1320 |
| cagcaggacc agtttgactt cacttttgtt gaagttcatc gcgtcaagaa attccagttt | 1380 |
| acgtcgaagc acatggagga tgaggacagc gacctcaagg agggggggaa gaagcgcttt | 1440 |
| gggcacattt gcagcagcca cccctcctgc tgctgcaccg tctccaacag ctcctggaac | 1500 |
| tgcgacgggg aggtcctgca cagccctgcc atcgaggtca gagtccactg ccagctggtt | 1560 |
| cgactctttg cacgaggaat tgaagagaat ccgaagccag actcacacag ctga | 1614 |

### Sequence Accession No.: NM_{—}016073

Gene: Homo sapiens hepatoma-derived growth factor, related protein 3 (HDGFRP3)

| (SEQ ID NO: 108) | |
|---|---|
| atggcgcgtc cgcggccccg cgagtacaaa gcgggcgacc tggtcttcgc caagatgaag | 60 |
| ggctacccgc actggccggc ccggattgat gaactcccag agggcgctgt gaagcctcca | 120 |
| gcaaacaagt atcctatctt cttttttggc acccatgaaa ctgcatttct aggtcccaaa | 180 |
| gacctttttc catataagga gtacaaagac aagtttggaa agtcaaacaa acggaaagga | 240 |
| tttaacgaag gattgtggga aatagaaaat aacccaggag taaagtttac tggctaccag | 300 |
| gcaattcagc aacagagctc ttcagaaact gagggagaag gtggaaatac tgcagatgca | 360 |
| agcagtgagg aagaaggtga tagagtagaa gaagatggaa aaggcaaaag aaagaatgaa | 420 |
| aaagcaggct caaaacggaa aaagtcatat acttcaaaga aatcctctaa acagtcccgg | 480 |
| aaatctccag gagatgaaga tgacaaagac tgcaaagaag aggaaaacaa aagcagctct | 540 |
| gagggtggag atgcgggcaa cgacacaaga aacacaactt cagacttgca gaaaaccagt | 600 |
| gaagggacct aa | 612 |

### Sequence Accession No.: NM_{—}003199

Gene: Homo sapiens transcription factor 4 (TCF4)

| (SEQ ID NO: 109) | |
|---|---|
| atgcatcacc aacagcgaat ggctgcctta gggacggaca aagagctgag tgatttactg | 60 |
| gatttcagtg cgatgttttc acctcctgtg agcagtggga aaaatggacc aacttctttg | 120 |
| gcaagtggac attttactgg ctcaaatgta gaagacagaa gtagctcagg gtcctggggg | 180 |
| aatggaggac atccaagccc gtccaggaac tatggagatg ggactcccta tgaccacatg | 240 |
| accagcaggg accttgggtc acatgacaat ctctctccac cttttgtcaa ttccagaata | 300 |
| caaagtaaaa cagaaagggg ctcatactca tcttatggga gagaatcaaa cttacagggt | 360 |
| tgccaccagc agagtctcct tggaggtgac atggatatgg gcaacccagg aaccctttcg | 420 |
| cccaccaaac ctggttccca gtactatcag tattctagca ataatccccg aaggaggcct | 480 |
| cttcacagta gtgccatgga ggtacagaca aagaaagttc gaaaagttcc tccaggtttg | 540 |
| ccatcttcag tctatgctcc atcagcaagc actgccgact acaataggga ctcgccaggc | 600 |
| tatccttcct ccaaaccagc aaccagcact ttccctagct ccttcttcat gcaagatggc | 660 |
| catcacagca gtgacccttg gagctcctcc agtgggatga atcagcctgg ctatgcagga | 720 |
| atgttgggca actcttctca tattccacag tccagcagct actgtagcct gcatccacat | 780 |
| gaacgtttga gctatccatc acactcctca gcagacatca attccagtct tcctccgatg | 840 |
| tccactttcc atcgtagtgg tacaaaccat tacagcacct cttcctgtac gcctcctgcc | 900 |
| aacgggacag acagtataat ggcaaataga ggaagcgggg cagccggcag ctcccagact | 960 |
| ggagatgctc tggggaaagc acttgcttcg atctattctc cagatcacac taacaacagc | 1020 |
| ttttcatcaa acccttcaac tcctgttggc tctcctccat ctctctcagc aggcacagct | 1080 |
| gtttggtcta gaaatggagg acaggcctca tcgtctccta attatgaagg acccttacac | 1140 |
| tctttgcaaa gccgaattga agatcgttta gaaagactgg atgatgctat tcatgttctc | 1200 |
| cggaaccatg cagtgggccc atccacagct atgcctggtg gtcatgggga catgcatgga | 1260 |
| atcattggac cttctcataa tggagccatg ggtggtctgg gctcagggta tggaaccggc | 1320 |
| cttctttcag ccaacagaca ttcactcatg gtggggaccc atcgtgaaga tggcgtggcc | 1380 |
| ctgagaggca gccattctct tctgccaaac caggttccgg ttccacagct tcctgtccag | 1440 |
| tctgcgactt cccctgacct gaacccaccc caggaccctt acagaggcat gccaccagga | 1500 |
| ctacaggggc agagtgtctc ctctggcagc tctgagatca aatccgatga cgagggtgat | 1560 |
| gagaacctgc aagacacgaa atcttcggag gacaagaaat tagatgacga caagaaggat | 1620 |
| atcaaatcaa ttactagcaa taatgacgat gaggacctga caccagagca gaaggcagag | 1680 |
| cgtgagaagg agcggaggat ggccaacaat gcccgagagc gtctgcgggt ccgtgacatc | 1740 |
| aacgaggctt tcaaagagct cggccgcatg gtgcagctcc acctcaagag tgacaagccc | 1800 |
| cagaccaagc tcctgatcct ccaccaggcg gtggccgtca tcctcagtct ggagcagcaa | 1860 |
| gtccgagaaa ggaatctgaa tccgaaagct gcgtgtctga aaagaaggga ggaagagaag | 1920 |
| gtgtcctcag agcctccccc tctctccttg gccggcccac accctggaat gggagacgca | 1980 |
| tcgaatcaca tgggacagat gtaa | 2004 |

### Sequence Accession No.: NM_{—}002399

Gene: Homo sapiens Meis homeobox 2 (MEIS2)

| (SEQ ID NO: 110) | |
|---|---|
| atggacggag taggggttcc cgcttccatg tacggagacc ctcacgcgcc gcggccgatc | 60 |
| cccccggttc accacctgaa ccacgggccg ccgctccacg ccacacagca ctacggcgcg | 120 |
| cacgccccgc accccaatgt catgccggcc agtatgggat ccgctgtcaa cgacgccttg | 180 |
| aagcgggaca aggacgcgat ctatgggcac ccgttgtttc ctctgttagc tctggtcttt | 240 |
| gagaagtgcg agctggcgac ctgcactccc cgggaacctg gagtggctgg cggagacgtc | 300 |
| tgctcctccg actccttcaa cgaggacatc gcggtcttcg ccaagcaggt tcgcgccgaa | 360 |
| aagccacttt tttcctcaaa tccagagctg gacaatttga tgatacaagc aatacaagta | 420 |
| ctaaggtttc atcttttgga gttagaaaag gtccacgaac tgtgcgataa cttctgccac | 480 |
| cgatacatta gctgtttgaa ggggaaaatg cccatcgacc tcgtcattga tgaaagagac | 540 |
| ggcagctcca agtcagatca tgaagaactt tcaggctcct ccacaaatct cgctgaccat | 600 |
| aacccttctt cttggcgaga ccacgatgat gcaacctcaa cccactcagc aggcacccca | 660 |
| gggccctcca gtgggggcca tgcttcccag agcggagaca acagcagtga gcaaggggat | 720 |
| ggtttagaca acagtgtagc ttcacctggt acaggtgacg atgatgatcc ggataaggac | 780 |
| aaaaaacgcc agaagaaaag aggcattttc cccaaagtag caacaaatat catgagagca | 840 |
| tggctcttcc agcatctcac acatccgtac ccttccgaag agcagaagaa acagttagcg | 900 |
| caagacacag gacttacaat tctccaagta aacaactggt ttattaatgc cagaagaaga | 960 |
| atagtacagc ccatgattga ccagtcaaat cgagcagtga gccaaggagc agcatatagt | 1020 |
| ccagagggtc agcccatggg gagctttgtg ttggatggtc agcaacacat ggggatccgg | 1080 |
| cctgcaggac ctatgagtgg aatgggcatg aatatgggca tggatgggca atggcactac | 1140 |
| atgtaa | 1146 |

### Sequence Accession No.: NM_{—}019063

Gene: Homo sapiens echinoderm microtubule associated protein like 4 (EML4)

| (SEC) ID NO: 111) | |
|---|---|
| atggacggtt tcgccggcag tctcgatgat agtatttctg ctgcaagtac ttctgatgtt | 60 |
| caagatcgcc tgtcagctct tgagtcacga gttcagcaac aagaagatga aatcactgtg | 120 |
| ctaaaggcgg ctttggctga tgttttgagg cgtcttgcaa tctctgaaga tcatgtggcc | 180 |
| tcagtgaaaa aatcagtctc aagtaaaggc caaccaagcc ctcgagcagt tattcccatg | 240 |
| tcctgtataa ccaatggaag tggtgcaaac agaaaaccaa gtcataccag tgctgtctca | 300 |
| attgcaggaa aagaaactct ttcatctgct gctaaaagtg gtacagaaaa aaagaaagaa | 360 |
| aaaccacaag gacagagaga aaaaaaagag gaatctcatt ctaatgatca aagtccacaa | 420 |
| attcgagcat caccttctcc ccagccctct tcacaacctc tccaaataca cagacaaact | 480 |
| ccagaaagca agaatgctac tcccaccaaa agcataaaac gaccatcacc agctgaaaag | 540 |
| tcacataatt cttgggaaaa ttcagatgat agccgtaata aattgtcgaa aataccttca | 600 |
| acacccaaat taataccaaa agttaccaaa actgcagaca agcataaaga tgtcatcatc | 660 |
| aaccaagaag gagaatatat taaaatgttt atgcgcggtc ggccaattac catgttcatt | 720 |
| ccttccgatg ttgacaacta tgatgacatc agaacggaac tgcctcctga gaagctcaaa | 780 |
| ctggagtggg catatggtta tcgaggaaag gactgtagag ctaatgttta ccttcttccg | 840 |
| accggggaaa tagtttattt cattgcatca gtagtagtac tatttaatta tgaggagaga | 900 |
| actcagcgac actacctggg ccatacagac tgtgtgaaat gccttgctat acatcctgac | 960 |
| aaaattagga ttgcaactgg acagatagct ggcgtggata aagatggaag gcctctacaa | 1020 |
| ccccacgtca gagtgtggga ttctgttact ctatccacac tgcagattat tggacttggc | 1080 |
| acttttgagc gtggagtagg atgcctggat ttttcaaaag cagattcagg tgttcattta | 1140 |
| tgtgttattg atgactccaa tgagcatatg cttactgtat gggactggca gaagaaagca | 1200 |
| aaaggagcag aaataaagac aacaaatgaa gttgttttgg ctgtggagtt tcacccaaca | 1260 |
| gatgcaaata ccataattac atgcggtaaa tctcatattt tcttctggac ctggagcggc | 1320 |
| aattcactaa caagaaaaca gggaattttt gggaaatatg aaaagccaaa atttgtgcag | 1380 |
| tgtttagcat tcttggggaa tggagatgtt cttactggag actcaggtgg agtcatgctt | 1440 |
| atatggagca aaactactgt agagcccaca cctgggaaag gacctaaagg tgtatatcaa | 1500 |
| atcagcaaac aaatcaaagc tcatgatggc agtgtgttca cactttgtca gatgagaaat | 1560 |
| gggatgttat taactggagg agggaaagac agaaaaataa ttctgtggga tcatgatctg | 1620 |
| aatcctgaaa gagaaataga ggttcctgat cagtatggca caatcagagc tgtagcagaa | 1680 |
| ggaaaggcag atcaattttt agtaggcaca tcacgaaact ttattttacg aggaacattt | 1740 |
| aatgatggct tccaaataga agtacagggt catacagatg agctttgggg tcttgccaca | 1800 |
| catcccttca aagatttgct cttgacatgt gctcaggaca ggcaggtgtg cctgtggaac | 1860 |
| tcaatggaac acaggctgga atggaccagg ctggtagatg aaccaggaca ctgtgcagat | 1920 |
| tttcatccaa gtggcacagt ggtggccata ggaacgcact caggcaggtg gtttgttctg | 1980 |
| gatgcagaaa ccagagatct agtttctatc cacacagacg ggaatgaaca gctctctgtg | 2040 |
| atgcgctact caatagatgg taccttcctg gctgtaggat ctcatgacaa ctttatttac | 2100 |
| ctctatgtag tctctgaaaa tggaagaaaa tatagcagat atggaaggtg cactggacat | 2160 |
| tccagctaca tcacacacct tgactggtcc ccagacaaca agtatataat gtctaactcg | 2220 |
| ggagactatg aaatattgta ctgggacatt ccaaatggct gcaaactaat caggaatcga | 2280 |
| tcggattgta aggacattga ttggacgaca tatacctgtg tgctaggatt tcaagtattt | 2340 |
| ggtgtctggc cagaaggatc tgatgggaca gatatcaatg cactggtgcg atcccacaat | 2400 |
| agaaaggtga tagctgttgc cgatgacttt tgtaaagtcc atctgtttca gtatccctgc | 2460 |
| tccaaagcaa aggctcccag tcacaagtac agtgcccaca gcagccatgt caccaatgtc | 2520 |
| agttttactc acaatgacag tcacctgata tcaactggtg gaaaagacat gagcatcatt | 2580 |
| cagtggaaac ttgtggaaaa gttatctttg cctcagaatg agactgtagc ggatactact | 2640 |
| ctaaccaaag cccccgtctc ttccactgaa agtgtcatcc aatctaatac tcccacaccg | 2700 |
| cctccttctc agcccttaaa tgagacagct gaagaggaaa gtagaataag cagttctccc | 2760 |
| acacttctgg agaacagcct ggaacaaact gtggagccaa gtgaagacca cagcgaggag | 2820 |
| gagagtgaag agggcagcgg agaccttggt gagcctcttt atgaagagcc atgcaacgag | 2880 |
| ataagcaagg agcaggccaa agccaccctt ctggaggacc agcaagaccc ttcgccctcg | 2940 |
| tcctaa | 2946 |

### Sequence Accession No.: NM_{—}152793

Gene: Homo sapiens chromosome 7 open reading frame 41 (C7orf41; ELLS1)

| (SEQ ID NO: 112) | |
|---|---|
| atggatttcc agcagctggc cgacgttgcg gagaaatggt gctccaacac gcccttcgag | 60 |
| ctcatcgcca ccgaggagac cgaacgcagg atggatttct acgccgaccc cggcgtctcc | 120 |
| ttctatgtgc tgtgtccgga caacggctgc ggcgacaatt ttcacgtgtg gagtgagagc | 180 |
| gaggactgcc tgcctttctt gcagctagca caggattaca tctcctcctg cggcaagaag | 240 |
| acgctccacg aagtcctgga aaaagtcttc aagtctttca gacctttact ggggcttccg | 300 |
| gatgcagatg acgatgcgtt tgaagagtac agtgctgacg tggaagaaga ggagccagag | 360 |
| gcggaccacc cccagatggg ggtcagccag cagtaa | 396 |

### Sequence Accession No.: AB040883

Gene: Homo sapiens mRNA for KIAA1450 protein

```
(SEQ ID NO: 113)
      1 gcggccgccg cccccggctg cgcgctgagc cgccggcccc ccgagcgcca cggccggagc
    61 tgcggcggcg gcatcatggc cccgaccctg ctccagaagc tcttcaacaa aaggggcagc
  121 agcggcagct ccgcggcggc gtctgcccag ggcagggctc ctaaggaagg acccgccttt
  181 agttggtcat gttcggagtt tgacctgaat gagattcgcc tgatagttta ccaggactgt
  241 gacaggagag gcagacaagt cttgtttgac tctaaagctg ttcaaaagat tgaggaggtg
  301 acagctcaga aaacagagga tgttcctatt aaaatatcag ccaagtgctg ccagggaagc
  361 agcagtgtca gcagcagtag cagcagcagc atctcttccc acagttcttc tgggggatct
  421 tcacatcatg ctaaggaaca gcttccaaag taccagtaca caagaccagc ttccgatgtc
  481 aacatgttag gggaaatgat gtttggctca gttgccatga gttacaaagg ctccacctta
  541 aagatacact acatacgttc tcctccacaa ctgatgatta gtaaagtctt ctctgctaga
  601 atgggcagct tctgtggaag tacaaataac ttgcaagaca gctttgagta catcaaccaa
  661 gatcctaatt tgggaaaact gaacacaaat caaaatagtt tgggtccttg tcgtactgga
  721 agtaacctag cacacagcac accagttgat atgccaagca gaggacagaa tgaagacagg
  781 gacagtggca ttgctcgatc agcctcacta agcagtcttt tgatcacacc tttcccatct
  841 ccaagctcct ctacatcttc ttccagcagt taccagcgcc gctggcttcg aagtcagaca
  901 acaagtttgg aaaatggcat catcccaaga aggtcaactg atgagacatt cagcttggct
  961 gaagaaacct gtagctctaa tccagctatg gttaggagga agaaaattgc cataagcatc
1021 atcttttccc tatgtgagaa agaagaagca caaaggaatt tccaggactt cttcttttct
1081 cattttcccc tgtttgaatc tcacatgaac aggctgaaga gtgcaattga aaaggctatg
1141 atctcctgta ggaaaatagc agaatcaagt ctccgagtcc agttttatgt cagccgtttg
1201 atggaagctc tgggagaatt cagaggaact atctggaact tatattctgt tccaaggata
1261 gctgaacctg tatggcttac tatgatgtcc ggcactttgg aaaaaaacca gctctgccag
1321 cgctttctca aggagtttac acttctgata gaacagataa ataaaaacca gttttttgct
1381 gccttactga ctgcggtgtt aacctaccac ctggcctggg tcccaactgt catgcctgtg
1441 gatcaccctc ccatcaaagc cttctcagag aaacgtacct cccagtcagt gaacatgctg
1501 gccaaaacac atccgtataa tcctctttgg gcacagctgg gtgatcttta cggagccata
1561 ggctctccag tgagactgac tcgcaccgta gtggtaggga agcagaagga cttagtccag
1621 cgaatacttt atgtcctgac ctactttctc cgttgctctg agctacaaga gaaccagctg
1681 acctggagtg gcaatcatgg tgaaggtgac caagttttaa atgggagcaa gatcataaca
1741 gccttggaga aaggagaggt ggaggagtct gagtatgtgg tcattacggt gaggaacgag
1801 cccgctcttg taccccccat cctaccacca acagcagcag agagacacaa cccctggccg
1861 acagggtttc ctgagtgccc agagggcact gacagtagag acctgggtct taaacctgac
1921 aaagaagcta acaggaggcc agagcagggt tctgaggctt gcagcgcagg gtgcctgggg
1981 ccagcatcag acgcttcctg gaaacctcag aatgcatttt gtggggatga gaaaaataaa
2041 gaggcaccgc aagatggctc ttcaagactt cccagctgtg aagttttggg ggcaggaatg
2101 aagatggacc agcaagctgt ctgtgagctg ttgaaagtgg agatgcctac aagactgcca
2161 gaccggtcag tggcctggcc ttgccctgac agacatctcc gggagaaacc ttccttagaa
2221 aaggtcactt tccagattgg aagctttgca tctccagagt ctgactttga aagccgcatg
2281 aaaaaaatgg aggaacgggt gaaggcctgt ggcccctcct tggaggccag tgaggctgct
2341 gatgtggctc aggacccgca ggtttctagg agccctttta aacctggctt tcaggagaat
2401 gtttgctgtc ctcagaatcg gctttcagag ggggatgaag gcgagtctga caagggtttt
2461 gcagaggaca gaggcagcag aaacgacatg gcagcagata ttgctgggca gctcagccac
2521 gctgctgact tgggcacagc ctcccacggt gcaggaggaa cgggagggag gaggctggag
2581 gccactagag gtttgtatgt gaaggctgcg gaaggacctg tgctggagcc tgttgccccc
2641 aggtgtgtcc agcggggccc tggcctcgtg gctggtgcga atatcccctg tggggatgac
2701 aacaagaagg ccaacttcag gactgaagga gacattcccc gaaatgaaag ctcagatagc
2761 gccctgggag acagtgacga cgaagcctgc gcttcagcca tgctagatct gggtcacggt
2821 ggtgacagga ctggagggtc cttggaagtg gagctgcctc tgccaaggtc tcagagcatc
2881 agcacccaga atgtaaggaa ctttggccgc tcacttctgg cgggctactg ccccacatac
2941 atgcctgatc ttgtgcttca tgggaccggc agtgatgaga agctgaagca gtgcctggtg
3001 gccgaccttg tccacacagt ccatcatcca gtcctggatg agccaatagc tgaagctgtc
3061 tgtattatcg cagacacgga taaatggagt gtgcaggtag ctacaagtca gaggaaagtg
3121 acggacaaca tgaaactagg ccaggatgtc ctggtctcta gtcaggtgtc cagtttgctt
3181 cagtccattt tacagctcta taagcttcac ctccctgctg atttttgcat catgcatctt
3241 gaagatagac tacaggagat gtaccttaaa agtaaaatgc tatctgaata tctccgggga
3301 cacacacgag tccatgtgaa agaattaggt gtcgtactgg ggattgaatc caacgacctg
3361 cctctgttga ctgctattgc cagtactcat tctccttatg tggctcaaat actcttataa
```

### Sequence Accession No.: NM_{—}003437

Gene: Homo sapiens zinc finger protein 136 (ZNF136)

```
(SEQ ID NO: 114)
      1 atggactcgg tggcttttga ggatgtagat gtgaacttca cccaggagga gtgggctttg
    61 ctagatcctt cccagaagaa tctctacaga gatgtgatgt gggaaaccat gaggaatctg
  121 gcctctatag ggaaaaaatg gaaggaccag aacattaaag atcactacaa acaccgaggg
  181 agaaatctaa gaagtcatat gttagaaaga ctctatcaaa ctaaggatgg tagtcagcgt
  241 ggaggaattt ttagccagtt tgcaaatcag aatctgagca agaaaatccc tggagtgaaa
  301 ctctgtgaaa gcattgtata tggagaagtc agcatgggtc agtcatccct taatagacac
  361 atcaaagatc acagtggaca tgaaccaaag gaatatcagg aatatggaga gaagccagat
  421 acacgtaacc agtgttggaa acccttcagt tctcaccact cctttcgaac acatgagata
  481 attcacactg gagagaaact ctatgattgt aaggaatgtg gaaaaacctt cttttctctc
  541 aaaagaatta gaagacacat catcacacac agtggatata caccatataa atgtaaggtg
  601 tgtgggaaag cttttgatta tcccagtaga tttcgaacac atgaaagaag tcacactgga
  661 gagaaaccct atgaatgtca ggaatgtgga aaagccttca cttgtatcac aagtgttcga
  721 agacacatga taaagcacac tggagatgga ccttataaat gtaaggtatg tgggaaaccc
  781 tttcattctc tgagttcatt tcaagtgcat gaaagaattc acactggaga aaaacccttt
  841 aaatgtaagc aatgtggtaa agccttcagt tgttccccaa ccttacgaat acatgaaaga
  901 acccatactg gagagaaacc ttatgaatgc aagcagtgtg ggaaggcctt cagttatctc
  961 ccctcccttc gactacatga aagaattcac actggtgaga aacccttcgt atgtaaacaa
1021 tgtggtaaag cctttagatc tgccagtacc tttcaaatac atgaaaggac tcacactgga
1081 gaaaaacctt atgaatgtaa ggaatgtggg gaagcattca gttgtatccc aagtatgcga
1141 agacacatga taaaacatac tggagaagga ccttataaat gtaaggtatg tgggaaaccc
1201 tttcattctc tgagtccatt tcgaatacat gaaagaactc acactggaga gaaaccttat
1261 gtatgtaaac attgtggtaa agctttcgtt tcttcaacat caattcgaat acatgaaaga
1321 actcatactg gagagaaacc ctatgagtgt aagcaatgtg ggaaagcctt cagttatctc
1381 aactcctttc gaacacatga aatgattcac actggtgaga aaccctttga atgtaagcga
1441 tgtggtaaag cctttagatc ttctagttcc tttcgactac atgaaaggac tcacactgga
1501 cagaaaccct atcattgcaa ggaatgtggg aaagcctatt cttgccgtgc cagctttcag
1561 agacacatgt taacacatgc tgaagatgga ccaccttata aatgcatgtg ggaaagcctt
1621 taa
```

### Sequence Accession No.: NM_{—}001024681

Gene: Homo sapiens D15F37 gene (D15F37)

```
(SEQ ID NO: 116)
      1 atgcacgcat tttgtgttgg ccagtatttg gagcctgacc aagaaggcgt caccatacca
    61 gatctgggga gtctctcctc acctctgata gacacagaga ggaatctggg cctgcttctc
  121 ggattacacg cttcctattt agcaatgagc acaccgctgt ctcctgtcga gattgaatgt
  181 gacaaatggc ttcagtcatc catcttctct ggaggcctgc agaccagcca gatccactac
  241 agctacaacg aggagaaaga cgaggaccac tgcagctccc cagggggcac acctgccagc
  301 aaatctcgac tctgctccca cagacgggcc ctgggggacc attcccaggc atttctgcaa
  361 gccattgcag acaacaacat tcaggatcac aacgtgaagg actttttgtg tcaaatagaa
  421 aggtactgta ggcagtgcca tttgaccaca ccgatcatgt ttccccccga gcatcccgtg
  481 gaagaggtcg gtcgcttgct gttatgttgc ctcttaaaac atgaagattt aggtcatgtg
  541 gcattatctt tagttcatgc aggtgcactt gatattgagc aagtaaagca cagaacgttg
  601 cctaagtcag tggtggatgt ttgtagagtt gtctaccaag caaaatgttc gctcattaag
  661 actcatcaag aacagggccg ttcttacaag gaggtctgcg ctcctgtcat caaacgtttg
  721 agattcctct ttaatgaatt gagacctgct gtttgtaatg acctctctat aatgtctaag
  781 tttaaattgt taagttcttt gccccattgg aggaggatag ctcagaagat aattcgagaa
  841 ccaaggaaaa agagagttcc taagaagcca gaatctacgg atgatgaaga aaaaattgga
  901 aacgaagaga gtgatttaga agaagcttgc attttgcctc atagtccaat aaatgtggac
  961 aagagaccca ttgcaattaa atcacccaag gacaaatggc agccgctgtt gagtactgtt
1021 acagatgttc acaaatacaa gtggttgaag cagaatgtgc agggtcttta tccgcagtct
1081 ccactcctca gtacaattgc tgaatttgcc cttaaagaag agccagtgga tgtggaaaag
1141 agaaagtgcc tactaaaaca gttggagaga gcagaggttc gcctggaagg gatagataca
1201 attttaaaat tgtatctggt gagcaagaat ttcttacttc catctgtgcc gtatgcgatg
1261 ttttgtggat ggcaaagact tattcctgag ggaatcgata taggggaacc tcttactgat
1321 tgtttaaagg atgttgattt gatcccgcct tttaatcgga tgctgctgga agtcaccttt
1381 ggcaagctgt acgcttgggc tgttcagaac attcgaaatg ttttggtgga tgccagtgcc
1441 aaatttaaag agcttggtat ccagccggtt cccctgcaaa ccatcaccaa tgagaaccca
1501 tcgggaccga gcctggggac catcccgcaa gcccacttcc tcctggtgat gctcagcatg
1561 ctcaccctgc agcacagcgc aaacaacctt gacctcctgc tcaattccgg cacgctggcc
1621 ctcgctcaga cggcactgcg cctgattggc cccagttgtg acagcgttga ggaagatatg
1681 aatgcttctg cccaaggtgc ttctgccaca gttttggaag aaacaaggaa ggaaacggct
1741 cctgtgcagc tccctgtttc agggccagaa ctggctgcca tgatgaagat tggaacaagg
1801 gtcatgagag gtgtggactg gaaatggggc gatcaggatg ggcctcctcc aggcctaggc
1861 cgagtgattg gtgagctggg agaggacggg tggataagag tccagtggga cacaggcagc
1921 accaactcct acaggatggg gaaagaagga aaatacgacc tcaagctggc agagctgcca
1981 gcccctgcac agccctcagc agaggattcg gacacagagg acgactctga agccgaacaa
2041 actgaaagga acattcaccc cactgcaatg atgtttacca gcactattaa cttactgcag
2101 actctttgtc tgtctgctgg agttcatgct gagatcatgc agagtgaagc caccaagact
2161 ttatgcggac tgctgcgaat gttagtggaa agcggaacga cggacaagac atcttctcca
2221 aacaggctgg tgtacaggga gcaacaccgg agctggtgca cgctggggtt tgtgcagagc
2281 atcgctctca cgctgcaggt gtgcggcgcc ctcagctccc cgcagtggat cacgctgctc
2341 atgaaggttg tggaagggca cgcacccttc actgccacct cgctgcagag gcagatctta
2401 gctgtgcatt tgttgcaagc agtccttccg tcatgggaca agaccgaaag ggtgagggac
2461 atgaaatgcc tcatggagaa gctgtttgac ttcttgggga gcttgctcac tatgtgctcc
2521 tctgacgtgc cgttactcag agagtccacg ctgaggcggc gcagggtgtg cccgcaggcc
2581 tcgctgactg ccacccacag cagcacactg gcggaggagg tggtggcact gctgcacacg
2641 ctgcactccc tgactcggtg gaatgggttc atcaacaagt acatcaactc ccagctccgc
2701 tccatcaccc acagctttgc gggaaggcct tccaaagggg cccagttaga tgactacttc
2761 cctgattccg agaaccctga agtggggggc ctcatggcgg tcctggctgt ggttggaggc
2821 atcgatggtc gcctgtgcct gggcggccaa gttgtgcacg atgactttgg agaagtcacc
2881 atgactcgca tcaccctgaa gggcaaaatc accgtgcagt tctctgacat gcggacgtgt
2941 cgcgtttgcc cattgaatca gctgaaacca ctccctgccg tggcctttaa tgtgaacaac
3001 ctgcccttca cagagcccat gctgtctgtc tgggctcagt tggtgaacct cgctggaagc
3061 aagttagaaa agcacaaaat aaagaaatcg actaaacagg cctttgcagg acaagtggac
3121 ctggacctgc tgcggcgcca gcagttgaag ctatacatcc tgaaagcagg tcgggcgctg
3181 ttctcccacc aggataaact gcggcagatc ctgtctcagc cagctgttca ggagactgga
3241 actgttcaca cagatgatgg agcagtggta tcacctgacc ttggggacat gtctcctgaa
3301 gggccgcagc cccccatgat cctcttgcag cagctgctgg cctcggccac ccagccgtct
3361 cctgtgaagg ccatatttga taaacaggaa cttgagactg ctgcactggc cgttgtggag
3421 tccactcacc cttcgagccc aggatttgaa gactgcagct ccagtgaggc caccacgcct
3481 gtcaacgtgc agcacatccg ccctgccaga gtgaagaggc gcaagcagtc acccgttccc
3541 gctctgccga tcgtggtgca gctcatggag atgggatttc ccagaaggaa catcgagttt
3601 gccctgaagt ctctcactgg tgcttccggg aatgcgtccg gcttgcctgg tgtggaagcc
3661 ttggtcgggt ggctgctgga ccactccgac atacaggtca cggagctctc agatgcagac
3721 acggtgtccg acgagtattc tgacgaggag gtggtggagg acatggatga tgccgcctac
3781 tccatgtcta ctggtgctgt tgtgacggag agccagacgt acaaaaaccg agctggtttc
3841 ttgggtaatg atgattatgc tgtatatgtg agagagaata ttcaggtggg aatgatggtt
3901 agatgctgcc gaacatacga agaagtgtgc gaaggtgatg tgatgttggc aaagtcatca
3961 agctggacag agatggattg catgatctca atgtgcagtg tgactggcag cagaaagggg
4021 gcatctactg gtttaggtac attcatgtgg aacttatag
```

### Sequence Accession No.: NM_{—}001259

Gene: Homo sapiens cyclin-dependent kinase 6 (CDK6)

(SEQ ID NO: 115)
ATGGAGAAGGACGGCCTGTGCCGCGCTGACCAGCAGTACGAATGCGTG
GCGGAGATCGGGGAGGGCGCCTATGGGAAGGTGTTCAAGGCCCGCGAC
TTGAAGAACGGAGGCCGTTTCGTGGCGTTGAAGCGCGTGCGGGTGCAG
ACCGGCGAGGAGGGCATGCCGCTCTCCACCATCCGCGAGGTGGCGGTG
CTGAGGCACCTGGAGACCTTCGAGCACCCCAACGTGGTCAGGTTGTTT
GATGTGTGCACAGTGTCACGAACAGACAGAGAAACCAAACTAACTTTA
GTGTTTGAACATGTCGATCAAGACTTGACCACTTACTTGGATAAAGTT
CCAGAGCCTGGAGTGCCCACTGAAACCATAAAGGATATGATGTTTCAG
CTTCTCCGAGGTCTGGACTTTCTTCATTCACACCGAGTAGTGCATCGC
GATCTAAAACCACAGAACATTCTGGTGACCAGCAGCGGACAAATAAAA
CTCGCTGACTTCGGCCTTGCCCGCATCTATAGTTTCCAGATGGCTCTA
ACCTCAGTGGTCGTCACGCTGTGGTACAGAGCACCCGAAGTCTTGCTC
CAGTCCAGCTACGCCACCCCCGTGGATCTCTGGAGTGTTGGCTGCATA
TTTGCAGAAATGTTTCGTAGAAAGCCTCTTTTTCGTGGAAGTTCAGAT
GTTGATCAACTAGGAAAAATCTTGGACGTGATTGGACTCCCAGGAGAA
GAAGACTGGCCTAGAGATGTTGCCCTTCCCAGGCAGGCTTTTCATTCA
AAATCTGCCCAACCAATTGAGAAGTTTGTAACAGATATCGATGAACTA
GGCAAAGACCTACTTCTGAAGTGTTTGACATTTAACCCAGCCAAAAGA
ATATCTGCCTACAGTGCCCTGTCTCACCCATACTTCCAGGACCTGGAA
AGGTGCAAAGAAAACCTGGATTCCCACCTGCCGCCCAGCCAGAACACC
TCGGAGCTGAATACAGCCTGA

### Sequence Accession No.: NM_{—}003463

Gene: Homo sapiens protein tyrosine phosphatase type IVA, member 1 (PTP4A1)

(SEQ ID NO: 163)
ATGGCTCGAATGAACCGCCCAGCTCCTGTGGAAGTCACATACAAGAAC
ATGAGATTTCTTATTACACACAATCCAACCAATGCGACCTTAAACAAA
TTTATAGAGGAACTTAAGAAGTATGGAGTTACCACAATAGTAAGAGTA
TGTGAAGCAACTTATGACACTACTCTTGTGGAGAAAGAAGGTATCCAT
GGTTCTTGATTGGCCTTTTATGATGGTGCACCACCATCCAACCAGATT
GTTGATGACTGGTTAAGTCTTGTGAAAATTAAGTTTCGTGAAGAACCT
GGTTGTTGTATTGCTGTTCATTGCGTTGCAGGCCTTGGGAGAGCTCCA
GTACTTGTTGCCCTAGCATTAATTGAAGGTGGAATGAAATACGAAGAT
GCAGTACAATTCATAAGACAAAAGCGGCGTGGAGCTTTTAACAGCAAG
CAACTTCTGTATTTGGAGAAGTATCGTCCTAAAATGCGGCTGCGTTTC
AAAGATTCCAACGGTCATAGAAACAACTGTTGCATTCAATAA

### Sequence Accession No.: NM_{—}003254

Gene: Homo sapiens TIMP metallopeptidase inhibitor 1 (TIMP1)

(SEQ ID NO: 164)
ATGGCCCCCTTTGAGCCCCTGGCTTCTGGCATCCTGTTGTTGCTGTGG
CTGATAGCCCCCAGCAGGGCCTGCACCTGTGTCCCACCCCACCCACAG
ACGGCCTTCTGCAATTCCGACCTCGTCATCAGGGCCAAGTTCGTGGGG
ACACCAGAAGTCAACCAGACCACCTTATACCAGCGTTATGAGATCAAG
ATGACCAAGATGTATAAAGGGTTCCAAGCCTTAGGGGATGCCGCTGAC
ATCCGGTTCGTCTACACCCCCGCCATGGAGAGTGTCTGCGGATACTTC
CACAGGTCCCACAACCGCAGCGAGGAGTTTCTCATTGCTGGAAAACTG
CAGGATGGACTCTTGCACATCACTACCTGCAGTTTTGTGGCTCCCTGG
AACAGCCTGAGCTTAGCTCAGCGCCGGGGCTTCACCAAGACCTACACT
GTTGGCTGTGAGGAATGCACAGTGTTTCCCTGTTTATCCATCCCCTGC
AAACTGCAGAGTGGCACTCATTGCTTGTGGACGGACCAGCTCCTCCAA
GGCTCTGAAAAGGGCTTCCAGTCCCGTCACCTTGCCTGCCTGCCTCGG
GAGCCAGGGCTGTGCACCTGGCAGTCCCTGCGGTCCCAGATAGCCTGA

### Sequence Accession No.: NM_{—}001831

Gene: Homo sapiens clusterin (CLU)

(SEQ ID NO: 165)
ATGCAGGTTTGCAGCCAGCCCCAAAGGGGGTGTGTGCGCGAGCAGAGC
GCTATAAATACGGCGCCTCCCAGTGCCCACAACGCGGCGTCGCCAGGA
GGAGCGCGCGGGCACAGGGTGCCGCTGACCGAGGCGTGCAAAGACTCC
AGAATTGGAGGCATGATGAAGACTCTGCTGCTGTTTGTGGGGCTGCTG
CTGACCTGGGAGAGTGGGCAGGTCCTGGGGGACCAGACGGTCTCAGAC
AATGAGCTCCAGGAAATGTCCAATCAGGGAAGTAAGTACGTCAATAAG
GAAATTCAAAATGCTGTCAACGGGGTGAAACAGATAAAGACTCTCATA
GAAAAAACAAACGAAGAGCGCAAGACACTGCTCAGCAACCTAGAAGAA
GCCAAGAAGAAGAAAGAGGATGCCCTAAATGAGACCAGGGAATCAGAG
ACAAAGCTGAAGGAGCTCCCAGGAGTGTGCAATGAGACCATGATGGCC
CTCTGGGAAGAGTGTAAGCCCTGCCTGAAACAGACCTGCATGAAGTTC
TACGCACGCGTCTGCAGAAGTGGCTCAGGCCTGGTTGGCCGCCAGCTT
GAGGAGTTCCTGAACCAGAGCTCGCCCTTCTACTTCTGGATGAATGGT
GACCGCATCGACTCCCTGCTGGAGAACGACCGGCAGCAGACGCACATG
CTGGATGTCATGCAGGACCACTTCAGCCGCGCGTCCAGCATCATAGAC
GAGCTCTTCCAGGACAGGTTCTTCACCCGGGAGCCCCAGGATACCTAC
CACTACCTGCCCTTCAGCCTGCCCCACCGGAGGCCTCACTTCTTCTTT
CCCAAGTCCCGCATCGTCCGCAGCTTGATGCCCTTCTCTCCGTACGAG
CCCCTGAACTTCCACGCCATCTTCCAGCCCTTCCTTGAGATGATACAC
GAGGCTCAGCAGGCCATGGACATCCACTTCCATAGCCCGGCCTTCCAG
CACCCGCCAACAGAATTCATACGAGAAGGCGACGATGACCGGACTGTG
TGCCGGGAGATCCGCCACAACTCCACGGGCTGCCTGCGGATGAAGGAC
CAGTGTGACAAGTGCCGGGAGATCTTGTCTGTGGACTGTTCCACCAAC
AACCCCTCCCAGGCTAAGCTGCGGCGGGAGCTCGACGAATCCCTCCAG
GTCGCTGAGAGGTTGACCAGGAAATACAACGAGCTGCTAAAGTCCTAC
CAGTGGAAGATGCTCAACACCTCCTCCTTGCTGGAGCAGCTGAACGAG
CAGTTTAACTGGGTGTCCCGGCTGGCAAACCTCACGCAAGGCGAAGAC
CAGTACTATCTGCGGGTCACCACGGTGGCTTCCCACACTTCTGACTCG
GACGTTCCTTCCGGTGTCACTGAGGTGGTCGTGAAGCTCTTTGACTCT
GATCCCATCACTGTGACGGTCCCTGTAGAAGTCTCCAGGAAGAACCCT
AAATTTATGGAGACCGTGGCGGAGAAAGCGCTGCAGGAATACCGCAAA
AAGCACCGGGAGGAGTGA

### Sequence Accession No.: NM_{—}000019

Gene: Homo sapiens acetyl-Coenzyme A acetyltransferase 1 (acetoacetyl Coenzyme A thiolase) (ACAT1), nuclear gene encoding mitochondrial protein

```
(SEQ ID NO: 117)
      1 atggctgtgc tggcggcact tctgcgcagc ggcgcccgca gccgcagccc cctgctccgg
    61 aggctggtgc aggaaataag atatgtggaa cggagttatg tatcaaaacc cactttgaag
  121 gaagtggtca tagtaagtgc tacaagaaca cccattggat cttttttagg cagcctttcc
  181 ttgctgccag ccactaagct tggttccatt gcaattcagg gagccattga aaaggcaggg
  241 attccaaaag aagaagtgaa agaagcatac atgggtaatg ttctacaagg aggtgaagga
  301 caagctccta caaggcaggc agtattgggt gcaggcttac ctatttctac tccatgtacc
  361 accataaaca aagtttgtgc ttcaggaatg aaagccatca tgatggcctc tcaaagtctt
  421 atgtgtggac atcaggatgt gatggtggca ggtgggatgg agagcatgtc caatgttcca
  481 tatgtaatga acagaggatc aacaccatat ggtggggtaa agcttgaaga tttgattgta
  541 aaagacgggc taactgatgt ctacaataaa attcatatgg gcagctgtgc tgagaataca
  601 gcaaagaagc tgaatattgc acgaaatgaa caggacgctt atgctattaa ttcttatacc
  661 agaagtaaag cagcatggga agctgggaaa tttggaaatg aagttattcc tgtcacagtt
  721 acagtaaaag gtcaaccaga tgtagtggtg aaagaagatg aagaatataa acgtgttgat
  781 tttagcaaag ttccaaagct gaagacagtt ttccagaaag aaaatggcac agtaacagct
  841 gccaatgcca gtacactgaa tgatggagca gctgctctgg ttctcatgac ggcagatgca
  901 gcgaagaggc tcaatgttac accactggca agaatagtag catttgctga cgctgctgta
  961 gaacctattg attttccaat tgctcctgta tatgctgcat ctatggttct taaagatgtg
1021 ggattgaaaa aagaagatat tgcaatgtgg gaagtaaatg aagcctttag tctggttgta
1081 ctagcaaaca ttaaaatgtt ggagattgat ccccaaaaag tgaatatcaa tggaggagct
1141 gtttctctgg gacatccaat tgggatgtct ggagccagga ttgttggtca tttgactcat
1201 gccttgaagc aaggagaata cggtcttgcc agtatttgca atggaggagg aggtgcttct
1261 gccatgctaa ttcagaagct gtag
```

### Sequence Accession No.: NM_{—}005165

Gene: Homo sapiens aldolase C, fructose-bisphosphate (ALDOC)

```
(SEC) ID NO: 118)
      1 atgcctcact cgtacccagc cctttctgct gagcagaaga aggagttgtc tgacattgcc
    61 ctgcggattg tagccccggg caaaggcatt ctggctgcgg atgagtctgt aggcagcatg
  121 gccaagcggc tgagccaaat tggggtggaa aacacagagg agaaccgccg gctgtaccgc
  181 caggtcctgt tcagtgctga tgaccgtgtg aaaaagtgca ttggaggcgt cattttcttc
  241 catgagaccc tctaccagaa agatgataat ggtgttccct tcgtccgaac catccaggat
  301 aagggcatcg tcgtgggcat caaggttgac aagggtgtgg tgcctctagc tgggactgat
  361 ggagaaacca ccactcaagg gctggatggg ctctcagaac gctgtgccca atacaagaag
  421 gatggtgctg actttgccaa gtggcgctgt gtgctgaaaa tcagtgagcg tacaccctct
  481 gcacttgcca ttctggagaa cgccaacgtg ctggcccgtt atgccagtat ctgccagcag
  541 aatggcattg tgcctattgt ggaacctgaa atattgcctg atggagacca cgacctcaaa
  601 cgttgtcagt atgttacaga gaaggtcttg gctgctgtgt acaaggccct gagtgaccat
  661 catgtatacc tggaggggac cctgctcaag cccaacatgg tgaccccggg ccatgcctgt
  721 cccatcaagt ataccccaga ggagattgcc atggcaactg tcactgccct gcgtcgcact
  781 gtgcccccag ctgtcccagg agtgaccttc ctgtctgggg gtcagagcga agaagaggca
  841 tcattcaacc tcaatgccat caaccgctgc ccccttcccc gaccctgggc gcttaccttc
  901 tcctatgggc gtgccctgca agcctctgca ctcaatgcct ggcgagggca acgggacaat
  961 gctggggctg ccactgagga gttcatcaag cgggctgagg tgaatgggctt gcagcccag
1021 ggcaagtatg aaggcagtgg agaagatggt ggagcagcag cacagtcact ctacattgcc
1081 aaccatgcct actga
```

### Sequence Accession No.: NM_{—}052831

Gene: Homo sapiens chromosome 6 open reading frame 192 (C6orf192),

```
(SEQ ID NO: 119)
      1 atggaggcgc tgggtgacct ggagggacca cgcgcaccag gaggtgatga tcctgcagga
    61 agtgcaggag agacccccgg gtggctttcg agagaacagg tttttgtact gatatcggca
  121 gcttcggtga acttaggttc catgatgtgc tattctatac ttggaccgtt tttccccaaa
  181 gaggctgaaa agaagggagc cagcaataca attatcggta tgatctttgg atgttttgct
  241 ttgttcgagt tgctggcatc cttggtattt ggaaactatc ttgtacatat tggagcaaaa
  301 tttatgtttg tagcaggaat gtttgtctca ggaggagtta caattctctt tggtgtattg
  361 gaccgagttc cagatgggcc agtatttatt gctatgtgtt ttctagtgag agtaatggat
  421 gcagttagct ttgctgcagc aatgactgca tcttcttcta tcctggcaaa ggcttttcca
  481 aataacgtgg ctacggtatt gggaagtctt gagacttttt ctggactggg gctaatacta
  541 ggtcctcctg taggtggctt tttgtatcaa tcctttggct atgaagtgcc ttttattgtt
  601 ctgggatgcg tcgttttgct gatggtacca ctcaatatgt atattttacc caattacgag
  661 tctgatccag gtgaacactc attctggaaa ctgatcgctt tacccaaagt tggccttata
  721 gccttcgtca tcaactcact cagctcgtgt tttggcttcc tcgatcctac tctgtctctc
  781 tttgttttgg agaagttcaa tttaccagct ggatatgtgg gactagtatt cctgggtatg
  841 gcactgtcct atgccatctc ttcaccacta tttggtctcc taagtgataa aaggccacct
  901 ctaaggaaat ggcttctggt gtttggcaac ttaatcacag ccgggtgcta catgctctta
  961 gggcctgtcc caatcttgca tattaaaagt cagctctggc tgctggtgct gatattagtt
1021 gtaagtggcc tctctgctgg aatgagtata attccaactt tcccggaaat tctcagttgt
1081 gcacatgaaa atgggtttga agagggatta agtacattgg gacttgtatc aggtcttttt
1141 agtgcaatgt ggtcaattgg tgcttttatg ggaccaacgc tgggtggatt tctgtatgag
1201 aaaattggtt ttgaatgggc agcagctata caaggtctat gggctctgat aagtggatta
1261 gccatgggct tgttttatct actggagtat tcaaggagaa aaaggtctaa atctcaaaac
1321 atcctcagca cagaggagga acgaactact ctcttgccta atgaaaccta g
```

### Sequence Accession No.: NM_{—}000495

Gene: Homo sapiens collagen, type IV, alpha 5 (Alport syndrome) (COL4A5)

| (SEQ ID NO: 120) | |
|---|---|
| 1 | atgaaactgc gtggagtcag cctggctgcc ggcttgttct tactggccct gagtctttgg |
| 61 | gggcagcctg cagaggctgc ggcttgctat gggtgttctc caggatcaaa gtgtgactgc |
| 121 | agtggcataa aaggggaaaa gggagagaga gggtttccag gtttggaagg acacccagga |
| 181 | ttgcctggat ttccaggtcc agaagggcct ccggggcctc ggggacaaaa gggtgatgat |
| 241 | ggaattccag ggccaccagg accaaaagga atcagaggtc ctcctggact tcctggattt |
| 301 | ccagggacac caggtcttcc tggaatgcca ggccacgatg gggccccagg acctcaaggt |
| 361 | attcccggat gcaatggaac caagggagaa cgtggatttc caggcagtcc cggttttcct |
| 421 | ggtttacagg gtcctccagg accccctggg atcccaggta tgaagggtga accaggtagt |
| 481 | ataattatgt catcactgcc aggaccaaag ggtaatccag gatatccagg tcctcctgga |
| 541 | atacaaggcc tacctggtcc cactggtata ccagggccaa ttggtccccc aggaccacca |
| 601 | ggtttgatgg gccctcctgg tccaccagga cttccaggac ctaaggggaa tatgggctta |
| 661 | aatttccagg gacccaaagg tgaaaaaggt gagcaaggtc ttcagggccc acctgggcca |
| 721 | cctgggcaga tcagtgaaca gaaaagacca attgatgtag agtttcagaa aggagatcag |
| 781 | ggacttcctg gtgaccgagg gcctcctgga cctccaggga tacgtggtcc tccaggtccc |
| 841 | ccaggtggtg agaaaggtga gaagggtgag caaggagagc caggcaaaag aggtaaacca |
| 901 | ggcaaagatg gagaaaatgg ccaaccagga attcctggtt tgcctggtga tcctggttac |
| 961 | cctggtgaac ccggaaggga tggtgaaaag ggccaaaaag gtgacactgg cccacctgga |
| 1021 | cctcctggac ttgtaattcc tagacctggg actggtataa ctataggaga aaaaggaaac |
| 1081 | attgggttgc ctgggttgcc tggagaaaaa ggagagcgag gatttcctgg aatacagggt |
| 1141 | ccacctggcc ttcctggacc tccaggggct gcagttatgg gtcctcctgg ccctcctgga |
| 1201 | tttcctggag aaaggggtca gaaaggtgat gaaggaccac ctggaatttc cattcctgga |
| 1261 | cctcctggac ttgacggaca gcctggggct cctgggcttc cagggcctcc tggccctgct |
| 1321 | ggccctcaca ttcctcctag tgatgagata tgtgaaccag gccctccagg ccccccagga |
| 1381 | tctccaggtg ataaaggact ccaaggagaa caaggagtga aaggtgacaa aggtgacact |
| 1441 | tgcttcaact gcattggaac tggtatttca gggcctccag gtcaacctgg tttgccaggt |
| 1501 | ctcccaggtc ctccaggatc tcttggtttc cctggacaga aaggggaaaa aggacaagct |
| 1561 | ggtgcaactg gtcccaaagg attaccaggc attccaggag ctccaggtgc tccaggcttt |
| 1621 | cctggatcta aaggtgaacc tggtgatatc ctcacttttc caggaatgaa gggtgacaaa |
| 1681 | ggagagttgg gttcccctgg agctccaggg cttcctggtt tacctggcac tcctggacag |
| 1741 | gatggattgc cagggcttcc tggcccgaaa ggagagcctg gtggaattac ttttaagggt |
| 1801 | gaaagaggtc cccctgggaa cccaggttta ccaggcctcc cagggaatat agggcctatg |
| 1861 | ggtccccctg gtttcggccc tccaggccca gtaggtgaaa aaggcataca aggtgtggca |
| 1921 | ggaaatccag gccagccagg aataccaggt cctaaagggg atccaggtca gactataacc |
| 1981 | cagccgggga agcctggctt gcctggtaac ccaggcagag atggtgatgt aggtcttcca |
| 2041 | ggtgaccctg gacttccagg gcaaccaggc ttgccaggga tacctggtag caaaggagaa |
| 2101 | ccaggtatcc ctggaattgg gcttcctgga ccacctggtc ccaaaggctt tcctggaatt |
| 2161 | ccaggacctc caggagcacc tgggacacct ggaagaattg gtctagaagg ccctcctggg |
| 2221 | ccacccggct ttccaggacc aaagggtgaa ccaggatttg cattacctgg gccacctggg |
| 2281 | ccaccaggac ttccaggttt caaaggagca cttggtccaa aaggtgatcg tggtttccca |
| 2341 | ggacctccgg gtcctccagg acgcactggc ttagatgggc tccctggacc aaaaggtgat |
| 2401 | gttggaccaa atggacaacc tggaccaatg ggacctcctg ggctgccagg aataggtgtt |
| 2461 | cagggaccac caggaccacc agggattcct gggccaatag gtcaacctgg tttacatgga |
| 2521 | ataccaggag agaaggggga tccaggacct cctggacttg atgttccagg acccccaggt |
| 2581 | gaaagaggca gtccagggat ccccggagca cctggtccta taggacctcc aggatcacca |
| 2641 | gggcttccag gaaaagcagg tgcctctgga tttccaggta ccaaaggtga aatgggtatg |
| 2701 | atgggacctc caggcccacc aggacctttg ggaattcctg gcaggagtgg tgtacctggt |
| 2761 | cttaaaggtg atgatggctt gcagggtcag ccaggacttc ctggccctac aggagaaaaa |
| 2821 | ggtagtaaag gagagcctgg ccttccaggc cctcctggac caatggatcc aaatcttctg |
| 2881 | ggctcaaaag gagagaaggg ggaacctggc ttaccaggta tacctggagt ttcagggcca |
| 2941 | aaaggttatc agggtttgcc tggagaccca gggcaacctg gactgagtgg acaacctgga |
| 3001 | ttaccaggac caccaggtcc caaaggtaac cctggtctcc ctggacagcc aggtcttata |
| 3061 | ggacctcctg gacttaaagg aaccatcggt gatatgggtt ttccagggcc tcagggtgtg |
| 3121 | gaagggcctc ctggaccttc tggagttcct ggacaacctg gctccccagg attacctgga |
| 3181 | cagaaaggcg acaaaggtga tcctggtatt tcaagcattg gtcttccagg tcttcctggt |
| 3241 | ccaaagggtg agcctggtct gcctggatac ccagggaacc ctggtatcaa aggttctgtg |
| 3301 | ggagatcctg gtttgcccgg attaccagga acccctggag caaaaggaca accaggcctt |
| 3361 | cctggattcc caggaacccc aggccctcct ggaccaaaag gtattagtgg ccctcctggg |
| 3421 | aaccccggcc ttccaggaga acctggtcct gtaggtggtg gaggtcatcc tgggcaacca |
| 3481 | gggcctccag gcgaaaaagg caaacccggt caagatggta ttcctggacc agctggacag |
| 3541 | aagggtgaac caggtcaacc aggctttgga aacccaggac cccctggact tccaggactt |
| 3601 | tctggccaaa agggtgatgg aggattacct gggattccag gaaatcctgg ccttccaggt |
| 3661 | ccaaagggcg aaccaggctt tcacggtttc cctggtgtgc agggtccccc aggccctcct |
| 3721 | ggttctccgg gtccagctct ggaaggacct aaaggcaacc ctgggcccca aggtcctcct |
| 3781 | gggagaccag gtctaccagg tccagaaggt cctccaggtc tccctggaaa tggaggtatt |
| 3841 | aaaggagaga agggaaatcc aggccaacct gggctacctg gcttgcctgg tttgaaagga |
| 3901 | gatcaaggac caccaggact ccagggtaat cctggccggc cgggtctcaa tggaatgaaa |
| 3961 | ggagatcctg gtctccctgg tgttccagga ttcccaggca tgaaaggacc cagtggagta |
| 4021 | cctggatcag ctggccctga gggggaaccg ggacttattg gtcctccagg tcctcctgga |
| 4081 | ttacctggtc cttcaggaca gagtatcata attaaaggag atgctggtcc tccaggaatc |
| 4141 | cctggccagc ctgggctaaa gggtctacca ggaccccaag gacctcaagg cttaccaggt |
| 4201 | ccaactggcc ctccaggaga tcctggacgc aatggactcc ctggctttga tggtgcagga |
| 4261 | gggcgcaaag gagacccagg tctgccagga cagccaggta cccgtggttt ggatggtccc |
| 4321 | cctggtccag atggattgca aggtccccca ggtccccctg gaacctcctc tgttgcacat |
| 4381 | ggatttctta ttacacgcca cagccagaca acggatgcac cacaatgccc acagggaaca |
| 4441 | cttcaggtct atgaaggctt ttctctcctg tatgtacaag gaaataaaag agcccacggt |
| 4501 | caagacttgg ggacggctgg cagctgcctt cgtcgcttta gtaccatgcc tttcatgttc |
| 4561 | tgcaacatca ataatgtttg caactttgct tcaagaaatg actattctta ctggctctct |
| 4621 | accccagagc ccatgccaat gagcatgcaa cccctaaagg gccagagcat ccagccattc |
| 4681 | attagtcgat gtgcagtatg tgaagctcca gctgtggtga tcgcagttca cagtcagacg |
| 4741 | atccagattc cccattgtcc tcagggatgg gattctctgt ggattggtta ttccttcatg |
| 4801 | atgcatacaa gtgcaggggc agaaggctca ggtcaagccc tagcctcccc tggttcctgc |
| 4861 | ttggaagagt ttcgttcagc tcccttcatc gaatgtcatg ggaggggtac ctgtaactac |
| 4921 | tatgccaact cctacagctt ttggctggca actgtagatg tgtcagacat gttcagtaaa |
| 4981 | cctcagtcag aaacgctgaa agcaggagac ttgaggacac gaattagccg atgtcaagtg |
| 5041 | tgcatgaaga ggacataa |

### Sequence Accession No.: NM_{—}001212

Gene: Homo sapiens complement component 1, q subcomponent binding protein (C1QBP), nuclear gene encoding mitochondrial protein

| (SEQ ID NO: 121) | |
|---|---|
| 1 | atgctgcctc tgctgcgctg cgtgccccgt gtgctgggct cctccgtcgc cggcctccgc |
| 61 | gctgccgcgc ccgcctcgcc tttccggcag ctcctgcagc cggcaccccg gctgtgcacc |
| 121 | cggcccttcg ggctgctcag cgtgcgcgca ggttccgagc ggcggccggg cctcctgcgg |
| 181 | cctcgcggac cctgcgcctg tggctgtggc tgcggctcgc tgcacaccga cggagacaaa |
| 241 | gcttttgttg atttcctgag tgatgaaatt aaggaggaaa gaaaaattca gaagcataaa |
| 301 | accctcccta agatgtctgg aggttgggag ctggaactga atgggacaga agcgaaatta |
| 361 | gtgcggaaag ttgccgggga aaaaatcacg gtcactttca acattaacaa cagcatccca |
| 421 | ccaacatttg atggtgagga ggaaccctcg caagggcaga aggttgaaga acaggagcct |
| 481 | gaactgacat caactcccaa tttcgtggtt gaagttataa agaatgatga tggcaagaag |
| 541 | gcccttgtgt tggactgtca ttatccagag gatgaggttg gacaagaaga cgaggctgag |
| 601 | agtgacatct tctctatcag ggaagttagc tttcagtcca ctggcgagtc tgaatggaag |
| 661 | gatactaatt atacactcaa cacagattcc ttggactggg ccttatatga ccacctaatg |
| 721 | gatttccttg ccgaccgagg ggtggacaac acttttgcag atgagctggt ggagctcagc |
| 781 | acagccctgg agcaccagga gtacattact tttcttgaag acctcaagag ttttgtcaag |
| 841 | agccagtag |

### Sequence Accession No.: NM_{—}001311

Gene: Homo sapiens cysteine-rich protein 1 (intestinal) (CRIP1)

| (SEQ ID NO: 122) | |
|---|---|
| 1 | atgcccaagt gtcccaagtg caacaaggag gtgtacttcg ccgagagggt gacctctctg |
| 61 | ggcaaggact ggcatcggcc ctgcctgaag tgcgagaaat gtgggaagac gctgacctct |
| 121 | gggggccacg ctgagcacga aggcaaaccc tactgcaacc acccctgcta cgcagccatg |
| 181 | tttgggccta aaggctttgg gcggggcgga gccgagagcc acactttcaa gtaa |

### Sequence Accession No.: NM_{—}182503

Gene: Gene: Homo sapiens deaminase domain containing 1 (DEADC1)

| (SEQ ID NO: 123) | |
|---|---|
| 1 | atggaggcga aggcggcacc caagccagct gcaagcggcg cgtgctcggt gtcggcagag |
| 61 | gagaccgaaa agtggatgga ggaggcgatg cacatggcca aagaagccct cgaaaatact |
| 121 | gaagttcctg ttggctgtct tatggtctac aacaatgaag ttgtagggaa ggggagaaat |
| 181 | gaagttaacc aaaccaaaaa tgctactcga catgcagaaa tggtggccat cgatcaggtc |
| 241 | ctcgattggt gtcgtcaaag tggcaagagt ccctctgaag tatttgaaca cactgtgttg |
| 301 | tatgtcactg tggagccgtg cattatgtgt gcagctgctc tccgcctgat gaaaatcccg |
| 361 | ctggttgtat atggctgtca gaatgaacga tttggtggtt gtggctctgt tctaaatatt |
| 421 | gcctctgctg acctaccaaa cactgggaga ccatttcagt gtatccctgg atatcgggct |
| 481 | gaggaagcag tggaaatgtt aaagaccttc tacaaacaag aaaatccaaa tgcaccaaaa |
| 541 | tcgaaagttc ggaaaaagga atgtcagaaa tcttga |

### Sequence Accession No.: NM_{—}015917

Gene: Homo sapiens glutathione S-transferase kappa 1 (GSTK1),

| (SEQ ID NO: 124) | |
|---|---|
| 1 | atggggcccc tgccgcgcac cgtggagctc ttctatgacg tgctgtcccc ctactcctgg |
| 61 | ctgggcttcg agatcctgtg ccggtatcag aatatctgga acatcaacct gcagttgcgg |
| 121 | cccagcctca taacagggat catgaaagac agtggaaaca agcctccagg tctgcttccc |
| 181 | cgcaaaggac tatacatggc aaatgactta aagctcctga gacaccatct ccagattccc |
| 241 | atccacttcc ccaaggattt cttgtctgtg atgcttgaaa aaggaagttt gtctgccatg |
| 301 | cgtttcctca ccgccgtgaa cttggagcat ccagagatgc tggagaaagc gtcccgggag |
| 361 | ctgtggatgc gcgtctggtc aaggaatgaa gacatcaccg agccgcagag catcctggcg |
| 421 | gctgcagaga aggctggtat gtctgcagaa caagcccagg gacttctgga aaagatcgca |
| 481 | acgccaaagg tgaagaacca gctcaaggag accactgagg cagcctgcag atacggagcc |
| 541 | tttgggttgc ccatcaccgt ggcccatgtg gatggccaaa cccacatgtt atttggctct |
| 601 | gaccggatgg agctgctggc gcacctgctg ggagagaagt ggatgggccc tatacctcca |
| 661 | gccgtgaatg ccagacttta a |

### Sequence Accession No.: NM_{—}183239

Gene: Homo sapiens glutathione S-transferase omega 2 (GSTO2)

| (SEQ ID NO: 125) | |
|---|---|
| 1 | atgtctgggg atgcgaccag gaccctgggg aaaggaagcc agcccccagg gccagtcccg |
| 61 | gaggggctga tccgcatcta cagcatgagg ttctgcccct attctcacag gacccgcctc |
| 121 | gtcctcaagg ccaaagacat cagacatgaa gtggtcaaca ttaacctgag aaacaagcct |
| 181 | gaatggtact atacaaagca cccttttggc cacattcctg tcctggagac cagccaatgt |
| 241 | caactgatct atgaatctgt tattgcttgt gagtacctgg atgatgctta tccaggaagg |
| 301 | aagctgtttc catatgaccc ttatgaacga gctcgccaaa agatgttatt ggagctattt |
| 361 | tgtaaggtcc cacatttgac caaggagtgc ctggtagcgt tgagatgtgg gagagaatgc |
| 421 | actaatctga aggcagccct gcgtcaggaa ttcagcaacc tggaagagat tcttgagtat |
| 481 | cagaacacca ccttctttgg tggaacctgt atatccatga ttgattacct cctctggccc |
| 541 | tggtttgagc ggctggatgt gtatgggata ctggactgtg tgagccacac gccagccctg |
| 601 | cggctctgga tatcagccat gaagtgggac cccacagtct gtgctcttct catggataag |
| 661 | agcattttcc agggcttctt gaatctctat tttcagaaca accctaatgc ctttgacttt |
| 721 | gggctgtgct ga |

### Sequence Accession No.: NM_{—}032483

Gene: Homo sapiens phosphatidic acid phosphatase type 2 domain containing 1B (PPAPDC1B)

| (SEQ ID NO: 126) | |
|---|---|
| 1 | atgtggctct accggaaccc ctacgtggag gcggagtatt tccccaccaa gccgatgttt |
| 61 | gttattgcat ttctctctcc actgtctctg atcttcctgg ccaaatttct caagaaggca |
| 121 | gacacaagag acagcagaca agcctgcctg gctgccagcc ttgccctggc tctgaatggc |
| 181 | gtctttacca acacaataaa actgatcgta gggaggccac gcccagattt cttctaccgc |
| 241 | tgcttccctg atgggctagc ccattctgac ttgatgtgta caggggataa ggacgtggtg |
| 301 | aatgagggcc gaaagagctt ccccagtgga cattcttcct ttgcatttgc tggtctggcc |
| 361 | tttgcgtcct tctacctggc agggaagtta cactgcttca caccacaagg ccgtgggaaa |
| 421 | tcttggaggt tctgtgcctt tctgtcacct ctactttttg cagctgtgat tgcactgtcc |
| 481 | cgcacatgtg actacaagca tcactggcaa ggacccttta aatggtga |

### Sequence Accession No.: NM_{—}032354

Gene: Homo sapiens transmembrane protein 107 (TMEM107)

| (SEQ ID NO: 127) | |
|---|---|
| 1 | atgggccggg tctcagggct tgtgccctct cgcttcctga cgctcctggc gcatctggtg |
| 61 | gtcgtcatca ccttattctg gtcccgggac agcaacatac aggcctgcct gcctctcacg |
| 121 | ttcacccccg aggagtatga caagcaggac attcatccac ttcctctctg caggctggtg |
| 181 | gccgcgctct ctgtcaccct gggcctcttt gcagtggagc tggccggttt cctctcagga |
| 241 | gtctccatgt tcaacagcac ccagagcctc atctccattg gggctcactg tagtgcatcc |
| 301 | gtggccctgt ccttcttcat attcgagcgt tgggagtgca ctacgtattg gtacattttt |
| 361 | gtcttctgca gtgcccttcc agctgtcact gaaatggctt tattcgtcac cgtctttggg |
| 421 | ctgaaaaaga aacccttctg a |

### Sequence Accession No.: NM_{—}024116

Gene: Homo sapiens Josephin domain containing 3 (JOSD3)

| (SEQ ID NO: 129) | |
|---|---|
| 1 | atggataaat caggaataga ttctcttgac catgtgacat ctgatgctgt ggaacttgca |
| 61 | aatcgaagtg ataactcttc tgatagcagc ttatttaaaa ctcagtgtat cccttactca |
| 121 | cctaaagggg agaaaagaaa ccccattcga aaatttgttc gtacacctga aagtgttcac |
| 181 | gcaagtgatt catcaagtga ctcatctttt gaaccaatac cattgactat aaaagctatt |
| 241 | tttgaaagat tcaagaacag gaaaaagaga tataaaaaaa agaaaaagag gaggtaccag |
| 301 | ccaacaggaa gaccacgggg aagaccagaa ggaaggagaa atcctatata ctcactaata |
| 361 | gataagaaga aacaatttag aagcagagga tctggcttcc catttttaga atcagagaat |
| 421 | gaaaaaaacg caccttggag aaaaatttta acgtttgagc aagctgttgc aagaggattt |
| 481 | tttaactata ttgaaaaact gaagtatgaa caccacctga aagaatcatt gaagcaaatg |
| 541 | aatgttggtg aagatttaga aaatgaagat tttgacagtc gtagatacaa atttttggat |
| 601 | gatgatggat ccatttctcc tattgaggag tcaacagcag aggatgagga tgcaacacat |
| 661 | cttgaagata acgaatgtga tatcaaattg gcaggggata gtttcatagt aagttctgaa |
| 721 | ttccctgtaa gactgagtgt atacttagaa gaagaggata ttactgaaga agctgctttg |
| 781 | tctaaaaaga gagctacaaa agccaaaaat actggacaga gaggcctgaa aatgtga |

### Sequence Accession No.: NM_{—}178454

Gene: Homo sapiens transmembrane protein 77 (TMEM77)

| (SEQ ID NO: 130) | |
|---|---|
| 1 | atgtggtggt ttcagcaagg cctcagtttc cttccttcag cccttgtaat ttggacatct |
| 61 | gctgctttca tattttcata cattactgca gtaacactcc accatataga cccggcttta |
| 121 | ccttatatca gtgacactgg tacagtagct ccagaaaaat gcttatttgg ggcaatgcta |
| 181 | aatattgcgg cagttttatg cattgctacc atttatgttc gttataagca agttcatgct |
| 241 | ctgagtcctg aagagaacgt tatcatcaaa ttaaacaagg ctggccttgt acttggaata |
| 301 | ctgagttgtt tagggctttc tattgtggca aacttccaga aaacaaccct ttttgctgca |
| 361 | catgtaagtg gagctgtgct tacctttggt atgggctcat tatatatgtt tgttcagacc |
| 421 | atcctttcct accaaatgca gcccaaaatc catggcaaac aagtcttctg gatcagactg |
| 481 | ttgttggtta tctggtgtgg agtaagtgca cttagcatgc tgacttgctc atcagttttg |
| 541 | cacagtggca attttgggac tgatttagaa cagaaactcc attggaaccc cgaggacaaa |
| 601 | ggttatgtgc ttcacatgat cactactgca gcagaatggt ctatgtcatt ttccttcttt |
| 661 | gtttttttcc tgacttacat tcgtgatttt cagaaaattt ctttacgggt ggaagccaat |
| 721 | ttacatggat taaccctcta tgacactgca ccttgcccta ttaacaatga acgaacacgg |
| 781 | ctactttcca gagatatttg a |

### Sequence Accession No.: NM_{—}020998

Gene: Homo sapiens macrophage stimulating 1 (hepatocyte growth factor-like) (MST1)

| (SEQ ID NO: 131) | |
|---|---|
| 1 | atggggtggc tcccactcct gctgcttctg actcaatgct taggggtccc tgggcagcgc |
| 61 | tcgccattga atgacttcca agtgctccgg ggcacagagc tacagcacct gctacatgcg |
| 121 | gtggtgcccg ggccttggca ggaggatgtg gcagatgctg aagagtgtgc tggtcgctgt |
| 181 | gggcccttaa tggactgccg ggccttccac tacaacgtga gcagccatgg ttgccaactg |
| 241 | ctgccatgga ctcaacactc gccccacacg aggctgcggc gttctgggcg ctgtgacctc |
| 301 | ttccagaaga aagactacgt acggacctgc atcatgaaca atggggttgg gtaccggggc |
| 361 | accatggcca cgaccgtggg tggcctgccc tgccaggctt ggagccacaa gttcccgaat |
| 421 | gatcacaagt acacgcccac tctccggaat ggcctggaag agaacttctg ccgtaaccct |
| 481 | gatggcgacc ccggaggtcc ttggtgctac acaacagacc ctgctgtgcg cttccagagc |
| 541 | tgcggcatca aatcctgccg ggaggccgcg tgtgtctggt gcaatggcga ggaataccgc |
| 601 | ggcgcggtag accgcacgga gtcagggcgc gagtgccagc gctgggatct tcagcacccg |
| 661 | caccagcacc ccttcgagcc gggcaagttc ctcgaccaag gtctggacga caactattgc |
| 721 | cggaatcctg acggctccga gcggccatgg tgctacacta cggatccgca gatcgagcga |
| 781 | gagttctgtg acctcccccg ctgcgggtcc gaggcacagc cccgccaaga ggccacaact |
| 841 | gtcagctgct tccgcgggaa gggtgagggc taccggggca cagccaatac caccactgcg |
| 901 | ggcgtacctt gccagcgttg ggacgcgcaa atccctcatc agcaccgatt tacgccagaa |
| 961 | aaatacgcgt gcaaagacct tcgggagaac ttctgccgga accccgacgg ctcagaggcg |
| 1021 | ccctggtgct tcacactgcg gcccggcatg cgcgcggcct tttgctacca gatccggcgt |
| 1081 | tgtacagacg acgtgcggcc ccaggactgc taccacggcg caggggagca gtaccgcggc |
| 1141 | acggtcagca agacccgcaa gggtgtccag tgccagcgct ggtccgctga gacgccgcac |
| 1201 | aagccgcagt tcacgtttac ctccgaaccg catgcacaac tggaggagaa cttctgccgg |
| 1261 | aacccagatg gggatagcca tgggccctgg tgctacacga tggacccaag gaccccattc |
| 1321 | gactactgtg ccctgcgacg ctgcgctgat gaccagccgc catcaatcct ggacccccca |
| 1381 | gaccaggtgc agtttgagaa gtgtggcaag agggtggatc ggctggatca gcggcgttcc |
| 1441 | aagctgcgcg tggttggggg ccatccgggc aactcaccct ggacagtcag cttgcggaat |
| 1501 | cggcagggcc agcatttctg cggggggtct ctagtgaagg agcagtggat actgactgcc |
| 1561 | cggcagtgct tctcctcctg ccatatgcct ctcacgggct atgaggtatg gttgggcacc |
| 1621 | ctgttccaga acccacagca tggagagcca agcctacagc gggtcccagt agccaagatg |
| 1681 | gtgtgtgggc cctcaggctc ccagcttgtc ctgctcaagc tggagagatc tgtgaccctg |
| 1741 | aaccagcgtg tggccctgat ctgcctgccc cctgaatggt atgtggtgcc tccagggacc |
| 1801 | aagtgtgaga ttgcaggctg gggtgagacc aaaggtacgg gtaatgacac agtcctaaat |
| 1861 | gtggccttgc tgaatgtcat ctctaaccag gagtgtaaca tcaagcaccg aggacgtgtg |
| 1921 | cgggagagtg agatgtgcac tgagggactg ttggcccctg tgggggcctg tgagggtgac |
| 1981 | tacgggggcc cacttgcctg ctttacccac aactgctggg tcctggaagg aattataatc |
| 2041 | cccaaccgag tatgcgcaag gtcccgctgg ccagctgtct tcacgcgtgt ctctgtgttt |
| 2101 | gtggactgga ttcacaaggt catgagactg ggttag |

### Sequence Accession No.: NM_{—}175617

Gene: Homo sapiens metallothionein 1E (MT1E)

| (SEQ ID NO: 132) | |
|---|---|
| 1 | atggacccca actgctcttg cgccactggt ggctcctgca cgtgcgccgg ctcctgcaag |
| 61 | tgcaaagagt gcaaatgcac ctcctgcaag aagagctgct gttcctgctg ccccgtgggc |
| 121 | tgtgccaagt gtgcccaggg ctgcgtctgc aaaggggcat cggagaagtg cagctgctgt |
| 181 | gcctga |

### Sequence Accession No.: NM_{—}001003828

Gene: Homo sapiens parvin, beta (PARVB)

| (SEQ ID NO: 128) | |
|---|---|
| 1 | atgcaccatg tgtttaaaga tcaccaaaga ggagagaaaa ggggattcct tagtccagag |
| 61 | aacaaaaact gcaggaggct ggagctgaga cgtgggtgtt cctgcagctg gggcctgtgc |
| 121 | tcccaggcac tcatggcttc tctggctggt tcacttctcc ctggctcaga cagatcagga |
| 181 | gtggaaacat ctgaatatgc tcaaggagga gtgagtgacc tgcaggaaga aggcaagaat |
| 241 | gccatcaact caccgatgtc ccccgccctg gtggatgttc accctgaaga cacccagctt |
| 301 | gaggagaacg aggagcgcac gatgattgac cccacttcca aggaagaccc caagttcaag |
| 361 | gaactggtca aggtcctcct cgactggatt aatgacgtgc tggtggagga gaggatcatt |
| 421 | gtgaagcagc tggaggaaga cctgtatgac ggccaggtgc tgcagaagct cttggaaaaa |
| 481 | ctggcagggt gcaagctgaa tgtggctgag gtgacacagt ccgaaatagg gcagaaacag |
| 541 | aagctgcaga cggtgctgga agcagtacat gacctgctgc ggccccgagg ctgggcgctc |
| 601 | cggtggagcg tggactcaat tcacgggaag aacctggtgg ccatcctcca cctgctggtc |
| 661 | tctctggcca tgcacttcag ggcccccatc cgccttcctg agcatgtaac ggtgcaggtg |
| 721 | gtggtcgtgc ggaaacggga aggcctgctg cattccagcc acatctcgga ggagctgacc |
| 781 | acaactacag agatgatgat gggccggttc gagcgggatg ccttcgacac gctgttcgac |
| 841 | cacgccccgg ataagctcag cgtggtgaag aagtctctca tcacttttgt gaacaagcac |
| 901 | ctgaacaagc tgaatttgga ggtgacggaa ctggagaccc agtttgcaga tggcgtgtac |
| 961 | ctggttctgc tcatgggcct tctggaagac tactttgttc ctctccacca cttctacctg |
| 1021 | actccggaaa gcttcgatca gaaggtccac aatgtgtcct tcgcctttga gctgatgctg |
| 1081 | gacggaggcc tcaagaaacc caaggctcgt cctgaagacg tggttaactt ggacctcaaa |
| 1141 | tccaccctga gggttcttta caacctgttc accaagtaca agaacgtgga gtga |

### Sequence Accession No.: NM_{—}006406

Gene: Homo sapiens peroxiredoxin 4 (PRDX4)

| (SEQ ID NO: 133) | |
|---|---|
| 1 | atggaggcgc tgccgctgct agccgcgaca actccggacc acggccgcca ccgaaggctg |
| 61 | cttctgctgc cgctactgct gttcctgctg ccggctggag ctgtgcaggg ctgggagaca |
| 121 | gaggagaggc cccggactcg cgaagaggag tgccacttct acgcgggtgg acaagtgtac |
| 181 | ccgggagagg catcccgggt atcggtcgcc gaccactccc tgcacctaag caaagcgaag |
| 241 | atttccaagc cagcgcccta ctgggaagga acagctgtga tcgatggaga atttaaggag |
| 301 | ctgaagttaa ctgattatcg tgggaaatac ttggttttct tcttctaccc acttgatttc |
| 361 | acatttgtgt gtccaactga aattatcgct tttggcgaca gacttgaaga attcagatct |
| 421 | ataaatactg aagtggtagc atgctctgtt gattcacagt ttacccattt ggcctggatt |
| 481 | aatacccctc gaagacaagg aggacttggg ccaataagga ttccacttct ttcagatttg |
| 541 | acccatcaga tctcaaagga ctatggtgta tacctagagg actcaggcca cactcttaga |
| 601 | ggtctcttca ttattgatga caaaggaatc ctaagacaaa ttactctgaa tgatcttcct |
| 661 | gtgggtagat cagtggatga gacactacgt ttggttcaag cattccagta cactgacaaa |
| 721 | cacggagaag tctgccctgc tggctggaaa cctggtagtg aaacaataat cccagatcca |
| 781 | gctggaaagc tgaagtattt cgataaactg aattga |

### Sequence Accession No.: NM_{—}145313

Gene: Homo sapiens RasGEF domain family, member 1A (RASGEF1A)

| (SEQ ID NO: 134) | |
|---|---|
| 1 | atgccccaga cgtccgttgt cttctccagc atccttgggc ccagctgtag cggacaggtg |
| 61 | cagcctggca tgggggagcg tggaggcggg gccggtggcg gctccgggga cctcatcttc |
| 121 | caagatggac acctcatctc tgggtccctg gaggccctga tggagcacct tgttcccacg |
| 181 | gtggactatt accccgatag gacgtacatc ttcacctttc tcctgagctc ccgggtcttt |
| 241 | atgccccctc atgacctgct ggcccgcgtg gggcagatct gcgtggagca gaagcagcag |
| 301 | ctggaagccg ggcctgaaaa ggccaagctg aagtctttct cagccaagat cgtgcagctc |
| 361 | ctgaaggagt ggaccgaggc cttcccctat gacttccagg atgagaaggc catggccgag |
| 421 | ctgaaagcca tcacacaccg tgtcacccag tgtgatgagg agaatggcac agtgaagaag |
| 481 | gccattgccc agatgacaca gagcctgttg ctgtccttgg ctgcccggag ccagctccag |
| 541 | gaactgcgag agaagctccg gccaccggct gtagacaagg ggcccatcct caagaccaag |
| 601 | ccaccagccg cccagaagga catcctgggc gtgtgctgcg accccctggt gctggcccag |
| 661 | cagctgactc acattgagct ggacagggtc agcagcattt accctgagga cttgatgcag |
| 721 | atcgtcagcc acatggactc cttggacaac cacaggtgcc gaggggacct gaccaagacc |
| 781 | tacagcctgg aggcctatga caactggttc aactgcctga gcatgctggt ggccactgag |
| 841 | gtgtgccggg tggtgaagaa gaaacaccgg acccgcatgt tggagttctt cattgatgtg |
| 901 | gcccgggagt gcttcaacat cgggaacttc aactccatga tggccatcat ctctggcatg |
| 961 | aacctcagtc ctgtggcaag gctgaagaaa acttggtcca aggtcaagac agccaagttt |
| 1021 | gatgtcttgg agcatcacat ggacccgtcc agcaacttct gcaactaccg tacagccctg |
| 1081 | cagggggcca cgcagaggtc ccagatggcc aacagcagcc gtgaaaagat cgtcatccct |
| 1141 | gtgttcaacc tcttcgttaa ggacatctac ttcctgcaca aaatccatac caaccacctg |
| 1201 | cccaacgggc acattaactt taagaaattc tgggagatct ccagacagat ccatgagttc |
| 1261 | atgacatgga cacaggtaga gtgtcctttc gagaaggaca agaagattca gagttacctg |
| 1321 | ctcacggcgc ccatctacag cgaggaagct ctcttcgtcg cctcctttga aagtgagggt |
| 1381 | cccgagaacc acatggaaaa agacagctgg aagaccctca ggaccaccct tctgaacaga |
| 1441 | gcctga |

### Sequence Accession No.: NM_{—}003973

Gene: Homo sapiens ribosomal protein L14 (RPL14)

| (SEQ ID NO: 135) | |
|---|---|
| 1 | atggtgttca ggcgcttcgt ggaggttggc cgggtggcct atgtctcctt tggacctcat |
| 61 | gccggaaaat tggtcgcgat tgtagatgtt attgatcaga acagggcttt ggtcgatgga |
| 121 | ccttgcactc aagtgaggag acaggccatg cctttcaagt gcatgcagct cactgatttc |
| 181 | atcctcaagt ttccgcacag tgcccaccag aagtatgtcc gacaagcctg gcagaaggca |
| 241 | gacatcaata caaaatgggc agccacacga tgggccaaga agattgaagc cagagaaagg |
| 301 | aaagccaaga tgacagattt tgatcgtttt aaagttatga aggcaaagaa aatgaggaac |
| 361 | agaataatca agaatgaagt taagaagctt caaaaggcag ctctcctgaa agcttctccc |
| 421 | aaaaaagcac ctggtactaa gggtactgct gctgctgctg ctgctgctgc tgctgctaaa |
| 481 | gttccagcaa aaaagatcac cgccgcgagt aaaaaggctc cagcccagaa ggttcctgcc |
| 541 | cagaaagcca caggccagaa agcagcgcct gctccaaaag ctcagaaggg tcaaaaagct |
| 601 | ccagcccaga aagcacctgc tccaaaggca tctggcaaga aagcataa |

### Sequence Accession No.: NM_{—}006332

Gene: Homo sapiens interferon, gamma-inducible protein 30 (IFI30)

| (SEQ ID NO: 136) | |
|---|---|
| 1 | atgaccctgt cgccacttct gctgttcctg ccaccgctgc tgctgctgct ggacgtcccc |
| 61 | acggcggcgg tgcaggcgtc ccctctgcaa gcgttagact tctttgggaa tgggccacca |
| 121 | gttaactaca agacaggcaa tctatacctg cgggggcccc tgaagaagtc caatgcaccg |
| 181 | cttgtcaatg tgaccctcta ctatgaagca ctgtgcggtg gctgccgagc cttcctgatc |
| 241 | cgggagctct tcccaacatg gctgttggtc atggagatcc tcaatgtcac gctggtgccc |
| 301 | tacggaaacg cacaggaaca aaatgtcagt ggcaggtggg agttcaagtg ccagcatgga |
| 361 | gaagaggagt gcaaattcaa caaggtggag gcctgcgtgt tggatgaact tgacatggag |
| 421 | ctagccttcc tgaccattgt ctgcatggaa gagtttgagg acatggagag aagtctgcca |
| 481 | ctatgcctgc agctctacgc cccagggctg tcgccagaca ctatcatgga gtgtgcaatg |
| 541 | ggggaccgcg gcatgcagct catgcacgcc aacgcccagc ggacagatgc tctccagcca |
| 601 | ccacacgagt atgtgccctg ggtcaccgtc aatgggaaac ccttggaaga tcagacccag |
| 661 | ctccttaccc ttgtctgcca gttgtaccag ggcaagaagc cggatgtctg cccttcctca |
| 721 | accagctccc tcaggagtgt ttgcttcaag tga |

### Sequence Accession No.: NM_{—}005171

Gene: Homo sapiens activating transcription factor 1 (ATF1)

| (SEQ ID NO: 137) | |
|---|---|
| 1 | atggaagatt cccacaagag taccacgtca gagacagcac ctcaacctgg ttcagcagtt |
| 61 | cagggagctc acatttctca tattgctcaa caggtatcat ctttatcaga aagtgaggag |
| 121 | tcccaggact catccgacag cataggctcc tcacagaaag cccacgggat cctagcacgg |
| 181 | cgcccatctt acagaaaaat tttgaaagac ttatcttctg aagatacacg gggcagaaaa |
| 241 | ggagacggag aaaattctgg agtttctgct gctgtcactt ctatgtctgt tccaactccc |
| 301 | atctatcaga ctagcagcgg acagtacatt gccattgccc caaatggagc cttacagttg |
| 361 | gcaagtccag gcacagatgg agtacaggga cttcagacat taaccatgac aaattcaggc |
| 421 | agtactcagc aaggtacaac tattcttcag tatgcacaga cctctgatgg acagcagata |
| 481 | cttgtgccca gcaatcaggt ggtcgtacaa actgcatcag gagatatgca aacatatcag |
| 541 | atccgaacta caccttcagc tacttctctg ccacaaactg tggtgatgac atctcctgtg |
| 601 | actctcacct ctcagacaac taagacagat gacccccaat tgaaaagaga aataaggtta |
| 661 | atgaaaaaca gagaagctgc tcgagaatgt cgcagaaaga agaaagaata tgtgaaatgc |
| 721 | ctggaaaacc gagttgcagt cctggaaaat caaaataaaa ctctaataga agagttaaaa |
| 781 | actttgaagg atctttattc caataaaagt gtttga |

### Sequence Accession No.: NM_{—}000018

Gene: Homo sapiens acyl-Coenzyme A dehydrogenase, very long chain (ACADVL), nuclear gene encoding mitochondrial protein

| (SEQ ID NO: 138) | |
|---|---|
| 1 | atgcaggcgg ctcggatggc cgcgagcttg gggcggcagc tgctgaggct cgggggcgga |
| 61 | agctcgcggc tcacggcgct cctggggcag ccccggcccg gccctgcccg gcggccctat |
| 121 | gccgggggtg ccgctcagct ggctctggac aagtcagatt cccacccctc tgacgctctg |
| 181 | accaggaaaa aaccggccaa ggcggaatct aagtcctttg ctgtgggaat gttcaaaggc |
| 241 | cagctcacca cagatcaggt gttcccatac ccgtccgtgc tcaacgaaga gcagacacag |
| 301 | tttcttaaag agctggtgga gcctgtgtcc cgtttcttcg aggaagtgaa cgatcccgcc |
| 361 | aagaatgacg ctctggagat ggtggaggag accacttggc agggcctcaa ggagctgggg |
| 421 | gcctttggtc tgcaagtgcc cagtgagctg ggtggtgtgg gcctttgcaa cacccagtac |
| 481 | gcccgtttgg tggagatcgt gggcatgcat gaccttggcg tgggcattac cctgggggcc |
| 541 | catcagagca tcggtttcaa aggcatcctg ctctttggca caaaggccca gaaagaaaaa |
| 601 | tacctcccca agctggcatc tggggagact gtggccgctt tctgtctaac cgagccctca |
| 661 | agcgggtcag atgcagcctc catccgaacc tctgctgtgc ccagcccctg tggaaaatac |
| 721 | tataccctca atggaagcaa gctttggatc agtaatgggg gcctagcaga catcttcacg |
| 781 | gtctttgcca agacaccagt tacagatcca gccacaggag ccgtgaagga gaagatcaca |
| 841 | gcttttgtgg tggagagggg cttcgggggc attacccatg ggccccctga gaagaagatg |
| 901 | ggcatcaagg cttcaaacac agcagaggtg ttctttgatg gagtacgggt gccatcggag |
| 961 | aacgtgctgg gtgaggttgg gagtggcttc aaggttgcca tgcacatcct caacaatgga |
| 1021 | aggtttggca tggctgcggc cctggcaggt accatgagag gcatcattgc taaggcggta |
| 1081 | gatcatgcca ctaatcgtac ccagtttggg gagaaaattc acaactttgg gctgatccag |
| 1141 | gagaagctgg cacggatggt tatgctgcag tatgtaactg agtccatggc ttacatggtg |
| 1201 | agtgctaaca tggaccaggg agccacggac ttccagatag aggccgccat cagcaaaatc |
| 1261 | tttggctcgg aggcagcctg gaaggtgaca gatgaatgca tccaaatcat ggggggtatg |
| 1321 | ggcttcatga aggaacctgg agtagagcgt gtgctccgag atcttcgcat cttccggatc |
| 1381 | tttgagggga caaatgacat tcttcggctg tttgtggctc tgcagggctg tatggacaaa |
| 1441 | ggaaaggagc tctctgggct tggcagtgct ctaaagaatc cctttgggaa tgctggcctc |
| 1501 | ctgctaggag aggcaggcaa acagctgagg cggcgggcag ggctgggcag cggcctgagt |
| 1561 | ctcagcggac ttgtccaccc ggagttgagt cggagtggcg agctggcagt acgggctctg |
| 1621 | gagcagtttg ccactgtggt ggaggccaag ctgataaaac acaagaaggg gattgtcaat |
| 1681 | gaacagtttc tgctgcagcg gctggcagac ggggccatcg acctctatgc catggtggtg |
| 1741 | gttctctcga gggcctcaag atccctgagt gagggccacc ccacggccca gcatgagaaa |
| 1801 | atgctctgtg acacctggtg tatcgaggct gcagctcgga tccgagaggg catggccgcc |
| 1861 | ctgcagtctg acccctggca gcaagagctc taccgcaact tcaaaagcat ctccaaggcc |
| 1921 | ttggtggagc ggggtggtgt ggtcaccagc aacccacttg gcttctga |

### Sequence Accession No.: NM_{—}018438

Gene: Homo sapiens F-box protein 6 (FBXO6)

| (SEQ ID NO: 139) | |
|---|---|
| 1 | atggatgctc cccactccaa agcagccctg gacagcatta acgagctgcc cgagaacatc |
| 61 | ctgctggagc tgttcacgca cgtgcccgcc cgccagctgc tgctgaactg ccgcctggtc |
| 121 | tgcagcctct ggcgggacct catcgacctc atgaccctct ggaaacgcaa gtgcctgcga |
| 181 | gagggcttca tcaccaagga ctgggaccag cccgtggccg actggaaaat cttctacttc |
| 241 | ctacggagcc tgcataggaa cctcctgcgc aacccgtgtg ctgaagagga tatgtttgca |
| 301 | tggcaaattg atttcaatgg tggggaccgc tggaaggtgg agagcctccc tggagcccac |
| 361 | gggacagatt ttcctgaccc caaagtcaag aagtattttg tcacatccta cgaaatgtgc |
| 421 | ctcaagtccc agctggtgga ccttgtagcc gagggctact gggaggagct actagacaca |
| 481 | ttccggccgg acatcgtggt taaggactgg tttgctgcca gagccgactg tggctgcacc |
| 541 | taccaactca aagtgcagct ggcctcggct gactacttcg tgttggcctc cttcgagccc |
| 601 | ccacctgtga ccatccaaca gtggaacaat gccacatgga cagaggtctc ctacaccttc |
| 661 | tcagactacc cccggggtgt ccgctacatc ctcttccagc atgggggcag ggacacccag |
| 721 | tactgggcag gctggtatgg gccccgagtc accaacagca gcattgtcgt cagccccaag |
| 781 | atgaccagga accaggcctc ctccgaggct cagcctgggc agaagcatgg acaggaggag |
| 841 | gctgcccaat cgccctaccg agctgttgtc cagattttct ga |

### Sequence Accession No.: NM_{—}000904

Gene: Homo sapiens NAD(P)H dehydrogenase, quinone 2 (NQO2)

| (SEQ ID NO: 140) | |
|---|---|
| 1 | atggcaggta agaaagtact cattgtctat gcacaccagg aacccaagtc tttcaacgga |
| 61 | tccttgaaga atgtggctgt agatgaactg agcaggcagg gctgcaccgt cacagtgtct |
| 121 | gatttgtatg ccatgaactt tgagccgagg gccacagaca aagatatcac tggtactctt |
| 181 | tctaatcctg aggttttcaa ttatggagtg gaaacccacg aagcctacaa gcaaaggtct |
| 241 | ctggctagcg acatcactga tgagcagaaa aaggttcggg aggctgacct agtgatattt |
| 301 | cagttcccgc tgtactggtt cagcgtgccg gccatcctga agggctggat ggatagggtg |
| 361 | ctgtgccagg gctttgcctt tgacatccca ggattctacg attccggttt gctccagggt |
| 421 | aaactagcgc tcctttccgt aaccacggga ggcacggccg agatgtacac gaagacagga |
| 481 | gtcaatggag attctcgata cttcctgtgg ccactccagc atggcacatt acacttctgt |
| 541 | ggatttaaag tccttgcccc tcagatcagc tttgctcctg aaattgcatc cgaagaagaa |
| 601 | agaaagggga tggtggctgc gtggtcccag aggctgcaga ccatctggaa ggaagagccc |
| 661 | atcccctgca cagcccactg gcacttcggg caataa |

### Sequence Accession No.: NM_{—}001008495

Gene: Homo sapiens transmembrane protein 64 (TMEM64)

| (SEQ ID NO: 141) | |
|---|---|
| 1 | atgggtctga tgatggtggg cgtcctcatc ggcaccttca tcgcccatgt ggtctgcaag |
| 61 | cggctcctca ccgcctgggt ggccgccagg atccagagca gcgagaagct gagcgcggtt |
| 121 | attcgcgtag tggagggagg aagcggcctg aaagtggtgg cgctggccag actgacaccc |
| 181 | ataccttttg ggcttcagaa tgcagtgttt tcgattactg atctctcatt acccaactat |
| 241 | ctgatggcat cttcggttgg actgcttcct acccagcttc tgaattctta cttgggtacc |
| 301 | accctgcgga caatggaaga tgtcattgca gaacagagtg ttagtggata ttttgttttt |
| 361 | tgtttacaga ttattataag tataggcctc atgttttatg tagttcatcg agctcaagtg |
| 421 | gaattgaatg cagctattgt agcttgtgaa atggaactga aatcttctct ggttaaaggc |
| 481 | aatcaaccaa ataccagtgg ctcttcattc tacaacaaga ggaccctaac attttctgga |
| 541 | ggtggaatca atgttgtatg a |

### Sequence Accession No.: NM_{—}024699

Gene: Homo sapiens zinc finger, AN1-type domain 1 (ZFAND1)

```
(SEQ ID NO: 142)
    1 atggcggagt tggacatcgg gcagcactgc caggtggagc attgccggca gcgagatttt
  61 cttccatttg tgtgtgatga ttgttcagga atattttgcc ttgaacacag aagcagggag
121 tctcatggtt gtcctgaggt gactgtaatc aatgagagac tgaagacaga tcaacataca
181 tcttacccat gctctttcaa agactgtgct gagagagaac ttgtggcagt tatatgtcct
241 tattgtgaga agaatttttg cctgagacac cgtcatcagt cagatcatga gtgtgaaaaa
301 ctggaaatcc caaagcctcg aatggctgcc actcagaaac ttgttaaaga cattattgat
361 tccaagacag gagaaacagc aagtaaacga tggaaaggtg ccaaaaatag tgaaacagct
421 gcaaaggttg cattgatgaa attaaagatg catgctgatg gcgataagtc attaccacag
481 acagaaagaa tttactttca ggttttctta cctaaaggga gcaaagagaa gagcaaacca
541 atgctctttt gccaccgatg gagcattgga aaggccatag actttgccgc ttctctagcc
601 aggcttaaaa atgacaataa caaatttaca gctaagaaat taaggctgtg tcacattact
661 tcaggagaag ccttaccctt ggatcatact ttggaaacct ggattgctaa ggaggattgt
721 cctttatata atggtggaaa tataatcttg gaatatctca atgatgaaga acaattctgt
781 aaaaatgttg aatcttactt ggaatag
```

### Sequence Accession No.: NM_{—}016040

Gene: Homo sapiens transmembrane emp24 protein transport domain containing 5 (TMED5)

```
(SEQ ID NO: 143)
    1 atgggcgaca agatctggct gcccttcccc gtgctccttc tggccgctct gcctccggtg
  61 ctgctgcctg gggcggccgg cttcacacct tccctcgata gcgacttcac ctttaccctt
121 cccgccggcc agaaggagtg cttctaccag cccatgcccc tgaaggcctc gctggagatc
181 gagtaccaag ttttagatgg agcaggatta gatattgatt tccatcttgc ctctccagaa
241 ggcaaaacct tagtttttga acaaagaaaa tcagatggag ttcacactgt agagactgaa
301 gttggtgatt acatgttctg ctttgacaat acattcagca ccatttctga gaaggtgatt
361 ttctttgaat taatcctgga taatatggga gaacaggcac aagaacaaga agattggaag
421 aaatatatta ctggcacaga tatattggat atgaaactgg aagacatcct ggaatccatc
481 aacagcatca agtccagact aagcaaaagt gggcacatac aaattctgct tagagcattt
541 gaagctcgtg atcgaaacat acaagaaagc aactttgata gagtcaattt ctggtctatg
601 gttaatttag tggtcatggt ggtggtgtca gccattcaag tttatatgct gaagagtctg
661 tttgaagata agaggaaaag tagaacttaa
```

### Sequence Accession No.: NM_{—}006810

Gene: Homo sapiens protein disulfide isomerase family A, member 5 (PDIA5)

```
(SEQ ID NO: 144)
      1 atggcgcggg ccgggccggc gtggctgctg ctggcaatct gggtggtcct gccatcatgg
    61 ctgtcctctg caaaggtctc ctcgctcatt gagagaatct ctgaccccaa ggacttgaaa
  121 aaactgctca gaacccggaa taatgtactg gtgctttact ccaaatctga ggtggcagct
  181 gaaaatcatc tcaggttact gtccacagtg gcccaggcgg tgaaaggaca agggaccatc
  241 tgctgggtgg actgtggtga tgcagagagt agaaaattgt gcaagaagat gaaagttgac
  301 ctgagcccga aggacaaaaa ggttgaatta ttccattacc aggatggtgc atttcatact
  361 gaatataacc gagctgtgac atttaagtcc atagtggcct ttttgaagga tccaaaaggg
  421 cccccactgt gggaggaaga tcctggagcc aaagatgttg tccaccttga cagtgaaaag
  481 gacttcagac ggctcctgaa gaaggaagag aagccgctcc tgatcatgtt ttatgccccc
  541 tggtgcagca tgtgcaagag gatgatgccg catttccaga aggctgcgac tcagctgcga
  601 ggccacgccg tgctggccgg gatgaatgtc tactcctctg aatttgaaaa catcaaggag
  661 gagtacagcg tgcgcggctt ccccaccatc tgctattttg agaaaggacg gttcttgttc
  721 cagtatgaca actatgggtc cacagctgag gacattgtgg agtggctgaa gaatccgcag
  781 ccgccacagc cccaggtccc tgagactccc tgggcagatg agggcggctc cgtttatcac
  841 ctgaccgatg aagactttga ccagtttgtg aaggaacact cctctgtcct cgtcatgttc
  901 cacgccccat ggtgtggcca ctgtaagaaa atgaagccgg agtttgagaa ggcagcagaa
  961 gccctccatg gagaagcgga tagctctggt gtccttgcag ctgtcgatgc cactgtcaac
1021 aaggccctgg cagaaagatt ccacatctca gagtttccta cgttgaagta ttttaagaat
1081 ggagagaaat acgcagtgcc tgtgctcagg acaaagaaga agtttctcga gtggatgcaa
1141 aaccctgagg cccccccgcc cccagagccc acgtgggaag agcagcagac aagcgtgttg
1201 cacctggtgg gggacaactt ccgggagacc ctgaagaaga agaaacacac cttggtcatg
1261 ttctacgccc cttggtgccc acactgtaag aaggtcattc cgcactttac tgctactgct
1321 gatgccttca aagatgaccg aaagattgcc tgtgccgctg ttgactgtgt caaagacaag
1381 aaccaagacc tgtgccagca ggaggcggtc aagggctacc ccactttcca ctactaccac
1441 tatgggaagt tcgcagaaaa gtatgacagc gaccgcacag aattgggatt taccaattat
1501 attcgagccc tccgggaggg agaccatgaa agactaggga aaaagaagga agagttataa
```

### Sequence Accession No.: NM_{—}033375

Gene: Homo sapiens myosin IC (MYO1C)

```
(SEQ ID NO: 145)
      1 atggagagtg cgctcaccgc ccgtgaccgg gtgggggtgc aggatttcgt gctgctggag
    61 aacttcacca gcgaggccgc cttcatcgag aacctgcggc ggcgatttcg ggagaatctc
  121 atctacacct acattggccc cgtcctggtc tctgtcaatc cctaccggga cctgcagatc
  181 tacagccggc agcatatgga gcgttaccgt ggcgtcagct tctatgaagt gccccctcac
  241 ctgtttgccg tggcggacac tgtgtaccga gcactgcgca cggagcgtcg ggaccaggct
  301 gtgatgatct ctggggagag cggggcaggc aagaccgagg ccaccaagag gctgctgcag
  361 ttctatgcag agacctgccc agcccccgag cgcggaggtg ccgtgcggga ccggctgcta
  421 cagagcaacc cggtgctgga ggcctttgga aatgccaaga ccctccggaa cgataactcc
  481 agcaggttcg ggaagtacat ggatgtgcag tttgacttca agggtgcccc cgtgggtggc
  541 cacatcctca gttacctcct ggaaaagtca cgagtggtgc accagaatca tggggagcgg
  601 aacttccaca tcttctacca gctgctggag gggggcgagg aggagactct tcgcaggctg
  661 ggcttggaac ggaaccccca gagctacctg tacctggtga agggccagtg tgccaaagtc
  721 tcctccatca acgacaagag tgactggaag gtcgtcagga aggctctgac agtcattgat
  781 ttcaccgagg atgaagtgga ggacctgctg agcatcgtgg ccagcgtcct tcatttgggc
  841 aacatccact ttgctgccaa cgaggagagc aatgcccagg tcaccaccga gaaccagctc
  901 aagtatctga ccaggctcct cagcgtggaa ggctcgacgc tgcgagaagc cctgacacac
  961 aggaagatca tcgccaaggg ggaggagctc ctgagcccgc tgaacctgga gcaggccgcg
1021 tacgcacgag acgccctcgc caaggctgtg tacagccgca cttttacctg gctcgtcggg
1081 aagatcaaca ggtcgctggc ctccaaggac gtggagagcc ccagctggcg gagcaccacg
1141 gttctcgggc tcctggatat ttatggcttt gaagtgtttc agcataacag ctttgagcag
1201 ttctgcatca attactgcaa cgagaagctg cagcagctct tcatcgagct cacgctcaag
1261 tcggagcagg aggagtacga ggcagagggc atcgcgtggg agcccgtcca gtatttcaac
1321 aacaaaatca tctgtgatct ggtggaggag aagtttaagg gcatcatctc gattttggat
1381 gaggagtgtc tgcgccccgg ggaggccaca gacctgacct tcctggagaa gctggaggat
1441 actgtcaagc accatccaca cttcctgacg cacaagctgg ctgaccagcg gaccaggaaa
1501 tctctgggcc gaggggaatt ccgccttctg cactatgcgg gggaggtgac ctacagcgtg
1561 accgggtttc tggacaaaaa caatgacctt ctcttccgga accttaagga gaccatgtgt
1621 agctcaaaga atcccattat gagccagtgc tttgaccgga gcgagctcag tgacaagaag
1681 cggccagaga cggtcgccac ccagttcaag atgagcctcc tgcagctggt ggagatcctg
1741 cagtctaagg agcccgccta cgtccgctgc atcaaaccca atgatgccaa acagcccggc
1801 cgctttgacg aggtgctgat ccgccaccag gtgaagtacc tggggctgtt ggaaaacctg
1861 cgcgtgcgca gagccggctt tgcctatcgc cgcaaatacg aagctttcct gcaaaggtac
1921 aagtcactgt gcccagagac gtggcccacg tgggcaggac ggccgcagga tggggtggct
1981 gtgctggtcc gacacctggg ctacaagcca gaagagtaca agatgggcag gaccaagatc
2041 ttcatccgct tccccaagac cctgtttgcc acagaggatg ccctggaggt ccggcggcag
2101 agcctggcca caaagatcca agctgcctgg aggggctttc actggcggca gaaattcctc
2161 cgggtgaaga gatcagccat ctgcatccag tcgtggtggc gtggaacact gggccggagg
2221 aaggcagcca agaggaagtg ggcggcacag accatccggc ggctcatccg aggcttcgtc
2281 ctgcgccacg ccgcccgctg ccccgagaac gccttcttcc tggaccatgt gcgcacctct
2341 tttttgctaa acctgaggcg gcagctgccc cagaatgtcc tggacacctc gtggcccacg
2401 cccccacctg ccgtgcggga ggcctcagag cttctgcggg agttgtgcat aaagaacatg
2461 gtgtggaaat actgccggag tatcagccct gagtggaagc agcagctgca gcagaaggcc
2521 gtggctagtg agatcttcaa gggcaagaag gataattacc ctcagagtgt acccaggctc
2581 ttcatcagca ctcggcttgg tacagatgag atcagccccc gagtgctgca ggccttgggc
2641 tctgagccca ttcagtatgc ggtgcctgtt gtgaaatacg accgcaaggg ctacaagcct
2701 cgctcccggc agctgctgct cacgcccaac gccgtcgtca tcgtggagga cgccaaagtc
2761 aagcagagga ttgattacgc caacctgacc ggaatctctg tcagcagcct gagcgacagt
2821 ctttttgtgc ttcatgtaca gcgtgcggac aataagcaaa agggagatgt ggtgctgcag
2881 agtgaccacg tgattgagac gctgaccaag acagccctca gtgccaaccg cgtgaacagc
2941 atcaacatca accagggcag catcacgttt gcagggggcc ccggcaggga tggcaccatt
3001 gacttcacac ccggctcgga gctgctcatc accaaggcca agaacgggca cctggctgtg
3061 gtcgccccac ggctgaattc tcggtga
```

### Sequence Accession No.: NM_{—}024312

Gene: Homo sapiens N-acetylglucosamine-1-phosphate transferase, alpha and beta subunits (GNPTAB)

```
(SEQ ID NO: 146)
      1 atgctgttca agctcctgca gagacagacc tatacctgcc tgtcccacag gtatgggctc
    61 tacgtgtgct tcttgggcgt cgttgtcacc atcgtctccg ccttccagtt cggagaggtg
  121 gttctggaat ggagccgaga tcaataccat gttttgtttg attcctatag agacaatatt
  181 gctggaaagt cctttcagaa tcggctttgt ctgcccatgc cgattgacgt tgtttacacc
  241 tgggtgaatg gcacagatct tgaactactg aaggaactac agcaggtcag agaacagatg
  301 gaggaggagc agaaagcaat gagagaaatc cttgggaaaa acacaacgga acctactaag
  361 aagagtgaga agcagttaga gtgtttgcta acacactgca ttaaggtgcc aatgcttgtc
  421 ctggacccag ccctgccagc caacatcacc ctgaaggacc tgccatctct ttatccttct
  481 tttcattctg ccagtgacat tttcaatgtt gcaaaaccaa aaaacccttc taccaatgtc
  541 tcagttgttg tttttgacag tactaaggat gttgaagatg cccactctgg actgcttaaa
  601 ggaaatagca gacagacagt atggaggggc tacttgacaa cagataaaga agtccctgga
  661 ttagtgctaa tgcaagattt ggctttcctg agtggatttc caccaacatt caaggaaaca
  721 aatcaactaa aaacaaaatt gccagaaaat ctttcctcta aagtcaaact gttgcagttg
  781 tattcagagg ccagtgtagc gcttctaaaa ctgaataacc ccaaggattt tcaagaattg
  841 aataagcaaa ctaagaagaa catgaccatt gatggaaaag aactgaccat aagtcctgca
  901 tatttattat gggatctgag cgccatcagc cagtctaagc aggatgaaga catctctgcc
  961 agtcgttttg aagataacga agaactgagg tactcattgc gatctatcga gaggcatgca
1021 ccatgggttc ggaatatttt cattgtcacc aacgggcaga ttccatcctg gctgaacctt
1081 gacaatcctc gagtgacaat agtaacacac caggatgttt ttcgaaattt gagccacttg
1141 cctaccttta gttcacctgc tattgaaagt cacattcatc gcatcgaagg gctgtcccag
1201 aagtttattt acctaaatga tgatgtcatg tttgggaagg atgtctggcc agatgatttt
1261 tacagtcact ccaaaggcca gaaggtttat ttgacatggc ctgtgccaaa ctgtgccgag
1321 ggctgcccag gttcctggat taaggatggc tattgtgaca aggcttgtaa taattcagcc
1381 tgcgattggg atggtgggga ttgctctgga aacagtggag ggagtcgcta tattgcagga
1441 ggtggaggta ctgggagtat tggagttgga cagccctggc agtttggtgg aggaataaac
1501 agtgtctctt actgtaatca gggatgtgcg aattcctggc tcgctgataa gttctgtgac
1561 caagcatgca atgtcttgtc ctgtgggttt gatgctggcg actgtgggca agatcatttt
1621 catgaattgt ataaagtgat ccttctccca aaccagactc actatattat tccaaaaggt
1681 gaatgcctgc cttatttcag ctttgcagaa gtagccaaaa gaggagttga aggtgcctat
1741 agtgacaatc caataattcg acatgcttct attgccaaca agtggaaaac catccacctc
1801 ataatgcaca gtggaatgaa tgccaccaca atacatttta atctcacgtt tcaaaataca
1861 aacgatgaag agttcaaaat gcagataaca gtggaggtgg acacaaggga gggaccaaaa
1921 ctgaattcta cagcccagaa gggttacgaa aatttagtta gtcccataac acttcttcca
1981 gaggcggaaa tcctttttga ggatattccc aaagaaaaac gcttcccgaa gtttaagaga
2041 catgatgtta actcaacaag gagagcccag gaagaggtga aaattcccct ggtaaatatt
2101 tcactccttc caaaagacgc ccagttgagt ctcaatacct tggatttgca actggaacat
2161 ggagacatca ctttgaaagg atacaatttg tccaagtcag ccttgctgag atcatttctg
2221 atgaactcac agcatgctaa aataaaaaat caagctataa taacagatga aacaaatgac
2281 agtttggtgg ctccacagga aaaacaggtt cataaaagca tcttgccaaa cagcttagga
2341 gtgtctgaaa gattgcagag gttgactttt cctgcagtga gtgtaaaagt gaatggtcat
2401 gaccagggtc agaatccacc cctggacttg gagaccacag caagatttag agtggaaact
2461 cacacccaaa aaaccatagg cggaaatgtg acaaaagaaa agcccccatc tctgattgtt
2521 ccactggaaa gccagatgac aaaagaaaag aaaatcacag ggaaagaaaa agagaacagt
2581 agaatggagg aaaatgctga aaatcacata ggcgttactg aagtgttact tggaagaaag
2641 ctgcagcatt acacagatag ttacttgggc tttttgccat gggagaaaaa aaagtatttc
2701 caagatcttc tcgacgaaga agagtcattg aagacacaat tggcatactt cactgatagc
2761 aaaaatactg ggaggcaact aaaagataca tttgcagatt ccctcagata tgtaaataaa
2821 attctaaata gcaagtttgg attcacatcg cggaaagtcc ctgctcacat gcctcacatg
2881 attgaccgga ttgttatgca agaactgcaa gatatgttcc ctgaagaatt tgacaagacg
2941 tcatttcaca aagtgcgcca ttctgaggat atgcagtttg ccttctctta tttttattat
3001 ctcatgagtg cagtgcagcc actgaatata tctcaagtct ttgatgaagt tgatacagat
3061 caatctggtg tcttgtctga cagagaaatc cgaacactgg ctaccagaat tcacgaactg
3121 ccgttaagtt tgcaggattt gacaggtctg gaacacatgc taataaattg ctcaaaaatg
3181 cttcctgctg atatcacgca gctaaataat attccaccaa ctcaggaatc ctactatgat
3241 cccaacctgc caccggtcac taaaagtcta gtaacaaact gtaaaccagt aactgacaaa
3301 atccacaaag catataagga caaaaacaaa tataggtttg aaatcatggg agaagaagaa
3361 atcgctttta aaatgattcg taccaacgtt tctcatgtgg ttggccagtt ggatgacata
3421 agaaaaaacc ctaggaagtt tgtttgcctg aatgacaaca ttgaccacaa tcataaagat
3481 gctcagacag tgaaggctgt tctcagggac ttctatgaat ccatgttccc cataccttcc
3541 caatttgaac tgccaagaga gtatcgaaac cgtttccttc atatgcatga gctgcaggaa
3601 tggagggctt atcgagacaa attgaagttt tggacccatt gtgtactagc aacattgatt
3661 atgtttacta tattctcatt ttttgctgag cagttaattg cacttaagcg gaagatattt
3721 cccagaagga ggatacacaa agaagctagt cccaatcgaa tcagagtata g
```

### Sequence Accession No.: NM_{—}016027

Gene: Homo sapiens lactamase, beta 2 (LACTB2).

```
(SEQ ID NO: 147)
    1 atggctgctg tactgcagcg cgtcgagcgg ctgtccaatc gagtcgtgcg tgtgttgggc
  61 tgtaacccgg gtcccatgac cctccaaggc accaacacct acctagtggg gaccggcccc
121 aggagaatcc tcattgacac tggagaacca gcaattccag aatacatcag ctgtttaaag
181 caggctctaa ctgaatttaa cacagcaatc caggaaattg tagtgactca ctggcaccga
241 gatcattctg gaggcatagg agatatttgt aaaagcatca ataatgacac tacctattgc
301 attaaaaaac tcccacggaa tcctcagaga gaagaaatta taggaaatgg agagcaacaa
361 tatgtttatc tgaaagatgg agatgtgatt aagactgagg gagccactct aagagttcta
421 tatacccctg gccacactga tgatcacatg gctctactct tagaagagga aaatgctatc
481 ttttctggag attgcatcct aggggaagga acaacggtat ttgaagacct ctatgattat
541 atgaactctt taaaagagtt attgaaaatc aaagctgata ttatatatcc aggacatggc
601 ccagtaattc ataatgctga agctaaaatt caacaataca tttctcacag aaatattcga
661 gagcagcaaa ttcttacatt atttcgtgag aactttgaga aatcatttac agtaatggag
721 cttgtaaaaa ttatttacaa gaatactcct gagaatttac atgaaatggc taaacataat
781 ctcttacttc atttgaaaaa actagaaaaa gaaggaaaaa tatttagcaa cacagatcct
841 gacaagaaat ggaaagctca tctttag
```

### Sequence Accession No.: NM_{—}000983

Gene: Homo sapiens ribosomal protein L22 (RPL22)

```
(SEQ ID NO: 148)
    1 atggctcctg tgaaaaagct tgtggtgaag gggggcaaaa aaaagaagca agttctgaag
  61 ttcactcttg attgcaccca ccctgtagaa gatggaatca tggatgctgc caattttgag
121 cagtttttgc aagaaaggat caaagtgaac ggaaaagctg ggaaccttgg tggaggggtg
181 gtgaccatcg aaaggagcaa gagcaagatc accgtgacat ccgaggtgcc tttctccaaa
241 aggtatttga aatatctcac caaaaaatat ttgaagaaga ataatctacg tgactggttg
301 cgcgtagttg ctaacagcaa agagagttac gaattacgtt acttccagat taaccaggac
361 gaagaagagg aggaagacga ggattaa
```

### Sequence Accession No.: NM_{—}001025234

Gene: Homo sapiens tetraspanin 4 (TSPAN4)

```
(SEQ ID NO: 149)
    1 atggcgcgcg cctgcctcca ggccgtcaag tacctcatgt tcgccttcaa cctgctcttc
  61 tggctgggag gctgtggcgt gctgggtgtc ggcatctggc tggccgccac acaggggagc
121 ttcgccacgc tgtcctcttc cttcccgtcc ctgtcggctg ccaacttgct catcatcacc
181 ggcgcctttg tcatggccat cggcttcgtg ggctgcctgg gtgccatcaa ggagaacaag
241 tgcctcctgc tcactttctt cctgctgctg ctgctggtgt tcctgctgga ggccaccatc
301 gccatcctct tcttcgccta cacggacaag attgacaggt atgcccagca agacctgaag
361 aaaggcttgc acctgtacgg cacgcagggc aacgtgggcc tcaccaacgc ctggagcatc
421 atccagaccg acttccgctg ctgtggcgtc tccaactaca ctgactggtt cgaggtgtac
481 aacgccacgc gggtacctga ctcctgctgc ttggagttca gtgagagctg tgggctgcac
541 gcccccggca cctggtggaa ggcgccgtgc tacgagacgg tgaaggtgtg gcttcaggag
601 aacctgctgg ctgtgggcat ctttgggctg tgcacggcgc tggtgcagat cctgggcctg
661 accttcgcca tgaccatgta ctgccaagtg gtcaaggcag acacctactg cgcgtag
```

### Sequence Accession No.: NM_{—}002948

Gene: Homo sapiens ribosomal protein L15 (RPL15)

```
(SEQ ID NO: 150)
    1 atgggtgcat acaagtacat ccaggagcta tggagaaaga agcagtctga tgtcatgcgc
  61 tttcttctga gggtccgctg ctggcagtac cgccagctct ctgctctcca cagggctccc
121 cgccccaccc ggcctgataa agcgcgccga ctgggctaca aggccaagca aggttacgtt
181 atatatagga ttcgtgttcg ccgtggtggc cgaaaacgcc cagttcctaa gggtgcaact
241 tacggcaagc ctgtccatca tggtgttaac cagctaaagt ttgctcgaag ccttcagtcc
301 gttgcagagg agcgagctgg acgccactgt ggggctctga gagtcctgaa ttcttactgg
361 gttggtgaag attccacata caaatttttt gaggttatcc tcattgatcc attccataaa
421 gctatcagaa gaaatcctga cacccagtgg atcaccaaac cagtccacaa gcacagggag
481 atgcgtgggc tgacatctgc aggccgaaag agccgtggcc ttggaaaggg ccacaagttc
541 caccacacta ttggtggctc tcgccgggca gcttggagaa ggcgcaatac tctccagctc
601 caccgttacc gctaa
```

### Sequence Accession No.: NM_{—}000532

Gene: Homo sapiens propionyl Coenzyme A carboxylase, beta polypeptide (PCCB)

```
(SEQ ID NO: 151)
      1 atggcggcgg cattacgggt ggcggcggtc ggggcaaggc tcagcgttct ggcgagcggt
    61 ctccgcgccg cggtccgcag cctttgcagc caggccacct ctgttaacga acgcatcgaa
  121 aacaagcgcc ggaccgcgct gctgggaggg ggccaacgcc gtattgacgc gcagcacaag
  181 cgaggaaagc taacagccag ggagaggatc agtctcttgc tggaccctgg cagctttgtt
  241 gagagcgaca tgtttgtgga acacagatgt gcagattttg gaatggctgc tgataagaat
  301 aagtttcctg gagacagcgt ggtcactgga cgaggccgaa tcaatggaag attggtttat
  361 gtcttcagtc aggattttac agtttttgga ggcagtctgt caggagcaca tgcccaaaag
  421 atctgcaaaa tcatggacca ggccataacg gtgggggctc cagtgattgg gctgaatgac
  481 tctgggggag cacggatcca agaaggagtg gagtctttgg ctggctatgc agacatcttt
  541 ctgaggaatg ttacggcatc cggagtcatc cctcagattt ctctgatcat gggcccatgt
  601 gctggtgggg ccgtctactc cccagcccta acagacttca cgttcatggt aaaggacacc
  661 tcctacctgt tcatcactgg ccctgatgtt gtgaagtctg tcaccaatga ggatgttacc
  721 caggaggagc tcggtggtgc caagacccac accaccatgt caggtgtggc ccacagagct
  781 tttgaaaatg atgttgatgc cttgtgtaat ctccgggatt tcttcaacta cctgcccctg
  841 agcagtcagg acccggctcc cgtccgtgag tgccacgatc ccagtgaccg tctggttcct
  901 gagcttgaca caattgtccc tttggaatca accaaagcct acaacatggt ggacatcata
  961 cactctgttg ttgatgagcg tgaatttttt gagatcatgc ccaattatgc caagaacatc
1021 attgttggtt ttgcaagaat gaatgggagg actgttggaa ttgttggcaa ccaacctaag
1081 gtggcctcag gatgcttgga tattaattca tctgtgaaag gggctcgttt tgtcagattc
1141 tgtgatgcat tcaatattcc actcatcact tttgttgatg tccctggctt tctacctggc
1201 acagcacagg aatacggggg catcatccgg catggtgcca agcttctcta cgcatttgct
1261 gaggcaactg tacccaaagt cacagtcatc accaggaagg cctatggagg tgcctatgat
1321 gtcatgagct ctaagcacct ttgtggtgat accaactatg cctggcccac cgcagagatt
1381 gcagtcatgg gagcaaaggg cgctgtggag atcatcttca aagggcatga gaatgtggaa
1441 gctgctcagg cagagtacat cgagaagttt gccaaccctt tccctgcagc agtgcgaggg
1501 tttgtggatg acatcatcca accttcttcc acacgtgccc gaatctgctg tgacctggat
1561 gtcttggcca gcaagaaggt acaacgtcct tggagaaaac atgcaaatat tccattgtaa
```

### Sequence Accession No.: NM_{—}001889

Gene: Homo sapiens crystallin, zeta (quinone reductase) (CRYZ)

```
(SEQ ID NO: 152)
    1 atggcgactg gacagaagtt gatgagagct gttagagttt ttgaatttgg tgggccagaa
  61 gtcctgaaat tgcgatcaga tattgcagta ccgattccaa aagaccatca ggttctaatc
121 aaggtccatg catgtggtgt caaccccgtg gagacataca ttcgctctgg tacttatagt
181 agaaaaccac tcttacccta tactcctggc tcagatgtgg ctggggtgat agaagctgtt
241 ggagataatg catctgcttt caagaaaggt gacagagttt tcactagcag cacgatctct
301 gggggttatg cagagtatgc tcttgcagca gaccacactg tttacaaact acctgaaaaa
361 ctggacttta aacaaggagc tgccatcggc attccatatt ttactgctta tcgagctctg
421 atccacagtg cctgtgtgaa agctggagag agtgttctgg ttcatggggc aagtggagga
481 gttggattag cagcatgcca aattgctaga gcttatggct taaagatttt gggcactgct
541 ggtactgagg aaggacaaaa gattgttttg caaaatggag cccatgaagt gttcaatcac
601 agagaagtga attacattga taaaattaag aagtatgttg gtgagaaagg aattgatata
661 attattgaaa tgttagctaa tgtaaatctt agtaaagact tgagtcttct gtcacatgga
721 ggacgagtga tagttgttgg cagcagaggt actattgaaa taaacccacg agacaccatg
781 gcaaaggagt cgagtataat tggagttact ctcttttcct caaccaagga ggaatttcag
841 caatatgcag cagcccttca agctggaatg gaaattggct ggttgaaacc tgtgataggt
901 tctcaatatc cattggagaa ggtggccgag gctcatgaaa atatcattca tggtagtggg
961 gctactggaa aaatgattct tctcttatga
```

### Sequence Accession No.: NM_{—}018981

Gene: Homo sapiens DnaJ (Hsp40) homolog, subfamily C, member 10 (DNAJC10)

```
(SEQ ID NO: 153)
      1 atgggagtct ggttaaataa agatgactat atcagagact tgaaaaggat cattctctgt
    61 tttctgatag tgtatatggc cattttagtg ggcacagatc aggattttta cagtttactt
  121 ggagtgtcca aaactgcaag cagtagagaa ataagacaag ctttcaagaa attggcattg
  181 aagttacatc ctgataaaaa cccgaataac ccaaatgcac atggcgattt tttaaaaata
  241 aatagagcat atgaagtact caaagatgaa gatctacgga aaaagtatga caaatatgga
  301 gaaaagggac ttgaggataa tcaaggtggc cagtatgaaa gctggaacta ttatcgttat
  361 gattttggta tttatgatga tgatcctgaa atcataacat tggaaagaag agaatttgat
  421 gctgctgtta attctggaga actgtggttt gtaaattttt actccccagg ctgttcacac
  481 tgccatgatt tagctcccac atggagagac tttgctaaag aagtggatgg gttacttcga
  541 attggagctg ttaactgtgg tgatgataga atgctttgcc gaatgaaagg agtcaacagc
  601 tatcccagcc tcttcatttt tcggtctgga atggccccag tgaaatatca tggagacaga
  661 tcaaaggaga gtttagtgag ttttgcaatg cagcatgtta gaagtacagt gacagaactt
  721 tggacaggaa attttgtcaa ctccatacaa actgcctttg ctgctggtat tggctggctg
  781 atcacttttt gttcaaaagg aggagattgt ttgacttcac agacacgact caggcttagt
  841 ggcatgttgg atggtcttgt taatgtagga tggatggact gtgccaccca ggataacctt
  901 tgtaaaagct tagatattac aacaagtact actgcttatt ttcctcctgg agccacttta
  961 aataacaaag agaaaaacag tattttgttt ctcaactcat tggatgctaa agaaatatat
1021 ttggaagtaa tacataatct tccagatttt gaactacttt cggcaaacac actagaggat
1081 cgtttggctc atcatcggtg gctgttattt tttcattttg gaaaaaatga aaattcaaat
1141 gatcctgagc tgaaaaaact aaaaactcta cttaaaaatg atcatattca agttggcagg
1201 tttgactgtt cctctgcacc agacatctgt agtaatctgt atgtttttca gccgtctcta
1261 gcagtattta aaggacaagg aaccaaagaa tatgaaattc atcatggaaa gaagattcta
1321 tatgatatac ttgcctttgc caaagaaagt gtgaattctc atgttaccac gcttggacct
1381 caaaattttc ctgccaatga caaagaacca tggcttgttg atttctttgc cccctggtgt
1441 ccaccatgtc gagctttact accagagtta cgaagagcat caaatcttct ttatggtcag
1501 cttaagtttg gtacactaga ttgtacagtt catgagggac tctgtaacat gtataacatt
1561 caggcttatc caacgacagt ggtattcaac cagtccaaca ttcatgagta tgaaggacat
1621 cactctgctg aacaaatctt ggagttcata gaggatctta tgaatccttc agtggtctcc
1681 cttacaccca ccaccttcaa cgaactagtt acacaaagaa aacacaacga agtctggatg
1741 gttgatttct attctccgtg gtgtcatcct tgccaagtct taatgccaga atggaaaaga
1801 atggcccgga cattaactgg actgatcaac gtgggcagta tagattgcca acagtatcat
1861 tctttttgtg cccaggaaaa cgttcaaaga taccctgaga taagattttt tcccccaaaa
1921 tcaaataaag cttatcatta tcacagttac aatggttgga atagggatgc ttattccctg
1981 agaatctggg gtctaggatt tttacctcaa gtatccacag atctaacacc tcagactttc
2041 agtgaaaaag ttctacaagg gaaaaatcat tgggtgattg atttctatgc tccttggtgt
2101 ggaccttgcc agaattttgc tccagaattt gagctcttgg ctaggatgat taaaggaaaa
2161 gtgaaagctg gaaaagtaga ctgtcaggct tatgctcaga catgccagaa agctgggatc
2221 agggcctatc caactgttaa attttatttc tacgaaagag caaagagaaa ttttcaagaa
2281 gagcagataa ataccagaga tgcaaaagca atcgctgcct taataagtga aaaattggaa
2341 actctccgaa atcaaggcaa gaggaataag gatgaacttt ga
```

### Sequence Accession No.: NM_{—}198476

Gene: Homo sapiens chromosome 19 open reading frame 54 (C19orf54)

```
(SEQ ID NO: 154)
    1 atggcaggta ctctcctgtc gccccccagt ggcgtccccc tggagagact catacgggtg
  61 gccacggaaa gaggctacac ggcccaggga gagatgttct cagtggccga tatgggcagg
121 ctggcccagg aggtgctggg ctgccaggcc aagctgctct ctggtggcct gggcggtccc
181 aacagagacc tcgtcctgca gcacctggtc actggacatc ccctgctcat cccctacgac
241 gaggacttca accatgagcc gtgtcagagg aagggccaca aggcacactg ggcggtgagt
301 gcaggggtcc tgctgggtgt tcgggctgtg cccagtctcg gctacactga ggaccctgag
361 ctgccgggcc tgttccaccc agtgctgggc acgccctgcc aaccaccatc cctgccagag
421 gagggctccc cgggagctgt ctacctgctg tccaagcagg gcaagagttg gcactatcag
481 ctgtgggact acgaccaggt ccgggagagc aacctgcagc tgacggactt ctcgccctca
541 cgggccactg acggccgggt gtacgtggtg cccgtgggtg gggtacgggc tggcctctgt
601 ggccaggccc tgctcctcac accacaggac tgcagccatt ag
```

### Sequence Accession No.: NM_{—}002157

Gene: Homo sapiens heat shock 10 kDa protein 1 (chaperonin 10) (HSPE1)

```
(SEQ ID NO: 155)
    1 atggcaggac aagcgtttag aaagtttctt ccactctttg accgagtatt ggttgaaagg
  61 agtgctgctg aaactgtaac caaaggaggc attatgcttc cagaaaaatc tcaaggaaaa
121 gtattgcaag caacagtagt cgctgttgga tcgggttcta aaggaaaggg tggagagatt
181 caaccagtta gcgtgaaagt tggagataaa gttcttctcc cagaatatgg aggcaccaaa
241 gtagttctag atgacaagga ttatttccta tttagagatg gtgacattct tggaaagtac
301 gtagactga
```

### Sequence Accession No.: AF078844

Gene: Homo sapiens hqp0376 protein

```
(SEQ ID NO: 156)
    1 atgttgccag tattgccaaa gcctgggatg cccttggcgg cactggtgac ggggctgtca
  61 ggactgttat ggccctgttg tgctgagtta gttggaacag aattcaagct ccctgcacta
121 gtccacctgc cccactgctt cttcgcttct ctcttggaaa gtccagtctc tcctcggctt
181 gcaatggacc ccaactgctc ctgcgccgct ggtgtctcct gcacctgcgc tggttcctgc
241 aagtgcaaag agtgcaaatg cacctcctgc aagaagagct gctgctcctg ctgccccgtg
301 ggctgtagca agtgtgccca gggctgtgtt tgcaaagggg cgtcagagaa gtgcagctgc
361 tgcgactga
```

The present invention comprises methods by which a practitioner may evaluate the level of expression of any of the foregoing biomarkers or any allele or variant or fragment thereof for the purpose of predicting the sensitivity of a cell from which the biomarker was measured to any IGF1R inhibitor.

In an embodiment of the invention, the method comprises determining that a cell is sensitive if it expresses higher levels of one or more of the biomarkers taken from table 1 (e.g., ELLS1 and/or AUTS2 and/or TCF4 and/or TLE; e.g., all 4, 3, 2 or 1 in any combination whatsoever) than that of any cell known to be resistant to the IGF1R inhibitor. Similarly, in an embodiment of the invention, the method comprises determining that a cell is sensitive if it expresses lower levels of one or more of the biomarkers taken from table 2 than that of any cell known to be resistant to the IGF1R inhibitor. In an embodiment of the invention, a cell characterized by one of such genes exhibiting said comparatively high or low expression is characterized as possessing one biomarker for IGF1R inhibitor sensitivity; similarly, a cell characterized by, e.g., four or five or more of such genes exhibiting said comparatively high or low expression is characterized as possessing biomarkers for IGF1R inhibitor sensitivity.

In an embodiment of the invention, the level of expression of a gene in table 1 (e.g., ELLS1 and/or AUTS2 and/or TCF4 and/or TLE; e.g., all 4, 3, 2 or 1 in any combination whatsoever) or table 3 in a sensitive cell is, e.g., higher or lower, respectively, by any detectable and/or significant degree, e.g., at least about 1% (e.g., at least about 2%, 3%, 4%, 5%, 10%, 25%, 50%, 75%, 100%, 200%, 300%, 500% or 700%) higher or lower, respectively, than that of a resistant cell. In an embodiment of the invention, a sensitive cell possesses more than one biomarker for IGF1R inhibitor sensitivity. For example, in an embodiment of the invention, the sensitive cell comprises all of the biomarkers for IGF1R inhibitor sensitivity described in tables 1 and 3. In an embodiment of the invention, one or more of the biomarkers for IGF1R inhibitor sensitivity possessed by a sensitive cell exhibit levels of expression, when compared to that of a resistant cell, similar to that set forth in any of tables 1, 3, 5, or 7 (a-k).

In an embodiment of the invention, the magnitude of overexpression of one or more of the biomarkers in table 1 (e.g., ELLS1 and/or AUTS2 and/or TCF4 and/or TLE; e.g., all 4, 3, 2 or 1 in any combination whatsoever), relative to that of an IGF1R resistant cell is approximately as set forth in table 1 or more (i.e., greater magnitude of overexpression). For example, in an embodiment of the invention, a malignant cell is determined to be sensitive to an IGF1R inhibitor if the ratio of the TRE2 expression level in the cell being evaluated divided by the TRE2 expression level of an IGF1R inhibitor resistant cell is at least about 3.8. In an embodiment of the invention, the resistant cell used in this comparison is 22rv1, 2774 or H838. In an embodiment of the invention, a one or more genes other than TRE2 or one or more other genes in addition to TRE2 are evaluated. Similarly, the magnitude of underexpression of one or more of the biomarkers in table 3, relative to that of an IGF1R inhibitor resistant cell is approximately as set forth in table 3 or more (i.e., greater magnitude of underexpression).

In an embodiment of the invention, a sensitive cell can be evaluated for possession of one or more biomarkers for IGF1R inhibitor sensitivity by comparison of the expression levels of one or more of the genes set forth in Tables 1 and 3 to that of any of the following resistant cell lines: 22rv1, 2774 and H838. In an embodiment of the invention, a sensitive cell overexpresses one or more of the genes set forth in table 1 (e.g., ELLS1 and/or AUTS2 and/or TCF4 and/or TLE; e.g., all 4, 3, 2 or 1 in any combination whatsoever) and/or underexpresses one or more of the genes set forth in table 3 when compared to that of 22rv1, 2774 and H838. Cell line 22rv1 is a human prostate carcinoma cell line (see ATCC deposit no. CRL-2505). Cell line 2774 is an ovarian cancer cell line. H838 is a non-small cell lung cancer cell line (see ATCC deposit no. CRL-5844). These cells are known in the art.

The term “overexpress” or “high expression”, when used on the context of a comparison of gene expression levels in a cell and a reference cell, relates to cells characterized by expression of a given gene at a higher level than that of a reference cell. For example, in the present invention, IGF1R sensitive cells express the TRE2 gene at a higher level than that of resistant cells; thus, the TRE2 is overexpressed or exhibits high expression in sensitive cells. Similarly, the acetyl-coenzyme A acetyltransferase 1 gene is “underexpressed” or exhibits “low expression” in IGF1R inhibitor sensitive cells. In an embodiment of the invention, the terms overexpress, underexpress, high expression or low expression refer to expression of mRNA encoded by the biomarker gene. In an embodiment of the invention, the terms refers to expression of protein encoded by the biomarker gene.

Specifically, the present invention includes a method for evaluating sensitivity of malignant cells to an IGF1R inhibitor (e.g., an anti-IGF1R antibody) comprising determining if said cells exhibit high expression (e.g., RNA or protein expression (transcription or translation)) of one or more genes set forth in table 1 (e.g., ELLS1 and/or AUTS2 and/or TCF4 and/or TLE; e.g., all 4, 3, 2 or 1 in any combination whatsoever) or low expression of one or more genes set forth in table 3 relative to that of a cell resistant to said inhibitor. The present method may be used to evaluate sensitivity of in vitro cells, e.g., a cell line, or to evaluate sensitivity of cells derived from the body of a subject suffering from cancer. The cells evaluated under this method are determined to be sensitive if said high expression or said low expression is observed. In a more specific embodiment of the invention, the method comprises the steps of (a) obtaining a sample of one or more malignant cells from the body of a subject (e.g., a biopsy of tumor tissue or a blood sample from a suffering from a blood cancer such as leukemia); optionally transferring such a sample to a testing facility such as a laboratory for: (b) evaluating expression of one or more genes set forth in table 1 (e.g., ELLS1 and/or AUTS2 and/or TCF4 and/or TLE; e.g., all 4, 3, 2 or 1 in any combination whatsoever) or table 3 in the malignant cells; and (c) comparing said expression level to that of cells resistant to said IGF1R inhibitor; wherein the cells are determined to be sensitive to the inhibitor if expression of one or more genes in table 1 is higher than that of a cell resistant to said inhibitor or if expression of one or more genes in table 3 is lower than that of a cell resistant to said inhibitor.

Patient selection methods are also within the scope of the present invention. Such methods are beneficial, e.g., for the efficient targeting of subjects with cancer that is likely to be responsive to a given IGF1R inhibitor therapy. Specifically, the present invention provides a method for selecting a subject with malignant cells for treatment with an IGF1R inhibitor comprising evaluating sensitivity of the malignant cells to said inhibitor, e.g., by the method discussed above; wherein said subject is selected if said cells are determined to be sensitive. Moreover, the present invention provides a method for identifying a subject with malignant cells sensitive to an IGF1R inhibitor comprising evaluating sensitivity of the malignant cells to said inhibitor, e.g., by the method discussed above; wherein said subject is identified if said cells are determined to be sensitive.

Methods of treating cancer with an IGF1R inhibitor including selecting, e.g., pre-selecting, subjects with cancers sensitive or likely to be sensitive to the inhibitor are also provided herein. For example, the present invention provides a method for treating a tumor or cancerous condition with an IGF1R inhibitor comprising evaluating sensitivity of malignant cells, which are in said tumor or which mediate said cancerous condition, to said inhibitor, e.g., by the method discussed above, and, if said cells are determined to be sensitive, commencing or continuing treatment by administering, to the subject, a therapeutically effective dose of the inhibitor. In an embodiment of the invention, the evaluation may be performed after treatment has been commenced and, if the malignant cells in the body of the subject being tested are determined to be sensitive, treatment may be continued at the same or a different dose.

The present invention also provides methods for selecting a therapy suitable for treatment of cancer by prescreening the subject's malignant cells for IGF1R inhibitor sensitivity. For example, the present invention provides a method for selecting a therapy for a subject with one or more malignant cells comprising evaluating sensitivity of the cells to an IGF1R inhibitor, e.g., by the method discussed above; wherein said inhibitor is selected as the therapy if said cells are determined to be sensitive to the inhibitor.

The scope of the present invention also provides a method of advertising an IGF1R inhibitor or a pharmaceutically acceptable composition thereof or a therapeutic regimen comprising administration of said inhibitor or composition comprising promoting, to a target audience, the use of the inhibitor or composition for treating a patient or patient population whose tumors or cancerous conditions are mediated by malignant cells that exhibit increased expression of one or more genes set forth in table 1 (e.g., ELLS1 and/or AUTS2 and/or TCF4 and/or TLE; e.g., all 4, 3, 2 or 1 in any combination whatsoever), relative to cells resistant to said inhibitor; or that exhibit decreased expression of one or more genes set forth in table 3, relative to cells resistant to said inhibitor. Such uses may be promoted by any medium including, e.g., television, print or radio.

The present invention also provides articles of manufacture including one or more IGF1R inhibitors and literature explaining the relationship between the biomarkers of the present invention and sensitivity of a subject's cancer to the inhibitor. Specifically, the present invention provides an article of manufacture comprising, packaged together, an IGF1R inhibitor or a pharmaceutical composition thereof comprising a pharmaceutically acceptable carrier; and a label stating that the agent or pharmaceutical composition is indicated for treating patients having a tumor comprising malignant cells or a cancerous condition mediated by malignant cells that exhibit increased expression of one or more genes set forth in table 1 (e.g., ELLS1 and/or AUTS2 and/or TCF4 and/or TLE; e.g., all 4, 3, 2 or 1 in any combination whatsoever), relative to cells resistant to said inhibitor; or that exhibit decreased expression of one or more genes set forth in table 3, relative to cells resistant to said inhibitor. Methods of making such articles also form a part of the present invention. For example, the present invention provides a method for manufacturing an IGF1R inhibitor or a pharmaceutical composition thereof comprising a pharmaceutically acceptable carrier said method comprising combining, in a package, the inhibitor or composition; and a label conveying that the inhibitor or composition is indicated for treating patients having a tumor comprising malignant cells or a cancerous condition mediated by malignant cells that exhibit increased expression of one or more genes set forth in table 1, relative to cells resistant to said inhibitor; or that exhibit decreased expression of one or more genes set forth in table 3, relative to cells resistant to said inhibitor.

### Analysis and Determination of Expression Levels

An aspect of the invention includes determining whether a patient exhibits elevated or decreased levels of RNA or protein encoding various genes. Gene expression can be quantitated in a patient by any of the numerous methods known in the art. Expression can be quantited, for example, by simply hiring or contracting with a commercial laboratory to perform an assay wherein the patient's or subject's sample is harvested/biopsied and transferred to the lab. Alternatively, the practitioner can perform the assay himself. In an embodiment of the invention, expression is quantitated by a northern blot analysis, gene chip expression analysis, RT-PCR (real-time polymerase chain reaction), radioimmunoassay (RIA) (see e.g., Smith et al., J. Clin. Endocrin. Metab. 77(5): 1294-1299 (1993); Cohen et al., J. Clin. Endocrin. Metab. 76(4): 1031-1035 (1993); Dawczynski et al., Bone Marrow Transplant. 37:589-594 (2006); and Clemmons et al., J. Clin. Endocrin. Metab. 73:727-733 (1991)), western blot (WLB) or by ELISA (enzyme linked immunosorbent assay).

Any method for determining biomarker expression, e.g., as discussed herein, may be used to compare the expression level of the sample being evaluated (e.g., malignant or cancerous cells or an extract thereof) and the expression level of a resistant cell sample or an extract thereof so as to determine if the biomarker is overexpressed or underexpressed in the sample relative to that of the resistant cell. The quantity of cell samples being evaluated may be normalized against e.g., total cellular protein or RNA to ensure an accurate and meaningful comparison.

Northern blot analysis of biomarker transcription in a sample is, in an embodiment of the invention, performed. Northern analysis is a standard method for detection and quantitation of mRNA levels in a sample. Initially, RNA is isolated from a sample to be assayed (e.g., tumor tissue). In the analysis, the RNA samples are first separated by size via electrophoresis in an agarose gel under denaturing conditions. The RNA is then transferred to a membrane, crosslinked and hybridized with a labeled probe. In an embodiment of the invention, Northern hybridization involves polymerizing radiolabeled or nonisotopically detectably labeled DNA, in vitro, or generation of oligonucleotides as hybridization probes. In an embodiment of the invention, the membrane holding the RNA sample is prehybridized or “blocked” prior to probe hybridization to prevent the probe from coating the membrane and, thus, to reduce non-specific background signal. After hybridization, typically, unhybridized probe is removed by washing in several changes of buffer. Stringency of the wash and hybridization conditions can be designed, selected and implemented by any practitioner of ordinary skill in the art. If a radiolabeled probe was used, the blot can be wrapped in plastic wrap to keep it from drying out and then immediately exposed to film for autoradiography e.g, in the presence of a scintillant. If a nonisotopic probe was used, the blot must, generally, be treated with nonisotopic detection reagents, to develop the detectable probe signal, prior to film exposure. The relative levels of expression of the genes being assayed can be quantified using, for example, densitometry or visual estimation.

In an embodiment of the invention, expression of one or more biomarkers is determined in a gene chip analysis procedure. Such a procedure, in an embodiment of the invention, includes the following steps: target preparation, target hybridization, probe array washing and staining, probe array scan and data analysis. Target preparation entails, in an embodiment of the invention, preparing a biotinylated target RNA obtained from the sample to be tested. In an embodiment of the invention, the target hybridization step includes preparing a hybridization cocktail, including the fragmented target, probe array controls, BSA, and herring sperm DNA. It is then hybridized to the probe array during a 16-hour incubation. In an embodiment of the invention, immediately following hybridization, the probe array undergoes an automated washing and staining. In an embodiment of the invention, in the scanning and analysis step, the hybridized probe array is stained with streptavidin phycoerythrin conjugate and scanned for light emission at 570 nm wavelength. The amount of light emitted at 570 nm is proportional to the bound target at each location on the probe array. Computer analysis using commercially available equipment and software is possible (Affymetrix; Santa Clara, Calif.). Modifications to this general scheme, which are known in the art, form part of the present invention.

Biomarker expression is determined, in an embodiment of the invention, using RT-PCR. RT-PCR allows detection of the progress of a PCR amplification of a target gene in real time. Design of the primers and probes required to detect expression of a biomarker of the invention is within the skill of a practitioner of ordinary skill in the art. RT-PCR can be used to determine the level of RNA encoding a biomarker of the invention in a sample. In an embodiment of the invention, RNA from the tissue sample is isolated, under RNAse free conditions, then converted to DNA by treatment with reverse transcriptase. Methods for reverse transcriptase conversion of RNA to DNA are well known in the art. Reverse transcription may be performed prior to RT-PCR analysis or simultaneously, within a single reaction vessel (e.g., tube).

RT-PCR probes depend on the 5′-3′ nuclease activity of the DNA polymerase used for PCR to hydrolyze an oligonucleotide that is hybridized to the target amplicon (biomarker gene). RT-PCR probes are oligonucleotides that have a fluorescent reporter dye attached to the 5′ end and a quencher moiety coupled to the 3′ end (or vice versa). These probes are designed to hybridize to an internal region of a PCR product. In the unhybridized state, the proximity of the fluor and the quench molecules prevents the detection of fluorescent signal from the probe. During PCR amplification, when the polymerase replicates a template on which an RT-PCR probe is bound, the 5′-3′ nuclease activity of the polymerase cleaves the probe. This decouples the fluorescent and quenching dyes and FRET no longer occurs. Thus, fluorescence increases in each cycle, in a manner proportional to the amount of probe cleavage. Fluorescence signal emitted from the reaction can be measured or followed over time using equipment which is commercially available using routine and conventional techniques. Quantitation of biomarker RNA in a sample being evaluated may be performed by comparison of the amplification signal to that of one or more standard curves wherein known quantities of RNA were evaluated in a similar manner. Such methods are known in the art.

In an embodiment of the invention, western blots are performed as follows: A sample comprising an extract of a tumor tissue source is electrophoresed on 10% polyacrylamide-sodium dodecyl sulfate (SDS-PAGE) gel and electroblotted onto nitrocellulose or some other suitable membrane. The membrane is then incubated with a primary antibody which binds to the protein product of the gene being evaluated, optionally washed and then incubated with a detectably labeled secondary antibody that binds to the primary antibody and optionally washed again. The presence of the secondary antibody is then detected. For example, if the secondary antibody is labeled with a chemilluminescence label, the label is developed with a developing agent, then the membrane is exposed to film and then the film is developed. In an embodiment of the invention, each lane of the autoradiograph is scanned and analyzed by densitometer.

In an embodiment of the invention, an ELISA assay employs an antibody specific for a biomarker coated on a 96-well plate. Standards and samples are pipetted into the wells and biomarker present in a sample is bound to the wells by the immobilized antibody. The wells are washed and biotinylated anti-biomarker antibody is added. After washing away unbound biotinylated antibody, HRP-conjugated streptavidin is pipetted to the wells. The wells are again washed, a TMB substrate solution is added to the wells and color develops in proportion to the amount of biomarker bound. Stop solution added to the reaction changes the color from blue to yellow, and the intensity of the color is measured at 450 nm (see e.g., Human IGF-BP-2 ELISA Kit from RayBiotech, Inc.; Norcross, Ga.; and Angervo M et al., Biochemical and Biophysical Research Communications 189: 1177-83 (1992); Kratz et al., Experimental Cell Research 202: 381-5 (1992); and Frost et al. Journal of Biological Chemistry 266: 18082-8 (1991)). A standard ELISA curve using known concentrations of biomarker can be plotted and the concentration of biomarker in the unknown sample (e.g., the serum of a patient) can be determined by comparing the signal observed therein with the signal observed in the standard.

Radioimmunoassay (RIA) is a scientific method used to detect the presence of a given antigen, e.g., encoded by a biomarker gene. RIA involves mixing known quantities of radioactive antigen (e.g., labeled with gamma-radioactive isotopes of iodine attached to tyrosine) with antibody to that antigen, then adding unlabeled or “cold” antigen and measuring the amount of labeled antigen displaced. Initially, the radioactive antigen is bound to the antibodies. When “cold” (unlabeled) antigen is added, the two compete for antibody binding sites—at higher concentrations of “cold” antigen, more of it binds to the antibody, displacing the radioactive variant. The bound antigens are then separated from the unbound ones and the quantitiy of labeled bound antigen is then quantitated. The bound antigen can be separated from unbound antigen in several ways; for example, by precipitating the antigen-antibody complexes by adding a secondary antibody directed against the primary antibody. In another embodiment of the invention, the antigen-specific antibodies can be coupled to the inner walls of a test tube or microtiter well or to some other solid substrate. After incubation, the contents are removed and the tube, well or substrate, which is washed leaving bound, labeled antibody/antigen complexes; and, then, the radioactive label present in the tube or well of both is measured.

### EXAMPLES

This section is intended to further describe the present invention and should not be construed to further limit the invention. Any composition or method set forth herein constitutes part of the present invention.

### Example 1

### Identification of Biomarkers

Xenograft samples. The xenografts used in this study (and their tissue of origin) are: H322 and H838 (both derived from non-small cell lung carcinoma), SK-N-AS and SK-N-FI (both derived from neuroblastomas), 22rv1 (derived from prostate), 2774 (derived from ovarian) and SJSA-1 (derived from an osteosarcoma). The 22rv1, 2774 and H838 cell lines are resistant to anti-IGF1R antibody (mature Ig fragments of SEQ ID NOs: 8 and 10/κ light chain, γ1 heavy chain) mediated growth inhibition. Cells were injected into nude mice and tumors were allowed to reach approximately 200 mm³ in size before harvesting. Mice were treated with one intraperitoneal injection of either 0.1 mg of anti-IGF-1R antibody (mature Ig fragments of SEQ ID NOs: 8 and 10/κ light chain, γ1 heavy chain) (for xenografted mice bearing SK-N-AS and SK-N-FI tumors) or 0.5 mg of the anti-IGF1R antibody (for xenografted mice bearing SJSA-, H322, H388, 22rv1 and 2774 tumors). Tumors were harvested 48 hrs after antibody treatment and were cut in half and snap-frozen. Half were processed for RNA as described below and the other half were processed for protein identification.

Chip hybridization. RNA was made from cells using the Trizol reagent (Molecular Research Center, Inc.; Cincinnati, Ohio) followed by purification over an RNAeasy column (Qiagen; Valencia, Calif.). Five micrograms of total RNA was used to make probes as described in the Affymetric Expression Analysis Technical Manual (www.affymetrix.com/support/technical/manual/expression_manual.affx) (Affymetrix, Inc; Santa Clara, Calif.). Probes were hybridized to the Affymetrix human (U133 Plus 2.0) high-density oligonucletide arrays as described in the manual.

Microarray analysis. Data analysis was performed using an S+ based program licensed from Insightful Corp. (Seattle, Wash.). Data was filtered so that measurements for probe sets with the prefix AFFX (Affymetrix control probe sets), probe sets which were called “absent” in all experiments, or where less than 7 probe pairs registered data were dropped from the analysis. All data was Log 2 transformed. The data was then normalized to the median inter-quartile range for each probe set. The normalized data was then filtered as follows: an expression percentage restriction was applied so that only conditions where the raw data had a value of 100 in at least three chips were included. Statistical analysis was done in a pair-wise fashion using the multiple testing t-test (with a p value of <0.05) in conjunction with a Benjamin-Hochberg multi-test correction. Data from each sensitive xenograft model was compared to data from each resistant model. Statistically significant gene lists generated from each of the pair-wise comparisons were then overlapped using Venn diagrams to find the genes that were up or down regulated in all sensitive xenografts when compared to all resistant xenografts.

The data from these analyses are set forth below in tables 1-4. In these tables, the name of the gene/biomarker analyzed is set forth along with the Genbank accession number for each gene. Also shown in tables 1 and 3 are the average expression levels of each biomarker in the sensitive (Savg) and resistant (Ravg) cell lines analyzed along with a ratio thereof (Savg/Ravg). The normalized expression levels of each biomarker in each of the resistant (R) and sensitive (S) cell lines are set forth below in tables 2 and 4. The normalized data in table 2 corresponds to the data set forth in table 1 and the normalized data in table 4 corresponds to the data in table 3.

TABLE 1
Genes upregulated in sensitive xenografts relative to resistant xenografts

| Probe Description | Acc. Num | Savg/Ravg | R avg | S avg |
|---|---|---|---|---|
| ubiquitin specific protease 6 (Tre-2 | NM_004505 | 3.837782111 | 56.83746 | 218.1298 |
| oncogene) | | | | |
| SMC4 structural maintenance of | NM_001002799 | 2.209888459 | 1499.261 | 3313.199 |
| chromosomes 4-like 1 (yeast) | | | | |
| tribbles homolog 2 (Drosophila) | NM_021643 | 17.41172933 | 229.5332 | 3996.57 |
| transducin-like enhancer of split 4 | NM_007005 | 21.31625909 | 120.4993 | 2568.595 |
| (E(sp1) homolog, Drosophila) | | | | |
| Bone morphogenetic protein 7 | NM_001719 | 5.434476936 | 197.6966 | 1074.377 |
| (osteogenic protein 1) | | | | |
| protocadherin gamma subfamily C, | NM_002588 | 2.555349516 | 948.1301 | 2422.804 |
| 3 /// protocadherin gamma | | | | |
| subfamily C, 3 /// protocadherin | | | | |
| gamma subfamily B, 4 /// | | | | |
| protocadherin gamma subfamily B, | | | | |
| 4 /// protocadherin gamma | | | | |
| subfamily A, 8 /// protocadherin | | | | |
| gamma subfamily A, 8 /// | | | | |
| protocadherin ga | | | | |
| autism susceptibility candidate 2 | NM_015570 | 7.815854047 | 433.5968 | 3388.929 |
| chromosome 14 open reading | NM_020215 | 14.12906043 | 85.42421 | 1206.964 |
| frame 132 | | | | |
| ceramide kinase | NM_022766 | 3.172632848 | 377.3073 | 1197.058 |
| hepatoma-derived growth factor, | NM_016073 | 3.099552834 | 636.7927 | 1973.773 |
| related protein 3 | | | | |
| transcription factor 4 | NM_003199 | 13.69711801 | 230.8982 | 3162.64 |
| Meis1, myeloid ecotropic viral | NM_002399 | 5.15772508 | 293.4639 | 1513.606 |
| integration site 1 homolog 2 | | | | |
| (mouse) | | | | |
| echinoderm microtubule | NM_019063 | 2.34104038 | 795.2575 | 1861.73 |
| associated protein like 4 | | | | |
| hypothetical protein Ells1 | NM_152793 | 3.817830274 | 103.0596 | 393.4642 |
| KIAA 1450 protein | AB040883 | 3.690315901 | 276.5982 | 1020.735 |
| zinc finger protein 136 (clone pHZ- | NM_003437 | 2.697125849 | 230.2471 | 621.0055 |
| 20) | | | | |
| protocadherin gamma subfamily C, | NM_002588 | 2.970723128 | 413.2103 | 1227.533 |
| 3 /// protocadherin gamma | | | | |
| subfamily B, 4 /// protocadherin | | | | |
| gamma subfamily A, 8 /// | | | | |
| protocadherin gamma subfamily A, | | | | |
| 12 /// protocadherin gamma | | | | |
| subfamily C, 5 /// protocadherin | | | | |
| gamma subfamily C, 4 /// | | | | |
| protocadherin g | | | | |
| D15F37 gene /// hypothetical | NM_001024681 | 1.943720268 | 759.0605 | 1475.401 |
| LOC440248 | | | | |

TABLE 2
Normalized average values (3 replicates for each xenograft type)

| | R | R | R | S | S | S | S |
|---|---|---|---|---|---|---|---|
| Probe Description | 22rv1 | 2774 | H838 | H322 | SJSA | SK-N-AS | SK-N-F1 |
| ubiquitin specific protease 6 (Tre-2 | 56.44211 | 42.18622 | 71.88406 | 214.5991 | 238.7406 | 196.4884 | 222.6911 |
| oncogene) | | | | | | | |
| SMC4 structural maintenance of | 1790.31781 | 1062.318 | 1645.14611 | 2355.798 | 4554.257 | 3403.964 | 2938.777 |
| chromosomes 4-like 1 (yeast) | | | | | | | |
| tribbles homolog 2 (Drosophila) | 40.58009 | 206.3572 | 441.66234 | 3278.909 | 3808.481 | 2533.647 | 6365.244 |
| transducin-like enhancer of split 4 | 83.08983 | 234.1031 | 44.30507 | 776.8003 | 1327.044 | 2957.167 | 5213.368 |
| (E(sp1) homolog, Drosophila) | | | | | | | |
| Bone morphogenetic protein 7 | 50.9673 | 168.406 | 373.71638 | 901.8227 | 1557.482 | 727.7 | 1110.505 |
| (osteogenic protein 1) | | | | | | | |
| protocadherin gamma subfamily C, | 828.69649 | 1089.16 | 926.53393 | 2042.33 | 2811.258 | 3057.21 | 1780.418 |
| 3 /// protocadherin gamma | | | | | | | |
| subfamily C, 3 /// protocadherin | | | | | | | |
| gamma subfamily B, 4 /// | | | | | | | |
| protocadherin gamma subfamily B, | | | | | | | |
| 4 /// protocadherin gamma | | | | | | | |
| subfamily A, 8 /// protocadherin | | | | | | | |
| gamma subfamily A, 8 /// | | | | | | | |
| protocadherin ga | | | | | | | |
| autism susceptibility candidate 2 | 627.51233 | 518.2484 | 155.02961 | 3782.174 | 2269.25 | 3850.954 | 3653.339 |
| chromosome 14 open reading | 66.79184 | 107.3814 | 82.09941 | 246.4925 | 375.508 | 2069.405 | 2136.45 |
| frame 132 | | | | | | | |
| ceramide kinase | 145.56301 | 575.6707 | 410.68829 | 801.1341 | 1279.177 | 1089.915 | 1618.005 |
| hepatoma-derived growth factor, | 260.38454 | 1006.269 | 643.72469 | 1694.918 | 1646.287 | 1685.546 | 2868.339 |
| related protein 3 | | | | | | | |
| transcription factor 4 | 335.11211 | 162.2079 | 195.37472 | 526.2484 | 4925.314 | 3406.324 | 3792.675 |
| Meis1, myeloid ecotropic viral | 53.10589 | 287.8154 | 539.4704 | 1020.033 | 971.7221 | 2922.526 | 1140.144 |
| integration site 1 homolog 2 | | | | | | | |
| (mouse) | | | | | | | |
| echinoderm microtubule | 748.57341 | 849.2821 | 787.91708 | 1339.056 | 1661.188 | 1746.196 | 2700.48 |
| associated protein like 4 | | | | | | | |
| hypothetical protein Ells1 | 104.04761 | 114.8972 | 90.23405 | 168.8268 | 375.4039 | 463.4918 | 566.1341 |
| KIAA1450 protein | 323.00216 | 304.2472 | 202.54519 | 550.1953 | 1089.915 | 1297.933 | 1144.895 |
| zinc finger protein 136 (clone pHZ- | 192.97899 | 172.4459 | 325.31646 | 526.9053 | 749.1963 | 543.524 | 664.3962 |
| 20) | | | | | | | |
| protocadherin gamma subfamily C, | 362.36505 | 506.0023 | 371.26358 | 841.4886 | 1578.13 | 1578.13 | 912.3857 |
| 3 /// protocadherin gamma | | | | | | | |
| subfamily B, 4 /// protocadherin | | | | | | | |
| gamma subfamily A, 8 /// | | | | | | | |
| protocadherin gamma subfamily A, | | | | | | | |
| 12 /// protocadherin gamma | | | | | | | |
| subfamily C, 5 /// protocadherin | | | | | | | |
| gamma subfamily C, 4 /// | | | | | | | |
| protocadherin g | | | | | | | |
| D15F37 gene /// hypothetical | 802.07871 | 792.7378 | 682.36508 | 1353.989 | 1619.127 | 1398.825 | 1529.665 |
| LOC440248 | | | | | | | |

TABLE 3
Genes downregulated in sensitive xenografts relative to resistant xenografts.

| Probe Description | Acc. Num | Savg/Ravg | R avg | S avg |
|---|---|---|---|---|
| acetyl-Coenzyme A acetyltransferase 1 | NM_000019 | −3.245379956 | 2575.89063 | 793.710034 |
| (acetoacetyl Coenzyme A thiolase) | | | | |
| aldolase C, fructose-bisphosphate | NM_005165 | −5.768075828 | 1159.3523 | 200.994636 |
| chromosome 6 open reading frame 192 | NM_052831 | −5.329368752 | 917.785933 | 172.212879 |
| collagen, type IV, alpha 5 (Alport | NM_000495 | −3.324838486 | 1852.4099 | 557.142823 |
| syndrome) | | | | |
| complement component 1, q | NM_001212 | −2.321386792 | 5219.38693 | 2248.39176 |
| subcomponent binding protein | | | | |
| cysteine-rich protein 1 (intestinal) | NM_001311 | −33.05700201 | 3016.02373 | 91.2370618 |
| deaminase domain containing 1 | NM_182503 | −2.470378364 | 262.9737 | 106.450779 |
| glutathione S-transferase kappa 1 | NM_015917 | −2.994296286 | 1629.43823 | 544.180695 |
| glutathione S-transferase omega 2 | NM_183239 | −5.844624181 | 1030.33563 | 176.287748 |
| HTPAP protein | NM_032483 | −6.537193516 | 1753.02323 | 268.161441 |
| hypothetical protein MGC10744 | NM_032354 | −3.677723087 | 813.142267 | 221.099373 |
| hypothetical protein MGC5306 | NM_024116 | −3.305463087 | 1841.13437 | 556.997407 |
| hypothetical protein MGC54289 | NM_178454 | −3.250328663 | 1260.2607 | 387.733313 |
| macrophage stimulating 1 (hepatocyte | NM_020998 | −3.430093203 | 335.100633 | 97.694323 |
| growth factor-like) | | | | |
| metallothionein 1E (functional) | NM_175617 | −9.533035275 | 3054.9655 | 320.460946 |
| parvin, beta | NM_001003828 | −3.194142531 | 514.0407 | 160.932299 |
| peroxiredoxin 4 | NM_006406 | −2.90324185 | 6608.63977 | 2276.29667 |
| phosphatidic acid phosphatase type 2 | NM_032483 | −2.298947498 | 266.0359 | 115.720738 |
| domain containing 1B | | | | |
| RasGEF domain family, member 1A | NM_145313 | −8.464065301 | 752.942533 | 88.9575525 |
| L14 /// ribosomal protein L14-like /// | NM_003973 | −2.103792894 | 9681.4598 | 4601.90726 |
| ribosomal protein L14-like | | | | |
| interferon, gamma-inducible protein 30 | NM_006332 | −6.574905274 | 871.058067 | 132.482223 |
| Activating transcription factor 1 | NM_005171 | −2.508647643 | 1327.30717 | 529.092705 |
| acyl-Coenzyme A dehydrogenase, very | NM_000018 | −2.779952512 | 3286.15267 | 1182.0895 |
| long chain | | | | |
| F-box protein 6 | NM_018438 | −6.608973602 | 624.026833 | 94.4211418 |
| NAD(P)H dehydrogenase, quinone 2 | NM_000904 | −4.212543705 | 1258.5278 | 298.757209 |
| transmembrane protein 64 | NM_001008495 | −3.429705418 | 1715.79683 | 500.275279 |
| zinc finger, AN1-type domain 1 | NM_024699 | −2.618678085 | 1328.24477 | 507.219568 |
| transmembrane emp24 protein transport | NM_016040 | −3.219110394 | 529.700333 | 164.54867 |
| domain containing 5 | | | | |
| protein disulfide isomerase family A, | NM_006810 | −2.453429551 | 935.6381 | 381.359269 |
| member 5 | | | | |
| Myosin IC | NM_033375 | −3.051964172 | 1685.95957 | 552.417877 |
| MGC4170 protein | NM_024312 | −2.75015402 | 1595.90433 | 580.296348 |
| lactamase, beta 2 | NM_016027 | −4.511312257 | 1020.94993 | 226.30886 |
| ribosomal protein L22 | NM_000983 | −2.271871908 | 5464.91127 | 2405.46628 |
| tetraspanin 4 | NM_001025234 | −4.602614344 | 1048.2456 | 227.75004 |
| ribosomal protein L15 /// similar to | NM_002948 | −2.398236835 | 1063.05883 | 443.266827 |
| ribosomal protein L15 | | | | |
| propionyl Coenzyme A carboxylase, beta | NM_000532 | −2.605125082 | 981.0221 | 376.573895 |
| polypeptide | | | | |
| chromosome 6 open reading frame 192 | NM_052831 | −5.329368752 | 917.785933 | 172.212879 |
| hypothetical protein MGC10744 | NM_032354 | −3.677723087 | 813.142267 | 221.099373 |
| hypothetical protein MGC54289 | NM_178454 | −3.250328663 | 1260.2607 | 387.733313 |
| crystallin, zeta (quinone reductase) | NM_001889 | −2.870869801 | 2340.67357 | 815.318607 |
| DnaJ (Hsp40) homolog, subfamily C, | NM_018981 | −2.180174843 | 2168.3292 | 994.566655 |
| member 10 | | | | |
| FLJ41131 protein | NM_198476 | −2.206539945 | 912.135067 | 413.377999 |
| heat shock 10 kDa protein 1 (chaperonin | NM_002157 | −2.521036542 | 4409.95823 | 1749.26391 |
| 10) | | | | |
| Homo sapiens hqp0376 protein mRNA, | AF078844 | −3.120461907 | 891.404633 | 285.664322 |
| complete cds. | | | | |
| hypothetical protein MGC5306 | NM_024116 | −3.305463087 | 1841.13437 | 556.997407 |

TABLE 4
Normalized average values (3 replicates for each xenograft type).

| | R | R | R | S | S | S | S |
|---|---|---|---|---|---|---|---|
| Probe Description | 22rv1 | 2774 | H838 | H322 | SJSA | SK-N-AS | SK-N-F1 |
| acetyl-Coenzyme A acetyltransferase 1 (acetoacetyl | 3010.944 | 2957.167 | 1759.561 | 1036.138 | 338.0986 | 755.0875 | 1045.516 |
| Coenzyme A thiolase) | | | | | | | |
| aldolase C, fructose-bisphosphate | 1439.149 | 935.3095 | 1103.599 | 180.6934 | 97.1741 | 308.0029 | 218.1082 |
| chromosome 6 open reading frame 192 | 769.9388 | 1067.485 | 915.9341 | 342.9371 | 330.4527 | 9.270197 | 6.191547 |
| collagen, type IV, alpha 5 (Alport syndrome) | 1952.355 | 1744.986 | 1859.888 | 1051.33 | 706.3797 | 432.3932 | 38.46835 |
| complement component 1, q subcomponent binding | 5434.758 | 5728.732 | 4494.672 | 1961.851 | 1889.777 | 2644.905 | 2497.034 |
| protein | | | | | | | |
| cysteine-rich protein 1 (intestinal) | 667.0264 | 2715.496 | 5665.549 | 59.18665 | 81.56057 | 213.7084 | 10.4926 |
| deaminase domain containing 1 | 256.1775 | 281.2287 | 251.5149 | 122.6497 | 117.8902 | 121.1877 | 64.07546 |
| glutathione S-transferase kappa 1 | 1435.164 | 1810.284 | 1642.867 | 238.1621 | 642.7438 | 696.3651 | 599.4518 |
| glutathione S-transferase omega 2 | 1177.899 | 1115.133 | 797.9751 | 511.0781 | 43.33918 | 91.56346 | 59.17025 |
| HTPAP protein | 3260.777 | 1000.912 | 997.3804 | 319.5062 | 235.666 | 270.2967 | 247.1769 |
| hypothetical protein MGC10744 | 937.6463 | 808.8341 | 692.9464 | 296.3791 | 211.7473 | 114.6744 | 261.5966 |
| hypothetical protein MGC5306 | 1430.199 | 2910.397 | 1182.808 | 365.3917 | 546.3191 | 589.8891 | 726.3897 |
| hypothetical protein MGC54289 | 863.768 | 983.5128 | 1933.501 | 458.0305 | 395.4907 | 386.815 | 310.597 |
| macrophage stimulating 1 (hepatocyte growth factor- | 455.6556 | 303.3417 | 246.3046 | 108.3759 | 37.27923 | 120.4842 | 124.638 |
| like) | | | | | | | |
| metallothionein 1E (functional) | 3401.605 | 3864.323 | 1898.968 | 479.737 | 342.3908 | 204.5486 | 255.1674 |
| parvin, beta | 399.458 | 623.9125 | 518.7516 | 71.50638 | 198.9552 | 164.6091 | 208.6585 |
| peroxiredoxin 4 | 7553.869 | 6204.073 | 6067.977 | 2774.477 | 1861.178 | 3408.686 | 1060.846 |
| phosphatidic acid phosphatase type 2 domain | 270.3904 | 238.5421 | 289.1752 | 90.09031 | 135.9564 | 115.1603 | 121.6759 |
| containing 1B | | | | | | | |
| RasGEF domain family, member 1A | 826.9177 | 668.5075 | 763.4024 | 128.9618 | 40.76616 | 2.084643 | 184.0176 |
| ribosomal protein L14 /// ribosomal protein L14 /// | 13363.4 | 7241.134 | 8439.84 | 4482.227 | 4894.684 | 4067.707 | 4963.011 |
| ribosomal protein L14-like /// ribosomal protein | | | | | | | |
| L14-like | | | | | | | |
| interferon, gamma-inducible protein 30 | 501.5327 | 603.0359 | 1508.606 | 284.9569 | 77.82167 | 80.30957 | 86.84073 |
| Activating transcription factor 1 | 1249.383 | 816.3817 | 1916.157 | 598.9949 | 565.7026 | 405.2027 | 546.4706 |
| acyl-Coenzyme A dehydrogenase, very long chain | 3371.091 | 2514.403 | 3972.964 | 1956.419 | 1138.564 | 619.474 | 1013.9 |
| F-box protein 6 | 419.0562 | 635.5668 | 817.4575 | 249.6218 | 71.18 | 24.06059 | 32.8222 |
| NAD(P)H dehydrogenase, quinone 2 | 1337.2 | 854.7745 | 1583.609 | 263.4344 | 521.817 | 217.0525 | 192.725 |
| transmembrane protein 64 | 1233.892 | 1409.532 | 2503.967 | 572.924 | 686.6352 | 563.7063 | 177.8356 |
| zinc finger, AN1-type domain 1 | 1827.936 | 1061.582 | 1095.218 | 431.7643 | 508.1815 | 630.1712 | 458.7613 |
| transmembrane emp24 protein transport domain | 378.9595 | 392.5139 | 817.6276 | 153.5644 | 201.2995 | 229.3489 | 73.98188 |
| containing 5 | | | | | | | |
| protein disulfide isomerase family A, member 5 | 822.9723 | 753.4667 | 1230.475 | 334.7871 | 387.9428 | 534.7052 | 268.002 |
| Myosin IC | 1697.27 | 1685.546 | 1675.063 | 926.8551 | 659.5325 | 272.1202 | 351.1637 |
| MGC4170 protein | 1221.129 | 1151.262 | 2415.322 | 678.0745 | 550.615 | 437.7915 | 654.7045 |
| lactamase, beta 2 | 1270.341 | 1269.461 | 523.0482 | 343.1986 | 210.9563 | 190.8506 | 160.2299 |
| ribosomal protein L22 | 7696.57 | 3937.325 | 4760.839 | 2747.683 | 2231.812 | 2354.165 | 2288.204 |
| tetraspanin 4 | 490.157 | 624.9513 | 2029.629 | 168.6163 | 308.3874 | 235.6334 | 198.3631 |
| ribosomal protein L15 /// similar to ribosomal protein | 1103.599 | 1250.249 | 835.3286 | 383.2123 | 506.5286 | 415.4123 | 467.9141 |
| L15 /// similar to ribosomal protein L15 | | | | | | | |
| propionyl Coenzyme A carboxylase, beta polypeptide | 1162.488 | 761.6055 | 1018.973 | 509.3099 | 235.715 | 517.8175 | 243.4531 |
| chromosome 6 open reading frame 192 | 769.9388 | 1067.485 | 915.9341 | 342.9371 | 330.4527 | 9.270197 | 6.191547 |
| hypothetical protein MGC10744 | 937.6463 | 808.8341 | 692.9464 | 296.3791 | 211.7473 | 114.6744 | 261.5966 |
| hypothetical protein MGC54289 | 863.768 | 983.5128 | 1933.501 | 458.0305 | 395.4907 | 386.815 | 310.597 |
| crystallin, zeta (quinone reductase) | 1934.842 | 2872.319 | 2214.86 | 792.1885 | 883.6309 | 1254.59 | 330.8653 |
| OnaJ (Hsp40) homolog, subfamily C, member 10 | 1821.612 | 2291.378 | 2391.997 | 1102.834 | 1251.116 | 772.5583 | 851.7581 |
| FLJ41131 protein | 1135.412 | 793.7275 | 807.2658 | 281.6774 | 388.2925 | 583.5854 | 399.9567 |
| heat shock 10 kDa protein 1 (chaperonin 10) | 4928.729 | 3209.205 | 5091.941 | 1725.741 | 1507.56 | 2142.382 | 1621.373 |
| Homo sapiens hqp0376 protein mRNA, complete cds. | 577.3891 | 1258.073 | 838.7517 | 363.3208 | 220.3419 | 208.8032 | 350.1914 |
| hypothetical protein MGC5306 | 1430.199 | 2910.397 | 1182.808 | 365.3917 | 546.3191 | 589.8891 | 726.3897 |

In addition to the biomarkers set forth above, the expression levels of the additional biomarkers, CDK6, T1MP, CLu and PRL1, were evaluated in all 7 xenografts discussed above. The results of these analyses are set forth below in tables 5 and 6. The normalized data in table 6 corresponds to the data in table 5.

TABLE 5
Analysis of cdk6, TIMP, CLu, PRL1 gene
expression in all 7 xenografts.

| Probe Description | Acc. Num | Savg/Ravg | R avg | S avg |
|---|---|---|---|---|
| cyclin-dependent kinase 6 | NM_001259 | 1.40 | 2531.52 | 3544.02 |
| cyclin-dependent kinase 6 | NM_001259 | 1.99 | 1475.72 | 2930.52 |
| protein tyrosine | NM_003463 | 0.20 | 2303.37 | 461.79 |
| phosphatase type IVA, | | | | |
| member 1 | | | | |
| protein tyrosine | NM_003463 | 0.15 | 3145.51 | 485.65 |
| phosphatase type IVA, | | | | |
| member 1 | | | | |
| protein tyrosine | NM_003463 | 0.18 | 5693.46 | 1000.04 |
| phosphatase type IVA, | | | | |
| member 1 | | | | |
| protein tyrosine | NM_003463 | 0.29 | 4391.44 | 1283.80 |
| phosphatase type IVA, | | | | |
| member 1 | | | | |
| tissue inhibitor of | NM_003254 | 0.67 | 5306.41 | 3535.24 |
| metalloproteinase 1 | | | | |
| (erythroid potentiating | | | | |
| activity, collagenase | | | | |
| inhibitor) | | | | |
| clusterin (complement lysis | NM_001831 | 0.14 | 4268.88 | 579.80 |
| inhibitor, SP-40, 40, | | | | |
| sulfated glycoprotein 2, | | | | |
| testosterone-repressed | | | | |
| prostate message 2, | | | | |
| apolipoprotein J) | | | | |
| clusterin (complement lysis | NM_001831 | 0.18 | 2785.49 | 503.36 |
| inhibitor, SP-40, 40, | | | | |
| sulfated glycoprotein 2, | | | | |
| testosterone-repressed | | | | |
| prostate message 2, | | | | |
| apolipoprotein J) | | | | |

TABLE 6
Normalized average values for cdk6, TIMP, CLu, PRL1 gene expression in all
7 xenografts.

| | R | R | R | S | S | S | S |
|---|---|---|---|---|---|---|---|
| Probe Description | 22rv1 | 2774 | H838 | H322 | SJSA1 | SK-N-AS | SK-N-F1 |
| cyclin-dependent kinase 6 | 189.02 | 6347.62 | 1057.91 | 2969.49 | 1507.56 | 6105.95 | 3593.07 |
| cyclin-dependent kinase 6 | 102.01 | 3896.60 | 428.54 | 3036.09 | 2438.87 | 3548.52 | 2698.61 |
| protein tyrosine phosphatase | 2836.70 | 659.99 | 3413.41 | 420.69 | 175.16 | 697.43 | 553.91 |
| type IVA, member 1 | | | | | | | |
| protein tyrosine phosphatase | 4371.76 | 628.91 | 4435.87 | 325.20 | 307.58 | 714.31 | 595.52 |
| type IVA, member 1 | | | | | | | |
| protein tyrosine phosphatase | 9429.73 | 1338.13 | 6312.52 | 728.61 | 791.26 | 1265.07 | 1215.22 |
| type IVA, member 1 | | | | | | | |
| protein tyrosine phosphatase | 4807.26 | 2493.58 | 5873.48 | 1305.15 | 907.91 | 1341.84 | 1580.32 |
| type IVA, member 1 | | | | | | | |
| tissue inhibitor of | 4466.72 | 6557.82 | 4894.68 | 1344.64 | 10682.78 | 1100.54 | 1012.99 |
| metalloproteinase 1 (erythroid | | | | | | | |
| potentiating activity, | | | | | | | |
| collagenase inhibitor) | | | | | | | |
| clusterin (complement lysis | 6321.28 | 463.49 | 6021.89 | 693.14 | 480.90 | 43.87 | 1101.31 |
| inhibitor, SP-40,40, sulfated | | | | | | | |
| glycoprotein 2, testosterone- | | | | | | | |
| repressed prostate message | | | | | | | |
| 2, apolipoprotein J) | | | | | | | |
| clusterin (complement lysis | 3940.05 | 362.79 | 4053.63 | 647.93 | 530.86 | 44.71 | 789.94 |
| inhibitor, SP-40,40, sulfated | | | | | | | |
| glycoprotein 2, testosterone- | | | | | | | |
| repressed prostate message | | | | | | | |
| 2, apolipoprotein J) | | | | | | | |

The analyses of the genes set forth above (cdk6, TIMP, CLu, PRL1) were extended to a larger panel of 24 xenografts. Seven of the xenograft data points used in the studies above were repeated in this study. Expression of each gene analyzed was determined by RT-PCR (Taqman). Expression levels of each gene analyzed in the 22 xenografts which are shown, below, in Tables 7a-7k, are relative to the expression level in the known anti-IGF1R sensitive cell line, H322. The tables set forth the cell line name, its origin and the resistant/sensitive status of the cell line along with the % tumor growth inhibition (% TGI) associated with each cell line.

TABLE 7a
Fold change relative to expression level of
H322.

| | Tissue of | sens or | | | | | | |
|---|---|---|---|---|---|---|---|---|
| Xenograft | origin | resistant | % TGI | CLU | TIMP | CDK6 | BMP7 | CERK |
| 05FTI-05-2774 | ovarian | R | 10 | 0.51 | 7.69 | 1.53 | 0.05 | 0.19 |
| A375-SM | melanoma | R | 10 | 0.18 | 62.92 | 0.41 | 0.003 | 1.774 |
| 22rv1 | prostate | R | 20 | 24.18 | 3.37 | 0.20 | 0.00 | 0.14 |
| MCF7BL | breast | R | 22 | 0.25 | 1.05 | 0.06 | 1.28 | 0.05 |
| H838 | NSCLC | R | 24 | 11.16 | 7.83 | 0.28 | 0.13 | 0.37 |
| ES2 | ovarian | R | 30 | 0.26 | 7.20 | 1.72 | 0.000 | 2.994 |
| Colo205 | colon | R | 35 | 0.18 | 2.50 | 0.75 | 0.72 | 0.91 |
| WiDr | colon | R | 35 | 0.17 | 1.32 | 0.23 | 0.00 | 0.14 |
| MB231 | breast | R | 38 | 0.03 | 0.01 | 0.00 | 0.00 | 0.00 |
| MiaPaCa | pancreas | S | 50 | 1.46 | 8.83 | 0.75 | 0.13 | 0.27 |
| HT29 | colon | S | 50 | 0.08 | 1.55 | 0.19 | 0.00 | 0.24 |
| DU145 | prostate | S | 50 | 7.05 | 3.22 | 0.05 | 0.01 | 1.99 |
| RH30 | rhabdo | S | 53 | 0.01 | 0.44 | 0.83 | 0.07 | 1.71 |
| SW527 | breast | S | 56 | 0.25 | 1.90 | 0.51 | 0.66 | 0.66 |
| RD | rhabdo | S | 58 | 3.10 | 15.45 | 11.35 | 0.09 | 6.32 |
| SK-N-MC | rhabdo | S | 59 | 0.24 | 0.29 | 0.01 | 0.00 | 0.87 |
| Hs700T | pancreas | S | 59 | 4.49 | 0.36 | 1.41 | 1.14 | 0.13 |
| A498 | renal | S | 59 | 20.69 | 6.30 | 0.95 | 0.000 | 0.600 |
| BxPC3 | prostate | S | 60 | 7.45 | 8.52 | 1.25 | 0.337 | 1.480 |
| H322 | NSCLC | S | 80 | 1.00 | 1.00 | 1.00 | 1.00 | 1.00 |
| SK-N-AS | neuro | S | 85 | 0.08 | 2.22 | 1.50 | 0.84 | 2.60 |
| SJSA | osteo | S | 88 | 0.88 | 5.92 | 0.98 | 0.77 | 0.54 |
| SK-N-FI | neuro | S | 100 | 2.11 | 0.84 | 1.50 | 0.80 | 2.55 |
| MX1-04SB | | | | 6.22 | 0.60 | 0.00 | 0.60 | 0.43 |

TABLE 7b
Fold change relative to expression level of H322.

| | Tissue of | sens or | | | | | | |
|---|---|---|---|---|---|---|---|---|
| Xenograft | origin | resistant | % TGI | AUTS2 | HDGF | PCDH | SMC4 | TLE |
| 05FTI-05-2774 | ovarian | R | 10 | 0.03 | 0.14 | 4.37 | 0.08 | 0.07 |
| A375-SM | melanoma | R | 10 | 0.000 | 0.716 | 31.326 | 1.643 | 0.418 |
| 22rv1 | prostate | R | 20 | 0.18 | 0.28 | 3.29 | 0.68 | 0.08 |
| MCF7BL | breast | R | 22 | 0.05 | 0.45 | 3.75 | 0.42 | 0.00 |
| H838 | NSCLC | R | 24 | 0.02 | 0.40 | 0.57 | 0.65 | 0.03 |
| ES2 | ovarian | R | 30 | 0.018 | 0.936 | 10.135 | 1.989 | 0.616 |
| Colo205 | colon | R | 35 | 0.11 | 0.00 | 0.60 | 0.27 | 0.09 |
| WiDr | colon | R | 35 | 0.01 | 0.00 | 1.25 | 0.09 | 0.07 |
| MB231 | breast | R | 38 | 0.00 | 0.04 | 14.44 | 0.00 | 0.00 |
| MiaPaCa | pancreas | S | 50 | 0.04 | 0.13 | 10.27 | 0.60 | 0.00 |
| HT29 | colon | S | 50 | 0.02 | 0.00 | 0.94 | 0.11 | 0.12 |
| DU145 | prostate | S | 50 | 0.00 | 1.37 | 7.64 | 2.89 | 0.83 |
| RH30 | rhabdo | S | 53 | 0.42 | 0.69 | 19.36 | 1.41 | 1.11 |
| SW527 | breast | S | 56 | 0.00 | 0.29 | 5.28 | 0.32 | 0.00 |
| RD | rhabdo | S | 58 | 1.40 | 2.08 | 47.51 | 4.91 | 7.92 |
| SK-N-MC | rhabdo | S | 59 | 0.26 | 0.25 | 3.25 | 0.90 | 0.00 |
| Hs700T | pancreas | S | 59 | 0.00 | 0.24 | 1.16 | 0.25 | 0.22 |
| A498 | renal | S | 59 | 0.004 | 0.402 | 17.242 | 0.795 | 1.191 |
| BxPC3 | prostate | S | 60 | 0.688 | 0.029 | 6.041 | 0.990 | 1.235 |
| H322 | NSCLC | S | 80 | 1.00 | 1.00 | 1.00 | 1.00 | 1.00 |
| SK-N-AS | neuro | S | 85 | 1.25 | 1.83 | 13.47 | 1.99 | 5.83 |
| SJSA | osteo | S | 88 | 0.13 | 0.32 | 24.07 | 0.45 | 0.50 |
| SK-N-FI | neuro | S | 100 | 0.66 | 2.03 | 4.97 | 1.17 | 5.08 |
| MX1-04SB | | | | 0.01 | 0.29 | 2.72 | 0.28 | 0.00 |

TABLE 7c
Fold change relative to expression level of H322.

| | Tissue of | sens or | | | | | | |
|---|---|---|---|---|---|---|---|---|
| Xenograft | origin | resistant | % TGI | TRIB | TMTEM77 | PPAP | RPL14 | GSTO |
| 05FTI-05-2774 | ovarian | R | 10 | 0.02 | 0.32 | 1.11 | 0.50 | 0.66 |
| A375-SM | melanoma | R | 10 | 2.007 | 0.600 | 4.474 | 2.263 | 0.062 |
| 22rv1 | prostate | R | 20 | 0.00 | 0.95 | 5.80 | 3.59 | 4.65 |
| MCF7BL | breast | R | 22 | 0.01 | 0.12 | 0.71 | 0.57 | 0.40 |
| H838 | NSCLC | R | 24 | 0.19 | 2.60 | 3.40 | 2.80 | 1.49 |
| ES2 | ovarian | R | 30 | 0.179 | 0.818 | 2.012 | 5.816 | 0.428 |
| Colo205 | colon | R | 35 | 0.38 | 0.32 | 1.73 | 0.36 | 0.33 |
| WiDr | colon | R | 35 | 0.01 | 0.37 | 0.61 | 0.26 | 0.31 |
| MB231 | breast | R | 38 | 0.00 | 0.00 | 0.00 | 0.00 | 0.00 |
| MiaPaCa | pancreas | S | 50 | 1.23 | 0.53 | 0.82 | 0.59 | 0.01 |
| HT29 | colon | S | 50 | 0.02 | 0.58 | 0.54 | 0.35 | 0.42 |
| DU145 | prostate | S | 50 | 0.14 | 2.70 | 7.99 | 2.62 | 6.07 |
| RH30 | rhabdo | S | 53 | 0.23 | 0.38 | 0.21 | 0.96 | 0.00 |
| SW527 | breast | S | 56 | 0.52 | 0.28 | 0.86 | 0.63 | 0.56 |
| RD | rhabdo | S | 58 | 7.72 | 2.95 | 5.74 | 6.12 | 0.08 |
| SK-N-MC | rhabdo | S | 59 | 0.50 | 0.31 | 1.96 | 0.51 | 0.00 |
| Hs700T | pancreas | S | 59 | 0.01 | 0.23 | 0.31 | 0.59 | 0.97 |
| A498 | renal | S | 59 | 0.108 | 1.426 | 3.248 | 1.716 | 0.272 |
| BxPC3 | prostate | S | 60 | 0.305 | 3.599 | 3.487 | 1.480 | 3.222 |
| H322 | NSCLC | S | 80 | 1.00 | 1.00 | 1.00 | 1.00 | 1.00 |
| SK-N-AS | neuro | S | 85 | 1.46 | 1.19 | 1.73 | 1.67 | 0.11 |
| SJSA | osteo | S | 88 | 0.59 | 0.20 | 0.55 | 0.21 | 0.01 |
| SK-N-FI | neuro | S | 100 | 2.64 | 0.79 | 2.34 | 1.04 | 0.02 |
| MX1-04SB | | | | 0.00 | 0.22 | 0.73 | 0.57 | 0.51 |

TABLE 7d
Fold change relative to expression level of H322.

| | Tissue of | sens or | | | | | | |
|---|---|---|---|---|---|---|---|---|
| Xenograft | origin | resistant | % TGI | PARVB | MST1 | JOSD | IFI30 | CRIP |
| 05FTI-05-2774 | ovarian | R | 10 | 30.38 | 2.19 | 0.63 | 0.88 | 31.47 |
| A375-SM | melanoma | R | 10 | 215.160 | 1.777 | 8.609 | 2.916 | 1.935 |
| 22rv1 | prostate | R | 20 | 82.14 | 7.00 | 5.26 | 1.74 | 22.62 |
| MCF7BL | breast | R | 22 | 5.21 | 0.70 | 0.36 | 0.59 | 0.17 |
| H838 | NSCLC | R | 24 | 57.36 | 1.34 | 3.58 | 6.92 | 160.57 |
| ES2 | ovarian | R | 30 | 136.270 | 1.065 | 13.616 | 0.040 | 0.397 |
| Colo205 | colon | R | 35 | 5.88 | 0.20 | 0.66 | 1.45 | 3.61 |
| WiDr | colon | R | 35 | 0.13 | 0.17 | 0.50 | 0.53 | 50.58 |
| MB231 | breast | R | 38 | 3.05 | 0.00 | 0.00 | 0.00 | 0.00 |
| MiaPaCa | pancreas | S | 50 | 25.97 | 0.48 | 0.89 | 1.98 | 0.37 |
| HT29 | colon | S | 50 | 0.17 | 0.24 | 0.86 | 0.55 | 64.85 |
| DU145 | prostate | S | 50 | 68.71 | 2.28 | 14.88 | 5.10 | 50.34 |
| RH30 | rhabdo | S | 53 | 0.21 | 0.24 | 1.11 | 0.02 | 0.05 |
| SW527 | breast | S | 56 | 0.17 | 0.27 | 1.61 | 1.38 | 5.59 |
| RD | rhabdo | S | 58 | 274.46 | 2.92 | 16.26 | 0.05 | 0.59 |
| SK-N-MC | rhabdo | S | 59 | 38.49 | 0.17 | 1.55 | 0.00 | 34.32 |
| Hs700T | pancreas | S | 59 | 7.32 | 0.12 | 0.53 | 1.38 | 97.28 |
| A498 | renal | S | 59 | 51.853 | 3.716 | 3.788 | 4.795 | 0.411 |
| BxPC3 | prostate | S | 60 | 32.550 | 1.734 | 9.817 | 1.542 | 169.156 |
| H322 | NSCLC | S | 80 | 1.00 | 1.00 | 1.00 | 1.00 | 1.00 |
| SK-N-AS | neuro | S | 85 | 45.79 | 0.79 | 3.91 | 0.05 | 8.67 |
| SJSA | osteo | S | 88 | 8.31 | 0.37 | 0.32 | 0.03 | 0.47 |
| SK-N-FI | neuro | S | 100 | 61.13 | 2.06 | 2.99 | 0.10 | 0.10 |
| MX1-04SB | | | | 4.51 | 0.74 | 0.51 | 1.34 | 0.28 |

TABLE 7e
Fold change relative to expression level of H322.

| | Tissue of | sens or | | | | | | |
|---|---|---|---|---|---|---|---|---|
| Xenograft | origin | resistant | % TGI | COL14 | ACAT1 | RASGE | PRDX | PRL1 Affy |
| 05FTI-05-2774 | ovarian | R | 10 | 0.38 | 0.80 | 1.28 | 2.63 | 1.55 |
| A375-SM | melanoma | R | 10 | 0.000 | 2.336 | 0.368 | 4.683 | 4.575 |
| 22rv1 | prostate | R | 20 | 1.55 | 3.22 | 6.73 | 3.07 | 9.54 |
| MCF7BL | breast | R | 22 | 0.14 | 0.18 | 0.07 | 1.94 | 4.20 |
| H838 | NSCLC | R | 24 | 1.57 | 2.10 | 7.90 | 2.65 | 8.34 |
| ES2 | ovarian | R | 30 | 0.264 | 2.102 | 0.000 | 3.342 | 2.116 |
| Colo205 | colon | R | 35 | 0.00 | 0.19 | 0.00 | 2.24 | 5.15 |
| WiDr | colon | R | 35 | 0.00 | 0.12 | 0.00 | 0.82 | 1.17 |
| MB231 | breast | R | 38 | 0.00 | 0.05 | 0.00 | 0.00 | 0.02 |
| MiaPaCa | pancreas | S | 50 | 0.00 | 1.03 | 0.42 | 3.26 | 2.04 |
| HT29 | colon | S | 50 | 0.00 | 0.16 | 0.00 | 0.85 | 1.67 |
| DU145 | prostate | S | 50 | 0.01 | 3.67 | 13.47 | 2.09 | 14.16 |
| RH30 | rhabdo | S | 53 | 0.01 | 0.41 | 0.00 | 3.06 | 2.14 |
| SW527 | breast | S | 56 | 0.00 | 0.67 | 0.02 | 3.38 | 2.40 |
| RD | rhabdo | S | 58 | 0.20 | 2.82 | 0.03 | 2.06 | 2.31 |
| SK-N-MC | rhabdo | S | 59 | 0.00 | 0.63 | 0.00 | 3.14 | 2.51 |
| Hs700T | pancreas | S | 59 | 0.11 | 0.13 | 1.45 | 2.88 | 12.71 |
| A498 | renal | S | 59 | 0.311 | 2.432 | 10.101 | 0.829 | 1.133 |
| BxPC3 | prostate | S | 60 | 3.012 | 0.324 | 0.000 | 0.590 | 0.996 |
| H322 | NSCLC | S | 80 | 1.00 | 1.00 | 1.00 | 1.00 | 1.00 |
| SK-N-AS | neuro | S | 85 | 0.56 | 1.39 | 0.00 | 1.11 | 1.96 |
| SJSA | osteo | S | 88 | 0.23 | 0.09 | 0.04 | 1.03 | 0.81 |
| SK-N-FI | neuro | S | 100 | 0.00 | 1.06 | 2.14 | 0.36 | 1.80 |
| MX1-04SB | | | | 0.05 | 0.25 | 2.46 | 2.05 | 2.48 |

TABLE 7f
Fold change relative to expression level of H322.

| | Tissue of | sens or | | | | | | | |
|---|---|---|---|---|---|---|---|---|---|
| Xenograft | origin | resistant | % TGI | GSTK | C6orf192 | c14orf132 | C1QBP | TMEM64 | TMEM5 |
| 05FTI-05-2774 | ovarian | R | 10 | 13.55 | 6.20 | 0.71 | 3.17 | 3.35 | 3.23 |
| A375-SM | melanoma | R | 10 | 4.07 | 1.63 | 0.30 | 2.28 | 2.15 | 0.82 |
| 22rv1 | prostate | R | 20 | 6.15 | 3.98 | 0.45 | 3.25 | 2.95 | 2.74 |
| MCF7BL | breast | R | 22 | 7.02 | 1.35 | 1.03 | 2.64 | 14.96 | 1.29 |
| H838 | NSCLC | R | 24 | 20.39 | 3.49 | 0.05 | 1.70 | 4.20 | 4.00 |
| ES2 | ovarian | R | 30 | 3.01 | 1.07 | 0.01 | 1.76 | 1.20 | 1.15 |
| Colo205 | colon | R | 35 | 13.46 | 2.22 | 0.00 | 1.25 | 0.05 | 1.54 |
| WiDr | colon | R | 35 | 7.47 | 3.11 | 0.00 | 0.83 | 1.16 | 1.38 |
| MB231 | breast | R | 38 | 0.01 | 0.00 | 0.00 | 0.04 | 0.00 | 0.00 |
| MiaPaCa | pancreas | S | 50 | 13.62 | 2.92 | 0.20 | 3.33 | 1.02 | 2.52 |
| HT29 | colon | S | 50 | 13.19 | 4.46 | 0.00 | 0.93 | 1.07 | 2.01 |
| DU145 | prostate | S | 50 | 3.30 | 0.77 | 0.79 | 0.68 | 2.42 | 1.21 |
| RH30 | rhabdo | S | 53 | 2.53 | 3.75 | 0.01 | 2.03 | 1.93 | 4.23 |
| SW527 | breast | S | 56 | 8.51 | 3.22 | 0.34 | 5.38 | 1.14 | 2.25 |
| RD | rhabdo | S | 58 | 2.74 | 0.93 | 1.31 | 0.85 | 0.96 | 1.50 |
| SK-N-MC | rhabdo | S | 59 | 2.47 | 3.55 | 1.96 | 3.01 | 4.38 | 2.03 |
| Hs700T | pancreas | S | 59 | 4.90 | 2.63 | 0.39 | 1.86 | 5.04 | 1.52 |
| A498 | renal | S | 59 | 20.39 | 1.16 | 0.20 | 0.92 | 2.49 | 2.00 |
| BxPC3 | prostate | S | 60 | 6.72 | 1.15 | 0.00 | 0.30 | 0.25 | 0.69 |
| H322 | NSCLC | S | 80 | 1.00 | 1.00 | 1.00 | 1.00 | 1.00 | 1.00 |
| SK-N-AS | neuro | S | 85 | 2.85 | 0.00 | 10.23 | 1.18 | 1.21 | 1.63 |
| SJSA | osteo | S | 88 | 4.66 | 1.79 | 4.86 | 1.25 | 2.59 | 1.97 |
| SK-N-FI | neuro | S | 100 | 3.93 | 0.00 | 9.49 | 1.18 | 0.51 | 1.06 |
| MX1-04SB | | | | 7.32 | 0.98 | 3.46 | 1.62 | 5.06 | 1.26 |

TABLE 7g
Fold change relative to expression level of H322.

| | Tissue of | sens or | | | | | | |
|---|---|---|---|---|---|---|---|---|
| Xenograft | origin | resistant | % TGI | TCF4 | PDIA5 | NQO2 | MEIS2 | FBXO |
| 05FTI-05-2774 | ovarian | R | 10 | 0.16 | 4.62 | 4.66 | 0.10 | 4.03 |
| A375-SM | melanoma | R | 10 | 0.07 | 4.33 | 1.45 | 0.19 | 0.56 |
| 22rv1 | prostate | R | 20 | 0.87 | 3.59 | 5.37 | 0.00 | 3.36 |
| MCF7BL | breast | R | 22 | 0.54 | 2.41 | 3.36 | 0.00 | 1.38 |
| H838 | NSCLC | R | 24 | 0.31 | 4.21 | 4.89 | 0.47 | 3.13 |
| ES2 | ovarian | R | 30 | 0.53 | 1.61 | 0.35 | 0.42 | 0.03 |
| Colo205 | colon | R | 35 | 0.00 | 5.57 | 3.52 | 0.50 | 0.36 |
| WiDr | colon | R | 35 | 0.03 | 2.98 | 1.24 | 0.00 | 0.05 |
| MB231 | breast | R | 38 | 0.04 | 0.00 | 0.00 | 0.00 | 0.00 |
| MiaPaCa | pancreas | S | 50 | 0.01 | 4.82 | 6.60 | 9.19 | 2.01 |
| HT29 | colon | S | 50 | 0.01 | 3.86 | 1.75 | 0.14 | 0.04 |
| DU145 | prostate | S | 50 | 0.01 | 1.08 | 0.93 | 1.11 | 0.65 |
| RH30 | rhabdo | S | 53 | 5.74 | 2.08 | 1.63 | 1.11 | 0.00 |
| SW527 | breast | S | 56 | 1.66 | 2.63 | 1.58 | 0.00 | 0.43 |
| RD | rhabdo | S | 58 | 7.95 | 0.84 | 0.33 | 0.43 | 0.45 |
| SK-N-MC | rhabdo | S | 59 | 7.03 | 9.09 | 7.37 | 0.61 | 0.43 |
| Hs700T | pancreas | S | 59 | 0.02 | 2.86 | 14.60 | 0.51 | 0.69 |
| A498 | renal | S | 59 | 0.01 | 2.13 | 1.99 | 0.05 | 0.68 |
| BxPC3 | prostate | S | 60 | 0.01 | 0.47 | 0.56 | 0.37 | 0.49 |
| H322 | NSCLC | S | 80 | 1.00 | 1.00 | 1.00 | 1.00 | 1.00 |
| SK-N-AS | neuro | S | 85 | 22.79 | 1.83 | 0.77 | 8.89 | 0.03 |
| SJSA | osteo | S | 88 | 9.80 | 2.91 | 3.05 | 0.85 | 0.41 |
| SK-N-FI | neuro | S | 100 | 18.56 | 0.66 | 0.83 | 2.15 | 0.13 |
| MX1-04SB | | | | 0.00 | 2.98 | 2.76 | 0.00 | 2.48 |

TABLE 7h
Fold change relative to expression level of H322.

| | Tissue of | sens or | | | | | | |
|---|---|---|---|---|---|---|---|---|
| Xenograft | origin | resistant | % TGI | ACAD1 | EML4 | ELLS1 | ATF1 | MYO1C |
| 05FTI-05-2774 | ovarian | R | 10 | 3.58 | 1.12 | 0.63 | 1.25 | 4.41 |
| A375-SM | melanoma | R | 10 | 1.51 | 0.57 | 0.67 | 0.52 | 0.88 |
| 22rv1 | prostate | R | 20 | 2.86 | 0.70 | 0.64 | 1.97 | 2.28 |
| MCF7BL | breast | R | 22 | 1.71 | 1.17 | 0.90 | 2.52 | 0.14 |
| H838 | NSCLC | R | 24 | 2.05 | 0.69 | 0.52 | 2.62 | 1.69 |
| ES2 | ovarian | R | 30 | 0.51 | 0.34 | 0.34 | 0.43 | 0.00 |
| Colo205 | colon | R | 35 | 2.34 | 2.28 | 0.82 | 0.96 | 1.00 |
| WiDr | colon | R | 35 | 1.25 | 2.75 | 0.69 | 0.31 | 2.18 |
| MB231 | breast | R | 38 | 0.00 | 0.00 | 0.00 | 0.00 | 0.00 |
| MiaPaCa | pancreas | S | 50 | 3.18 | 5.32 | 2.33 | 0.79 | 2.15 |
| HT29 | colon | S | 50 | 2.05 | 3.50 | 1.10 | 0.52 | 2.70 |
| DU145 | prostate | S | 50 | 0.81 | 0.46 | 0.68 | 0.51 | 0.73 |
| RH30 | rhabdo | S | 53 | 0.39 | 4.55 | 2.87 | 2.14 | 0.19 |
| SW527 | breast | S | 56 | 2.62 | 3.15 | 1.94 | 1.74 | 0.00 |
| RD | rhabdo | S | 58 | 0.51 | 1.92 | 6.47 | 0.55 | 0.43 |
| SK-N-MC | rhabdo | S | 59 | 1.09 | 3.01 | 1.20 | 1.45 | 0.68 |
| Hs700T | pancreas | S | 59 | 1.92 | 0.83 | 0.70 | 1.80 | 0.95 |
| A498 | renal | S | 59 | 3.60 | 1.38 | 0.38 | 0.20 | 0.98 |
| BxPC3 | prostate | S | 60 | 0.40 | 0.89 | 0.51 | 0.32 | 0.00 |
| H322 | NSCLC | S | 80 | 1.00 | 1.00 | 1.00 | 1.00 | 1.00 |
| SK-N-AS | neuro | S | 85 | 0.35 | 1.64 | 2.55 | 0.43 | 0.00 |
| SJSA | osteo | S | 88 | 1.28 | 2.10 | 3.99 | 0.99 | 1.30 |
| SK-N-FI | neuro | S | 100 | 0.48 | 2.05 | 2.92 | 0.55 | 0.68 |
| MX1-04SB | | | | 2.60 | 1.65 | 1.34 | 1.53 | 1.65 |

TABLE 7i
Fold change relative to expression level of H322.

| | Tissue of | sens or | | | | | | |
|---|---|---|---|---|---|---|---|---|
| Xenograft | origin | resistant | % TGI | PCDGH3 | HSPE | DNAJC | CRYZ | KIAA |
| 05FTI-05-2774 | ovarian | R | 10 | 16.06 | 4.03 | 2.94 | 6.14 | 1.17 |
| A375-SM | melanoma | R | 10 | 21.31 | 1.96 | 1.47 | 1.95 | 0.42 |
| 22rv1 | prostate | R | 20 | 3.77 | 4.89 | 1.85 | 3.67 | 1.51 |
| MCF7BL | breast | R | 22 | 10.44 | 3.43 | 1.91 | 1.31 | 1.48 |
| H838 | NSCLC | R | 24 | 0.64 | 5.25 | 3.07 | 3.68 | 0.40 |
| ES2 | ovarian | R | 30 | 6.00 | 2.00 | 0.94 | 0.69 | 0.30 |
| Colo205 | colon | R | 35 | 1.20 | 1.65 | 1.41 | 2.22 | 6.06 |
| WiDr | colon | R | 35 | 3.11 | 1.87 | 2.21 | 3.49 | 2.91 |
| MB231 | breast | R | 38 | 16.05 | 0.00 | 0.00 | 0.00 | 0.01 |
| MiaPaCa | pancreas | S | 50 | 20.70 | 4.16 | 1.98 | 3.41 | 0.82 |
| HT29 | colon | S | 50 | 2.76 | 2.57 | 2.07 | 4.88 | 3.85 |
| DU145 | prostate | S | 50 | 2.40 | 0.59 | 0.61 | 1.10 | 0.19 |
| RH30 | rhabdo | S | 53 | 28.60 | 5.27 | 1.38 | 1.62 | 2.69 |
| SW527 | breast | S | 56 | 10.25 | 3.93 | 2.26 | 2.12 | 1.46 |
| RD | rhabdo | S | 58 | 26.19 | 2.91 | 2.35 | 1.31 | 1.22 |
| SK-N-MC | rhabdo | S | 59 | 6.90 | 6.75 | 2.05 | 1.88 | 1.46 |
| Hs700T | pancreas | S | 59 | 3.36 | 5.26 | 1.79 | 2.52 | 0.50 |
| A498 | renal | S | 59 | 12.18 | 2.36 | 1.02 | 6.84 | 0.45 |
| BxPC3 | prostate | S | 60 | 3.02 | 0.43 | 0.27 | 0.38 | 0.21 |
| H322 | NSCLC | S | 80 | 1.00 | 1.00 | 1.00 | 1.00 | 1.00 |
| SK-N-AS | neuro | S | 85 | 10.86 | 1.90 | 1.13 | 1.73 | 3.36 |
| SJSA | osteo | S | 88 | 79.84 | 1.84 | 1.52 | 1.80 | 2.85 |
| SK-N-FI | neuro | S | 100 | 6.08 | 0.96 | 1.20 | 0.55 | 2.85 |
| MX1-04SB | | | | 6.85 | 2.46 | 1.79 | 1.57 | 0.52 |

TABLE 7j
Fold change relative to expression level of H322.

| | Tissue of | sens or | | | | | | |
|---|---|---|---|---|---|---|---|---|
| Xenograft | origin | resistant | % TGI | RPL22 | RPL15 | PCCB | ZNF136 | TMEM107 |
| 05FTI-05-2774 | ovarian | R | 10 | 1.32 | 2.13 | 1.48 | 0.85 | 4.63 |
| A375-SM | melanoma | R | 10 | 1.07 | 1.10 | 2.05 | 2.85 | 6.91 |
| 22rv1 | prostate | R | 20 | 2.44 | 3.37 | 3.62 | 1.17 | 2.58 |
| MCF7BL | breast | R | 22 | 1.94 | 7.04 | 2.73 | 0.80 | 2.06 |
| H838 | NSCLC | R | 24 | 2.20 | 1.69 | 1.67 | 0.86 | 1.80 |
| ES2 | ovarian | R | 30 | 1.44 | 2.44 | 1.06 | 0.29 | 4.22 |
| Colo205 | colon | R | 35 | 1.56 | 1.47 | 1.21 | 2.61 | 1.47 |
| WiDr | colon | R | 35 | 0.73 | 0.74 | 0.93 | 0.26 | 0.56 |
| MB231 | breast | R | 38 | 0.00 | 0.04 | 0.00 | 0.00 | 0.00 |
| MiaPaCa | pancreas | S | 50 | 1.90 | 1.96 | 1.28 | 3.07 | 3.03 |
| HT29 | colon | S | 50 | 0.79 | 0.79 | 0.73 | 0.29 | 0.53 |
| DU145 | prostate | S | 50 | 1.11 | 1.34 | 2.62 | 1.81 | 1.41 |
| RH30 | rhabdo | S | 53 | 8.77 | 14.79 | 1.93 | 4.00 | 0.17 |
| SW527 | breast | S | 56 | 3.64 | 3.94 | 2.01 | 1.06 | 0.91 |
| RD | rhabdo | S | 58 | 3.52 | 4.25 | 1.40 | 13.66 | 2.84 |
| SK-N-MC | rhabdo | S | 59 | 4.65 | 2.43 | 6.57 | 1.57 | 1.15 |
| Hs700T | pancreas | S | 59 | 0.96 | 1.30 | 1.13 | 0.05 | 1.23 |
| A498 | renal | S | 59 | 0.53 | 0.87 | 1.16 | 0.00 | 4.41 |
| BxPC3 | prostate | S | 60 | 1.27 | 0.91 | 0.94 | 2.70 | 1.15 |
| H322 | NSCLC | S | 80 | 1.00 | 1.00 | 1.00 | 1.00 | 1.00 |
| SK-N-AS | neuro | S | 85 | 1.05 | 1.55 | 1.76 | 2.93 | 0.58 |
| SJSA | osteo | S | 88 | 0.71 | 1.08 | 0.62 | 0.85 | 1.00 |
| SK-N-FI | neuro | S | 100 | 0.40 | 0.69 | 0.51 | 3.09 | 1.55 |
| MX1-04SB | | | | 1.08 | 2.63 | 5.02 | 1.91 | 1.64 |

TABLE 7k
Fold change relative to expression level of H322.

| | Tissue of | sens or | | | | | | | |
|---|---|---|---|---|---|---|---|---|---|
| Xenograft | origin | resistant | % TGI | DEADC | C19orf54 | LACTB | ALDOC | ZFAND | TSPAN |
| 05FTI-05-2774 | ovarian | R | 10 | 4.87 | 6.46 | 3.72 | 37.09 | 3.03 | 10.90 |
| A375-SM | melanoma | R | 10 | 3.31 | 4.61 | 3.41 | 119.68 | 3.72 | 28.58 |
| 22rv1 | prostate | R | 20 | 2.25 | 3.18 | 2.22 | 21.82 | 3.44 | 4.19 |
| MCF7BL | breast | R | 22 | 1.26 | 2.05 | 0.22 | 9.27 | 1.22 | 7.72 |
| H838 | NSCLC | R | 24 | 1.64 | 1.71 | 0.85 | 12.38 | 1.43 | 23.88 |
| ES2 | ovarian | R | 30 | 4.63 | 3.85 | 3.03 | 29.51 | 6.86 | 26.13 |
| Colo205 | colon | R | 35 | 2.87 | 5.63 | 2.34 | 57.82 | 3.80 | 0.21 |
| WiDr | colon | R | 35 | 0.62 | 1.89 | 2.23 | 8.42 | 1.08 | 2.58 |
| MB231 | breast | R | 38 | 0.00 | 0.00 | 0.00 | 0.00 | 0.01 | 0.01 |
| MiaPaCa | pancreas | S | 50 | 5.19 | 5.47 | 0.77 | 49.23 | 3.15 | 15.08 |
| HT29 | colon | S | 50 | 0.67 | 1.48 | 1.45 | 18.33 | 1.39 | 1.81 |
| DU145 | prostate | S | 50 | 2.61 | 4.12 | 22.05 | 0.11 | 5.63 | 15.81 |
| RH30 | rhabdo | S | 53 | 3.43 | 7.49 | 0.60 | 0.83 | 1.52 | 0.94 |
| SW527 | breast | S | 56 | 0.49 | 0.60 | 0.79 | 10.82 | 2.09 | 2.36 |
| RD | rhabdo | S | 58 | 5.30 | 14.39 | 6.72 | 35.29 | 9.41 | 14.09 |
| SK-N-MC | rhabdo | S | 59 | 1.55 | 2.61 | 0.32 | 20.67 | 2.91 | 9.05 |
| Hs700T | pancreas | S | 59 | 1.09 | 1.18 | 0.67 | 3.36 | 0.91 | 2.02 |
| A498 | renal | S | 59 | 1.59 | 5.51 | 9.52 | 15.61 | 3.05 | 24.83 |
| BxPC3 | prostate | S | 60 | 3.90 | 4.07 | 3.55 | 16.47 | 4.17 | 2.42 |
| H322 | NSCLC | S | 80 | 1.00 | 1.00 | 1.00 | 1.00 | 1.00 | 1.00 |
| SK-N-AS | neuro | S | 85 | 1.64 | 2.83 | 1.11 | 4.44 | 2.13 | 2.57 |
| SJSA | osteo | S | 88 | 0.56 | 1.97 | 0.48 | 0.58 | 1.01 | 2.54 |
| SK-N-FI | neuro | S | 100 | 1.29 | 3.07 | 0.76 | 4.08 | 1.22 | 1.48 |
| MX1-04SB | | | | 0.54 | 3.34 | 1.18 | 20.78 | 1.92 | 2.75 |

### Example 2

### Statistical Analysis of Biomarker Predictive Value

In this example, the biomarkers set forth herein were statistically analyzed in order to assess their value with respect to predicting the sensitivity of a cell to an IGF1R inhibitor. The biomarkers ELLS1, AUTS2, TCF4 and TLE were found to have a particularly high predictive value.

### Predictive Models.

Based on the gene expression data from RT-PCR (Taqman; see above in Tables 7a-7k), two classification methods, Diagonal Linear Discriminant Analysis (DLDA) and Support Vector Machines (SVM), were used to develop multiplex marker assays for prediction of cell line sensitivity to the IGF1R inhibitor (anti-IGF1R antibody LCF/HCA (κ/γ1)).

DLDA is the simplest case of the maximum likelihood discriminant rule, in which the class densities are supposed to have the same diagonal covariance matrix. In the special case of binary classification, the DLDA scheme can be viewed as the “weighted voting scheme” proposed by Golub et al. (Science (1999) 286:531-537).

SVM has been recognized as the most powerful classifier in various applications of pattern classification. For binary classification, SVM learns the classifier by mapping the input training samples into a possibly high-dimensional feature space and seeking a hyperplane in this space which separates the two types of examples with the largest possible margin, i.e. distance to the nearest points. If the training set is not linearly separable, SVM finds a hyperplane, which optimizes a trade-off between good classification and large margin. (Cristianini N, Shawe-Taylor J., An Introduction to Support Vector Machines, Cambridge University Press, Cambridge, UK 2000).

Both DLDA and SVM approaches use the feature selection criterion similar to that described in Golub et al. (Science (1999) 286:531-537) and Slonim et al (“Class Prediction and discovery using gene expression data”, in Proceedings of the 4th Annual International Conference on Computational Molecular Biology (RECOMD), Univeral Academy Press, Tokyo, Japan, pp. 263-272 (2000)). We started with a dataset S consisting of m expression vector xⁱ=(xⁱ₁, . . . , xⁱₙ), 1≦i≦m, where m is the number of xenografts (23) and n is the number of genes measured (57). Each sample is labeled with Yε{+1, −1} (e.g., sensitive vs resistant). In order to find genes that discriminant between the two classes, we calculated a score

$$F ( {x}_{j} ) = \vert \frac{{μ}_{j}^{+} - {μ}_{j}^{-}}{{σ}_{j}^{+} + {σ}_{j}^{-}} \vert$$

where μⱼ⁺ (resp. μⱼ⁻) is the mean expression for gene j using only the xenografts labeled +1 (resp. −1), and σⱼ⁺ and σⱼ⁻ are the standard deviations respectively. The F(xⱼ) score, which is closely related to Fisher criterion score, gave the highest score to those genes whose expression levels differ most on average in the two classes while also favoring those with small deviations in scores in the respective classes.

In order to evaluate the generalization power of each of the classification methods and to estimate their prediction capabilities for unknown samples, we used a standard 10-fold cross-validation technique and split the data randomly and repeatedly into training and test sets. The training sets consisted of randomly chosen subsets containing 90% of each class (resistant and sensitive); the remaining 10% of the samples from each class were left as test sets. The overall accuracy was defined by

$$overall accuracy = \frac{TP + TN}{TP + TN + FP + FN}$$

where TP, FP, TN and FN refer to the number of true positives, false positives, true negatives and false negatives proteins, respectively. In order to keep computing times reasonable, we reported the average of overall accuracy estimates over 100 runs.

We also used the cross validation method to optimize the feature selection and select models that maximize the classification performance. Genes were first ranked based on F(xⱼ) scores. Then, genes with high Pearson correlation coefficients (≧0.9) with top ranked genes were removed to reduce the redundancy. Genes with least F(xⱼ) scores were further recursively removed and predictive accuracies of classifiers were estimated.

For the DLDA approach, the best classification was achieved using four genes, ELLS1, AUTS2, TCF4 and TLE, in the model. The prediction accuracy was estimated as 72.5%. For the SVM approach, the best prediction accuracy was estimated as 75.7%, with three genes, ELLS1, AUTS2 and TCF4, included in the model.

Statistical analysis was performed using R package which is freely available for example at www.r-project.org.

These data demonstrate that ELLS1, AUTS2, TCF4 and TLE are highly useful biomarkers which can be used to predict sensitivity or resisance of any cell to an IGF1R inhibitor, e.g., with about 70% certainty (e.g., about 72.5% or about 75.5% certainty or a range of from about 72.5% to about 75.5% certainty). The present invention includes methods of evaluating expression of all 4, 3, 2 or just 1 of these biomarkers in any combination whatsoever. Methods of evaluating sensitivity can be used, in turn, e.g., for selecting a patient for IGF1R inhibitor therapy.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

Patents, patent applications, publications, product descriptions, and protocols are cited throughout this application, the disclosures of which are incorporated herein by reference in their entireties for all purposes.

We claim:

1. A method for evaluating sensitivity of malignant or neoplastic cells to an IGF1R inhibitor comprising determining if said cells exhibit high expression of one or more genes selected from the group consisting of:
TRE2; SMC4; TRIB2; TLE4; BMP7; PCDHGC3; AUTS2; C14orf132; CERK; HDGFRP3; TCF4; MEIS2; EML4; C7orf41; KIAA1450; ZNF136; D15F37; CDK6; TIMP; Clu; and PRL1;
or low expression of one or more selected from the group consisting of:
ACAT1; ALDOC; C6orf192; COL4A5; C1QBP; CRIP1; DEADC1; GSTK1; GSTO2; PPAPDC1B; TMEM107; JOSD3; TMEM77; MST1; MT1E;
PARVB; PRDX4; RASGEF1A; RPL14; IFI30; ATF1; ACADVL; FBXO6; NQO2; TMEM64; ZFAND1; TMED5; PDIA5; MYO1C; GNPTAB; LACTB2; RPL22; TSPAN4; RPL15; PCCB; CRYZ; DNAJC10; C19orf54; HSPE1; and hqp0376; or both
relative to that of a cell resistant to said inhibitor; wherein said cells are determined to be sensitive if said high expression or said low expression is observed.

2. The method of claim 1 comprising a method for evaluating sensitivity of malignant or neoplastic cells to an IGF1R inhibitor comprising determining if said cells exhibit high expression of one or more genes selected from the group consisting of: ELLS1, AUTS2, TCF4 and TLE.

3. The method of claim 2 comprising a method for evaluating sensitivity of malignant or neoplastic cells to an IGF1R inhibitor comprising determining if said cells exhibit high expression of ELLS1, AUTS2, TCF4 and TLE.

4. The method of claim 1 further comprising administering a therapeutically effective dose of said inhibitor, optionally in association with a further therapeutic agent, to the body of a mammalian subject comprising said malignant or neoplastic cells if the cells are determined to be sensitive.

5. The method of claim 4 wherein the further therapeutic agent is one or more members selected from the group consisting of everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAKISTAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR, KRX-0402, lucanthone, LY 317615, neuradiab, vitespan, Rta 744, Sdx 102, talampanel, atrasentan, Xr 311, romidepsin, 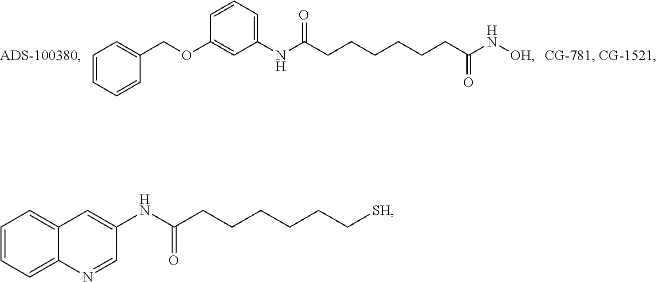
SB-556629, chiamydocin, JNJ-16241199, 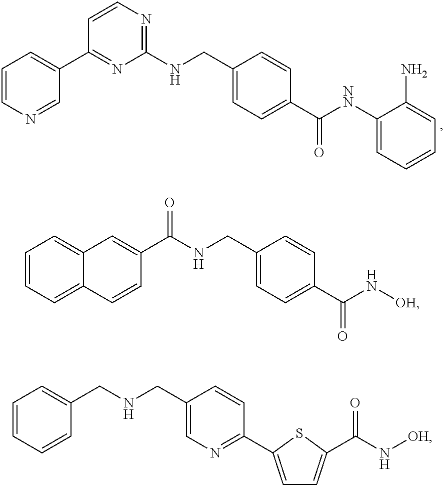
vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, irinotecan; a combination of irinotecan, 5-fluorouracil and leucovorin; PEG-labeled irinotecan, FOLFOX regimen, tamoxifen, toremifene citrate, anastrazole, exemestane, letrozole, DES (diethylstilbestrol), estradiol, estrogen, conjugated estrogen, bevacizumab, IMC-1C11, CHIR-258, 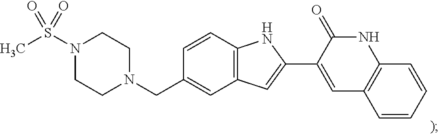
3-[5-(methylsulfonylpiperadinemethyl)-indolyl]-quinolone, vatalanib, AG-013736, AVE-0005, the acetate salt of [D-Ser(But) 6, Azgly 10] (pyro-Glu-His-Trp-Ser-Tyr-D-Ser(But)-Leu-Arg-Pro-Azgly-NH₂ acetate [C₅₉H₈₄N₁₈O₁₄.(C₂H₄O₂)ₓ where x=1 to 2.4], goserelin acetate, leuprolide acetate, triptorelin pamoate, sunitinib, sunitinib malate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, lonafarnib, 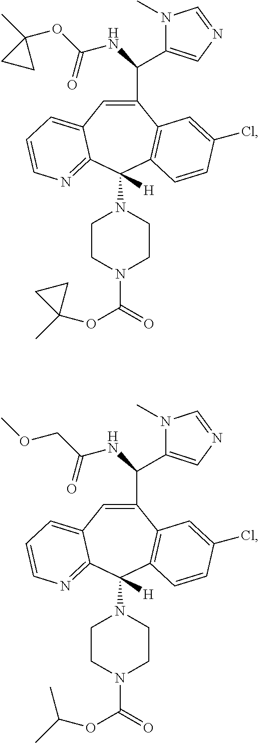
BMS-214662, tipifarnib; amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyclophosphamide, cyproterone, cytarabine, dacarbazine, dactinomycin, daunorubicin, diethylstilbestrol, epirubicin, fludarabine, fludrocortisone, fluoxymesterone, flutamide, hydroxyurea, idarubicin, ifosfamide, imatinib, leucovorin, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mercaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, 5-fluorouracil, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa and darbepoetin alfa.

6. The method of claim 1 wherein said inhibitor is a member selected from the group consisting of an antibody or antigen-binding fragment thereof which binds specifically to IGF1R;

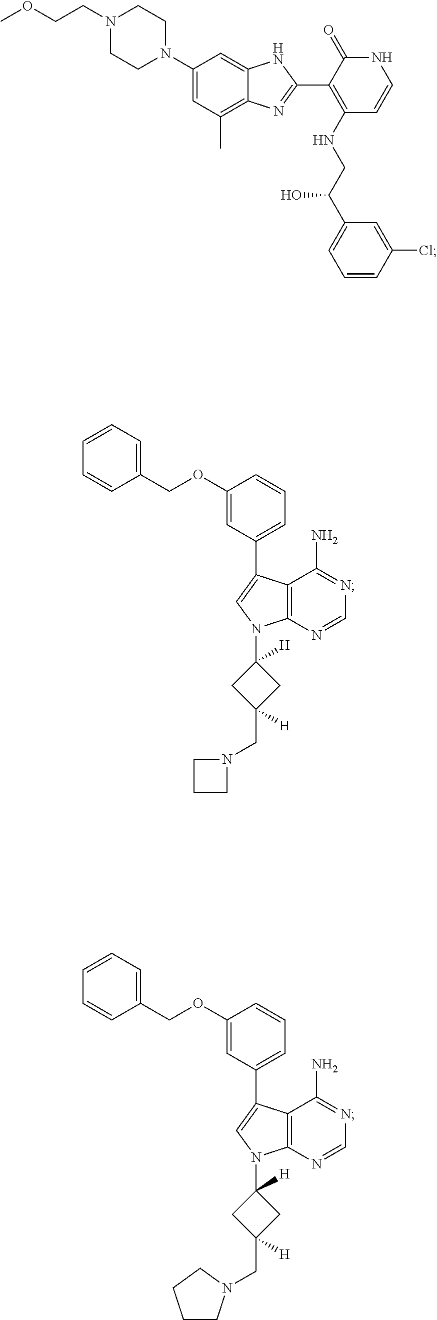

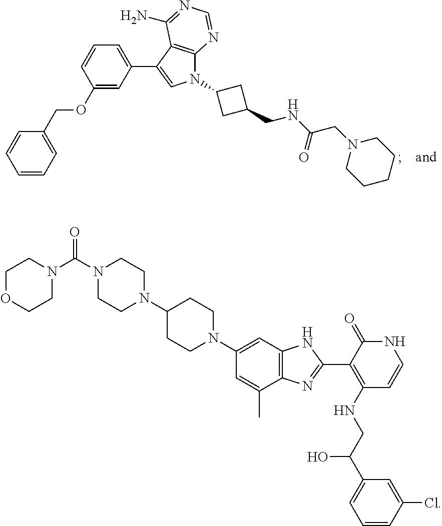

7. The method of claim 6 wherein the inhibitor is an antibody or fragment which comprises one or more complementarity determining regions (CDRs) selected from the group consisting of:
RASQSIGSSLH (SEQ ID NO: 99),
YASQSLS (SEQ ID NO: 100),
HQSSRLPHT (SEQ ID NO: 101),
SFAMH (SEQ ID NO:102),
GFTFSSFAMH (SEQ ID NO: 107),
VIDTRGATYYADSVKG (SEQ ID NO: 103), and
LGNFYYGMDV (SEQ ID NO: 104); or wherein the antibody or fragment comprises a mature fragment of a light chain immunoglobulin which comprises the amino acid sequence of SEQ ID NO: 2, 4, 6 or 8; and/or wherein the antibody or fragment comprises a mature fragment of a heavy chain immunoglobulin which comprises the amino acid sequence of SEQ ID NO: 10 or 12.

8. The method of claim 1 wherein the malignant or neoplastic cells are in a tumor or mediate a cancerous condition which tumor or condition is selected from the group consisting of osteosarcoma, rhabdomyosarcoma, neuroblastoma, any pediatric cancer, kidney cancer, leukemia, renal transitional cell cancer, Werner-Morrison syndrome, bladder cancer, Wilm's cancer, ovarian cancer, pancreatic cancer, breast cancer, prostate cancer, benign prostatic hyperplasia, bone cancer, lung cancer, gastric cancer, colorectal cancer, cervical cancer, synovial sarcoma, vasoactive intestinal peptide secreting tumors, tumor angiogenesis, head and neck cancer, squamous cell carcinoma, multiple myeloma, solitary plasmacytoma, renal cell cancer, retinoblastoma, germ cell tumor, hepatoblastoma, hepatocellular carcinoma, melanoma, rhabdoid tumor of the kidney, Ewing Sarcoma, chondrosarcoma, haemotological malignancy, chronic lymphoblastic leukemia, chronic myelomonocytic leukemia, acute lymphoblastic leukemia, acute lymphocytic leukemia, acute myelogenous leukemia, acute myeloblastic leukemia, chronic myeloblastic leukemia, Hodgekin's disease, non-Hodgekin's lymphoma, chronic lymphocytic leukemia, chronic myelogenous leukemia, myelodysplastic syndrome, hairy cell leukemia, mast cell leukemia, mast cell neoplasm, follicular lymphoma, diffuse large cell lymphoma, mantle cell lymphoma, Burkitt Lymphoma, mycosis fungoides, seary syndrome, cutaneous T-cell lymphoma, chronic myeloproliferative disorders, a central nervous system tumor, brain cancer, glioblastoma, non-glioblastoma brain cancer, meningioma, pituitary adenoma, vestibular schwannoma, a primitive neuroectodermal tumor, medulloblastoma, astrocytoma, anaplastic astrocytoma, oligodendroglioma, ependymoma, choroid plexus papilloma, a myeloproliferative disorder, polycythemia vera, thrombocythemia, idiopathic myelfibrosis, soft tissue sarcoma, thyroid cancer, endometrial cancer, carcinoid cancer and liver cancer.

9. The method of claim 1 wherein said malignant or neoplastic cells are obtained from an in vitro source.

10. The method of claim 1 wherein said malignant or neoplastic cells are obtained from an in vivo source.

11. The method of claim 1 wherein expression of one or more of said genes is identified by Northern blot analysis.

12. The method of claim 1 comprising:
(a) obtaining a sample of one or more malignant or neoplastic cells from the body of a mammalian subject;
(b) evaluating the expression level of one or more genes in group I:
TRE2; SMC4; TRIB2; TLE4; BMP7; PCDHGC3; AUTS2; C14 or 1132; CERK; HDGFRP3; TCF4; MEIS2; EML4; C7 orf41; KIAA1450; ZNF136; D15F37; CDK6; TIMP; Clu; PRL1; and/or one or more genes in group II: ACAT1; ALDOC; C6orf192; COL4A5; C1QBP; CRIP1; DEADC1; GSTK1; GSTO2; PPAPDC1B; TMEM107; JOSD3; TMEM77; MST1; MT1E; PARVB; PRDX4; RASGEF1A; RPL14; IFI30; ATF1; ACADVL; FBXO6; NQO2; TMEM64; ZFAND1; TMED5; PDIA5; MYO1C; GNPTAB; LACTB2; RPL22; TSPAN4; RPL15; PCCB; CRYZ; DNAJC10; C19orf54; HSPE1; and hqp0376; in the malignant or neoplastic cells; and
(c) comparing said expression level to that of cells resistant to said IGF1R inhibitor;
wherein the cells are determined to be sensitive to the inhibitor if expression of one or more genes in group I is higher than that of the cell resistant to said inhibitor and/or if expression of one or more genes in group II is lower than that of the cell resistant to said inhibitor.

13. The method of claim 12 wherein step (b) comprises evaluating the expression level of one or more genes selected from the group consisting of ELLS1, AUTS2, TCF4 and TLE.

14. The method of claim 13 wherein step (b) comprises evaluating the expression level of ELLS1, AUTS2, TCF4 and TLE.

15. The method of claim 12 further comprising administering a therapeutically effective dose of said IGF1R inhibitor, optionally in association with a further therapeutic agent, to said subject, if the cells are determined to be sensitive.

16. The method of claim 15 wherein the further therapeutic agent is one or more members selected from the group consisting of everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR, KRX-0402, lucanthone, LY 317615, neuradiab, vitespan, Rta 744, Sdx 102, talampanel, atrasentan, Xr 311, romidepsin, 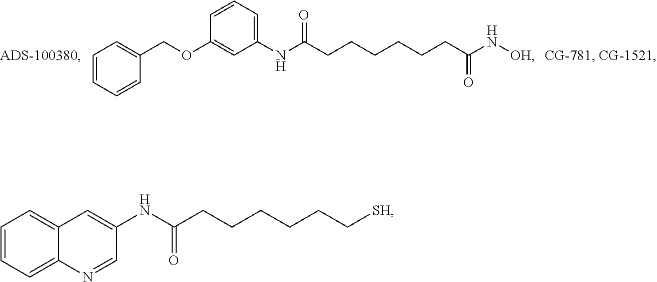
SB-556629, chiamydocin, JNJ-16241199, 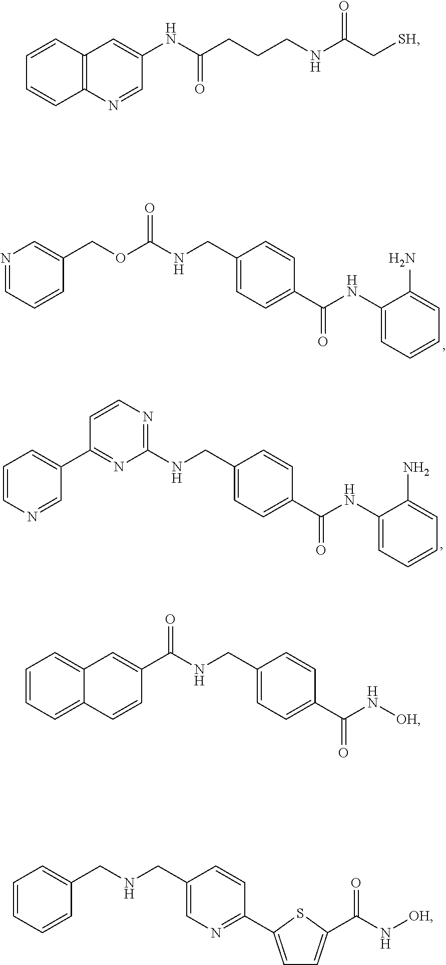
vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, irinotecan; a combination of irinotecan, 5-fluorouracil and leucovorin; PEG-labeled irinotecan, FOLFOX regimen, tamoxifen, toremifene citrate, anastrazole, exemestane, letrozole, DES (diethylstilbestrol), estradiol, estrogen, conjugated estrogen, bevacizumab, IMC-1C11, CHIR-258, 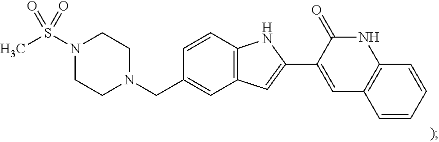
3-[5-(methylsulfonylpiperadinemethyl)-indolyl]-quinolone, vatalanib, AG-013736, AVE-0005, the acetate salt of [D-Ser(But) 6, Azgly 10] (pyro-Glu-His-Trp-Ser-Tyr-D-Ser(Bu t)-Leu-Arg-Pro-Azgly-NH₂ acetate [C₅₉H₈₄N₁₈O₁₄.(C₂H₄O₂)ₓ where x=1 to 2.4], goserelin acetate, leuprolide acetate, triptorelin pamoate, sunitinib, sunitinib malate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, lonafarnib, 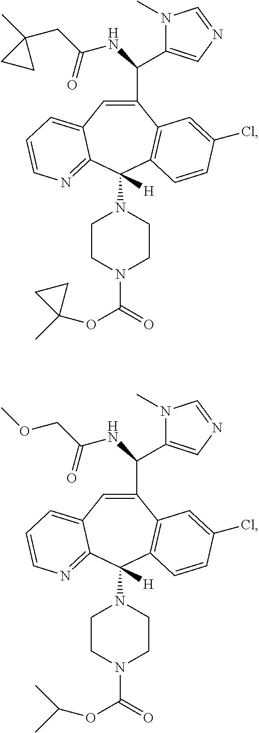
BMS-214662, tipifarnib; amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyclophosphamide, cyproterone, cytarabine, dacarbazine, dactinomycin, daunorubicin, diethylstilbestrol, epirubicin, fludarabine, fludrocortisone, fluoxymesterone, flutamide, hydroxyurea, idarubicin, ifosfamide, imatinib, leucovorin, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mercaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, 5-fluorouracil, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa and darbepoetin alfa.

17. The method of claim 12 wherein said inhibitor is a member selected from the group consisting of an antibody or antigen-binding fragment thereof which binds specifically to IGF1R;

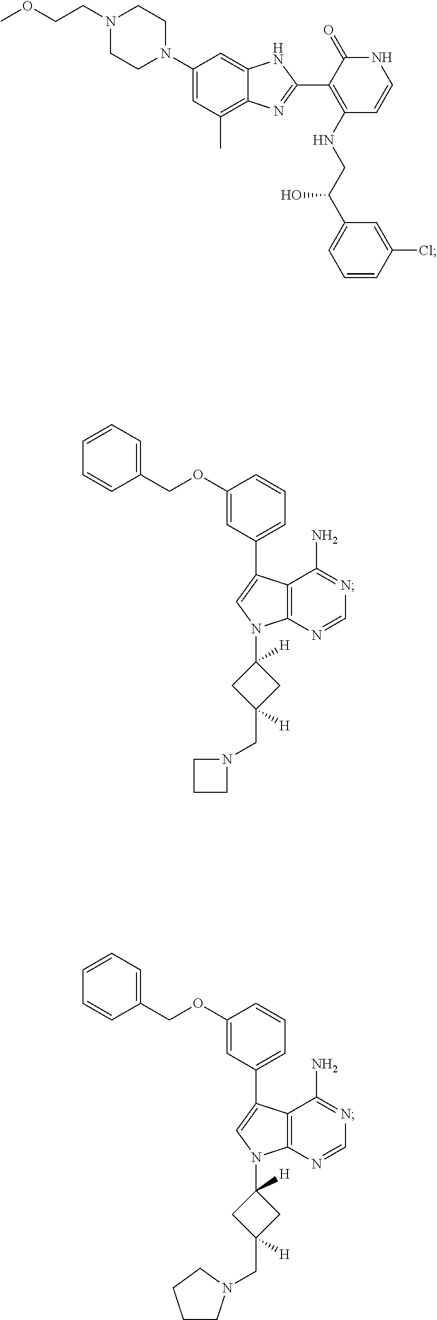

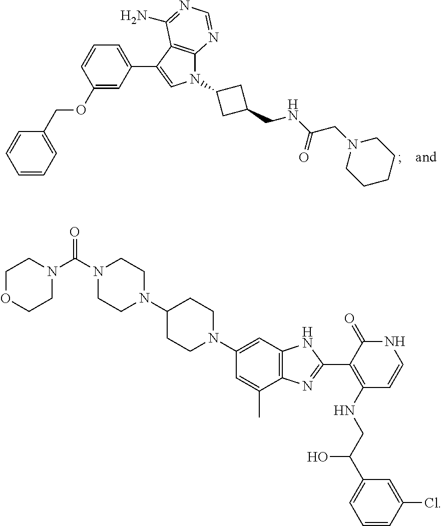

18. The method of claim 17 wherein the inhibitor is an antibody or fragment which comprises one or more complementarity determining regions (CDRs) selected from the group consisting of:
RASQSIGSSLH (SEQ ID NO: 99),
YASQSLS (SEQ ID NO: 100),
HQSSRLPHT (SEQ ID NO: 101),
SFAMH (SEQ ID NO:102),
GFTFSSFAMH (SEQ ID NO: 107),
VIDTRGATYYADSVKG (SEQ ID NO: 103), and
LGNFYYGMDV (SEQ ID NO: 104); or wherein the antibody or fragment comprises a mature fragment of a light chain immunoglobulin which comprises the amino acid sequence of SEQ ID NO: 2, 4, 6 or 8; and/or wherein the antibody or fragment comprises a mature fragment of a heavy chain immunoglobulin which comprises the amino acid sequence of SEQ ID NO: 10 or 12.

19. The method of claim 12 wherein the malignant or neoplastic cells are in a tumor or mediate a cancerous condition selected from the group consisting of osteosarcoma, rhabdomyosarcoma, neuroblastoma, any pediatric cancer, kidney cancer, leukemia, renal transitional cell cancer, Werner-Morrison syndrome, bladder cancer, Wilm's cancer, ovarian cancer, pancreatic cancer, breast cancer, prostate cancer, benign prostatic hyperplasia, bone cancer, lung cancer, gastric cancer, colorectal cancer, cervical cancer, synovial sarcoma, vasoactive intestinal peptide secreting tumors, tumor angiogenesis, head and neck cancer, squamous cell carcinoma, multiple myeloma, solitary plasmacytoma, renal cell cancer, retinoblastoma, germ cell tumor, hepatoblastoma, hepatocellular carcinoma, melanoma, rhabdoid tumor of the kidney, Ewing Sarcoma, chondrosarcoma, haematological malignancy, chronic lymphoblastic leukemia, chronic myelomonocytic leukemia, acute lymphoblastic leukemia, acute lymphocytic leukemia, acute myelogenous leukemia, acute myeloblastic leukemia, chronic myeloblastic leukemia, Hodgekin's disease, non-Hodgekin's lymphoma, chronic lymphocytic leukemia, chronic myelogenous leukemia, myelodysplastic syndrome, hairy cell leukemia, mast cell leukemia, mast cell neoplasm, follicular lymphoma, diffuse large cell lymphoma, mantle cell lymphoma, Burkitt Lymphoma, mycosis fungoides, seary syndrome, cutaneous T-cell lymphoma, chronic myeloproliferative disorders, a central nervous system tumor, brain cancer, glioblastoma, non-glioblastoma brain cancer, meningioma, pituitary adenoma, vestibular schwannoma, a primitive neuroectodermal tumor, medulloblastoma, astrocytoma, anaplastic astrocytoma, oligodendroglioma, ependymoma, choroid plexus papilloma, a myeloproliferative disorder, polycythemia vera, thrombocythemia, idiopathic myelfibrosis, soft tissue sarcoma, thyroid cancer, endometrial cancer, carcinoid cancer and liver cancer.

20. A method for selecting a mammalian subject with malignant or neoplastic cells for treatment with an IGF1R inhibitor comprising evaluating sensitivity of the malignant or neoplastic cells to said inhibitor by the method of claim 1; wherein said subject is selected if said cells are determined to be sensitive.

21. A method for selecting a mammalian subject with malignant or neoplastic cells for treatment with an IGF1R inhibitor comprising evaluating sensitivity of the malignant or neoplastic cells to said inhibitor by the method of claim 2; wherein said subject is selected if said cells are determined to be sensitive.

22. A method for selecting a mammalian subject with malignant or neoplastic cells for treatment with an IGF1R inhibitor comprising evaluating sensitivity of the malignant or neoplastic cells to said inhibitor by the method of claim 3; wherein said subject is selected if said cells are determined to be sensitive.

23. The method of claim 20 wherein said inhibitor is a member selected from the group consisting of an antibody or antigen-binding fragment thereof which binds specifically to IGF1R;

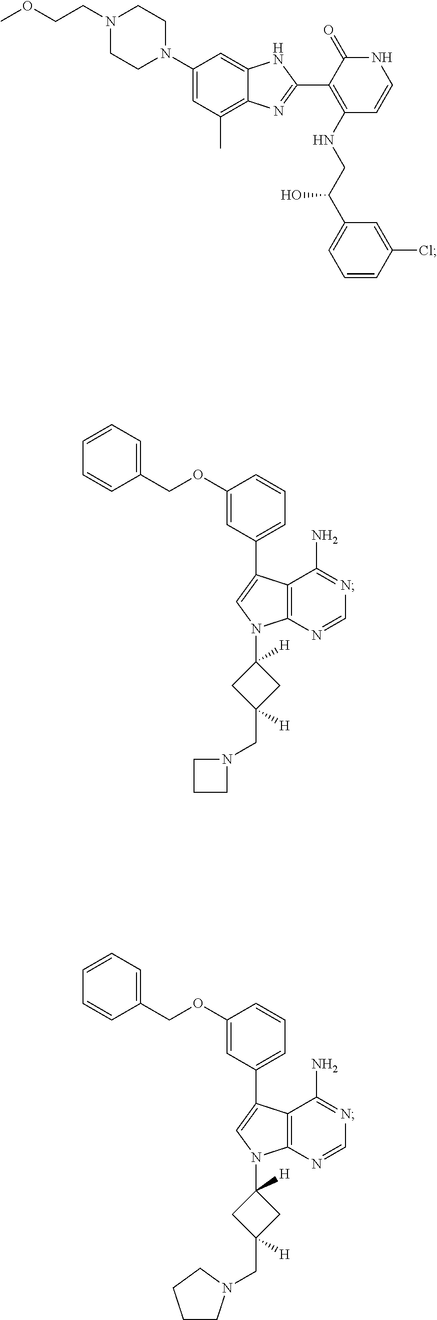

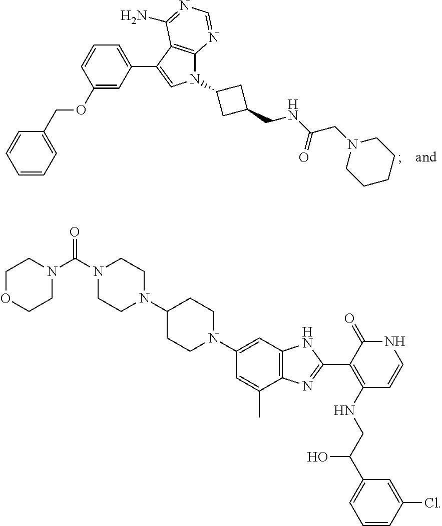

24. The method of claim 23 wherein the inhibitor is an antibody or fragment which comprises one or more complementarity determining regions (CDRs) selected from the group consisting of:
RASQSIGSSLH (SEQ ID NO: 99),
YASQSLS (SEQ ID NO: 100),
HQSSRLPHT (SEQ ID NO: 101),
SFAMH (SEQ ID NO:102),
GFTFSSFAMH (SEQ ID NO: 107),
VIDTRGATYYADSVKG (SEQ ID NO: 103), and
LGNFYYGMDV (SEQ ID NO: 104); or wherein the antibody or fragment comprises a mature fragment of a light chain immunoglobulin which comprises the amino acid sequence of SEQ ID NO: 2, 4, 6 or 8; and/or wherein the antibody or fragment comprises a mature fragment of a heavy chain immunoglobulin which comprises the amino acid sequence of SEQ ID NO: 10 or 12.

25. The method of claim 20 wherein, after the subject is selected, the subject is administered a therapeutically effective dose of said IGF1R inhibitor optionally in association with a further therapeutic agent.

26. The method of claim 25 wherein the further therapeutic agent is one or more members selected from the group consisting of everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR, KRX-0402, lucanthone, LY 317615, neuradiab, vitespan, Rta 744, Sdx 102, talampanel, atrasentan, Xr 311, romidepsin, 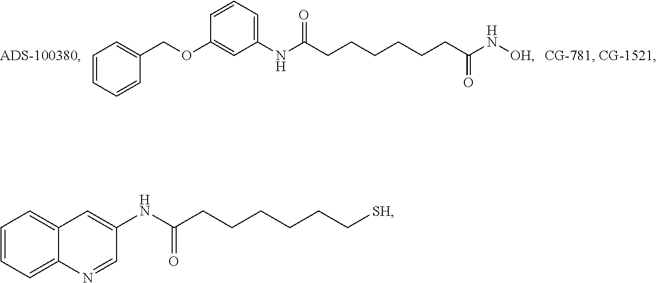
SB-556629, chlamydocin, JNJ-16241199, 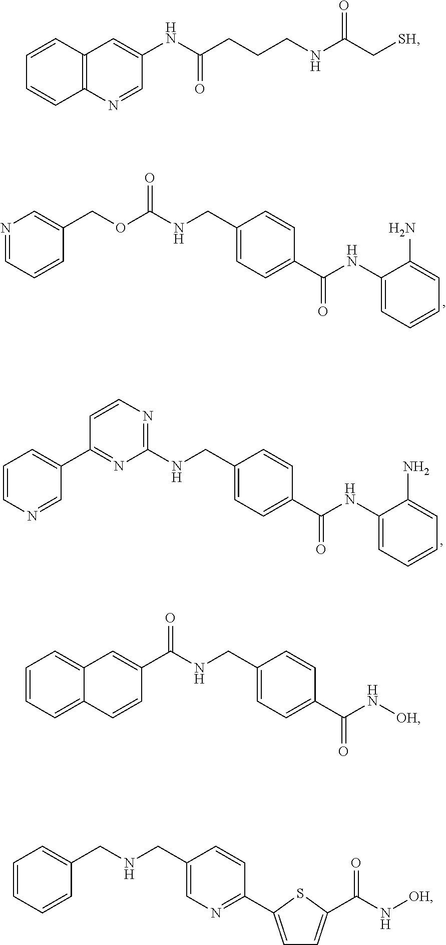
vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrim idin-5-yl)ethyl]benzoyl]-, disodium salt, heptahydrate, camptothecin, irinotecan; a combination of irinotecan, 5-fluorouracil and leucovorin; PEG-labeled irinotecan, FOLFOX regimen, tamoxifen, toremifene citrate, anastrazole, exemestane, letrozole, DES (diethylstilbestrol), estradiol, estrogen, conjugated estrogen, bevacizumab, IMC-1C11, CHIR-258, 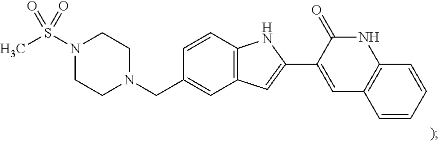
3-[5-(methylsulfonylpiperadinemethyl)-indolyl]-quinolone, vatalanib, AG-013736, AVE-0005, the acetate salt of [D-Ser(But) 6, Azgly 10] (pyro-Glu-His-Trp-Ser-Tyr-D-Ser(But)-Leu-Arg-Pro-Azgly-NH₂ acetate [C₅₉H₈₄N₁₈O₁₄.(C₂H₄O₂), where x=1 to 2.4], goserelin acetate, leuprolide acetate, triptorelin pamoate, sunitinib, sunitinib malate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, lonafarnib, 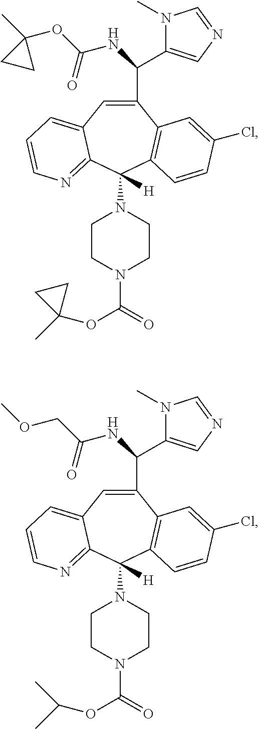
BMS-214662, tipifarnib; amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyclophosphamide, cyproterone, cytarabine, dacarbazine, dactinomycin, daunorubicin, diethylstilbestrol, epirubicin, fludarabine, fludrocortisone, fluoxymesterone, flutamide, hydroxyurea, idarubicin, ifosfamide, imatinib, leucovorin, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mercaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimetidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, 5-fluorouracil, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa and darbepoetin alfa.

27. The method of claim 20 wherein the malignant or neoplastic cells are in a tumor or mediate a cancerous condition selected from the group consisting of osteosarcoma, rhabdomyosarcoma, neuroblastoma, any pediatric cancer, kidney cancer, leukemia, renal transitional cell cancer, Werner-Morrison syndrome, bladder cancer, Wilm's cancer, ovarian cancer, pancreatic cancer, breast cancer, prostate cancer, benign prostatic hyperplasia, bone cancer, lung cancer, gastric cancer, colorectal cancer, cervical cancer, synovial sarcoma, vasoactive intestinal peptide secreting tumors, tumor angiogenesis, head and neck cancer, squamous cell carcinoma, multiple myeloma, solitary plasmacytoma, renal cell cancer, retinoblastoma, germ cell tumor, hepatoblastoma, hepatocellular carcinoma, melanoma, rhabdoid tumor of the kidney, Ewing Sarcoma, chondrosarcoma, haemotological malignancy, chronic lymphoblastic leukemia, chronic myelomonocytic leukemia, acute lymphoblastic leukemia, acute lymphocytic leukemia, acute myelogenous leukemia, acute myeloblastic leukemia, chronic myeloblastic leukemia, Hodgekin's disease, non-Hodgekin's lymphoma, chronic lymphocytic leukemia, chronic myelogenous leukemia, myelodysplastic syndrome, hairy cell leukemia, mast cell leukemia, mast cell neoplasm, follicular lymphoma, diffuse large cell lymphoma, mantle cell lymphoma, Burkitt Lymphoma, mycosis fungoides, seary syndrome, cutaneous T-cell lymphoma, chronic myeloproliferative disorders, a central nervous system tumor, brain cancer, glioblastoma, non-glioblastoma brain cancer, meningioma, pituitary adenoma, vestibular schwannoma, a primitive neuroectodermal tumor, medulloblastoma, astrocytoma, anaplastic astrocytoma, oligodendroglioma, ependymoma, choroid plexus papilloma, a myeloproliferative disorder, polycythemia vera, thrombocythemia, idiopathic myelfibrosis, soft tissue sarcoma, thyroid cancer, endometrial cancer, carcinoid cancer and liver cancer.

28. A method for identifying a mammalian subject with malignant or neoplastic cells sensitive to an IGF1R inhibitor comprising evaluating sensitivity of the malignant or neoplastic cells to said inhibitor by the method of claim 1; wherein said subject is identified if said cells are determined to be sensitive.

29. A method for identifying a mammalian subject with malignant or neoplastic cells sensitive to an IGF1R inhibitor comprising evaluating sensitivity of the malignant or neoplastic cells to said inhibitor by the method of claim 2; wherein said subject is identified if said cells are determined to be sensitive.

30. A method for identifying a mammalian subject with malignant or neoplastic cells sensitive to an IGF1R inhibitor comprising evaluating sensitivity of the malignant or neoplastic cells to said inhibitor by the method of claim 3 wherein said subject is identified if said cells are determined to be sensitive.

31. The method of claim 28 wherein said inhibitor is a member selected from the group consisting of an antibody or antigen-binding fragment thereof which binds specifically to IGF1R,

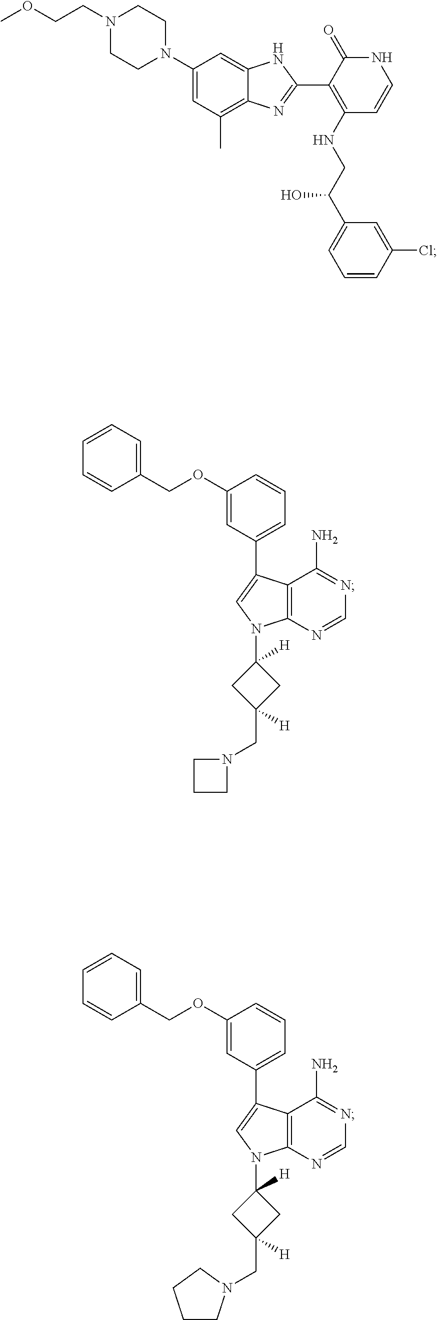

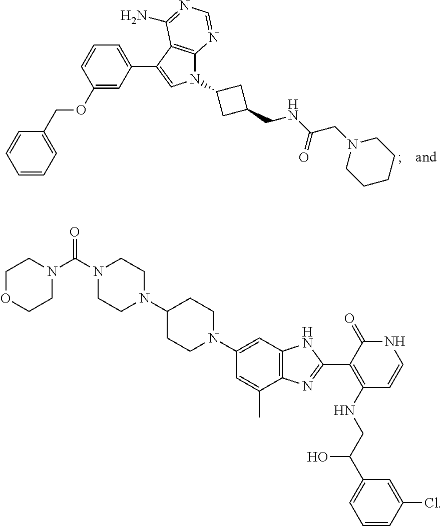

32. The method of claim 31 wherein the inhibitor is an antibody or fragment which comprises one or more complementarity determining regions (CDRs) selected from the group consisting of:
RASQSIGSSLH (SEQ ID NO: 99),
YASQSLS (SEQ ID NO: 100),
HQSSRLPHT (SEQ ID NO: 101),
SFAMH (SEQ ID NO:102),
GFTFSSFAMH (SEC) ID NO: 107),
VIDTRGATYYADSVKG (SEQ ID NO: 103), and
LGNFYYGMDV (SEQ ID NO: 104); or wherein the antibody or fragment comprises a mature fragment of a light chain immunoglobulin which comprises the amino acid sequence of SEQ ID NO: 2, 4, 6 or 8; and/or wherein the antibody or fragment comprises a mature fragment of a heavy chain immunoglobulin which comprises the amino acid sequence of SEQ ID NO: 10 or 12.

33. The method of claim 28 wherein, after the subject is identified, the subject is administered a therapeutically effective dose of an IGF1R inhibitor optionally in association with a further therapeutic agent.

34. The method of claim 33 wherein the further therapeutic agent is one or more members selected from the group consisting of everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, ID 3-PE38QQR, INO 1001, IPdR, KRX-0402, lucanthone, LY 317615, neuradiab, vitespan, Rta 744, Sdx 102, talampanel, atrasentan, Xr 311, romidepsin, 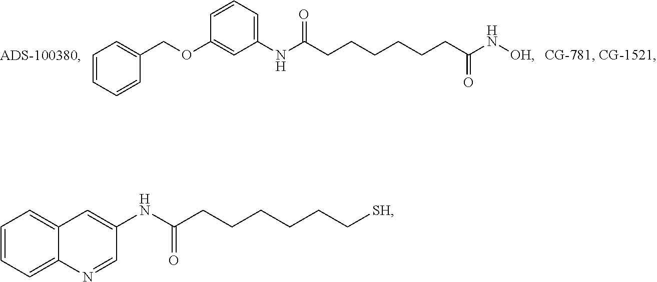
SB-556629, chlamydocin, JNJ-16241199, 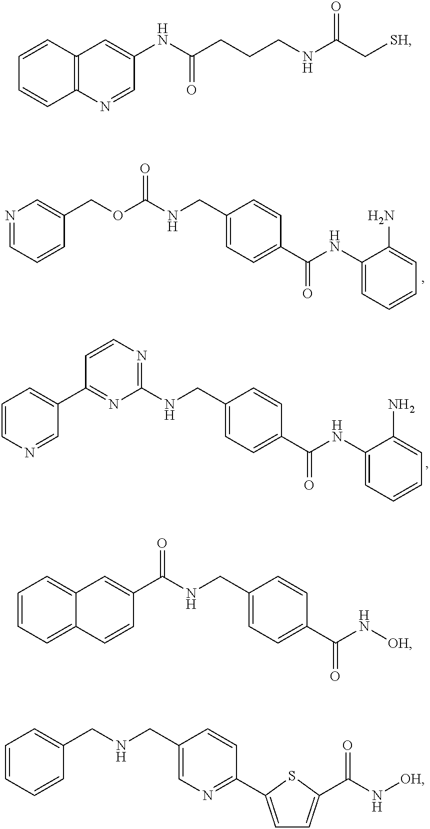
vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, irinotecan; a combination of irinotecan, 5-fluorouracil and leucovorin; PEG-labeled irinotecan, FOLFOX regimen, tamoxifen, toremifene citrate, anastrazole, exemestane, letrozole, DES (diethylstilbestrol), estradiol, estrogen, conjugated estrogen, bevacizumab, IMC-1C11, CHIR-258, 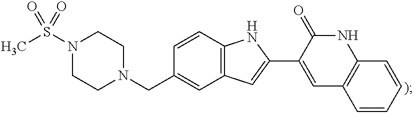
3-[5-(methylsulfonylpiperadinemethyl)-indolyl]-quinolone, vatalanib, AG-013736, AVE-0005, the acetate salt of [D-Ser(Bu t) 6, Azgly 10] (pyro-Glu-His-Trp-Ser-Tyr-D-Ser(Bu t)-Leu-Arg-Pro-Azgly-NH₂ acetate [C₅₉H₈₄N₁₈O₁₄.(C₂H₄O₂)ₓ where x=1 to 2.4], goserelin acetate, leuprolide acetate, triptorelin pamoate, sunitinib, sunitinib malate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, lonafarnib, 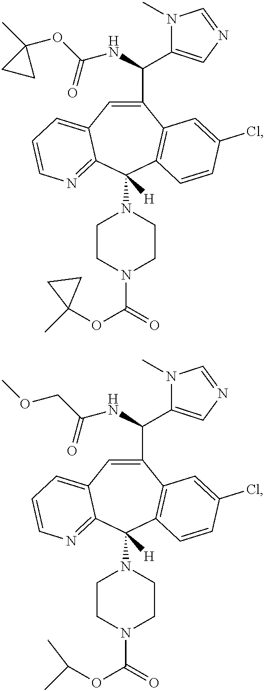
BMS-214662, tipifarnib; amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyclophosphamide, cyproterone, cytarabine, dacarbazine, dactinomycin, daunorubicin, diethylstilbestrol, epirubicin, fludarabine, fludrocortisone, fluoxymesterone, flutamide, hydroxyurea, idarubicin, ifosfamide, imatinib, leucovorin, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mercaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, 5-fluorouracil, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa and darbepoetin alfa.

35. The method of claim 28 wherein the malignant or neoplastic cells are in a tumor or mediate a cancerous condition, in a subject, selected from the group consisting of osteosarcoma, rhabdomyosarcoma, neuroblastoma, any pediatric cancer, kidney cancer, leukemia, renal transitional cell cancer, Werner-Morrison syndrome, bladder cancer, Wilm's cancer, ovarian cancer, pancreatic cancer, breast cancer, prostate cancer, benign prostatic hyperplasia, bone cancer, lung cancer, gastric cancer, colorectal cancer, cervical cancer, synovial sarcoma, vasoactive intestinal peptide secreting tumors, tumor angiogenesis, head and neck cancer, squamous cell carcinoma, multiple myeloma, solitary plasmacytoma, renal cell cancer, retinoblastoma, germ cell tumor, hepatoblastoma, hepatocellular carcinoma, melanoma, rhabdoid tumor of the kidney, Ewing Sarcoma, chondrosarcoma, haemotological malignancy, chronic lymphoblastic leukemia, chronic myelomonocytic leukemia, acute lymphoblastic leukemia, acute lymphocytic leukemia, acute myelogenous leukemia, acute myeloblastic leukemia, chronic myeloblastic leukemia, Hodgekin's disease, non-Hodgekin's lymphoma, chronic lymphocytic leukemia, chronic myelogenous leukemia, myelodysplastic syndrome, hairy cell leukemia, mast cell leukemia, mast cell neoplasm, follicular lymphoma, diffuse large cell lymphoma, mantle cell lymphoma, Burkitt Lymphoma, mycosis fungoides, seary syndrome, cutaneous T-cell lymphoma, chronic myeloproliferative disorders, a central nervous system tumor, brain cancer, glioblastoma, non-glioblastoma brain cancer, meningioma, pituitary adenoma, vestibular schwannoma, a primitive neuroectodermal tumor, medulloblastoma, astrocytoma, anaplastic astrocytoma, oligodendroglioma, ependymoma, choroid plexus papilloma, a myeloproliferative disorder, polycythemia vera, thrombocythemia, idiopathic myelfibrosis, soft tissue sarcoma, thyroid cancer, endometrial cancer, carcinoid cancer and liver cancer.

36. A method for treating a tumor or cancerous condition, in a mammalian subject, with an IGF1R inhibitor comprising evaluating sensitivity of malignant or neoplastic cells, which are in said tumor or which mediate said cancerous condition, to said inhibitor by the method of claim 1 and, if said cells are determined to be sensitive, continuing or commencing treatment by administering, to the subject, a therapeutically effective dose of the inhibitor.

37. A method for treating a tumor or cancerous condition, in a mammalian subject, with an IGF1R inhibitor comprising evaluating sensitivity of malignant or neoplastic cells, which are in said tumor or which mediate said cancerous condition, to said inhibitor by the method of claim 2 and, if said cells are determined to be sensitive, continuing or commencing treatment by administering, to the subject, a therapeutically effective dose of the inhibitor.

38. A method for treating a tumor or cancerous condition, in a mammalian subject, with an IGF1R inhibitor comprising evaluating sensitivity of malignant or neoplastic cells, which are in said tumor or which mediate said cancerous condition, to said inhibitor by the method of claim 3 and, if said cells are determined to be sensitive, continuing or commencing treatment by administering, to the subject, a therapeutically effective dose of the inhibitor.

39. The method of claim 36 wherein said inhibitor is a member selected from the group consisting of an antibody or antigen-binding fragment thereof which binds specifically to IGF1R;

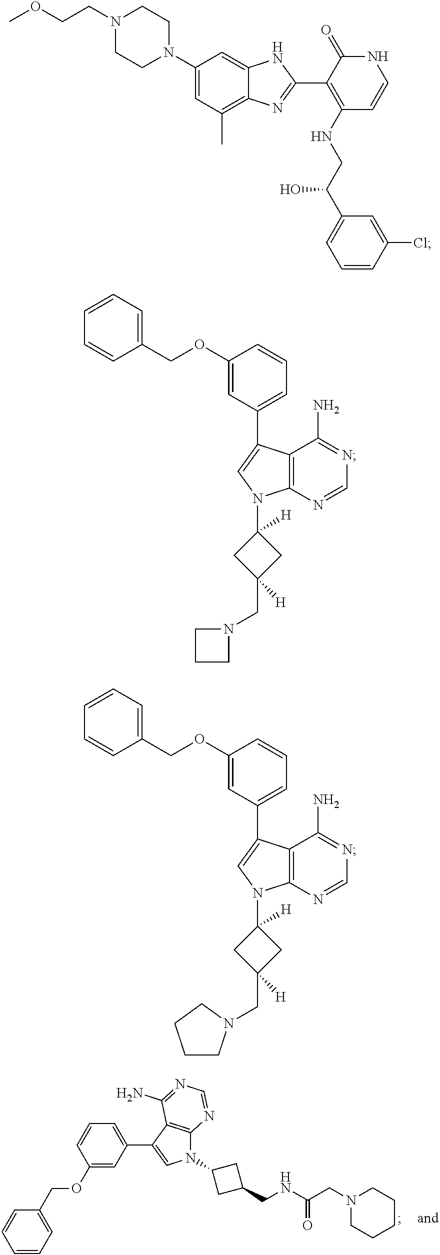

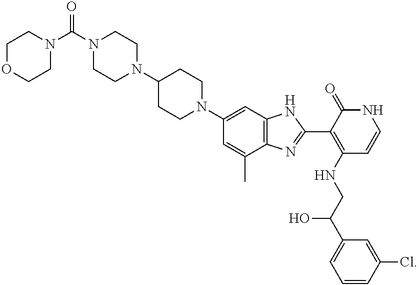

40. The method of claim 39 wherein the inhibitor is an antibody or fragment which comprises one or more complementarity determining regions (CDRs) selected from the group consisting of:
RASQSIGSSLH (SEQ ID NO: 99),
YASQSLS (SEQ ID NO: 100),
HQSSRLPHT (SEQ ID NO: 101),
SFAMH (SEQ ID NO:102),
GFTFSSFAMH (SEQ ID NO: 107),
VIDTRGATYYADSVKG (SEQ ID NO: 103), and
LGNFYYGMDV (SEQ ID NO: 104); or wherein the antibody or fragment comprises a mature fragment of a light chain immunoglobulin which comprises the amino acid sequence of SEQ ID NO: 2, 4, 6 or 8; and/or wherein the antibody or fragment comprises a mature fragment of a heavy chain immunoglobulin which comprises the amino acid sequence of SEQ ID NO: 10 or 12.

41. The method of claim 36 wherein the IGF1R inhibitor is administered in association with a further therapeutic agent.

42. The method of claim 41 wherein the further therapeutic agent is one or more members selected from the group consisting of everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR, KRX-0402, lucanthone, LY 317615, neuradiab, vitespan, Rta 744, Sdx 102, talampanel, atrasentan, Xr 311, romidepsin, 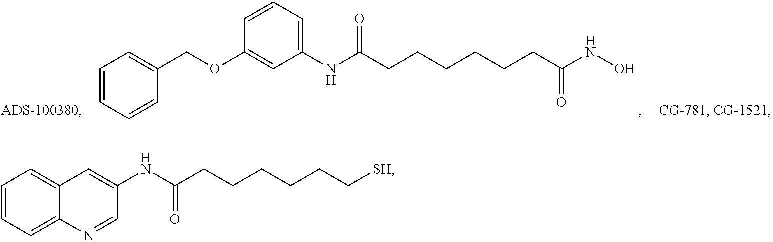
SB-556629, chlamydocin, JNJ-16241199, 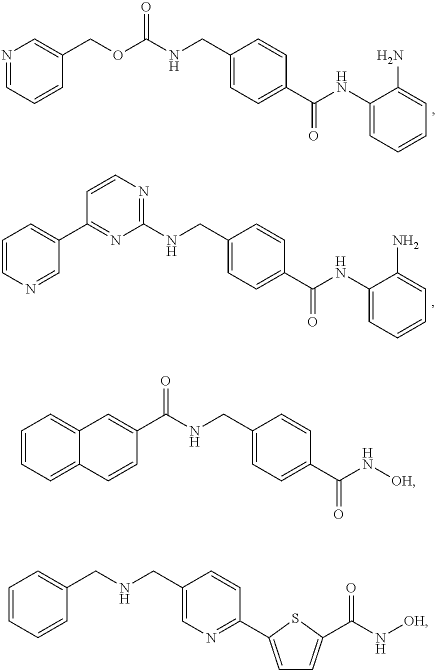
vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, irinotecan; a combination of irinotecan, 5-fluorouracil and leucovorin; PEG-labeled irinotecan, FOLFOX regimen, tamoxifen, toremifene citrate, anastrazole, exemestane, letrozole, DES (diethylstilbestrol), estradiol, estrogen, conjugated estrogen, bevacizumab, IMC-1C11, CHIR-258, 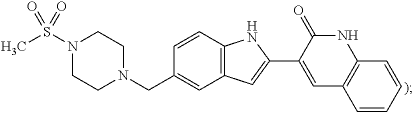
3-[5-(methylsulfonylpiperadinemethyl)-indolyl]-quinolone, vatalanib, AG-013736, AVE-0005, the acetate salt of [D-Ser(Bu t) 6, Azgly 10] (pyro-Glu-His-Trp-Ser-Tyr-D-Ser(Bu t)-Leu-Arg-Pro-Azgly-NH₂ acetate [C₅₉H₈₄N₁₈O₁₄.(C₂H₄O₂)ₓ where x=1 to 2.4], goserelin acetate, leuprolide acetate, triptorelin pamoate, sunitinib, sunitinib malate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, lonafarnib, 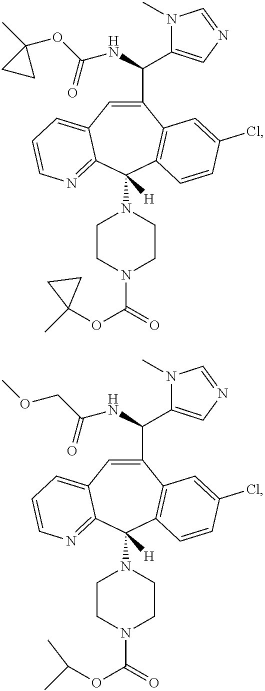
BMS-214662, tipifarnib; amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyclophosphamide, cyproterone, cytarabine, dacarbazine, dactinomycin, daunorubicin, diethylstilbestrol, epirubicin, fludarabine, fludrocortisone, fluoxymesterone, flutamide, hydroxyurea, idarubicin, ifosfamide, imatinib, leucovorin, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mercaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, 5-fluorouracil, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa and darbepoetin alfa.

43. The method of claim 36 wherein the tumor or cancerous condition is selected from the group consisting of osteosarcoma, rhabdomyosarcoma, neuroblastoma, any pediatric cancer, kidney cancer, leukemia, renal transitional cell cancer, Werner-Morrison syndrome, bladder cancer, Wilm's cancer, ovarian cancer, pancreatic cancer, breast cancer, prostate cancer, benign prostatic hyperplasia, bone cancer, lung cancer, gastric cancer, colorectal cancer, cervical cancer, synovial sarcoma, vasoactive intestinal peptide secreting tumors, tumor angiogenesis, head and neck cancer, squamous cell carcinoma, multiple myeloma, solitary plasmacytoma, renal cell cancer, retinoblastoma, germ cell tumor, hepatoblastoma, hepatocellular carcinoma, melanoma, rhabdoid tumor of the kidney, Ewing Sarcoma, chondrosarcoma, haemotological malignancy, chronic lymphoblastic leukemia, chronic myelomonocytic leukemia, acute lymphoblastic leukemia, acute lymphocytic leukemia, acute myelogenous leukemia, acute myeloblastic leukemia, chronic myeloblastic leukemia, Hodgekin's disease, non-Hodgekin's lymphoma, chronic lymphocytic leukemia, chronic myelogenous leukemia, myelodysplastic syndrome, hairy cell leukemia, mast cell leukemia, mast cell neoplasm, follicular lymphoma, diffuse large cell lymphoma, mantle cell lymphoma, Burkitt Lymphoma, mycosis fungoides, seary syndrome, cutaneous T-cell lymphoma, chronic myeloproliferative disorders, a central nervous system tumor, brain cancer, glioblastoma, non-glioblastoma brain cancer, meningioma, pituitary adenoma, vestibular schwannoma, a primitive neuroectodermal tumor, medulloblastoma, astrocytoma, anaplastic astrocytoma, oligodendroglioma, ependymoma, choroid plexus papilloma, a myeloproliferative disorder, polycythemia vera, thrombocythemia, idiopathic myelfibrosis, soft tissue sarcoma, thyroid cancer, endometrial cancer, carcinoid cancer and liver cancer.

44. A method for selecting a therapy for a mammalian subject with malignant or neoplastic cells comprising evaluating sensitivity of the cells to an IGF1R inhibitor by the method of claim 1; wherein said inhibitor is selected as the therapy if said cells are determined to be sensitive to the inhibitor.

45. A method for selecting a therapy for a mammalian subject with one or more malignant or neoplastic cells comprising evaluating sensitivity of the cells to an IGF1R inhibitor by the method of claim 2; wherein said inhibitor is selected as the therapy if said cells are determined to be sensitive to the inhibitor.

46. A method for selecting a therapy for a mammalian subject with one or more malignant or neoplastic cells comprising evaluating sensitivity of the cells to an IGF1R inhibitor by the method of claim 3; wherein said inhibitor is selected as the therapy if said cells are determined to be sensitive to the inhibitor.

47. The method of claim 44 wherein said inhibitor is a member selected from the group consisting of an antibody or antigen-binding fragment thereof which binds specifically to IGF1R;

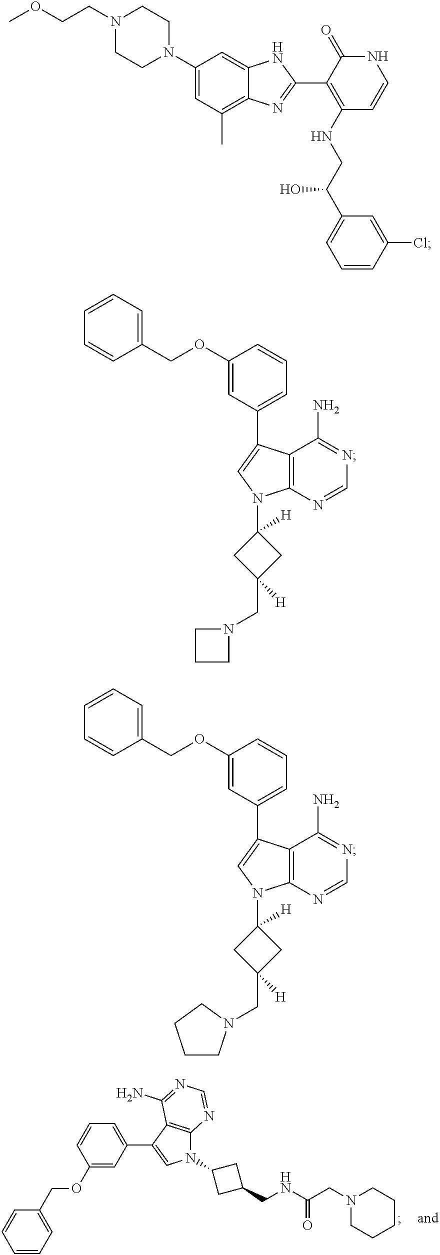

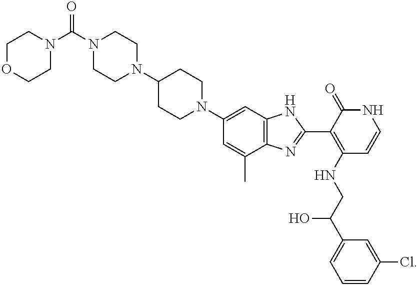

48. The method of claim 47 wherein the inhibitor is an antibody or fragment which comprises one or more complementarity determining regions (CDRs) selected from the group consisting of:
RASQSIGSSLH (SEQ ID NO: 99),
YASQSLS (SEQ ID NO: 100),
HQSSRLPHT (SEQ ID NO: 101),
SFAMH (SEQ ID NO:102),
GFTFSSFAMH (SEQ ID NO: 107),
VIDTRGATYYADSVKG (SEQ ID NO: 103), and
LGNFYYGMDV (SEQ ID NO: 104); or wherein the antibody or fragment comprises a mature fragment of a light chain immunoglobulin which comprises the amino acid sequence of SEQ ID NO: 2, 4, 6 or 8; and/or wherein the antibody or fragment comprises a mature fragment of a heavy chain immunoglobulin which comprises the amino acid sequence of SEQ ID NO: 10 or 12.

49. The method of claim 44 wherein the malignant or neoplastic cells are in a tumor or mediate a cancerous condition selected from the group consisting of osteosarcoma, rhabdomyosarcoma, neuroblastoma, any pediatric cancer, kidney cancer, leukemia, renal transitional cell cancer, Werner-Morrison syndrome, bladder cancer, Wilm's cancer, ovarian cancer, pancreatic cancer, breast cancer, prostate cancer, benign prostatic hyperplasia, bone cancer, lung cancer, gastric cancer, colorectal cancer, cervical cancer, synovial sarcoma, vasoactive intestinal peptide secreting tumors, tumor angiogenesis, head and neck cancer, squamous cell carcinoma, multiple myeloma, solitary plasmacytoma, renal cell cancer, retinoblastoma, germ cell tumor, hepatoblastoma, hepatocellular carcinoma, melanoma, rhabdoid tumor of the kidney, Ewing Sarcoma, chondrosarcoma, haemotological malignancy, chronic lymphoblastic leukemia, chronic myelomonocytic leukemia, acute lymphoblastic leukemia, acute lymphocytic leukemia, acute myelogenous leukemia, acute myeloblastic leukemia, chronic myeloblastic leukemia, Hodgekin's disease, non-Hodgekin's lymphoma, chronic lymphocytic leukemia, chronic myelogenous leukemia, myelodysplastic syndrome, hairy cell leukemia, mast cell leukemia, mast cell neoplasm, follicular lymphoma, diffuse large cell lymphoma, mantle cell lymphoma, Burkitt Lymphoma, mycosis fungoides, seary syndrome, cutaneous T-cell lymphoma, chronic myeloproliferative disorders, a central nervous system tumor, brain cancer, glioblastoma, non-glioblastoma brain cancer, meningioma, pituitary adenoma, vestibular schwannoma, a primitive neuroectodermal tumor, medulloblastoma, astrocytoma, anaplastic astrocytoma, oligodendroglioma, ependymoma, choroid plexus papilloma, a myeloproliferative disorder, polycythemia vera, thrombocythemia, idiopathic myelfibrosis, soft tissue sarcoma, thyroid cancer, endometrial cancer, carcinoid cancer and liver cancer.

50. The method of claim 44 wherein, after the inhibitor is selected as the therapy, the subject is administered a therapeutically effective dose of the inhibitor optionally in association with a further therapeutic agent.

51. The method of claim 50 wherein the further therapeutic agent is one or more members selected from the group consisting of everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a BcI-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR, KRX-0402, lucanthone, LY 317615, neuradiab, vitespan, Rta 744, Sdx 102, talampanel, atrasentan, Xr 311, romidepsin, 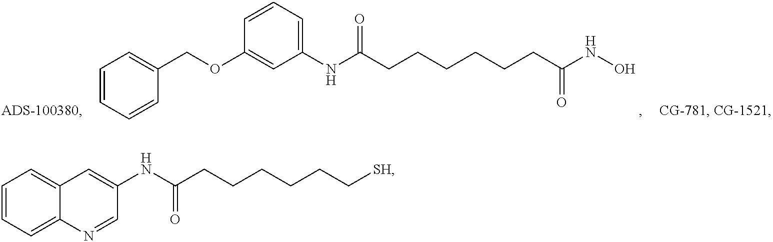
SB-556629, chlamydocin, JNJ-16241199, 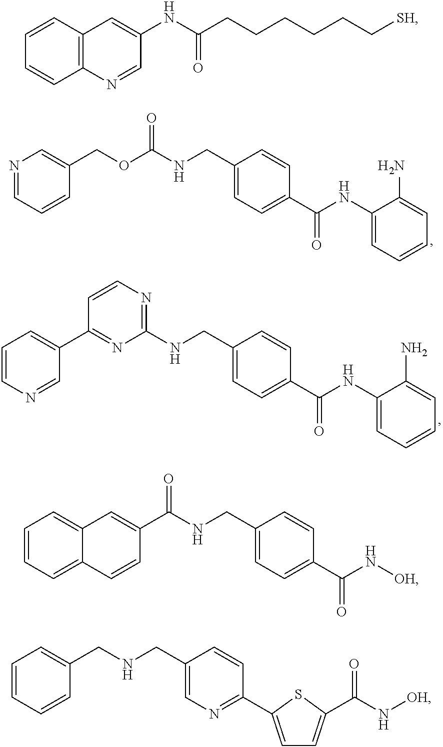
vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, irinotecan; a combination of irinotecan, 5-fluorouracil and leucovorin; PEG-labeled irinotecan, FOLFOX regimen, tamoxifen, toremifene citrate, anastrazole, exemestane, letrozole, DES (diethylstilbestrol), estradiol, estrogen, conjugated estrogen, bevacizumab, IMC-1C11, CHIR-258, 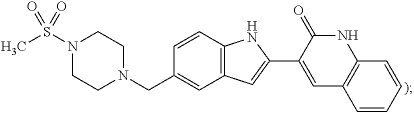
3-[5-(methylsulfonylpiperadinemethyl)-indolyl]-quinolone, vatalanib, AG-013736, AVE-0005, the acetate salt of [D-Ser(Bu t) 6, Azgly 10] (pyro-Glu-His-Trp-Ser-Tyr-D-Ser(Bu t)-Leu-Arg-Pro-Azgly-NH₂ acetate [C₅₉H₈₄N₁₈O₁₄.(C₂H₄O₂)ₓ where x=1 to 2.4], goserelin acetate, leuprolide acetate, triptorelin pamoate, sunitinib, sunitinib malate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, lonafarnib, 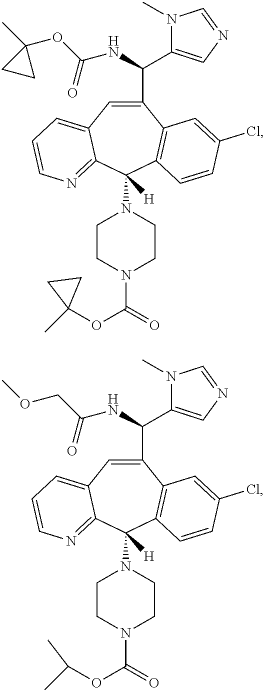
BMS-214662, tipifarnib; amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyclophosphamide, cyproterone, cytarabine, dacarbazine, dactinomycin, daunorubicin, diethylstilbestrol, epirubicin, fludarabine, fludrocortisone, fluoxymesterone, flutamide, hydroxyurea, idarubicin, ifosfamide, imatinib, leucovorin, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mercaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, 5-fluorouracil, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa and darbepoetin alfa.

52. A method of advertising an IGF1R inhibitor or a pharmaceutical composition thereof or a therapeutic regimen comprising administration of said inhibitor or composition comprising promoting, to a target audience, the use of the inhibitor or composition for treating a patient or patient population whose tumors or cancerous conditions are mediated by malignant or neoplastic cells that exhibit high expression of one or more genes selected from the group consisting of:
TRE2; SMC4; TRIB2; TLE4; BMP7; PCDHGC3; AUTS2; C14orf132; CERK; HDGFRP3; TCF4; MEIS2; EML4; C7orf41; KIAA1450; ZNF136; D15F37; CDK6; TIMP; Clu; and PRL1; relative to cells resistant to said inhibitor; and/or that exhibit low expression of one or more genes selected from the group consisting of:
ACAT1; ALDOC; C6orf192; COL4A5; C1QBP; CRIP1; DEADC1; GSTK1; GSTO2; PPAPDC1B; TMEM107; JOSD3; TMEM77; MST1; MT1E; PARVB; PRDX4; RASGEF1A; RPL14; IFI30; ATF1; ACADVL; FBXO6; NQO2; TMEM64; ZFAND1; TMED5; PDIA5; MYO1C; GNPTAB; LACTB2; RPL22; TSPAN4; RPL15; PCCB; CRYZ; DNAJC10; C19orf54; HSPE1; and hqp0376; relative to cells resistant to said inhibitor.

53. The method of claim 52 wherein said inhibitor is a member selected from the group consisting of an antibody or antigen-binding fragment thereof which binds specifically to IGF1R;

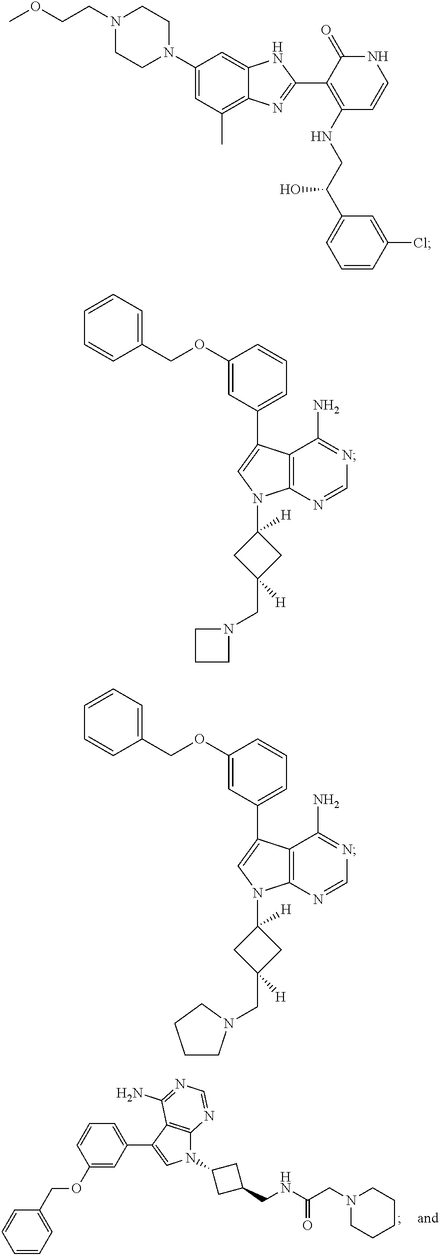

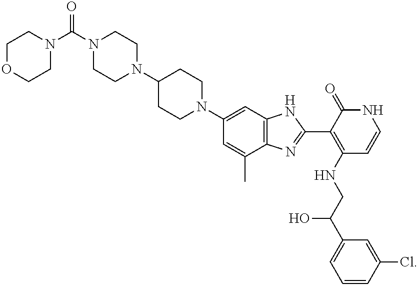

54. The method of claim 53 wherein the inhibitor is an antibody or fragment which comprises one or more complementarity determining regions (CDRs) selected from the group consisting of:
RASQSIGSSLH (SEQ ID NO: 99),
YASQSLS (SEQ ID NO: 100),
HQSSRLPHT (SEQ ID NO: 101),
SFAMH (SEQ ID NO:102),
GFTFSSFAMH (SEQ ID NO: 107),
VIDTRGATYYADSVKG (SEQ ID NO: 103), and
LGNFYYGMDV (SEQ ID NO: 104); or wherein the antibody or fragment comprises a mature fragment of a light chain immunoglobulin which comprises the amino acid sequence of SEQ ID NO: 2, 4, 6 or 8; and/or wherein the antibody or fragment comprises a mature fragment of a heavy chain immunoglobulin which comprises the amino acid sequence of SEQ ID NO: 10 or 12.

55. The method of claim 52 wherein the tumor or cancerous condition is selected from the group consisting of osteosarcoma, rhabdomyosarcoma, neuroblastoma, any pediatric cancer, kidney cancer, leukemia, renal transitional cell cancer, Werner-Morrison syndrome, bladder cancer, Wilm's cancer, ovarian cancer, pancreatic cancer, breast cancer, prostate cancer, benign prostatic hyperplasia, bone cancer, lung cancer, gastric cancer, colorectal cancer, cervical cancer, synovial sarcoma, vasoactive intestinal peptide secreting tumors, tumor angiogenesis, head and neck cancer, squamous cell carcinoma, multiple myeloma, solitary plasmacytoma, renal cell cancer, retinoblastoma, germ cell tumor, hepatoblastoma, hepatocellular carcinoma, melanoma, rhabdoid tumor of the kidney, Ewing Sarcoma, chondrosarcoma, haematological malignancy, chronic lymphoblastic leukemia, chronic myelomonocytic leukemia, acute lymphoblastic leukemia, acute lymphocytic leukemia, acute myelogenous leukemia, acute myeloblastic leukemia, chronic myeloblastic leukemia, Hodgekin's disease, non-Hodgekin's lymphoma, chronic lymphocytic leukemia, chronic myelogenous leukemia, myelodysplastic syndrome, hairy cell leukemia, mast cell leukemia, mast cell neoplasm, follicular lymphoma, diffuse large cell lymphoma, mantle cell lymphoma, Burkitt Lymphoma, mycosis fungoides, seary syndrome, cutaneous T-cell lymphoma, chronic myeloproliferative disorders, a central nervous system tumor, brain cancer, glioblastoma, non-glioblastoma brain cancer, meningioma, pituitary adenoma, vestibular schwannoma, a primitive neuroectodermal tumor, medulloblastoma, astrocytoma, anaplastic astrocytoma, oligodendroglioma, ependymoma, choroid plexus papilloma, a myeloproliferative disorder, polycythemia vera, thrombocythemia, idiopathic myelfibrosis, soft tissue sarcoma, thyroid cancer, endometrial cancer, carcinoid cancer and liver cancer.

56. An article of manufacture comprising, packaged together, an IGF1R inhibitor or a pharmaceutical composition thereof comprising a pharmaceutically acceptable carrier; and a label stating that the inhibitor or pharmaceutical composition is indicated for treating patients having a tumor comprising malignant or neoplastic cells or a cancerous condition mediated by malignant or neoplastic cells that exhibit high expression of one or more genes selected from the group consisting of:
TRE2; SMC4; TRIB2; TLE4; BMP7; PCDHGC3; AUTS2; C14orf132; CERK; HDGFRP3; TCF4; MEIS2; EML4; C7orf41; KIAA1450; ZNF136; D15F37; CDK6; TIMP; Clu; and PRL1; relative to cells resistant to said inhibitor; and/or that exhibit low expression of one or more genes selected from the group consisting of:
ACAT1; ALDOC; C6orf192; COL4A5; C1QBP; CRIP1; DEADC1; GSTK1; GSTO2; PPAPDC1B; TMEM107; JOSD3; TMEM77; MST1; MT1E;
PARVB; PRDX4; RASGEF1A; RPL14; IFI30; ATF1; ACADVL; FBXO6; NQO2; TMEM64; ZFAND1; TMED5; PDIA5; MYO1C; GNPTAB; LACTB2; RPL22; TSPAN4; RPL15; PCCB; CRYZ; DNAJC10; C19orf54; HSPE1; and hqp0376; relative to cells resistant to said inhibitor.

57. The article of manufacture of claim 56 wherein said inhibitor is a member selected from the group consisting of an antibody or antigen-binding fragment thereof which binds specifically to IGF1R;

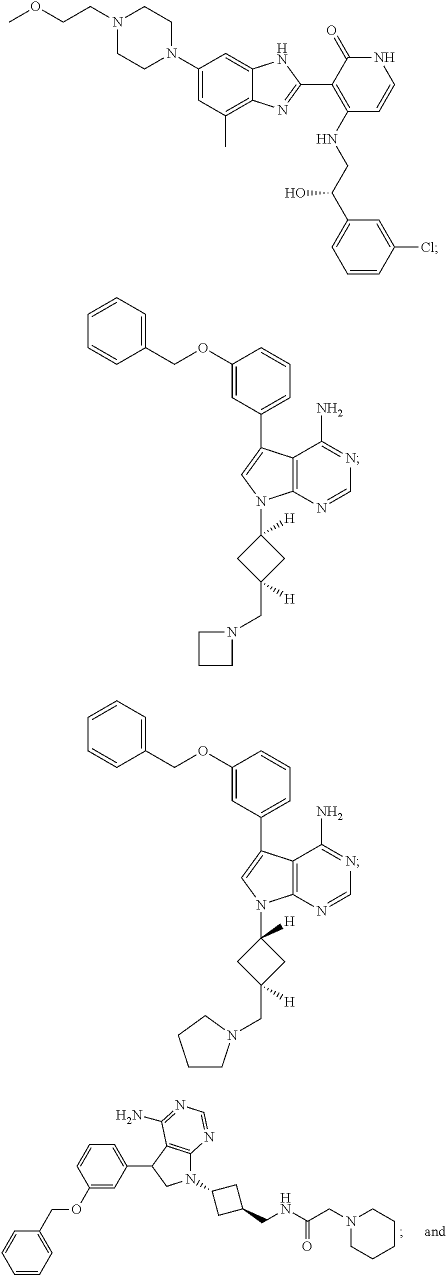

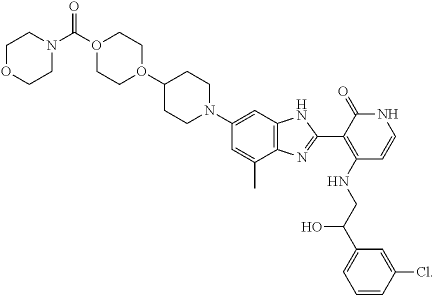

58. The article of manufacture of claim 57 wherein the inhibitor is an antibody or fragment which comprises one or more complementarity determining regions (CDRs) selected from the group consisting of:
RASQSIGSSLH (SEQ ID NO: 99),
YASQSLS (SEQ ID NO: 100),
HQSSRLPHT (SEQ ID NO: 101),
SFAMH (SEQ ID NO:102),
GFTFSSFAMH (SEQ ID NO: 107),
VIDTRGATYYADSVKG (SEQ ID NO: 103), and
LGNFYYGMDV (SEQ ID NO: 104); or wherein the antibody or fragment comprises a mature fragment of a light chain immunoglobulin which comprises the amino acid sequence of SEQ ID NO: 2, 4, 6 or 8; and/or wherein the antibody or fragment comprises a mature fragment of a heavy chain immunoglobulin which comprises the amino acid sequence of SEQ ID NO: 10 or 12.

59. The article of manufacture of claim 56 wherein the tumor or cancerous condition is selected from the group consisting of osteosarcoma, rhabdomyosarcoma, neuroblastoma, any pediatric cancer, kidney cancer, leukemia, renal transitional cell cancer, Werner-Morrison syndrome, bladder cancer, Wilm's cancer, ovarian cancer, pancreatic cancer, breast cancer, prostate cancer, benign prostatic hyperplasia, bone cancer, lung cancer, gastric cancer, colorectal cancer, cervical cancer, synovial sarcoma, vasoactive intestinal peptide secreting tumors, tumor angiogenesis, head and neck cancer, squamous cell carcinoma, multiple myeloma, solitary plasmacytoma, renal cell cancer, retinoblastoma, hepatoblastoma, hepatocellular carcinoma, melanoma, rhabdoid tumor of the kidney, Ewing Sarcoma, chondrosarcoma, haemotological malignancy, chronic lymphoblastic leukemia, chronic myelomonocytic leukemia, acute lymphoblastic leukemia, acute lymphocytic leukemia, acute myelogenous leukemia, acute myeloblastic leukemia, chronic myeloblastic leukemia, Hodgekin's disease, non-Hodgekin's lymphoma, chronic lymphocytic leukemia, chronic myelogenous leukemia, myelodysplastic syndrome, hairy cell leukemia, mast cell leukemia, mast cell neoplasm, follicular lymphoma, diffuse large cell lymphoma, mantle cell lymphoma, Burkitt Lymphoma, mycosis fungoides, seary syndrome, cutaneous T-cell lymphoma, chronic myeloproliferative disorders, a central nervous system tumor, brain cancer, glioblastoma, non-glioblastoma brain cancer, meningioma, pituitary adenoma, vestibular schwannoma, a primitive neuroectodermal tumor, medulloblastoma, astrocytoma, anaplastic astrocytoma, oligodendroglioma, ependymoma, choroid plexus papilloma, a myeloproliferative disorder, polycythemia vera, thrombocythemia, idiopathic myelfibrosis, soft tissue sarcoma, thyroid cancer, endometrial cancer, carcinoid cancer, germ cell tumor and liver cancer.

60. A method for manufacturing an IGF1R inhibitor or a pharmaceutical composition thereof comprising a pharmaceutically acceptable carrier said method comprising combining, in a package, the inhibitor or composition; and a label conveying that the inhibitor or composition is indicated for treating patients having a tumor comprising malignant or neoplastic cells or a cancerous condition mediated by malignant or neoplastic cells that exhibit high expression of one or more genes selected from the group consisting of:
TRE2; SMC4; TRIB2; TLE4; BMP7; PCDHGC3; AUTS2; C14orf132; CERK; HDGFRP3; TCF4; MEIS2; EML4; C7orf41; KIAA1450; ZNF136; D15F37; CDK6; TIMP; Clu; and PRL1; relative to cells resistant to said inhibitor; and/or that exhibit low expression of one or more genes selected from the group consisting of:
ACAT1; ALDOC; C6orf192; COL4A5; C1QBP; CRIP1; DEADC1; GSTK1; GSTO2; PPAPDC1B; TMEM107; JOSD3; TMEM77; MST1; MT1E;
PARVB; PRDX4; RASGEF1A; RPL14; IFI30; ATF1; ACADVL; FBXO6; NQO2; TMEM64; ZFAND1; TMED5; PDIA5; MYO1C; GNPTAB; LACTB2; RPL22; TSPAN4; RPL15; PCCB, CRYZ; DNAJC10; C19orf54; HSPE1; and hqp0376; relative to cells resistant to said inhibitor.

61. The method of claim 60 wherein said inhibitor is a member selected from the group consisting of an antibody or antigen-binding fragment thereof which binds specifically to IGF1R;

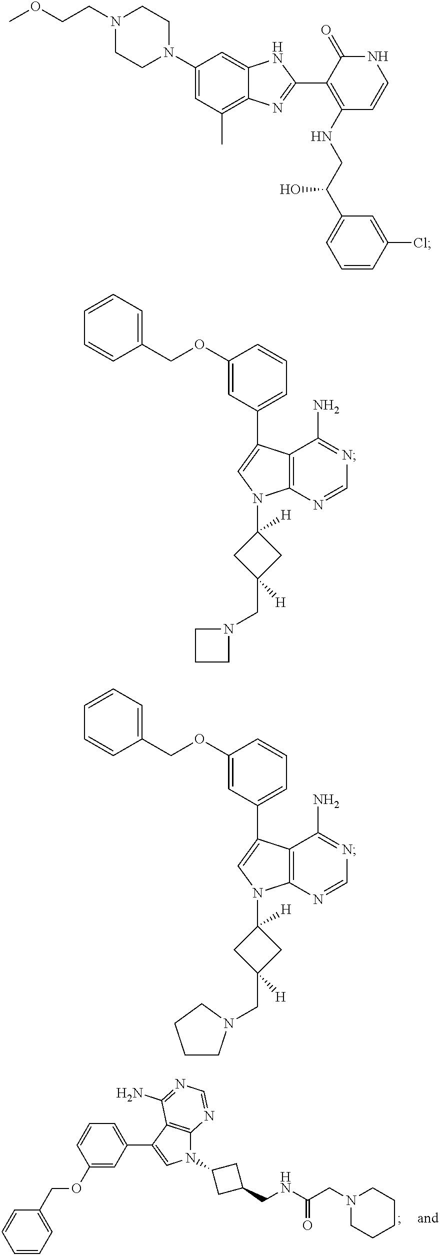

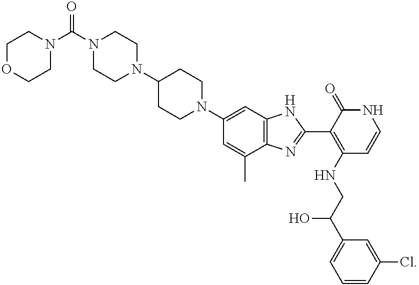

62. The method of claim 61 wherein the inhibitor is an antibody or fragment which comprises one or more complementarity determining regions (CDRs) selected from the group consisting of:
RASQSIGSSLH (SEQ ID NO: 99),
YASQSLS (SEQ ID NO: 100),
HQSSRLPHT (SEQ ID NO: 101),
SFAMH (SEQ ID NO:102),
GFTFSSFAMH (SEQ ID NO: 107),
VIDTRGATYYADSVKG (SEQ ID NO: 103), and
LGNFYYGMDV (SEQ ID NO: 104); or wherein the antibody or fragment comprises a mature fragment of a light chain immunoglobulin which comprises the amino acid sequence of SEQ ID NO: 2, 4, 6 or 8; and/or wherein the antibody or fragment comprises a mature fragment of a heavy chain immunoglobulin which comprises the amino acid sequence of SEQ ID NO: 10 or 12.

63. The method of claim 60 wherein the tumor or cancerous condition is selected from the group consisting of osteosarcoma, rhabdomyosarcoma, neuroblastoma, any pediatric cancer, kidney cancer, leukemia, renal transitional cell cancer, Werner-Morrison syndrome, bladder cancer, Wilm's cancer, ovarian cancer, pancreatic cancer, breast cancer, prostate cancer, benign prostatic hyperplasia, bone cancer, lung cancer, gastric cancer, colorectal cancer, cervical cancer, synovial sarcoma, vasoactive intestinal peptide secreting tumors, tumor angiogenesis, head and neck cancer, squamous cell carcinoma, multiple myeloma, solitary plasmacytoma, renal cell cancer, retinoblastoma, hepatoblastoma, hepatocellular carcinoma, melanoma, rhabdoid tumor of the kidney, Ewing Sarcoma, chondrosarcoma, haemotological malignancy, chronic lymphoblastic leukemia, chronic myelomonocytic leukemia, acute lymphoblastic leukemia, acute lymphocytic leukemia, acute myelogenous leukemia, acute myeloblastic leukemia, chronic myeloblastic leukemia, Hodgekin's disease, non-Hodgekin's lymphoma, chronic lymphocytic leukemia, chronic myelogenous leukemia, myelodysplastic syndrome, hairy cell leukemia, mast cell leukemia, mast cell neoplasm, follicular lymphoma, diffuse large cell lymphoma, mantle cell lymphoma, Burkitt Lymphoma, mycosis fungoides, seary syndrome, cutaneous T-cell lymphoma, chronic myeloproliferative disorders, a central nervous system tumor, brain cancer, glioblastoma, non-glioblastoma brain cancer, meningioma, pituitary adenoma, vestibular schwannoma, a primitive neuroectodermal tumor, medulloblastoma, astrocytoma, anaplastic astrocytoma, oligodendroglioma, ependymoma, choroid plexus papilloma, a myeloproliferative disorder, polycythemia vera, thrombocythemia, idiopathic myelfibrosis, soft tissue sarcoma, thyroid cancer, endometrial cancer, carcinoid cancer, germ cell tumor and liver cancer.


SEQUENCE LISTING (165 sequences)

SEQ ID NO: 1 - DNA, 384 bp, homo sapiens
  CDS (1)..(384)
atgtcgccatcacaactcattgggtttctgctgctctgggttccagcctccaggggtgaaattgtgctgactcagagcccagactctctgtctgtgactccaggcgagagagtcaccatcacctgccgggccagtcagagcattggtagtagcttacactggtaccagcagaaaccaggtcagtctccaaagcttctcatcaagtatgcatcccagtccctctcaggggtcccctcgaggttcagtggcagtggatctgggacagatttcaccctcaccatcagtagcctcgaggctgaagatgctgcagcgtattactgtcatcagagtagtcgtttacctcacactttcggccaagggaccaaggtggagatcaaacgtacg

SEQ ID NO: 2 - protein, 128 aa, homo sapiens
MSPSQLIGFLLLWVPASRGEIVLTQSPDSLSVTPGERVTITCRASQSIGSSLHWYQQKPGQSPKLLIKYASQSLSGVPSRFSGSGSGTDFTLTISSLEAEDAAAYYCHQSSRLPHTFGQGTKVEIKRT

SEQ ID NO: 3 - DNA, 384 bp, homo sapiens
  CDS (1)..(384)
atgtcgccatcacaactcattgggtttctgctgctctgggttccagcctccaggggtgaaattgtgctgactcagagcccagactctctgtctgtgactccaggcgagagagtcaccatcacctgccgggccagtcagagcattggtagtagcttacactggtaccagcagaaaccaggtcagtctccaaagcttctcatcaagtatgcatcccagtccctctcaggggtcccctcgaggttcagtggcagtggatctgggacagatttcaccctcaccatcagtagcctcgaggctgaagatttcgcagtgtattactgtcatcagagtagtcgtttacctcacactttcggccaagggaccaaggtggagatcaaacgtacg

SEQ ID NO: 4 - protein, 128 aa, homo sapiens
MSPSQLIGFLLLWVPASRGEIVLTQSPDSLSVTPGERVTITCRASQSIGSSLHWYQQKPGQSPKLLIKYASQSLSGVPSRFSGSGSGTDFTLTISSLEAEDFAVYYCHQSSRLPHTFGQGTKVEIKRT

SEQ ID NO: 5 - DNA, 384 bp, homo sapiens
  CDS (1)..(384)
atgtcgccatcacaactcattgggtttctgctgctctgggttccagcctccaggggtgaaattgtgctgactcagagcccaggtaccctgtctgtgtctccaggcgagagagccaccctctcctgccgggccagtcagagcattggtagtagcttacactggtaccagcagaaaccaggtcaggctccaaggcttctcatcaagtatgcatcccagtccctctcagggatccccgataggttcagtggcagtggatctgggacagatttcaccctcaccatcagtagactggagcctgaagatgctgcagcgtattactgtcatcagagtagtcgtttacctcacactttcggccaagggaccaaggtggagatcaaacgtaca

SEQ ID NO: 6 - protein, 128 aa, homo sapiens
MSPSQLIGFLLLWVPASRGEIVLTQSPGTLSVSPGERATLSCRASQSIGSSLHWYQQKPGQAPRLLIKYASQSLSGIPDRFSGSGSGTDFTLTISRLEPEDAAAYYCHQSSRLPHTFGQGTKVEIKRT

SEQ ID NO: 7 - DNA, 384 bp, homo sapiens
  CDS (1)..(384)
atgtcgccatcacaactcattgggtttctgctgctctgggttccagcctccaggggtgaaattgtgctgactcagagcccaggtaccctgtctgtgtctccaggcgagagagccaccctctcctgccgggccagtcagagcattggtagtagcttacactggtaccagcagaaaccaggtcaggctccaaggcttctcatcaagtatgcatcccagtccctctcagggatccccgataggttcagtggcagtggatctgggacagatttcaccctcaccatcagtagactggagcctgaagatttcgcagtgtattactgtcatcagagtagtcgtttacctcacactttcggccaagggaccaaggtggagatcaaacgtaca

SEQ ID NO: 8 - protein, 128 aa, homo sapiens
MSPSQLIGFLLLWVPASRGEIVLTQSPGTLSVSPGERATLSCRASQSIGSSLHWYQQKPGQAPRLLIKYASQSLSGIPDRFSGSGSGTDFTLTISRLEPEDFAVYYCHQSSRLPHTFGQGTKVEIKRT

SEQ ID NO: 9 - DNA, 411 bp, homo sapiens
  CDS (1)..(411)
atggagtttgggctgagctgggttttccttgttgctatattaaaaggtgtccagtgtgaggttcagctggtgcagtctgggggaggcttggtaaagcctggggggtccctgagactctcctgtgcagcctctggattcaccttcagtagctttgctatgcactgggttcgccaggctccaggaaaaggtctggagtggatatcagttattgatactcgtggtgccacatactatgcagactccgtgaagggccgattcaccatctccagagacaatgccaagaactccttgtatcttcaaatgaacagcctgagagccgaggacactgctgtgtattactgtgcaagactggggaacttctactacggtatggacgtctggggccaagggaccacggtcaccgtctcctca

SEQ ID NO: 10 - protein, 137 aa, homo sapiens
MEFGLSWVFLVAILKGVQCEVQLVQSGGGLVKPGGSLRLSCAASGFTFSSFAMHWVRQAPGKGLEWISVIDTRGATYYADSVKGRFTISRDNAKNSLYLQMNSLRAEDTAVYYCARLGNFYYGMDVWGQGTTVTVSS

SEQ ID NO: 11 - DNA, 411 bp, homo sapiens
  CDS (1)..(411)
atggagtttgggctgagctgggttttccttgttgctatattaaaaggtgtccagtgtgaggttcagctggtgcagtctgggggaggcttggtacagcccggggggtccctgagactctcctgtgcagcctctggattcaccttcagtagctttgctatgcactgggttcgccaggctccaggaaaaggtctggagtggatatcagttattgatactcgtggtgccacatactatgcagactccgtgaagggccgattcaccatctccagagacaatgccaagaactccttgtatcttcaaatgaacagcctgagagccgaggacactgctgtgtattactgtgcaagactggggaacttctactacggtatggacgtctggggccaagggaccacggtcaccgtctcctca

SEQ ID NO: 12 - protein, 137 aa, homo sapiens
MEFGLSWVFLVAILKGVQCEVQLVQSGGGLVQPGGSLRLSCAASGFTFSSFAMHWVRQAPGKGLEWISVIDTRGATYYADSVKGRFTISRDNAKNSLYLQMNSLRAEDTAVYYCARLGNFYYGMDVWGQGTTVTVSS

SEQ ID NO: 13 - protein, 174 aa, Artificial Sequence
  anti-IGF1R immunoglobulin
GRLGQAWRSLRLSCAASGFTFSDYYMSWIRQAPGKGLEWVSYISSSGSTRDYADSVKGRFTISRDNAKNSLYLQMNSLRAEDTAVYYCVRDGVETTFYYYYYGMDVWGQGTTVTVSSASTKGPSVFPLAPCSRSTSESTAALGCLVKDYFPEPVTVSWNSGALTSGVHTFPSCA

SEQ ID NO: 14 - protein, 124 aa, artificial sequence
  anti-IGF1R immunoglobulin
VQLLESGGGLVQPGGSLRLSCTASGFTFSSYAMNWVRQAPGKGLEWVSAISGSGGTTFYADSVKGRFTISRDNSRTTLYLQMNSLRAEDTAVYYCAKDLGWSDSYYYYYGMDVWGQGTTVTVSS

SEQ ID NO: 15 - protein, 112 aa, Artificial Sequence
  anti-IGF1R immunoglobulin
GPGLVKPSETLSLTCTVSGGSISNYYWSWIRQPAGKGLEWIGRIYTSGSPNYNPSLKSRVTMSVDTSKNQFSLKLNSVTAADTAVYYCAVTIFGVVIIFDYWGQGTLVTVSS

SEQ ID NO: 16 - protein, 125 aa, Artificial Sequence
  anti-IGF1R immunoglobulin
EVQLLESGGGLVQPGGSLRLSCAASGFTFSSYAMSWVRQAPGKGLEWVSAISGSGGITYYADSVKGRFTISRDNSKNTLYLQMNSLRAEDTAVYYCAKDLGYGDFYYYYYGMDVWGQGTTVTVSS

SEQ ID NO: 17 - protein, 113 aa, Artificial Sequence
  anti-IGF1R immunoglobulin
PGLVKPSETLSLTCTVSGGSISSYYWSWIRQPPGKGLEWIGYIYYSGSTNYNPSLKSRVTISVDTSKNQFSLKLSSVTAADTAVYYCARTYSSSFYYYGMDVWGQGTTVTVSS

SEQ ID NO: 18 - protein, 122 aa, Artificial Sequence
  anti-IGF1R immunoglobulin
EVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSS

SEQ ID NO: 19 - protein, 136 aa, Artificial Sequence
  anti-IGF1R immunoglobulin
ASVGDRVTFTCRASQDIRRDLGWYQQKPGKAPKRLIYAASRLQSGVPSRFSGSGSGTEFTLTISSLQPEDFATYYCLQHNNYPRTFGQGTEVEIIRTVAAPSVFIFPPSDEQLKSGTASVVCLLNNFYPREAKVQW

SEQ ID NO: 20 - protein, 107 aa, Artificial Sequence [sequence omitted]

SEQ ID NO: 21 - protein, 100 aa, Artificial Sequence
  anti-IGF1R immunoglobulin
SSLSASVGDRVTFTCRASQDIRRDLGWYQQKPGKAPKRLIYAASRLQSGVPSRFSGSGSGTEFTLTISSLQPEDFATYYCLQHNNYPRTFGQGTEVEIIR

SEQ ID NO: 22 - protein, 107 aa, Artificial Sequence
  anti-IGF1R immunoglobulin
DIQMTQSPSSLSASVGDRVTITCRASQGIRSDLGWFQQKPGKAPKRLIYAASKLHRGVPSRFSGSGSGTEFTLTISRLQPEDFATYYCLQHNSYPLTFGGGTKVEIK

SEQ ID NO: 23 - protein, 92 aa, Artificial Sequence
  anti-IGF1R immunoglobulin
GDRVTITCRASQSISTFLNWYQQKPGKAPKLLIHVASSLQGGVPSRFSGSGSGTDFTLTISSLQPEDFATYYCQQSYNAPLTFGGGTKVEIK

SEQ ID NO: 24 - protein, 91 aa, Artificial Sequence
  anti-IGF1R immunoglobulin
RATLSCRASQSVRGRYLAWYQQKPGQAPRLLIYGASSRATGIPDRFSGSGSGTDFTLTISRLEPEDFAVFYCQQYGSSPRTFGQGTKVEIK

SEQ ID NO: 25 - protein, 236 aa, Artificial Sequence
  anti-IGF1R immunoglobulin
MDMRVPAQLLGLLLLWFPGARCDIQMTQSPSSLSASVGDRVTITCRASQGIRNDLGWYQQKPGKAPKRLIYAASSLQSGVPSRFSGSGSGTEFTLTISSLQPEDFATYYCLQHNSYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 236 aa, Artificial Sequence
  anti-IGF1R immunoglobulin
MDMRVPAQLLGLLLLWFPGARCDIQMTQSPSSLSASVGDRVTFTCRASQDIRRDLGWYQQKPGKAPKRLIYAASRLQSGVPSRFSGSGSGTEFTLTISSLQPEDFATYYCLQHNNYPRTFGQGTEVEIIRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 236 aa, Artificial Sequence
  anti-IGF1R immunoglobulin
MDMRVPAQLLGLLLLWFPGARCDIQMTQSPSSLSASVGDRVTITCRASQGIRNDLGWYQQKPGKAPKRLIYAASSLQSGVPSRFSGSGSGTEFTLTISSLQPEDFATYYCLQHNSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 236 aa, Artificial Sequence
  anti-IGF1R immunoglobulin
MDMRVPAQLLGLLLLWFPGARCDIQMTQFPSSLSASVGDRVTITCRASQGIRNDLGWYQQKPGKAPKRLIYAASRLHRGVPSRFSGSGSGTEFTLTISSLQPEDFATYYCLQHNSYPCSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 473 aa, Artificial Sequence
  anti-IGF1R immunoglobulin
MEFGLSWVFLVAIIKGVQCQVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCARVLRFLEWLLYYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 30 - protein, 470 aa, Artificial Sequence
  anti-IGF1R immunoglobulin
MEFGLSWVFLVAIIKGVQCQAQLVESGGGLVKPGGSLRLSCAASGFTFSDYYMSWIRQAPGKGLEWVSYISSSGSTRDYADSVKGRFTISRDNAKNSLYLQMNSLRAEDTAVYYCVRDGVETTFYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 31 - protein, 470 aa, Artificial Sequence
  anti-IGF1R immunoglobulin
MEFGLSWLFLVAILKGVQCEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGYSSGWYYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 32 - protein, 470 aa, Artificial Sequence
  anti-IGF1R immunoglobulin
MEFGLSWLFLVAILKGVQCEVQLLESGGGLVQPGGSLRLSCTASGFTFSSYAMNWVRQAPGKGLEWVSAISGSGGTTFYADSVKGRFTISRDNSRTTLYLQMNSLRAEDTAVYYCAKDLGWSDSYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 33 - protein, 470 aa, Artificial Sequence
  anti-IGF1R immunoglobulin
MEFGLSWVFLVAIIKGVQCQVQLVESGGGLVKPGGSLRLSCAASGFTFSDYYMSWIRQAPGKGLEWVSYISSSGSTRDYADSVKGRFTISRDNAKNSLYLQMNSLRAEDTAVYYCARDGVETTFYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 34 - protein, 125 aa, Artificial Sequence
  anti-IGF1R immunoglobulin
QVQLVESGGGLVKPGGSLRLSCAASGFTFSDYYMSWIRQAPGKGLEWVSYISSSGSTRDYADSVKGRFTISRDNAKNSLYLQMNSLRAEDTAVYYCARDGVETTFYYYYYGMDVWGQGTTVTVSS

SEQ ID NO: 35 - protein, 236 aa, Artificial Sequence
  anti-IGF1R immunoglobulin
MDMRVPAQLLGLLLLWFPGARCDIQMTQSPSSLSASVGDRVTITCRASQDIRRDLGWYQQKPGKAPKRLIYAASRLQSGVPSRFSGSGSGTEFTLTISSLQPEDFATYYCLQHNNYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 108 aa, Artificial Sequence
  anti-IGF1R immunoglobulin
DIQMTQSPSSLSASVGDRVTITCRASQDIRRDLGWYQQKPGKAPKRLIYAASRLQSGVPSRFSGSGSGTEFTLTISSLQPEDFATYYCLQHNNYPRTFGQGTKVEIKR

SEQ ID NO: 37 - protein, 112 aa, Artificial Sequence
  anti-IGF1R immunoglobulin
DVVMTQSPLSLPVTPGEPASISCRSSQSIVHSNGNTYLQWYLQKPGQSPQLLIYKVSNRLYGVPDRFSGSGSGTDFTLKISRVEAEDVGVYYCFQGSHVPWTFGQGTKVEIK

SEQ ID NO: 38 - protein, 112 aa, Artificial Sequence [sequence omitted]

SEQ ID NO: 39 - protein, 117 aa, Artificial Sequence
  anti-IGF1R immunoglobulin
QVQLQESGPGLVKPSETLSLTCTVSGYSITGGYLWNWIRQPPGKGLEWMGYISYDGTNNYKPSLKDRITISRDTSKNQFSLKLSSVTAADTAVYYCARYGRVFFDYWGQGTLVTVSS

SEQ ID NO: 40 - protein, 117 aa, Artificial Sequence [sequence omitted]

SEQ ID NO: 41 - protein, 117 aa, Artificial Sequence
  anti-IGF1R immunoglobulin
QVQLQESGPGLVKPSETLSLTCTVSGYSISGGYLWNWIRQPPGKGLEWIGYISYDGTNNYKPSLKDRVTISVDTSKNQFSLKLSSVTAADTAVYYCARYGRVFFDYWGQGTLVTVSS

SEQ ID NO: 42 - protein, 130 aa, Artificial Sequence
  anti-IGF1R immunoglobulin
EVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARAPLRFLEWSTQDHYYYYYMDVWGKGTTVTVSS

SEQ ID NO: 43 - protein, 109 aa, Artificial Sequence
  anti-IGF1R immunoglobulin
SSELTQDPAVSVALGQTVRITCQGDSLRSYYASWYQQKPGQAPVLVIYGKNNRPSGIPDRFSGSSSGNTASLTITGAQAEDEADYYCNSRDNSDNRLIFGGGTKLTVLS

SEQ ID NO: 44 - protein, 119 aa, Artificial Sequence
  anti-IGF1R immunoglobulin
EVQLVQSGGGLVHPGGSLRLSCAGSGFTFRNYAMYWVRQAPGKGLEWVSAIGSGGGTYYADSVKGRFTISRDNAKNSLYLQMNSLRAEDMAVYYCARAPNWGSDAFDIWGQGTMVTVSS

SEQ ID NO: 45 - protein, 107 aa, Artificial Sequence
  anti-IGF1R immunoglobulin
DIQMTQSPSSLSASVGDRVTITCRASQGISSWLAWYQQKPEKAPKSLIYAASSLQSGVPSRFSGSGSGTDFTLTISSLQPEDFATYYCQQYNSYPPTFGPGTKVDIK

SEQ ID NO: 46 - protein, 251 aa, Artificial Sequence
  anti-IGF1R immunoglobulin
EVQLVQSGAEVKKPGESLTISCKGPGYNFFNYWIGWVRQMPGKGLEWMGIIYPTDSDTRYSPSFQGQVTISVDKSISTAYLQWSSLKASDTAMYYCARSIRYCPGGRCYSGYYGMDVWGQGTMVTVSSGGGGSGGGGSGGGGSSELTQDPAVSVALGQTVRITCQGDSLRSYYASWYQQKPGQAPVLVIYGKNNRPSGIPDRFSGSSSGNTASLTITGAQAEDEADYYCNSRDSSGNHVVFGGGTKLTVLG

SEQ ID NO: 47 - protein, 245 aa, Artificial Sequence
  anti-IGF1R immunoglobulin
QVQLVQSGAEVRKPGASVKVSCKTSGYTFRNYDINWVRQAPGQGLEWMGRISGHYGNTDHAQKFQGRFTMTKDTSTSTAYMELRSLTFDDTAVYYCARSQWNVDYWGRGTLVTVSSGGGGSGGGGSGGGGSALNFMLTQPHSVSESPGKTVTISCTRSSGSIASNYVQWYQQRPGSSPTTVIFEDNRRPSGVPDRFSGSIDTSSNSASLTISGLKTEDEADYYCQSFDSTNLVVFGGGTKVTVLG

SEQ ID NO: 48 - protein, 245 aa, Artificial Sequence
  anti-IGF1R immunoglobulin
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCASSPYSSRWYSFDPWGQGTMVTVSSGGGGSGGGGSGGGGSALSYELTQPPSVSVSPGQTATITCSGDDLGNKYVSWYQQKPGQSPVLVIYQDTKRPSGIPERFSGSNSGNIATLTISGTQAVDEADYYCQVWDTGTVVFGGGTKLTVLG

SEQ ID NO: 49 - protein, 251 aa, Artificial Sequence
  anti-IGF1R immunoglobulin
EVQLVQSGAEVKKPGESLTISCKGSGYNFFNYWIGWVRQMPGKDLEWMGIIYPTDSDTRYSPSFQGQVTISVDKSISTAYLQWSSLKASDTAMYYCARSIRYCPGGRCYSGYYGMDVWGQGTMVTVSSGGGSSGGGGSGGGGSSELTQDPAVSVALGQTVRITCRGDSLRNYYASWYQQKPGQAPVLVIYGKNNRPSGIPDRFSGSSSGNTASLTITGAQAEDEADYYCNSRDSSGNHMVFGGGTKLTVLG

SEQ ID NO: 50 - protein, 249 aa, Artificial Sequence
  anti-IGF1R immunoglobulin
EVQLVESGGGVVQPGRSLRLSCAASGFTFSDFAMHWVRQIPGKGLEWLSGLRHDGSTAYYAGSVKGRFTISRDNSRNTVYLQMNSLRAEDTATYYCVTGSGSSGPHAFPVWGKGTLVTVSSGGGGSGGGGSGGGGSALSYVLTQPPSASGTPGQRVTISCSGSNSNIGTYTVNWFQQLPGTAPKLLIYSNNQRPSGVPDRFSGSKSGTSASLAISGLQSEDEADYYCAAWDDSLNGPVFGGGTKVTVLG

SEQ ID NO: 51 - protein, 251 aa, Artificial Sequence
  anti-IGF1R immunoglobulin
EVQLVQSGAEVKKPGESLTISCKGSGYNFFNYWIGWVRQMPGKGLEWMGIIYPTDSDTRYSPSFQGQVTISVDKSISTAYLQWSSLKASDTAMYYCARSIRYCPGGRCYSGYYGMDVWGQGTLVTVSSGGGGSGGGGSGGGGSSELTQDPAVSVALGQTVRITCQGDSLRSYYTNWFQQKPGQAPLLVVYAKNKRPSGIPDRFSGSSSGNTASLTITGAQAEDEADYYCNSRDSSGNHVVFGGGTKLTVLG

SEQ ID NO: 52 - protein, 5 aa, Artificial Sequence
  anti-IGF1R immunoglobulin
SYWMH

SEQ ID NO: 53 - protein, 17 aa, Artificial Sequence
  anti-IGF1R immunoglobulin
EINPSNGRTNYNEKFKR

SEQ ID NO: 54 - protein, 15 aa, Artificial Sequence
  anti-IGF1R immunoglobulin
GRPDYYGSSKWYFDV

SEQ ID NO: 55 - protein, 16 aa, Artificial Sequence
  anti-IGF1R immunoglobulin
RSSQSIVHSNVNTYLE

SEQ ID NO: 56 - protein, 7 aa, Artificial Sequence
  anti-IGF1R immunoglobulin
KVSNRFS

SEQ ID NO: 57 - protein, 9 aa, Artificial Sequence
  anti-IGF1R immunoglobulin
FQGSHVPPT

SEQ ID NO: 58 - protein, 123 aa, Artificial Sequence
  anti-IGF1R immunoglobulin
QVQLVQSGAEVVKPGASVKLSCKASGYTFTSYWMHWVKQRPGQGLEWIGEINPSNGRTNYNQKFQGKATLTVDKSSSTAYMQLSSLTSEDSAVYYFARGRPDYYGSSKWYFDVWGQGTTVTVS

SEQ ID NO: 59 - protein, 118 aa, Artificial Sequence
  anti-IGF1R immunoglobulin
QVQFQQSGAELVKPGASVKLSCKASGYTFTSYLMHWIKQRPGRGLEWIGRIDPNNVVTKFNEKFKSKATLTVDKPSSTAYMELSSLTSEDSAVYYCARYAYCRPMDYWGQGTTVTVSS

SEQ ID NO: 60 - protein, 123 aa, Artificial Sequence
  anti-IGF1R immunoglobulin
QVQLQQSGAELVKPGASVKLSCKASGYTFTSYWMHWVKQRPGQGLEWIGEINPSNGRTNYNEKFKRKATLTVDKSSSTAYMQLSSLTSEDSAVYYFARGRPDYYGSSKWYFDVWGAGTTVTVS

SEQ ID NO: 61 - protein, 120 aa, Artificial Sequence
  anti-IGF1R immunoglobulin
QVQLQQSGAELMKPGASVKISCKATGYTFSSFWIEWVKQRPGHGLEWIGEILPGSGGTHYNEKFKGKATFTADKSSNTAYMQLSSLTSEDSAVYYCARGHSYYFYDGDYWGQGTSVTVSS

SEQ ID NO: 62 - protein, 120 aa, Artificial Sequence
  anti-IGF1R immunoglobulin
QVQLQQPGSVLVRPGASVKLSCKASGYTFTSSWIHWAKQRPGQGLEWIGEIHPNSGNTNYNEKFKGKATLTVDTSSSTAYVDLSSLTSEDSAVYYCARWRYGSPYYFDYWGQGTTLTVSS

SEQ ID NO: 63 - protein, 120 aa, Artificial Sequence
  anti-IGF1R immunoglobulin
QVQLQQPGAELVKPGASVKLSCKASGYTFTSYWMHWVKQRPGRGLEWIGRIDPNSGGTKYNEKFKSKATLTVDKPSSTAYMQLSSLTSEDSAVYYCARYDYYGSSYFDYWGQGTTLTVSS

SEQ ID NO: 64 - protein, 123 aa, Artificial Sequence; identical to SEQ ID NO: 58
  anti-IGF1R immunoglobulin

SEQ ID NO: 65 - protein, 124 aa, Artificial Sequence
  anti-IGF1R immunoglobulin
QVQLQQSGAELVKPGASVKLSCKASGYTFTSYWMHWVKQRPGQGLEWIGEINPSNGRTNYNEKFKRKATLTVDKSSSTAYMQLSSLTSEDSAVYYFARGRPDYYGSSKWYFDVWGAGTTVTVSS

SEQ ID NO: 66 - protein, 124 aa, Artificial Sequence
  anti-IGF1R immunoglobulin
QVQLVQSGAEVVKPGASVKLSCKASGYTFTSYWMHWVKQRPGQGLEWIGEINPSNGRTNYNQKFQGKATLTVDKSSSTAYMQLSSLTSEDSAVYYFARGRPDYYGSSKWYFDVWGQGTTVTVSS

SEQ ID NO: 67 - protein, 120 aa, Artificial Sequence
  anti-IGF1R immunoglobulin
QVQLQQSGAELVKPGASVKLSCKASGYTFTSYWMHWVKQRPGRGLEWIGRIDPNSGGTKYNEKFKSKATLTVDKPSSTAYMQLSSLTSEDSAVYYCARYDYYGSSYFDYWGQGTTVTVSS

SEQ ID NO: 68 - protein, 117 aa, Artificial Sequence
  anti-IGF1R immunoglobulin
QIQLQQSGPELVRPGASVKISCKASGYTFTDYYIHWVKQRPGEGLEWIGWIYPGSGNTKYNEKFKGKATLTVDTSSSTAYMQLSSLTSEDSAVYFCARGGKFAMDYWGQGTSVTVSS

SEQ ID NO: 69 - protein, 124 aa, Artificial Sequence; identical to SEQ ID NO: 65
  anti-IGF1R immunoglobulin

SEQ ID NO: 70 - protein, 120 aa, Artificial Sequence
  anti-IGF1R immunoglobulin
QIQLQQSGPELVKPGASVKISCKASGYTFTDYYINWMKQKPGQGLEWIGWIDPGSGNTKYNEKFKGKATLTVDTSSSTAYMQLSSLTSEDTAVYFCAREKTTYYYAMDYWGQGTSVTVSA

SEQ ID NO: 71 - protein, 115 aa, Artificial Sequence
  anti-IGF1R immunoglobulin
VQLQQSGAELMKPGASVKISCKASGYTFSDYWIEWVKQRPGHGLEWIGEILPGSGSTNYHERFKGKATFTADTSSSTAYMQLNSLTSEDSGVYYCLHGNYDFDGWGQGTTLTVSS

SEQ ID NO: 72 - protein, 120 aa, Artificial Sequence
  anti-IGF1R immunoglobulin
QVQLLESGAELMKPGASVKISCKATGYTFSSFWIEWVKQRPGHGLEWIGEILPGSGGTHYNEKFKGKATFTADKSSNTAYMQLSSLTSEDSAVYYCARGHSYYFYDGDYWGQGTSVTVSS

SEQ ID NO: 73 - protein, 113 aa, Artificial Sequence
  anti-IGF1R immunoglobulin
DVLMTQIPVSLPVSLGDQASISCRSSQIIVHNNGNTYLEWYLQKPGQSPQLLIYKVSNRFSGVPDRFSGSGSGTDFTLKISRVEAEDLGVYYCFQGSHVPFTFGSGTKLEIKR

SEQ ID NO: 74 - protein, 113 aa, Artificial Sequence
  anti-IGF1R immunoglobulin
DVLMTQTPLSLPVSLGDPASISCRSSQSIVHSNVNTYLEWYLQKPGQSPKLLIYKVSNRFSGVPDRFSGSGAGTDFTLRISRVEAEDLGIYYCFQGSHVPPTFGGGTKLEIKR

SEQ ID NO: 75 - protein, 113 aa, Artificial Sequence
  anti-IGF1R immunoglobulin
DVLMTQTPLSLPVSLGDPASISCRSSQSIVHSNVNTYLEWYLQKPGQSPRLLIYKVSNRFSGVPDRFSGSGAGTDFTLRISRVEAEDLGIYYCFQGSHVPPTFGGGTKLEIKR

SEQ ID NO: 76 - protein, 113 aa, Artificial Sequence; identical to SEQ ID NO: 74
  anti-IGF1R immunoglobulin

SEQ ID NO: 77 - protein, 113 aa, Artificial Sequence; identical to SEQ ID NO: 75
  anti-IGF1R immunoglobulin

SEQ ID NO: 78 - protein, 113 aa, Artificial Sequence
  anti-IGF1R immunoglobulin
  misc_feature (28)..(28): Xaa can be any naturally occurring amino acid
  misc_feature (101)..(101): Xaa can be any naturally occurring amino acid
DVLMTQTPLSLPVSLGDQASISCRSSQXIVHSNGNTYLEWYLQKPGQSPKLLIYKVSNRFSGVPDRFSGSGSGTDFTLKISRVEAEDLGVYYCFQGSHVPXTFGGGTKLEIKR

SEQ ID NO: 79 - protein, 113 aa, Artificial Sequence
  anti-IGF1R immunoglobulin
DVVMTQTPLSLPVSLGDPASISCRSSQSIVHSNVNTYLEWYLQKPGQSPKLLIYKVSNRFSGVPDRFSGSGAGTDFTLRISRVEAEDLGIYYCFQGSHVPPTFGGGTKLEIKR

SEQ ID NO: 80 - protein, 113 aa, Artificial Sequence
  anti-IGF1R immunoglobulin
DVVMTQTPLSLPVSLGDPASISCRSSQSIVHSNVNTYLEWYLQKPGQSPRLLIYKVSNRFSGVPDRFSGSGAGTDFTLRISRVEAEDLGIYYCFQGSHVPPTFGGGTKLEIKR

SEQ ID NO: 81 - protein, 113 aa, Artificial Sequence; identical to SEQ ID NO: 75
  anti-IGF1R immunoglobulin

SEQ ID NO: 82 - protein, 113 aa, Artificial Sequence; identical to SEQ ID NO: 73
  anti-IGF1R immunoglobulin

SEQ ID NO: 83 - protein, 113 aa, Artificial Sequence
  anti-IGF1R immunoglobulin
DVLMTQTPLSLPVSLGDQASISCRFSQSIVHSNGNTYLEWYLQKSGQSPKLLIYKVSNRFSGVPDRFSGSGSGTDFTLKISRVEAEDLGVYYCFQGSHVPRTFGGGTKLEIKR

SEQ ID NO: 84 - protein, 113 aa, Artificial Sequence
  anti-IGF1R immunoglobulin
DVLMTQTPLSLPVSLGDQASISCRSSQSIVHSNVNTYLEWYLQKPGQSPKLLIYKVSNRFSGVPDRFSGSGSGTDFTLRISRVEAEDLGIYYCFQGSHVPPTFGGGTKLEIKR

SEQ ID NO: 85 - protein, 113 aa, Artificial Sequence; identical to SEQ ID NO: 79
  anti-IGF1R immunoglobulin

SEQ ID NO: 86 - protein, 113 aa, Artificial Sequence
  anti-IGF1R immunoglobulin
ELVMTQTPLSLPVSLGDQASISCRSSQTIVHSNGDTYLDWFLQKPGQSPKLLIYKVSNRFSGVPDRFSGSGSGTDFTLKISRVEAEDLGVYYCFQGSHVPPTFGGGTKLEIKR

SEQ ID NO: 87 - protein, 113 aa, Artificial Sequence; identical to SEQ ID NO: 74
  anti-IGF1R immunoglobulin

SEQ ID NO: 88 - protein, 113 aa, Artificial Sequence; identical to SEQ ID NO: 80
  anti-IGF1R immunoglobulin

SEQ ID NO: 89 - protein, 113 aa, Artificial Sequence
  anti-IGF1R immunoglobulin
DVLMTQTPVSLSVSLGDQASISCRSSQSIVHSTGNTYLEWYLQKPGQSPKLLIYKISNRFSGVPDRFSGSGSGTDFTLKISRVEAEDLGVYYCFQASHAPRTFGGGTKLEIKR

SEQ ID NO: 90 - protein, 113 aa, Artificial Sequence
  anti-IGF1R immunoglobulin
DVLMTQTPLSLPVSLGDQASISCKSSQSIVHSSGNTYFEWYLQKPGQSPKLLIYKVSNRFSGVPDRFSGSGSGTDFTLKISRVEAEDLGVYYCFQGSHIPFTFGSGTKLEIKR

SEQ ID NO: 91 - protein, 113 aa, Artificial Sequence
  anti-IGF1R immunoglobulin
DIELTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPYTFGGGTKLEIKR

SEQ ID NO: 92 - protein, 113 aa, Artificial Sequence; identical to SEQ ID NO: 84
  anti-IGF1R immunoglobulin

SEQ ID NO: 93 - protein, 113 aa, Artificial Sequence; identical to SEQ ID NO: 80
  anti-IGF1R immunoglobulin

SEQ ID NO: 94 - protein, 113 aa, Artificial Sequence; identical to SEQ ID NO: 84
  anti-IGF1R immunoglobulin

SEQ ID NO: 95 - protein, 113 aa, Artificial Sequence; identical to SEQ ID NO: 79
  anti-IGF1R immunoglobulin

SEQ ID NO: 96 - protein, 113 aa, Artificial Sequence
  anti-IGF1R immunoglobulin
DVLMTQTPLSLPVSLGDQASISCRSNQTILLSDGDTYLEWYLQKPGQSPKLLIYKVSNRFSGVPDRFSGSGSGTDFTLKISRVEAEDLGVYYCFQGSHVPPTFGGGTKLEIKR

SEQ ID NO: 97 - protein, 113 aa, Artificial Sequence
  anti-IGF1R immunoglobulin
DVLMTQTPLSLPVSLGDQASISCRSSQTIVHSNGNTYLEWYLQKPGQSPKLLIYKVTNRFSGVPDRFSGSGSGTDFTLKISRVEAEDLGVYYCFQGTHAPYTFGGGTKLEIKR

SEQ ID NO: 98 - protein, 113 aa, Artificial Sequence
  anti-IGF1R immunoglobulin
DVLMTQTPLSLPVSLGDQASISCRSSQSIVHSNGNTYLEWYLQKPGQSPKLLIYSISSRFSGVPDRFSGSGSGTDFTLKISRVQAEDLGVYYCFQGSHVPYTFGGGTKLEIKR

SEQ ID NO: 99 - DNA, 4221 bp, Homo sapiens
atggacatggtagagaatgcagatagtttgcaggcacaggagcggaaggacatacttatgaagtatgacaagggacaccgagctgggctgccagaggacaaggggcctgagcccgttggaatcaacagcagcattgatcgttttggcattttgcatgagacggagctgcctcctgtgactgcacgggaggcgaagaaaattcggcgggagatgacacgaacgagcaagtggatggaaatgctgggagaatgggagacatataagcacagtagcaaactcatagatcgagtgtacaagggaattcccatgaacatccggggcccggtgtggtcagtcctcctgaacattcaggaaatcaagttgaaaaaccccggaagataccagatcatgaaggagaggggcaagaggtcatctgaacacatccaccacatcgacctggacgtgaggacgactctccggaaccatgtcttctttagggatcgatatggagccaagcagagggaactattctacatcctcctggcctattcggagtataacccggaggtgggctactgcagggacctgagccacatcaccgccttgttcctcctttatctgcctgaggaggacgcattctgggcactggtgcagctgctggccagtgagaggcactccctgccaggattccacagcccaaatggtgggacagtccaggggctccaagaccaacaggagcatgtggtacccaagtcacaacccaagaccatgtggcatcaggacaaggaaggtctatgcgggcagtgtgcctcgttaggctgccttctccggaacctgattgacgggatctctctcgggctcaccctgcgcctgtgggacgtgtatttggtggaaggagaacaggtgttgatgccaataaccagcattgctcttaaggttcagcagaagcgcctcatgaagacatccaggtgtggcctgtgggcacgtctgcggaaccaattcttcgatacctgggccatgaacgatgacaccgtgctcaagcatcttagggcctctacgaagaaactaacaaggaagcaaggggacctgccacccccagccaaacgcgagcaagggtccttggcacccaggcctgtgccggcttcacgtggtgggaagaccctctgcaaggggtataggcaggcccctccaggcccaccagcccagttccagcggcccatttgctcagcttccccgccatgggcatctcgtttttccacgccctgtcctggtggggctgtccgggaagacacgtaccctgtgggcactcagggtgtgcccagcctggccctggctcagggaggacctcagggttcctggagattcctggagtggaagtcaatgccccggctcccaacggacctggatatagggggcccttggttcccccattatgattttgaatggagctgctgggtccgtgccatatcccaggaggaccagctggccacctgctggcaggctgaacactgcggagaggttcacaacaaagatatgagttggcctgaggagatgtcttttacagcaaatagtagtaaaatagatagacaaaaggttcccacagaaaagggagccacaggtctaagcaacctgggaaacacatgcttcatgaactcaagcatccagtgcgttagtaacacacagccactgacacagtattttatctcagggagacatctttatgaactcaacaggacaaatcccattggtatgaaggggcatatggctaaatgctatggtgatttagtgcaggaactctggagtggaactcagaagagtgttgccccattaaagcttcggcggaccatagcaaaatatgctcccaagtttgatgggtttcagcaacaagactcccaagaacttctggcttttctcttggatggtcttcatgaagatctcaaccgagtccatgaaaagccatatgtggaactgaaggacagtgatggccgaccagactgggaagtagctgcagaggcctgggacaaccatctaagaagaaatagatcaattattgtggatttgttccatgggcagctaagatctcaagtcaaatgcaagacatgtgggcatataagtgtccgatttgaccctttcaattttttgtctttgccactaccaatggacagttacatggacttagaaataacagtgattaagttagatggtactacccctgtacggtatggactaagactgaatatggatgaaaagtacacaggtttaaaaaaacagctgagggatctctgtggacttaattcagaacaaatcctactagcagaagtacatgattccaacataaagaactttcctcaggataaccaaaaagtacaactctcagtgagcggatttttgtgtgcatttgaaattcctgtcccttcatctccaatttcagcttctagtccaacacaaatagatttctcctcttcaccatctacaaatggaatgttcaccctaactaccaatggggacctacccaaaccaatattcatccccaatggaatgccaaacactgttgtgccatgtggaactgagaagaacttcacaaatggaatggttaatggtcacatgccatctcttcctgacagcccctttacaggttacatcattgcagtccaccgaaaaatgatgaggacagaactgtatttcctgtcacctcaggagaatcgccccagcctctttggaatgccattgattgttccatgcactgtgcatacccggaagaaagacctatatgatgcggtttggattcaagtatcctggttagcaagaccactcccacctcaggaagctagtattcatgcccaggatcgtgataactgtatgggctatcaatatccattcactctacgagttgtgcagaaagatgggaactcctgtgcttggtgcccacagtatagattttgcagaggctgtaaaattgattgtggggaagacagagctttcattggaaatgcctatattgctgtggattggcaccccacagcccttcaccttcgctatcaaacatcccaggaaagggttgtagataagcatgagagtgtggagcagagtcggcgagcgcaagccgagcccatcaacctggacagctgtctccgtgctttcaccagtgaggaagagctaggggaaagtgagatgtactactgttccaagtgtaagacccactgcttagcaacaaagaagctggatctctggaggcttccacccttcctgattattcaccttaagcgatttcaatttgtaaatgatcagtggataaaatcacagaaaattgtcagatttcttcgggaaagttttgatccgagtgcttttttggtaccacgagacccggccctctgccagcataaaccactcacaccccagggggatgagctctccaagcccaggattctggcaagagaggtgaagaaagtggatgcgcagagttcggctggaaaagaggacatgctcctaagcaaaagcccatcctcactcagcgctaacatcagcagcagcccaaaaggttctccttcttcatcaagaaaaagtggaaccagctgtccctccagcaaaaacagcagccctaatagcagcccacggactttggggaggagcaaagggaggctccggctgccccagattggcagcaaaaataagccgtcaagtagtaagaagaacttggatgccagcaaagagaatggggctgggcagatctgtgagctggctgacgccttgagccgagggcatatgcgggggggcagccaaccagagctggtcactcctcaggaccatgaggtagctttggccaatggattcctttatgagcatgaagcatgtggcaatggctgtggcgatggctacagcaatggtcagcttggaaaccacagtgaagaagacagcactgatgaccaaagagaagacactcatattaagcctatttataatctatatgcaatttcatgccattcaggaattctgagtgggggccattacatcacttatgccaaaaacccaaactgcaagtggtactgttataatgacagcagctgtgaggaacttcaccctgatgaaattgacaccgactctgcctacattcttttctatgagcagcaggggatagactacgcacaatttctgccaaagattgatggcaaaaagatggcagacacaagcagtacggatgaagactctgagtctgattacgaaaagtactctatgttacagtaa

SEQ ID NO: 100 - DNA, 2778 bp, Homo sapiens
atgccccgtaaaggcacccagccctccactgcccggcgcagagaggaagggccgccgccgccgtcccctgacggcgccagcagcgacgcggagcctgagccgccgtccggccgcacggagagcccagccaccgccgcagcaatgaccaatgaagctggagctcctcggcttatgataactcatattgtaaaccagaacttcaaatcctatgctggggagaaaattctgggacctttccataagcgcttttcctgtattatcgggccaaatggcagtggcaaatccaatgttattgattctatgctttttgtgtttggctatcgagcacaaaaaataagatctaaaaaactctcagtattaatacataattctgatgaacacaaggacattcagagttgtacagtagaagttcattttcaaaagataattgataaggaaggggatgattatgaagtcattcctaacagtaatttctatgtatccagaacggcctgcagagataatacttctgtctatcacataagtggaaagaaaaagacatttaaggatgttggaaatcttcttcgaagccatggaattgacttggaccataatagatttttaattttacagggtgaagttgaacaaattgctatgatgaaaccaaaaggccagactgaacacgatgagggtatgcttgaatatttagaagatataattggttgtggacggctaaatgaacctattaaagtcttgtgtcggagagttgaaatattaaatgaacacagaggagagaagttaaacagggtaaagatggtggaaaaggaaaaggatgccttagaaggagagaaaaacatagctatcgaatttcttaccttggaaaatgaaatatttagaaaaaagaatcatgtttgtcaatattatatttatgagttgcagaaacgaattgctgaaatggaaactcaaaaggaaaaaattcatgaagataccaaagaaattaatgagaagagcaatatactatcaaatgaaatgaaagctaagaataaagatgtaaaagatacagaaaagaaactgaataaaattacaaaatttattgaggagaataaagaaaaatttacacagctagatttggaagatgttcaagttagagaaaagttaaaacatgccacgagtaaagccaaaaaactggagaaacaacttcaaaaagataaagaaaaggttgaagaatttaaaagtatacctgccaagagtaacaatatcattaatgaaacaacaaccagaaacaatgccctcgagaaggaaaaagagaaagaagaaaaaaaattaaaggaagttatggatagccttaaacaggaaacacaagggcttcagaaagaaaaagaaagtcgagagaaagaacttatgggtttcagcaaatcggtaaatgaagcacgttcaaagatggatgtagcccagtcagaacttgatatctatctcagtcgtcataatactgcagtgtctcaattaactaaggctaaggaagctctaattgcagcttctgagactctcaaagaaaggaaagctgcaatcagagatatagaaggaaaactccctcaaactgaacaagaattaaaggagaaagaaaaagaacttcaaaaacttacacaagaagaaacaaactttaaaagtttggttcatgatctctttcaaaaagttgaagaagcaaagagctcattagcaatgaatcgaagtagggggaaagtccttgatgcaataattcaagaaaaaaaatctggcaggattccaggaatatatggaagattgggggacttaggagccattgatgaaaaatacgacgtggctatatcatcctgttgtcatgcactggactacattgttgttgattctattgatatagcccaagaatgtgtaaacttccttaaaagacaaaatattggagttgcaacctttataggtttagataagatggctgtatgggcgaaaaagatgaccgaaattcaaactcctgaaaatactcctcgtttatttgatttagtaaaagtaaaagatgagaaaattcgccaagctttttattttgctttacgagataccttagtagctgacaacttggatcaagccacaagagtagcatatcaaaaagatagaagatggagagtggtaactttacagggacaaatcatagaacagtcaggtacaatgactggtggtggaagcaaagtaatgaaaggaagaatgggttcctcacttgttattgaaatctctgaagaagaggtaaacaaaatggaatcacagttgcaaaacgactctaaaaaagcaatgcaaatccaagaacagaaagtacaacttgaagaaagagtagttaagttacggcatagtgaacgagaaatgaggaacacactagaaaaatttactgcaagcatccagcgtttaatagagcaagaagaatatttgaatgtccaagttaaggaacttgaagctaatgtacttgctacagcccctgacaaaaaaaagcagaaattgctagaagaaaacgttagtgctttcaaaacagaatatgatgctgtggctgagaaagctggtaaagtagaagctgaggttaaacgcttacacaataccatcgtagaaatcaataatcataaactcaaggcccaacaagacaaacttgataaaataaataagcaattagatgaatgtgcttctgctattactaaagcccaagtagcaatcaagactgctgacagaccttcaaaaggcacaagactctgtcttgcgtacagagaaagaaataaaagatactga

SEQ ID NO: 101 - DNA, 1032 bp, Homo sapiens
atgaacatacacaggtctacccccatcacaatagcgagatatgggagatcgcggaacaaaacccaggatttcgaagagttgtcgtctataaggtccgcggagcccagccagagtttcagcccgaacctcggctccccgagcccgcccgagactccgaacttgtcgcattgcgtttcttgtatcgggaaatacttattgttggaacctctggagggagaccacgtttttcgtgccgtgcatctgcacagcggagaggagctggtgtgcaaggtgtttgatatcagctgctaccaggaatccctggcaccgtgcttttgcctgtctgctcatagtaacatcaaccaaatcactgaaattatcctgggtgagaccaaagcctatgtgttctttgagcgaagctatggggacatgcattccttcgtccgcacctgcaagaagctgagagaggaggaggcagccagactgttctaccagattgcctcggcagtggcccactgccatgacggggggctggtgctgcgggacctcaagctgcggaaattcatctttaaggacgaagagaggactcgggtcaagctggaaagcctggaagacgcctacattctgcggggagatgatgattccctctccgacaagcatggctgcccggcttacgtaagcccagagatcttgaacaccagtggcagctactcgggcaaagcagccgacgtgtggagcctgggggtgatgctgtacaccatgttggtggggcggtaccctttccatgacattgaacccagctccctcttcagcaagatccggcgtggccagttcaacattccagagactctgtcgcccaaggccaagtgcctcatccgaagcattctgcgtcgggagccctcagagcggctgacctcgcaggaaattctggaccatccttggttttctacagattttagcgtctcgaattcagcatatggtgctaaggaagtgtctgaccagctggtgccggacgtcaacatggaagagaacttggaccctttctttaactga

SEQ ID NO: 102 - DNA, 2322 bp, Homo sapiens
atgattcgcgacctgagcaagatgtacccgcagaccagacacccggcaccgcatcagcctgctcaaccctttaaatttacaatttccgaatcctgtgatcggattaaggaagagtttcagtttttacaggctcaataccacagtctgaagctggaatgtgagaaactcgccagtgagaagacagagatgcagcggcattatgtcatgtattatgaaatgtcctatgggttgaatatagaaatgcacaagcaggcagagattgtcaagaggctgaatgctatctgtgcacaagtcattcctttcctgtcccaagagcaccagcaacaagtggtgcaggctgtggaacgggccaagcaggtgaccatggcagaactgaacgccatcattgggcaacaactccaggcccagcatttatcacatggacatggtctccccgtacctctgactccacacccttcagggctccagccccctgccattccacccatcggtagcagtgccgggcttctggccctctccagtgctctaggaggtcagtcccatcttccaattaaagatgagaagaagcaccatgacaatgatcaccaaagagacagagactccatcaagagctcttcagtatccccatcagccagtttccgaggtgctgagaagcacagaaactccgcagactactcctcagagagcaaaaagcagaaaactgaagaaaaggaaattgcagctcgttatgacagcgatggtgagaaaagtgatgacaacttggtggttgacgtttccaatgaggatccatcttcccctcgagggagcccagcacattcccccagagagaatggcctagacaagacacgcctgctcaagaaagatgccccgattagtccagcctctattgcatcttccagcagtactccctcctccaaatccaaagaacttagccttaatgaaaaatctactactcccgtctcaaagtccaatacccctactccacgaactgatgcgcccaccccaggcagtaactctactcccggattgaggcctgtacctggaaaaccaccaggagttgaccctttggcctcaagcctaaggaccccaatggcagtaccttgtccatatccaactccatttgggattgtgccccatgctggaatgaacggagagctgaccagccccggagcggcctacgctgggctccacaacatctcccctcagatgagcgcagctgctgccgccgccgctgctgctgctgcctatgggagatcaccagtggtgggatttgatccacaccatcacatgcgtgtgccagcaatacctccaaacctgacaggcattccaggaggaaaaccagcatactccttccatgttagcgcagatggtcagatgcagcctgtcccttttccacccgacgccctcatcggacctggaatcccccggcatgctcgccagatcaacaccctcaaccacggggaggtggtgtgcgcggtgaccatcagcaaccccacgagacacgtgtacacgggtgggaagggctgcgtcaaggtctgggacatcagccacccaggcaataagagtcctgtctcccagctcgactgtctgaacagggataactacatccgttcctgcagattgctccctgatggtcgcaccctaattgttggaggggaagccagtactttgtccatttgggacctggcggctccaaccccacgcatcaaggcagagctgacatcctcggcccccgcctgctatgccctggccatcagccccgattccaaggtctgcttctcatgctgcagcgacggcaacatcgctgtgtgggatctgcacaaccagaccttggtgaggcaattccagggccacacagatggagccagctgtattgacatttctaatgatggcaccaagctctggacaggtggtttggacaacacggtcaggtcctgggacctgcgcgaggggcggcagctgcagcagcacgacttcacctcccagatcttttctctgggctactgcccaactggagagtggcttgcagtggggatggagaacagcaatgtggaagttttgcatgtcaccaagccagacaaataccaactacatcttcatgagagctgtgtgctgtcgctcaagtttgcccattgtggcaaatggtttgtaagcactggaaaggacaaccttctgaatgcctggagaacaccttatggggccagtatattccagtccaaagaatcctcatcggtgcttagctgtgacatctccgtggacgacaaatacattgtcactggctctggggataagaaggccacagtttatgaagttatttattaa

SEQ ID NO: 103 - DNA, 1296 bp, Homo sapiens
atgcacgtgcgctcactgcgagctgcggcgccgcacagcttcgtggcgctctgggcacccctgttcctgctgcgctccgccctggccgacttcagcctggacaacgaggtgcactcgagcttcatccaccggcgcctccgcagccaggagcggcgggagatgcagcgcgagatcctctccattttgggcttgccccaccgcccgcgcccgcacctccagggcaagcacaactcggcacccatgttcatgctggacctgtacaacgccatggcggtggaggagggcggcgggcccggcggccagggcttctcctacccctacaaggccgtcttcagtacccagggcccccctctggccagcctgcaagatagccatttcctcaccgacgccgacatggtcatgagcttcgtcaacctcgtggaacatgacaaggaattcttccacccacgctaccaccatcgagagttccggtttgatctttccaagatcccagaaggggaagctgtcacggcagccgaattccggatctacaaggactacatccgggaacgcttcgacaatgagacgttccggatcagcgtttatcaggtgctccaggagcacttgggcagggaatcggatctcttcctgctcgacagccgtaccctctgggcctcggaggagggctggctggtgtttgacatcacagccaccagcaaccactgggtggtcaatccgcggcacaacctgggcctgcagctctcggtggagacgctggatgggcagagcatcaaccccaagttggcgggcctgattgggcggcacgggccccagaacaagcagcccttcatggtggctttcttcaaggccacggaggtccacttccgcagcatccggtccacggggagcaaacagcgcagccagaaccgctccaagacgcccaagaaccaggaagccctgcggatggccaacgtggcagagaacagcagcagcgaccagaggcaggcctgtaagaagcacgagctgtatgtcagcttccgagacctgggctggcaggactggatcatcgcgcctgaaggctacgccgcctactactgtgagggggagtgtgccttccctctgaactcctacatgaacgccaccaaccacgccatcgtgcagacgctggtccacttcatcaacccggaaacggtgcccaagccctgctgtgcgcccacgcagctcaatgccatctccgtcctctacttcgatgacagctccaacgtcatcctgaagaaatacagaaacatggtggtccgggcctgtggctgccactag

SEQ ID NO: 104 - DNA, 2805 bp, Homo sapiens
atggtcccagaggcctggaggagcggactggtaagcaccgggagggtagtgggagttttgcttctgcttggtgccttgaacaaggcttccacggtcattcactatgagatcccggaggaaagagagaagggtttcgctgtgggcaacgtggtcgcgaaccttggtttggatctcggtagcctctcagcccgcaggttccgggtggtgtctggagctagccgaagattctttgaggtgaaccgggagaccggagagatgtttgtgaacgaccgtctggatcgagaggagctgtgtgggacactgccctcttgcactgtaactctggagttggtagtggagaacccgctggagctgttcagcgtggaagtggtgatccaggacatcaacgacaacaatcctgctttccctacccaggaaatgaaattggagattagcgaggccgtggctccggggacgcgctttccgctcgagagcgcgcacgatcccgatgtgggaagcaactctttacaaacctatgagctgagccgaaatgaatactttgcgcttcgcgtgcagacgcgggaggacagcaccaagtacgcggagctggtgttggagcgcgccctggaccgagaacgggagcctagtctccagttagtgctgacggcgttggacggagggaccccagctctctccgccagcctgcctattcacatcaaggtgctggacgcgaatgacaatgcgcctgtcttcaaccagtccttgtaccgggcgcgcgtcctggaggatgcaccctccggcacgcgcgtggtacaagtccttgcaacggatctggatgaaggccccaacggtgaaattatttactccttcggcagccacaaccgcgccggcgtgcggcaactattcgccttagaccttgtaaccgggatgctgacaatcaagggtcggctggacttcgaggacaccaaactccatgagatttacatccaggccaaagacaagggcgccaatcccgaaggagcacattgcaaagtgttggtggaggttgtggatgtgaatgacaacgccccggagatcacagtcacctccgtgtacagcccagtacccgaggatgcccctctggggactgtcatcgctttgctcagtgtgactgacctggatgctggcgagaacgggctggtgacctgcgaagttccaccgggtctccctttcagccttacttcttccctcaagaattacttcactttgaaaaccagtgcagacctggatcgggagactgtgccagaatacaacctcagcatcaccgcccgagacgccggaaccccttccctctcagcccttacaatagtgcgtgttcaagtgtccgacatcaatgacaaccctccacaatcttctcaatcttcctacgacgtttacattgaagaaaacaacctccccggggctccaatactaaacctaagtgtctgggaccccgacgccccgcagaatgctcggctttctttctttctcttggagcaaggagctgaaaccgggctagtgggtcgctatttcacaataaatcgtgacaatggcatagtgtcatccttagtgcccctagactatgaggatcggcgggaatttgaattaacagctcatatcagcgatgggggcaccccggtcctagccaccaacatcagcgtgaacatatttgtcactgatcgcaatgacaatgccccccaggtcctatatcctcggccaggtgggagctcggtggagatgctgcctcgaggtacctcagctggccacctagtgtcacgggtggtaggctgggacgcggatgcagggcacaatgcctggctctcctacagtctcttgggatcccctaaccagagcctttttgccatagggctgcacactggtcaaatcagtactgcccgtccagtccaagacacagattcacccaggcagactctcacggtcttgatcaaagacaatggggagccttcgctctccaccactgctaccctcactgtgtcagtaaccgaggactctcctgaagcccgagccgagttcccctctggctctgccccccgggagcagaaaaaaaatctcaccttttatctacttctttctctaatcctggtttctgtggggtttgtggtcacagtgttcggagtaatcatattcaaagtttacaagtggaagcagtctagagacctataccgagccccggtgagctcactgtaccgaacaccagggccctccttgcacgcggacgccgtgcggggaggcctgatgtcgccgcacctttaccatcaggtgtatctcaccacggactcccgccgcagcgacccgctgctgaagaaacctggtgcagccagtccactggccagccgccagaacacgctgcggagctgtgatccggtgttctataggcaggtgttgggtgcagagagcgcccctcccggacagcaagccccgcccaacacggactggcgtttctctcaggcccagagacccggcaccagcggctcccaaaatggcgatgacaccggcacctggcccaacaaccagtttgacacagagatgctgcaagccatgatcttggcgtccgccagtgaagctgctgatgggagctccaccctgggagggggtgccggcaccatgggattgagcgcccgctacggaccccagttcaccctgcagcacgtgcccgactaccgccagaatgtctacatcccaggcagcaatgccacactgaccaacgcagctggcaagcgggatggcaaggccccagcaggtggcaatggcaacaagaagaagtcgggcaagaaggagaagaagtaa

SEQ ID NO: 105 - DNA, 3780 bp, Homo sapiens
atggatggcccgacgcggggccatggactccgcaaaaagcggcggtcgcggtcgcagcgagaccgggagaggcgctcccggggcgggctgggggccggcgcggccggcggcggcggggctggccggacccgggcgctctcactcgcctcgtcgtcgggctccgacaaggaagacaatgggaagcccccgtcctccgccccgtcccggcccagacccccgcggaggaagcggagagagtccacctcggcagaagaggacatcattgatggatttgccatgaccagctttgtcacttttgaagcgctggagaaagatgtagcacttaagcctcaggaacgtgtggagaaacgccagacgcccctgaccaagaagaaacgagaagcacttaccaatggcttgtcctttcattcaaagaagagcagactcagccacccacaccactacagctcagatcgagaaaatgaccgcaatctctgccagcaccttgggaagagaaagaaaatgccgaaggcactcagacagctcaagccaggacagaacagctgcagggacagtgacagtgaaagtgccagtggagaatccaagggcttccaccggagcagctctcgggaaaggctcagtgatagttcagctccttccagcttgggaacaggctacttctgtgacagtgacagtgaccaggaagagaaggcatcagatgccagctctgaaaaactcttcaacactgttattgtaaacaaagatccggagttaggtgttggcacgctaccagaacatgacagccaggatgcagggccgattgtccccaagatatcgggtctagagagaagccaggagaagagccaggactgttgcaaagagccaatctttgagcctgtggtgcttaaagacccctgccctcaggtcgcacagccaataccccagccgcagacggagccccaactccgagctccttctccggaccctgacttggtgcagcgcacagaggccccacctcaacccccacctctgagtacacagccaccacagggccctcctgaggcccagctccagcctgccccgcagcctcaggtgcagaggccacccaggccacagtcccccacccagctgctccatcagaacctcccacctgtgcaggcccacccctctgctcagagcctctcccagccattgtcagcctacaacagcagtagcttaagcctcaacagtttaagcagcagcagaagcagcactccagcgaagactcagcccgccccacctcacatctcccaccacccctctgcctccccgttccccctctccctgcccaaccacagccccctgcacagcttcacacccaccctccagccccccgcacactcacatcaccccaatatgtttgcccctcccactgctctgcctcctccaccaccactgacatcaggaagtctgcaggtggccggacacccggccgggagcacttactcagagcaagacatcttgcgacaggaactgaacactcgttttttggcctctcagagtgctgaccgcggggcttccctgggccctccgccctacctgcggaccgagttccatcagcaccagcaccagcaccagcacacccaccagcacacgcaccagcacaccttcacgccgttcccccacgccatcccacccaccgccatcatgccgacgccagcacctcccatgtttgacaaataccctacaaaagttgacccattctaccggcacagtctcttccattcctatcctcctgcagtgtcgggcatcccccctatgatcccacccactggcccttttggttcactacaaggagcatttcagccgaagacatccaaccctatcgatgtcgctgctcggcctgggacagtcccacacactttactccaaaaggacccgaggttgacagatcctttcagacctatgttaaggaaaccagggaagtggtgtgctatgcatgttcacatcgcctggcagatttaccaccaccaacagaaagtcaagaaacagatgcagtcagacccacataagctggactttggactgaaacctgagttcctgagccgccctccaggccccagtctttttggagccatccaccacccccatgacctggcacggccttcaactttgttctctgccgctggtgctgcacacccaactgggaccccttttgggccacctcctcatcacagcaacttcctcaaccctgctgcccacctagagccttttaatcggccgtctacattcacaggcctagcagcagttggtggcaatgccttcgggggacttggaaatccttccgttacacccaactcaatgttcggccacaaggatggccccagtgtgcagaactttagcaaccctcacgaaccctggaaccggctgcaccgaacgcctccgtcgttcccgacccctccgccctggctgaagccaggggagctggagcgcagcgcgtccgctgcagctcatgacagagatagagatgtagataaacgagactcatctgttagtaaagatgacaaagaaagggaaagcgtcgagaagagacactccagccacccttcaccagcacctgtcctcccggtgaatgccctgggacatacccgcagctccactgaacagatccgggctcatctgaacactgaggctcgggagaaggacaaacccaaagagagggagagagaccactcggaatcccgcaaggacctggccgccgacgagcacaaggcgaaagagggccacctgcccgagaaggacgggcacggccacgaggggcgcgccgcgggcgaagaggccaagcagctggcccgggtgccgtctccctacgtgcggaccccggtggtggagagtgccaggcccaacagcacctcgagccgggaggccgagccgcgcaagggtgagccggcctacgagaaccccaagaagagctccgaggtcaaggtgaaggaggagcggaaggaagaccatgacctgcctccagaggccccgcagacccaccgggcctcggagccgccgcctcccaactcctcgtccagcgtgcacccggggcccctggcctcgatgcccatgacggtgggggtgacgggcattcaccccatgaacagcatcagcagcctggacaggactcgcatgatgacccccttcatgggcatcagccccctcccgggcggagagcgcttcccgtacccttctttccactgggaccccatccgggaccccttgagggatccttaccgagaacttgacattcaccggagagacccgctgggcagggacttcctgctaaggaacgacccgctccaccggctctcgactccccggctgtacgaagccgaccgctccttcagggaccgggagcctcacgactacagccaccaccaccaccaccaccaccacccgctgtctgtggaccctcggcgggagcacgagcggggaggccacctggacgagcgggagcgcttgcacatgctcagagaagactacgagcacacgcggctccactccgtgcaccccgcctccctcgacggacacctcccccaccccagcctcatcaccccgggactccccagcatgcactatccccgcatcagccccaccgcgggcaaccagaacggactcctcaacaagacccctccgacagcagcgctgagcgcacctcccccgctcatctccacgctggggggccgcccggtctctcccagaaggacgactcctctgtccgcagagataagggagaggcccccttcccacacgctgaaggatatcgaggcccgataa

SEQ ID NO: 106 - DNA, 522 bp, Homo sapiens
atgatggcgcagctcttcctactccaagccaacgctgtccttcccctttcccatgaaatcaaggtcaagaggcaaataagactccctgctccactctaccccccagagagaaatgattctcgctcctttcagatcccccaggatctgagggagaaaggatgggaggaggggcagcagcatttcgctggaaaggcagcagatgcttttccagccccggttcagctggaaggcttggaggccggccagaccactctggcgtctcctgaagtgggtccctggagaccgaagaggctcagtggagtctgtctgttgtcagcactgctgcctgatccctgcaagacaaatggcactttccttcttcagaagcatcatctgccttcattattagcagtaatattattcccagttattattcttaccggtgccagttttgcacatctttttgttgctctatttgtgtctcatttacttctcaaattgcccctgggggcaggaatgaggatgcagagagatgcacgttaa

SEQ ID NO: 107 - DNA, 1614 bp, Homo sapiens
atgggggcgacgggggcggcggagccgctgcaatccgtgctgtgggtgaagcagcagcgctgcgccgtgagcctggagcccgcgcgggctctgctgcgctggtggcggagcccggggcccggagccggcgcccccggcgcggatgcctgctctgtgcctgtatctgagatcatcgccgttgaggaaacagacgttcacgggaaacatcaaggcagtggaaaatggcagaaaatggaaaagccttacgcttttacagttcactgtgtaaagagagcacgacggcaccgctggaagtgggcgcaggtgactttctggtgtccagaggagcagctgtgtcacttgtggctgcagaccctgcgggagatgctggagaagctgacgtccagaccaaagcatttactggtatttatcaacccgtttggaggaaaaggacaaggcaagcggatatatgaaagaaaagtggcaccactgttcaccttagcctccatcaccactgacatcatcgttactgaacatgctaatcaggccaaggagactctgtatgagattaacatagacaaatacgacggcatcgtctgtgtcggcggagatggtatgttcagcgaggtgctgcacggtctgattgggaggacgcagaggagcgccggggtcgaccagaaccacccccgggctgtgctggtccccagtagcctccggattggaatcattcccgcagggtcaacggactgcgtgtgttactccaccgtgggcaccagcgacgcagaaacctcggcgctgcatatcgttgttggggactcgctggccatggatgtgtcctcagtccaccacaacagcacactccttcgctactccgtgtccctgctgggctacggcttctacggggacatcatcaaggacagtgagaagaaacggtggttgggtcttgccagatacgacttttcaggtttaaagaccttcctctcccaccactgctatgaagggacagtgtccttcctccctgcacaacacacggtgggatctccaagggataggaagccctgccgggcaggatgctttgtttgcaggcaaagcaagcagcagctggaggaggagcagaagaaagcactgtatggtttggaagctgcggaggacgtggaggagtggcaagtcgtctgtgggaagtttctggccatcaatgccacaaacatgtcctgtgcttgtcgccggagccccaggggcctctccccggctgcccacttgggagacgggtcttctgacctcatcctcatccggaaatgctccaggttcaattttctgagatttctcatcaggcacaccaaccagcaggaccagtttgacttcacttttgttgaagtttatcgcgtcaagaaattccagtttacgtcgaagcacatggaggatgaggacagcgacctcaaggagggggggaagaagcgctttgggcacatttgcagcagccacccctcctgctgctgcaccgtctccaacagctcctggaactgcgacggggaggtcctgcacagccctgccatcgaggtcagagtccactgccagctggttcgactctttgcacgaggaattgaagagaatccgaagccagactcacacagctga

SEQ ID NO: 108 - DNA, 612 bp, Homo sapiens
atggcgcgtccgcggccccgcgagtacaaagcgggcgacctggtcttcgccaagatgaagggctacccgcactggccggcccggattgatgaactcccagagggcgctgtgaagcctccagcaaacaagtatcctatcttcttttttggcacccatgaaactgcatttctaggtcccaaagacctttttccatataaggagtacaaagacaagtttggaaagtcaaacaaacggaaaggatttaacgaaggattgtgggaaatagaaaataacccaggagtaaagtttactggctaccaggcaattcagcaacagagctcttcagaaactgagggagaaggtggaaatactgcagatgcaagcagtgaggaagaaggtgatagagtagaagaagatggaaaaggcaaaagaaagaatgaaaaagcaggctcaaaacggaaaaagtcatatacttcaaagaaatcctctaaacagtcccggaaatctccaggagatgaagatgacaaagactgcaaagaagaggaaaacaaaagcagctctgagggtggagatgcgggcaacgacacaagaaacacaacttcagacttgcagaaaaccagtgaagggacctaa

SEQ ID NO: 109 - DNA, 2004 bp, Homo sapiens
atgcatcaccaacagcgaatggctgccttagggacggacaaagagctgagtgatttactggatttcagtgcgatgttttcacctcctgtgagcagtgggaaaaatggaccaacttctttggcaagtggacattttactggctcaaatgtagaagacagaagtagctcagggtcctgggggaatggaggacatccaagcccgtccaggaactatggagatgggactccctatgaccacatgaccagcagggaccttgggtcacatgacaatctctctccaccttttgtcaattccagaatacaaagtaaaacagaaaggggctcatactcatcttatgggagagaatcaaacttacagggttgccaccagcagagtctccttggaggtgacatggatatgggcaacccaggaaccctttcgcccaccaaacctggttcccagtactatcagtattctagcaataatccccgaaggaggcctcttcacagtagtgccatggaggtacagacaaagaaagttcgaaaagttcctccaggtttgccatcttcagtctatgctccatcagcaagcactgccgactacaatagggactcgccaggctatccttcctccaaaccagcaaccagcactttccctagctccttcttcatgcaagatggccatcacagcagtgacccttggagctcctccagtgggatgaatcagcctggctatgcaggaatgttgggcaactcttctcatattccacagtccagcagctactgtagcctgcatccacatgaacgtttgagctatccatcacactcctcagcagacatcaattccagtcttcctccgatgtccactttccatcgtagtggtacaaaccattacagcacctcttcctgtacgcctcctgccaacgggacagacagtataatggcaaatagaggaagcggggcagccggcagctcccagactggagatgctctggggaaagcacttgcttcgatctattctccagatcacactaacaacagcttttcatcaaacccttcaactcctgttggctctcctccatctctctcagcaggcacagctgtttggtctagaaatggaggacaggcctcatcgtctcctaattatgaaggacccttacactctttgcaaagccgaattgaagatcgtttagaaagactggatgatgctattcatgttctccggaaccatgcagtgggcccatccacagctatgcctggtggtcatggggacatgcatggaatcattggaccttctcataatggagccatgggtggtctgggctcagggtatggaaccggccttctttcagccaacagacattcactcatggtggggacccatcgtgaagatggcgtggccctgagaggcagccattctcttctgccaaaccaggttccggttccacagcttcctgtccagtctgcgacttcccctgacctgaacccaccccaggacccttacagaggcatgccaccaggactacaggggcagagtgtctcctctggcagctctgagatcaaatccgatgacgagggtgatgagaacctgcaagacacgaaatcttcggaggacaagaaattagatgacgacaagaaggatatcaaatcaattactagcaataatgacgatgaggacctgacaccagagcagaaggcagagcgtgagaaggagcggaggatggccaacaatgcccgagagcgtctgcgggtccgtgacatcaacgaggctttcaaagagctcggccgcatggtgcagctccacctcaagagtgacaagccccagaccaagctcctgatcctccaccaggcggtggccgtcatcctcagtctggagcagcaagtccgagaaaggaatctgaatccgaaagctgcgtgtctgaaaagaagggaggaagagaaggtgtcctcagagcctccccctctctccttggccggcccacaccctggaatgggagacgcatcgaatcacatgggacagatgtaa

SEQ ID NO: 110 - DNA, 1146 bp, Homo sapiens
atggacggagtaggggttcccgcttccatgtacggagaccctcacgcgccgcggccgatccccccggttcaccacctgaaccacgggccgccgctccacgccacacagcactacggcgcgcacgccccgcaccccaatgtcatgccggccagtatgggatccgctgtcaacgacgccttgaagcgggacaaggacgcgatctatgggcacccgttgtttcctctgttagctctggtctttgagaagtgcgagctggcgacctgcactccccgggaacctggagtggctggcggagacgtctgctcctccgactccttcaacgaggacatcgcggtcttcgccaagcaggttcgcgccgaaaagccacttttttcctcaaatccagagctggacaatttgatgatacaagcaatacaagtactaaggtttcatcttttggagttagaaaaggtccacgaactgtgcgataacttctgccaccgatacattagctgtttgaaggggaaaatgcccatcgacctcgtcattgatgaaagagacggcagctccaagtcagatcatgaagaactttcaggctcctccacaaatctcgctgaccataacccttcttcttggcgagaccacgatgatgcaacctcaacccactcagcaggcaccccagggccctccagtgggggccatgcttcccagagcggagacaacagcagtgagcaaggggatggtttagacaacagtgtagcttcacctggtacaggtgacgatgatgatccggataaggacaaaaaacgccagaagaaaagaggcattttccccaaagtagcaacaaatatcatgagagcatggctcttccagcatctcacacatccgtacccttccgaagagcagaagaaacagttagcgcaagacacaggacttacaattctccaagtaaacaactggtttattaatgccagaagaagaatagtacagcccatgattgaccagtcaaatcgagcagtgagccaaggagcagcatatagtccagagggtcagcccatggggagctttgtgttggatggtcagcaacacatggggatccggcctgcaggacctatgagtggaatgggcatgaatatgggcatggatgggcaatggcactacatgtaa

SEQ ID NO: 111 - DNA, 2946 bp, Homo sapiens
atggacggtttcgccggcagtctcgatgatagtatttctgctgcaagtacttctgatgttcaagatcgcctgtcagctcttgagtcacgagttcagcaacaagaagatgaaatcactgtgctaaaggcggctttggctgatgttttgaggcgtcttgcaatctctgaagatcatgtggcctcagtgaaaaaatcagtctcaagtaaaggccaaccaagccctcgagcagttattcccatgtcctgtataaccaatggaagtggtgcaaacagaaaaccaagtcataccagtgctgtctcaattgcaggaaaagaaactctttcatctgctgctaaaagtggtacagaaaaaaagaaagaaaaaccacaaggacagagagaaaaaaaagaggaatctcattctaatgatcaaagtccacaaattcgagcatcaccttctccccagccctcttcacaacctctccaaatacacagacaaactccagaaagcaagaatgctactcccaccaaaagcataaaacgaccatcaccagctgaaaagtcacataattcttgggaaaattcagatgatagccgtaataaattgtcgaaaataccttcaacacccaaattaataccaaaagttaccaaaactgcagacaagcataaagatgtcatcatcaaccaagaaggagaatatattaaaatgtttatgcgcggtcggccaattaccatgttcattccttccgatgttgacaactatgatgacatcagaacggaactgcctcctgagaagctcaaactggagtgggcatatggttatcgaggaaaggactgtagagctaatgtttaccttcttccgaccggggaaatagtttatttcattgcatcagtagtagtactatttaattatgaggagagaactcagcgacactacctgggccatacagactgtgtgaaatgccttgctatacatcctgacaaaattaggattgcaactggacagatagctggcgtggataaagatggaaggcctctacaaccccacgtcagagtgtgggattctgttactctatccacactgcagattattggacttggcacttttgagcgtggagtaggatgcctggatttttcaaaagcagattcaggtgttcatttatgtgttattgatgactccaatgagcatatgcttactgtatgggactggcagaagaaagcaaaaggagcagaaataaagacaacaaatgaagttgttttggctgtggagtttcacccaacagatgcaaataccataattacatgcggtaaatctcatattttcttctggacctggagcggcaattcactaacaagaaaacagggaatttttgggaaatatgaaaagccaaaatttgtgcagtgtttagcattcttggggaatggagatgttcttactggagactcaggtggagtcatgcttatatggagcaaaactactgtagagcccacacctgggaaaggacctaaaggtgtatatcaaatcagcaaacaaatcaaagctcatgatggcagtgtgttcacactttgtcagatgagaaatgggatgttattaactggaggagggaaagacagaaaaataattctgtgggatcatgatctgaatcctgaaagagaaatagaggttcctgatcagtatggcacaatcagagctgtagcagaaggaaaggcagatcaatttttagtaggcacatcacgaaactttattttacgaggaacatttaatgatggcttccaaatagaagtacagggtcatacagatgagctttggggtcttgccacacatcccttcaaagatttgctcttgacatgtgctcaggacaggcaggtgtgcctgtggaactcaatggaacacaggctggaatggaccaggctggtagatgaaccaggacactgtgcagattttcatccaagtggcacagtggtggccataggaacgcactcaggcaggtggtttgttctggatgcagaaaccagagatctagtttctatccacacagacgggaatgaacagctctctgtgatgcgctactcaatagatggtaccttcctggctgtaggatctcatgacaactttatttacctctatgtagtctctgaaaatggaagaaaatatagcagatatggaaggtgcactggacattccagctacatcacacaccttgactggtccccagacaacaagtatataatgtctaactcgggagactatgaaatattgtactgggacattccaaatggctgcaaactaatcaggaatcgatcggattgtaaggacattgattggacgacatatacctgtgtgctaggatttcaagtatttggtgtctggccagaaggatctgatgggacagatatcaatgcactggtgcgatcccacaatagaaaggtgatagctgttgccgatgacttttgtaaagtccatctgtttcagtatccctgctccaaagcaaaggctcccagtcacaagtacagtgcccacagcagccatgtcaccaatgtcagttttactcacaatgacagtcacctgatatcaactggtggaaaagacatgagcatcattcagtggaaacttgtggaaaagttatctttgcctcagaatgagactgtagcggatactactctaaccaaagcccccgtctcttccactgaaagtgtcatccaatctaatactcccacaccgcctccttctcagcccttaaatgagacagctgaagaggaaagtagaataagcagttctcccacacttctggagaacagcctggaacaaactgtggagccaagtgaagaccacagcgaggaggagagtgaagagggcagcggagaccttggtgagcctctttatgaagagccatgcaacgagataagcaaggagcaggccaaagccacccttctggaggaccagcaagacccttcgccctcgtcctaa

SEQ ID NO: 112 - DNA, 396 bp, Homo sapiens
atggatttccagcagctggccgacgttgcggagaaatggtgctccaacacgcccttcgagctcatcgccaccgaggagaccgaacgcaggatggatttctacgccgaccccggcgtctccttctatgtgctgtgtccggacaacggctgcggcgacaattttcacgtgtggagtgagagcgaggactgcctgcctttcttgcagctagcacaggattacatctcctcctgcggcaagaagacgctccacgaagtcctggaaaaagtcttcaagtctttcagacctttactggggcttccggatgcagatgacgatgcgtttgaagagtacagtgctgacgtggaagaagaggagccagaggcggaccacccccagatgggggtcagccagcagtaa

SEQ ID NO: 113 - DNA, 3420 bp, Homo sapiens
gcggccgccgcccccggctgcgcgctgagccgccggccccccgagcgccacggccggagctgcggcggcggcatcatggccccgaccctgctccagaagctcttcaacaaaaggggcagcagcggcagctccgcggcggcgtctgcccagggcagggctcctaaggaaggacccgcctttagttggtcatgttcggagtttgacctgaatgagattcgcctgatagtttaccaggactgtgacaggagaggcagacaagtcttgtttgactctaaagctgttcaaaagattgaggaggtgacagctcagaaaacagaggatgttcctattaaaatatcagccaagtgctgccagggaagcagcagtgtcagcagcagtagcagcagcagcatctcttcccacagttcttctgggggatcttcacatcatgctaaggaacagcttccaaagtaccagtacacaagaccagcttccgatgtcaacatgttaggggaaatgatgtttggctcagttgccatgagttacaaaggctccaccttaaagatacactacatacgttctcctccacaactgatgattagtaaagtcttctctgctagaatgggcagcttctgtggaagtacaaataacttgcaagacagctttgagtacatcaaccaagatcctaatttgggaaaactgaacacaaatcaaaatagtttgggtccttgtcgtactggaagtaacctagcacacagcacaccagttgatatgccaagcagaggacagaatgaagacagggacagtggcattgctcgatcagcctcactaagcagtcttttgatcacacctttcccatctccaagctcctctacatcttcttccagcagttaccagcgccgctggcttcgaagtcagacaacaagtttggaaaatggcatcatcccaagaaggtcaactgatgagacattcagcttggctgaagaaacctgtagctctaatccagctatggttaggaggaagaaaattgccataagcatcatcttttccctatgtgagaaagaagaagcacaaaggaatttccaggacttcttcttttctcattttcccctgtttgaatctcacatgaacaggctgaagagtgcaattgaaaaggctatgatctcctgtaggaaaatagcagaatcaagtctccgagtccagttttatgtcagccgtttgatggaagctctgggagaattcagaggaactatctggaacttatattctgttccaaggatagctgaacctgtatggcttactatgatgtccggcactttggaaaaaaaccagctctgccagcgctttctcaaggagtttacacttctgatagaacagataaataaaaaccagttttttgctgccttactgactgcggtgttaacctaccacctggcctgggtcccaactgtcatgcctgtggatcaccctcccatcaaagccttctcagagaaacgtacctcccagtcagtgaacatgctggccaaaacacatccgtataatcctctttgggcacagctgggtgatctttacggagccataggctctccagtgagactgactcgcaccgtagtggtagggaagcagaaggacttagtccagcgaatactttatgtcctgacctactttctccgttgctctgagctacaagagaaccagctgacctggagtggcaatcatggtgaaggtgaccaagttttaaatgggagcaagatcataacagccttggagaaaggagaggtggaggagtctgagtatgtggtcattacggtgaggaacgagcccgctcttgtaccccccatcctaccaccaacagcagcagagagacacaacccctggccgacagggtttcctgagtgcccagagggcactgacagtagagacctgggtcttaaacctgacaaagaagctaacaggaggccagagcagggttctgaggcttgcagcgcagggtgcctggggccagcatcagacgcttcctggaaacctcagaatgcattttgtggggatgagaaaaataaagaggcaccgcaagatggctcttcaagacttcccagctgtgaagttttgggggcaggaatgaagatggaccagcaagctgtctgtgagctgttgaaagtggagatgcctacaagactgccagaccggtcagtggcctggccttgccctgacagacatctccgggagaaaccttccttagaaaaggtcactttccagattggaagctttgcatctccagagtctgactttgaaagccgcatgaaaaaaatggaggaacgggtgaaggcctgtggcccctccttggaggccagtgaggctgctgatgtggctcaggacccgcaggtttctaggagcccttttaaacctggctttcaggagaatgtttgctgtcctcagaatcggctttcagagggggatgaaggcgagtctgacaagggttttgcagaggacagaggcagcagaaacgacatggcagcagatattgctgggcagctcagccacgctgctgacttgggcacagcctcccacggtgcaggaggaacgggagggaggaggctggaggccactagaggtttgtatgtgaaggctgcggaaggacctgtgctggagcctgttgcccccaggtgtgtccagcggggccctggcctcgtggctggtgcgaatatcccctgtggggatgacaacaagaaggccaacttcaggactgaaggagacattccccgaaatgaaagctcagatagcgccctgggagacagtgacgacgaagcctgcgcttcagccatgctagatctgggtcacggtggtgacaggactggagggtccttggaagtggagctgcctctgccaaggtctcagagcatcagcacccagaatgtaaggaactttggccgctcacttctggcgggctactgccccacatacatgcctgatcttgtgcttcatgggaccggcagtgatgagaagctgaagcagtgcctggtggccgaccttgtccacacagtccatcatccagtcctggatgagccaatagctgaagctgtctgtattatcgcagacacggataaatggagtgtgcaggtagctacaagtcagaggaaagtgacggacaacatgaaactaggccaggatgtcctggtctctagtcaggtgtccagtttgcttcagtccattttacagctctataagcttcacctccctgctgatttttgcatcatgcatcttgaagatagactacaggagatgtaccttaaaagtaaaatgctatctgaatatctccggggacacacacgagtccatgtgaaagaattaggtgtcgtactggggattgaatccaacgacctgcctctgttgactgctattgccagtactcattctccttatgtggctcaaatactcttataa

SEQ ID NO: 114 - DNA, 1623 bp, Homo sapiens
atggactcggtggcttttgaggatgtagatgtgaacttcacccaggaggagtgggctttgctagatccttcccagaagaatctctacagagatgtgatgtgggaaaccatgaggaatctggcctctatagggaaaaaatggaaggaccagaacattaaagatcactacaaacaccgagggagaaatctaagaagtcatatgttagaaagactctatcaaactaaggatggtagtcagcgtggaggaatttttagccagtttgcaaatcagaatctgagcaagaaaatccctggagtgaaactctgtgaaagcattgtatatggagaagtcagcatgggtcagtcatcccttaatagacacatcaaagatcacagtggacatgaaccaaaggaatatcaggaatatggagagaagccagatacacgtaaccagtgttggaaacccttcagttctcaccactcctttcgaacacatgagataattcacactggagagaaactctatgattgtaaggaatgtggaaaaaccttcttttctctcaaaagaattagaagacacatcatcacacacagtggatatacaccatataaatgtaaggtgtgtgggaaagcttttgattatcccagtagatttcgaacacatgaaagaagtcacactggagagaaaccctatgaatgtcaggaatgtggaaaagccttcacttgtatcacaagtgttcgaagacacatgataaagcacactggagatggaccttataaatgtaaggtatgtgggaaaccctttcattctctgagttcatttcaagtgcatgaaagaattcacactggagaaaaaccctttaaatgtaagcaatgtggtaaagccttcagttgttccccaaccttacgaatacatgaaagaacccatactggagagaaaccttatgaatgcaagcagtgtgggaaggccttcagttatctcccctcccttcgactacatgaaagaattcacactggtgagaaacccttcgtatgtaaacaatgtggtaaagcctttagatctgccagtacctttcaaatacatgaaaggactcacactggagaaaaaccttatgaatgtaaggaatgtggggaagcattcagttgtatcccaagtatgcgaagacacatgataaaacatactggagaaggaccttataaatgtaaggtatgtgggaaaccctttcattctctgagtccatttcgaatacatgaaagaactcacactggagagaaaccttatgtatgtaaacattgtggtaaagctttcgtttcttcaacatcaattcgaatacatgaaagaactcatactggagagaaaccctatgagtgtaagcaatgtgggaaagccttcagttatctcaactcctttcgaacacatgaaatgattcacactggtgagaaaccctttgaatgtaagcgatgtggtaaagcctttagatcttctagttcctttcgactacatgaaaggactcacactggacagaaaccctatcattgcaaggaatgtgggaaagcctattcttgccgtgccagctttcagagacacatgttaacacatgctgaagatggaccaccttataaatgcatgtgggaaagcctttaa

SEQ ID NO: 115 - DNA, 981 bp, Homo sapiens
atggagaaggacggcctgtgccgcgctgaccagcagtacgaatgcgtggcggagatcggggagggcgcctatgggaaggtgttcaaggcccgcgacttgaagaacggaggccgtttcgtggcgttgaagcgcgtgcgggtgcagaccggcgaggagggcatgccgctctccaccatccgcgaggtggcggtgctgaggcacctggagaccttcgagcaccccaacgtggtcaggttgtttgatgtgtgcacagtgtcacgaacagacagagaaaccaaactaactttagtgtttgaacatgtcgatcaagacttgaccacttacttggataaagttccagagcctggagtgcccactgaaaccataaaggatatgatgtttcagcttctccgaggtctggactttcttcattcacaccgagtagtgcatcgcgatctaaaaccacagaacattctggtgaccagcagcggacaaataaaactcgctgacttcggccttgcccgcatctatagtttccagatggctctaacctcagtggtcgtcacgctgtggtacagagcacccgaagtcttgctccagtccagctacgccacccccgtggatctctggagtgttggctgcatatttgcagaaatgtttcgtagaaagcctctttttcgtggaagttcagatgttgatcaactaggaaaaatcttggacgtgattggactcccaggagaagaagactggcctagagatgttgcccttcccaggcaggcttttcattcaaaatctgcccaaccaattgagaagtttgtaacagatatcgatgaactaggcaaagacctacttctgaagtgtttgacatttaacccagccaaaagaatatctgcctacagtgccctgtctcacccatacttccaggacctggaaaggtgcaaagaaaacctggattcccacctgccgcccagccagaacacctcggagctgaatacagcctga

SEQ ID NO: 116 - DNA, 4059 bp, Homo sapiens
atgcacgcattttgtgttggccagtatttggagcctgaccaagaaggcgtcaccataccagatctggggagtctctcctcacctctgatagacacagagaggaatctgggcctgcttctcggattacacgcttcctatttagcaatgagcacaccgctgtctcctgtcgagattgaatgtgccaaatggcttcagtcatccatcttctctggaggcctgcagaccagccagatccactacagctacaacgaggagaaagacgaggaccactgcagctccccagggggcacacctgccagcaaatctcgactctgctcccacagacgggccctgggggaccattcccaggcatttctgcaagccattgcagacaacaacattcaggatcacaacgtgaaggactttttgtgtcaaatagaaaggtactgtaggcagtgccatttgaccacaccgatcatgtttccccccgagcatcccgtggaagaggtcggtcgcttgctgttatgttgcctcttaaaacatgaagatttaggtcatgtggcattatctttagttcatgcaggtgcacttgatattgagcaagtaaagcacagaacgttgcctaagtcagtggtggatgtttgtagagttgtctaccaagcaaaatgttcgctcattaagactcatcaagaacagggccgttcttacaaggaggtctgcgctcctgtcatcaaacgtttgagattcctctttaatgaattgagacctgctgtttgtaatgacctctctataatgtctaagtttaaattgttaagttctttgccccattggaggaggatagctcagaagataattcgagaaccaaggaaaaagagagttcctaagaagccagaatctacggatgatgaagaaaaaattggaaacgaagagagtgatttagaagaagcttgcattttgcctcatagtccaataaatgtggacaagagacccattgcaattaaatcacccaaggacaaatggcagccgctgttgagtactgttacagatgttcacaaatacaagtggttgaagcagaatgtgcagggtctttatccgcagtctccactcctcagtacaattgctgaatttgcccttaaagaagagccagtggatgtggaaaagagaaagtgcctactaaaacagttggagagagcagaggttcgcctggaagggatagatacaattttaaaattgtatctggtgagcaagaatttcttacttccatctgtgccgtatgcgatgttttgtggatggcaaagacttattcctgagggaatcgatataggggaacctcttactgattgtttaaaggatgttgatttgatcccgccttttaatcggatgctgctggaagtcacctttggcaagctgtacgcttgggctgttcagaacattcgaaatgttttggtggatgccagtgccaaatttaaagagcttggtatccagccggttcccctgcaaaccatcaccaatgagaacccatcgggaccgagcctggggaccatcccgcaagcccacttcctcctggtgatgctcagcatgctcaccctgcagcacagcgcaaacaaccttgacctcctgctcaattccggcacgctggccctcgctcagacggcactgcgcctgattggccccagttgtgacagcgttgaggaagatatgaatgcttctgcccaaggtgcttctgccacagttttggaagaaacaaggaaggaaacggctcctgtgcagctccctgtttcagggccagaactggctgccatgatgaagattggaacaagggtcatgagaggtgtggactggaaatggggcgatcaggatgggcctcctccaggcctaggccgagtgattggtgagctgggagaggacgggtggataagagtccagtgggacacaggcagcaccaactcctacaggatggggaaagaaggaaaatacgacctcaagctggcagagctgccagcccctgcacagccctcagcagaggattcggacacagaggacgactctgaagccgaacaaactgaaaggaacattcaccccactgcaatgatgtttaccagcactattaacttactgcagactctttgtctgtctgctggagttcatgctgagatcatgcagagtgaagccaccaagactttatgcggactgctgcgaatgttagtggaaagcggaacgacggacaagacatcttctccaaacaggctggtgtacagggagcaacaccggagctggtgcacgctggggtttgtgcagagcatcgctctcacgctgcaggtgtgcggcgccctcagctccccgcagtggatcacgctgctcatgaaggttgtggaagggcacgcacccttcactgccacctcgctgcagaggcagatcttagctgtgcatttgttgcaagcagtccttccgtcatgggacaagaccgaaagggtgagggacatgaaatgcctcatggagaagctgtttgacttcttggggagcttgctcactatgtgctcctctgacgtgccgttactcagagagtccacgctgaggcggcgcagggtgtgcccgcaggcctcgctgactgccacccacagcagcacactggcggaggaggtggtggcactgctgcacacgctgcactccctgactcggtggaatgggttcatcaacaagtacatcaactcccagctccgctccatcacccacagctttgcgggaaggccttccaaaggggcccagttagatgactacttccctgattccgagaaccctgaagtggggggcctcatggcggtcctggctgtggttggaggcatcgatggtcgcctgtgcctgggcggccaagttgtgcacgatgactttggagaagtcaccatgactcgcatcaccctgaagggcaaaatcaccgtgcagttctctgacatgcggacgtgtcgcgtttgcccattgaatcagctgaaaccactccctgccgtggcctttaatgtgaacaacctgcccttcacagagcccatgctgtctgtctgggctcagttggtgaacctcgctggaagcaagttagaaaagcacaaaataaagaaatcgactaaacaggcctttgcaggacaagtggacctggacctgctgcggcgccagcagttgaagctatacatcctgaaagcaggtcgggcgctgttctcccaccaggataaactgcggcagatcctgtctcagccagctgttcaggagactggaactgttcacacagatgatggagcagtggtatcacctgaccttggggacatgtctcctgaagggccgcagccccccatgatcctcttgcagcagctgctggcctcggccacccagccgtctcctgtgaaggccatatttgataaacaggaacttgagactgctgcactggccgttgtggagtccactcacccttcgagcccaggatttgaagactgcagctccagtgaggccaccacgcctgtcaacgtgcagcacatccgccctgccagagtgaagaggcgcaagcagtcacccgttcccgctctgccgatcgtggtgcagctcatggagatgggatttcccagaaggaacatcgagtttgccctgaagtctctcactggtgcttccgggaatgcgtccggcttgcctggtgtggaagccttggtcgggtggctgctggaccactccgacatacaggtcacggagctctcagatgcagacacggtgtccgacgagtattctgacgaggaggtggtggaggacatggatgatgccgcctactccatgtctactggtgctgttgtgacggagagccagacgtacaaaaaccgagctggtttcttgggtaatgatgattatgctgtatatgtgagagagaatattcaggtgggaatgatggttagatgctgccgaacatacgaagaagtgtgcgaaggtgatgtgatgttggcaaagtcatcaagctggacagagatggattgcatgatctcaatgtgcagtgtgactggcagcagaaagggggcatctactggtttaggtacattcatgtggaacttatag

SEQ ID NO: 117 - DNA, 1284 bp, Homo sapiens
atggctgtgctggcggcacttctgcgcagcggcgcccgcagccgcagccccctgctccggaggctggtgcaggaaataagatatgtggaacggagttatgtatcaaaacccactttgaaggaagtggtcatagtaagtgctacaagaacacccattggatcttttttaggcagcctttccttgctgccagccactaagcttggttccattgcaattcagggagccattgaaaaggcagggattccaaaagaagaagtgaaagaagcatacatgggtaatgttctacaaggaggtgaaggacaagctcctacaaggcaggcagtattgggtgcaggcttacctatttctactccatgtaccaccataaacaaagtttgtgcttcaggaatgaaagccatcatgatggcctctcaaagtcttatgtgtggacatcaggatgtgatggtggcaggtgggatggagagcatgtccaatgttccatatgtaatgaacagaggatcaacaccatatggtggggtaaagcttgaagatttgattgtaaaagacgggctaactgatgtctacaataaaattcatatgggcagctgtgctgagaatacagcaaagaagctgaatattgcacgaaatgaacaggacgcttatgctattaattcttataccagaagtaaagcagcatgggaagctgggaaatttggaaatgaagttattcctgtcacagttacagtaaaaggtcaaccagatgtagtggtgaaagaagatgaagaatataaacgtgttgattttagcaaagttccaaagctgaagacagttttccagaaagaaaatggcacagtaacagctgccaatgccagtacactgaatgatggagcagctgctctggttctcatgacggcagatgcagcgaagaggctcaatgttacaccactggcaagaatagtagcatttgctgacgctgctgtagaacctattgattttccaattgctcctgtatatgctgcatctatggttcttaaagatgtgggattgaaaaaagaagatattgcaatgtgggaagtaaatgaagcctttagtctggttgtactagcaaacattaaaatgttggagattgatccccaaaaagtgaatatcaatggaggagctgtttctctgggacatccaattgggatgtctggagccaggattgttggtcatttgactcatgccttgaagcaaggagaatacggtcttgccagtatttgcaatggaggaggaggtgcttctgccatgctaattcagaagctgtag

SEQ ID NO: 118 - DNA, 1095 bp, Homo sapiens
atgcctcactcgtacccagccctttctgctgagcagaagaaggagttgtctgacattgccctgcggattgtagccccgggcaaaggcattctggctgcggatgagtctgtaggcagcatggccaagcggctgagccaaattggggtggaaaacacagaggagaaccgccggctgtaccgccaggtcctgttcagtgctgatgaccgtgtgaaaaagtgcattggaggcgtcattttcttccatgagaccctctaccagaaagatgataatggtgttcccttcgtccgaaccatccaggataagggcatcgtcgtgggcatcaaggttgacaagggtgtggtgcctctagctgggactgatggagaaaccaccactcaagggctggatgggctctcagaacgctgtgcccaatacaagaaggatggtgctgactttgccaagtggcgctgtgtgctgaaaatcagtgagcgtacaccctctgcacttgccattctggagaacgccaacgtgctggcccgttatgccagtatctgccagcagaatggcattgtgcctattgtggaacctgaaatattgcctgatggagaccacgacctcaaacgttgtcagtatgttacagagaaggtcttggctgctgtgtacaaggccctgagtgaccatcatgtatacctggaggggaccctgctcaagcccaacatggtgaccccgggccatgcctgtcccatcaagtataccccagaggagattgccatggcaactgtcactgccctgcgtcgcactgtgcccccagctgtcccaggagtgaccttcctgtctgggggtcagagcgaagaagaggcatcattcaacctcaatgccatcaaccgctgcccccttccccgaccctgggcgcttaccttctcctatgggcgtgccctgcaagcctctgcactcaatgcctggcgagggcaacgggacaatgctggggctgccactgaggagttcatcaagcgggctgaggtgaatgggcttgcagcccagggcaagtatgaaggcagtggagaagatggtggagcagcagcacagtcactctacattgccaaccatgcctactga

SEQ ID NO: 119 - DNA, 1371 bp, Homo sapiens
atggaggcgctgggtgacctggagggaccacgcgcaccaggaggtgatgatcctgcaggaagtgcaggagagacccccgggtggctttcgagagaacaggtttttgtactgatatcggcagcttcggtgaacttaggttccatgatgtgctattctatacttggaccgtttttccccaaagaggctgaaaagaagggagccagcaatacaattatcggtatgatctttggatgttttgctttgttcgagttgctggcatccttggtatttggaaactatcttgtacatattggagcaaaatttatgtttgtagcaggaatgtttgtctcaggaggagttacaattctctttggtgtattggaccgagttccagatgggccagtatttattgctatgtgttttctagtgagagtaatggatgcagttagctttgctgcagcaatgactgcatcttcttctatcctggcaaaggcttttccaaataacgtggctacggtattgggaagtcttgagactttttctggactggggctaatactaggtcctcctgtaggtggctttttgtatcaatcctttggctatgaagtgccttttattgttctgggatgcgtcgttttgctgatggtaccactcaatatgtatattttacccaattacgagtctgatccaggtgaacactcattctggaaactgatcgctttacccaaagttggccttatagccttcgtcatcaactcactcagctcgtgttttggcttcctcgatcctactctgtctctctttgttttggagaagttcaatttaccagctggatatgtgggactagtattcctgggtatggcactgtcctatgccatctcttcaccactatttggtctcctaagtgataaaaggccacctctaaggaaatggcttctggtgtttggcaacttaatcacagccgggtgctacatgctcttagggcctgtcccaatcttgcatattaaaagtcagctctggctgctggtgctgatattagttgtaagtggcctctctgctggaatgagtataattccaactttcccggaaattctcagttgtgcacatgaaaatgggtttgaagagggattaagtacattgggacttgtatcaggtctttttagtgcaatgtggtcaattggtgcttttatgggaccaacgctgggtggatttctgtatgagaaaattggttttgaatgggcagcagctatacaaggtctatgggctctgataagtggattagccatgggcttgttttatctactggagtattcaaggagaaaaaggtctaaatctcaaaacatcctcagcacagaggaggaacgaactactctcttgcctaatgaaacctag

SEQ ID NO: 120 - DNA, 5058 bp, Homo sapiens
atgaaactgcgtggagtcagcctggctgccggcttgttcttactggccctgagtctttgggggcagcctgcagaggctgcggcttgctatgggtgttctccaggatcaaagtgtgactgcagtggcataaaaggggaaaagggagagagagggtttccaggtttggaaggacacccaggattgcctggatttccaggtccagaagggcctccggggcctcggggacaaaagggtgatgatggaattccagggccaccaggaccaaaaggaatcagaggtcctcctggacttcctggatttccagggacaccaggtcttcctggaatgccaggccacgatggggccccaggacctcaaggtattcccggatgcaatggaaccaagggagaacgtggatttccaggcagtcccggttttcctggtttacagggtcctccaggaccccctgggatcccaggtatgaagggtgaaccaggtagtataattatgtcatcactgccaggaccaaagggtaatccaggatatccaggtcctcctggaatacaaggcctacctggtcccactggtataccagggccaattggtcccccaggaccaccaggtttgatgggccctcctggtccaccaggacttccaggacctaaggggaatatgggcttaaatttccagggacccaaaggtgaaaaaggtgagcaaggtcttcagggcccacctgggccacctgggcagatcagtgaacagaaaagaccaattgatgtagagtttcagaaaggagatcagggacttcctggtgaccgagggcctcctggacctccagggatacgtggtcctccaggtcccccaggtggtgagaaaggtgagaagggtgagcaaggagagccaggcaaaagaggtaaaccaggcaaagatggagaaaatggccaaccaggaattcctggtttgcctggtgatcctggttaccctggtgaacccggaagggatggtgaaaagggccaaaaaggtgacactggcccacctggacctcctggacttgtaattcctagacctgggactggtataactataggagaaaaaggaaacattgggttgcctgggttgcctggagaaaaaggagagcgaggatttcctggaatacagggtccacctggccttcctggacctccaggggctgcagttatgggtcctcctggccctcctggatttcctggagaaaggggtcagaaaggtgatgaaggaccacctggaatttccattcctggacctcctggacttgacggacagcctggggctcctgggcttccagggcctcctggccctgctggccctcacattcctcctagtgatgagatatgtgaaccaggccctccaggccccccaggatctccaggtgataaaggactccaaggagaacaaggagtgaaaggtgacaaaggtgacacttgcttcaactgcattggaactggtatttcagggcctccaggtcaacctggtttgccaggtctcccaggtcctccaggatctcttggtttccctggacagaaaggggaaaaaggacaagctggtgcaactggtcccaaaggattaccaggcattccaggagctccaggtgctccaggctttcctggatctaaaggtgaacctggtgatatcctcacttttccaggaatgaagggtgacaaaggagagttgggttcccctggagctccagggcttcctggtttacctggcactcctggacaggatggattgccagggcttcctggcccgaaaggagagcctggtggaattacttttaagggtgaaagaggtccccctgggaacccaggtttaccaggcctcccagggaatatagggcctatgggtccccctggtttcggccctccaggcccagtaggtgaaaaaggcatacaaggtgtggcaggaaatccaggccagccaggaataccaggtcctaaaggggatccaggtcagactataacccagccggggaagcctggcttgcctggtaacccaggcagagatggtgatgtaggtcttccaggtgaccctggacttccagggcaaccaggcttgccagggatacctggtagcaaaggagaaccaggtatccctggaattgggcttcctggaccacctggtcccaaaggctttcctggaattccaggacctccaggagcacctgggacacctggaagaattggtctagaaggccctcctgggccacccggctttccaggaccaaagggtgaaccaggatttgcattacctgggccacctgggccaccaggacttccaggtttcaaaggagcacttggtccaaaaggtgatcgtggtttcccaggacctccgggtcctccaggacgcactggcttagatgggctccctggaccaaaaggtgatgttggaccaaatggacaacctggaccaatgggacctcctgggctgccaggaataggtgttcagggaccaccaggaccaccagggattcctgggccaataggtcaacctggtttacatggaataccaggagagaagggggatccaggacctcctggacttgatgttccaggacccccaggtgaaagaggcagtccagggatccccggagcacctggtcctataggacctccaggatcaccagggcttccaggaaaagcaggtgcctctggatttccaggtaccaaaggtgaaatgggtatgatgggacctccaggcccaccaggacctttgggaattcctggcaggagtggtgtacctggtcttaaaggtgatgatggcttgcagggtcagccaggacttcctggccctacaggagaaaaaggtagtaaaggagagcctggccttccaggccctcctggaccaatggatccaaatcttctgggctcaaaaggagagaagggggaacctggcttaccaggtatacctggagtttcagggccaaaaggttatcagggtttgcctggagacccagggcaacctggactgagtggacaacctggattaccaggaccaccaggtcccaaaggtaaccctggtctccctggacagccaggtcttataggacctcctggacttaaaggaaccatcggtgatatgggttttccagggcctcagggtgtggaagggcctcctggaccttctggagttcctggacaacctggctccccaggattacctggacagaaaggcgacaaaggtgatcctggtatttcaagcattggtcttccaggtcttcctggtccaaagggtgagcctggtctgcctggatacccagggaaccctggtatcaaaggttctgtgggagatcctggtttgcccggattaccaggaacccctggagcaaaaggacaaccaggccttcctggattcccaggaaccccaggccctcctggaccaaaaggtattagtggccctcctgggaaccccggccttccaggagaacctggtcctgtaggtggtggaggtcatcctgggcaaccagggcctccaggcgaaaaaggcaaacccggtcaagatggtattcctggaccagctggacagaagggtgaaccaggtcaaccaggctttggaaacccaggaccccctggacttccaggactttctggccaaaagggtgatggaggattacctgggattccaggaaatcctggccttccaggtccaaagggcgaaccaggctttcacggtttccctggtgtgcagggtcccccaggccctcctggttctccgggtccagctctggaaggacctaaaggcaaccctgggccccaaggtcctcctgggagaccaggtctaccaggtccagaaggtcctccaggtctccctggaaatggaggtattaaaggagagaagggaaatccaggccaacctgggctacctggcttgcctggtttgaaaggagatcaaggaccaccaggactccagggtaatcctggccggccgggtctcaatggaatgaaaggagatcctggtctccctggtgttccaggattcccaggcatgaaaggacccagtggagtacctggatcagctggccctgagggggaaccgggacttattggtcctccaggtcctcctggattacctggtccttcaggacagagtatcataattaaaggagatgctggtcctccaggaatccctggccagcctgggctaaagggtctaccaggaccccaaggacctcaaggcttaccaggtccaactggccctccaggagatcctggacgcaatggactccctggctttgatggtgcaggagggcgcaaaggagacccaggtctgccaggacagccaggtacccgtggtttggatggtccccctggtccagatggattgcaaggtcccccaggtccccctggaacctcctctgttgcacatggatttcttattacacgccacagccagacaacggatgcaccacaatgcccacagggaacacttcaggtctatgaaggcttttctctcctgtatgtacaaggaaataaaagagcccacggtcaagacttggggacggctggcagctgccttcgtcgctttagtaccatgcctttcatgttctgcaacatcaataatgtttgcaactttgcttcaagaaatgactattcttactggctctctaccccagagcccatgccaatgagcatgcaacccctaaagggccagagcatccagccattcattagtcgatgtgcagtatgtgaagctccagctgtggtgatcgcagttcacagtcagacgatccagattccccattgtcctcagggatgggattctctgtggattggttattccttcatgatgcatacaagtgcaggggcagaaggctcaggtcaagccctagcctcccctggttcctgcttggaagagtttcgttcagctcccttcatcgaatgtcatgggaggggtacctgtaactactatgccaactcctacagcttttggctggcaactgtagatgtgtcagacatgttcagtaaacctcagtcagaaacgctgaaagcaggagacttgaggacacgaattagccgatgtcaagtgtgcatgaagaggacataa

SEQ ID NO: 121 - DNA, 849 bp, Homo sapiens
atgctgcctctgctgcgctgcgtgccccgtgtgctgggctcctccgtcgccggcctccgcgctgccgcgcccgcctcgcctttccggcagctcctgcagccggcaccccggctgtgcacccggcccttcgggctgctcagcgtgcgcgcaggttccgagcggcggccgggcctcctgcggcctcgcggaccctgcgcctgtggctgtggctgcggctcgctgcacaccgacggagacaaagcttttgttgatttcctgagtgatgaaattaaggaggaaagaaaaattcagaagcataaaaccctccctaagatgtctggaggttgggagctggaactgaatgggacagaagcgaaattagtgcggaaagttgccggggaaaaaatcacggtcactttcaacattaacaacagcatcccaccaacatttgatggtgaggaggaaccctcgcaagggcagaaggttgaagaacaggagcctgaactgacatcaactcccaatttcgtggttgaagttataaagaatgatgatggcaagaaggcccttgtgttggactgtcattatccagaggatgaggttggacaagaagacgaggctgagagtgacatcttctctatcagggaagttagctttcagtccactggcgagtctgaatggaaggatactaattatacactcaacacagattccttggactgggccttatatgaccacctaatggatttccttgccgaccgaggggtggacaacacttttgcagatgagctggtggagctcagcacagccctggagcaccaggagtacattacttttcttgaagacctcaagagttttgtcaagagccagtag

SEQ ID NO: 122 - DNA, 234 bp, Homo sapiens
atgcccaagtgtcccaagtgcaacaaggaggtgtacttcgccgagagggtgacctctctgggcaaggactggcatcggccctgcctgaagtgcgagaaatgtgggaagacgctgacctctgggggccacgctgagcacgaaggcaaaccctactgcaaccacccctgctacgcagccatgtttgggcctaaaggctttgggcggggcggagccgagagccacactttcaagtaa

SEQ ID NO: 123 - DNA, 576 bp, Homo sapiens
atggaggcgaaggcggcacccaagccagctgcaagcggcgcgtgctcggtgtcggcagaggagaccgaaaagtggatggaggaggcgatgcacatggccaaagaagccctcgaaaatactgaagttcctgttggctgtcttatggtctacaacaatgaagttgtagggaaggggagaaatgaagttaaccaaaccaaaaatgctactcgacatgcagaaatggtggccatcgatcaggtcctcgattggtgtcgtcaaagtggcaagagtccctctgaagtatttgaacacactgtgttgtatgtcactgtggagccgtgcattatgtgtgcagctgctctccgcctgatgaaaatcccgctggttgtatatggctgtcagaatgaacgatttggtggttgtggctctgttctaaatattgcctctgctgacctaccaaacactgggagaccatttcagtgtatccctggatatcgggctgaggaagcagtggaaatgttaaagaccttctacaaacaagaaaatccaaatgcaccaaaatcgaaagttcggaaaaaggaatgtcagaaatcttga

SEQ ID NO: 124 - DNA, 681 bp, Homo sapiens
atggggcccctgccgcgcaccgtggagctcttctatgacgtgctgtccccctactcctggctgggcttcgagatcctgtgccggtatcagaatatctggaacatcaacctgcagttgcggcccagcctcataacagggatcatgaaagacagtggaaacaagcctccaggtctgcttccccgcaaaggactatacatggcaaatgacttaaagctcctgagacaccatctccagattcccatccacttccccaaggatttcttgtctgtgatgcttgaaaaaggaagtttgtctgccatgcgtttcctcaccgccgtgaacttggagcatccagagatgctggagaaagcgtcccgggagctgtggatgcgcgtctggtcaaggaatgaagacatcaccgagccgcagagcatcctggcggctgcagagaaggctggtatgtctgcagaacaagcccagggacttctggaaaagatcgcaacgccaaaggtgaagaaccagctcaaggagaccactgaggcagcctgcagatacggagcctttgggttgcccatcaccgtggcccatgtggatggccaaacccacatgttatttggctctgaccggatggagctgctggcgcacctgctgggagagaagtggatgggccctatacctccagccgtgaatgccagactttaa

SEQ ID NO: 125 - DNA, 732 bp, Homo sapiens
atgtctggggatgcgaccaggaccctggggaaaggaagccagcccccagggccagtcccggaggggctgatccgcatctacagcatgaggttctgcccctattctcacaggacccgcctcgtcctcaaggccaaagacatcagacatgaagtggtcaacattaacctgagaaacaagcctgaatggtactatacaaagcacccttttggccacattcctgtcctggagaccagccaatgtcaactgatctatgaatctgttattgcttgtgagtacctggatgatgcttatccaggaaggaagctgtttccatatgacccttatgaacgagctcgccaaaagatgttattggagctattttgtaaggtcccacatttgaccaaggagtgcctggtagcgttgagatgtgggagagaatgcactaatctgaaggcagccctgcgtcaggaattcagcaacctggaagagattcttgagtatcagaacaccaccttctttggtggaacctgtatatccatgattgattacctcctctggccctggtttgagcggctggatgtgtatgggatactggactgtgtgagccacacgccagccctgcggctctggatatcagccatgaagtgggaccccacagtctgtgctcttctcatggataagagcattttccagggcttcttgaatctctattttcagaacaaccctaatgcctttgactttgggctgtgctga

SEQ ID NO: 126 - DNA, 528 bp, Homo sapiens
atgtggctctaccggaacccctacgtggaggcggagtatttccccaccaagccgatgtttgttattgcatttctctctccactgtctctgatcttcctggccaaatttctcaagaaggcagacacaagagacagcagacaagcctgcctggctgccagccttgccctggctctgaatggcgtctttaccaacacaataaaactgatcgtagggaggccacgcccagatttcttctaccgctgcttccctgatgggctagcccattctgacttgatgtgtacaggggataaggacgtggtgaatgagggccgaaagagcttccccagtggacattcttcctttgcatttgctggtctggcctttgcgtccttctacctggcagggaagttacactgcttcacaccacaaggccgtgggaaatcttggaggttctgtgcctttctgtcacctctactttttgcagctgtgattgcactgtcccgcacatgtgactacaagcatcactggcaaggaccctttaaatggtga

SEQ ID NO: 127 - DNA, 441 bp, Homo sapiens
atgggccgggtctcagggcttgtgccctctcgcttcctgacgctcctggcgcatctggtggtcgtcatcaccttattctggtcccgggacagcaacatacaggcctgcctgcctctcacgttcacccccgaggagtatgacaagcaggacattcatccacttcctctctgcaggctggtggccgcgctctctgtcaccctgggcctctttgcagtggagctggccggtttcctctcaggagtctccatgttcaacagcacccagagcctcatctccattggggctcactgtagtgcatccgtggccctgtccttcttcatattcgagcgttgggagtgcactacgtattggtacatttttgtcttctgcagtgcccttccagctgtcactgaaatggctttattcgtcaccgtctttgggctgaaaaagaaacccttctga

SEQ ID NO: 128 - DNA, 1194 bp, Homo sapiens
atgcaccatgtgtttaaagatcaccaaagaggagagaaaaggggattccttagtccagagaacaaaaactgcaggaggctggagctgagacgtgggtgttcctgcagctggggcctgtgctcccaggcactcatggcttctctggctggttcacttctccctggctcagacagatcaggagtggaaacatctgaatatgctcaaggaggagtgagtgacctgcaggaagaaggcaagaatgccatcaactcaccgatgtcccccgccctggtggatgttcaccctgaagacacccagcttgaggagaacgaggagcgcacgatgattgaccccacttccaaggaagaccccaagttcaaggaactggtcaaggtcctcctcgactggattaatgacgtgctggtggaggagaggatcattgtgaagcagctggaggaagacctgtatgacggccaggtgctgcagaagctcttggaaaaactggcagggtgcaagctgaatgtggctgaggtgacacagtccgaaatagggcagaaacagaagctgcagacggtgctggaagcagtacatgacctgctgcggccccgaggctgggcgctccggtggagcgtggactcaattcacgggaagaacctggtggccatcctccacctgctggtctctctggccatgcacttcagggcccccatccgccttcctgagcatgtaacggtgcaggtggtggtcgtgcggaaacgggaaggcctgctgcattccagccacatctcggaggagctgaccacaactacagagatgatgatgggccggttcgagcgggatgccttcgacacgctgttcgaccacgccccggataagctcagcgtggtgaagaagtctctcatcacttttgtgaacaagcacctgaacaagctgaatttggaggtgacggaactggagacccagtttgcagatggcgtgtacctggttctgctcatgggccttctggaagactactttgttcctctccaccacttctacctgactccggaaagcttcgatcagaaggtccacaatgtgtccttcgcctttgagctgatgctggacggaggcctcaagaaacccaaggctcgtcctgaagacgtggttaacttggacctcaaatccaccctgagggttctttacaacctgttcaccaagtacaagaacgtggagtga

SEQ ID NO: 129 - DNA, 837 bp, Homo sapiens
atggataaatcaggaatagattctcttgaccatgtgacatctgatgctgtggaacttgcaaatcgaagtgataactcttctgatagcagcttatttaaaactcagtgtatcccttactcacctaaaggggagaaaagaaaccccattcgaaaatttgttcgtacacctgaaagtgttcacgcaagtgattcatcaagtgactcatcttttgaaccaataccattgactataaaagctatttttgaaagattcaagaacaggaaaaagagatataaaaaaaagaaaaagaggaggtaccagccaacaggaagaccacggggaagaccagaaggaaggagaaatcctatatactcactaatagataagaagaaacaatttagaagcagaggatctggcttcccatttttagaatcagagaatgaaaaaaacgcaccttggagaaaaattttaacgtttgagcaagctgttgcaagaggattttttaactatattgaaaaactgaagtatgaacaccacctgaaagaatcattgaagcaaatgaatgttggtgaagatttagaaaatgaagattttgacagtcgtagatacaaatttttggatgatgatggatccatttctcctattgaggagtcaacagcagaggatgaggatgcaacacatcttgaagataacgaatgtgatatcaaattggcaggggatagtttcatagtaagttctgaattccctgtaagactgagtgtatacttagaagaagaggatattactgaagaagctgctttgtctaaaaagagagctacaaaagccaaaaatactggacagagaggcctgaaaatgtga

SEQ ID NO: 130 - DNA, 801 bp, Homo sapiens
atgtggtggtttcagcaaggcctcagtttccttccttcagcccttgtaatttggacatctgctgctttcatattttcatacattactgcagtaacactccaccatatagacccggctttaccttatatcagtgacactggtacagtagctccagaaaaatgcttatttggggcaatgctaaatattgcggcagttttatgcattgctaccatttatgttcgttataagcaagttcatgctctgagtcctgaagagaacgttatcatcaaattaaacaaggctggccttgtacttggaatactgagttgtttagggctttctattgtggcaaacttccagaaaacaaccctttttgctgcacatgtaagtggagctgtgcttacctttggtatgggctcattatatatgtttgttcagaccatcctttcctaccaaatgcagcccaaaatccatggcaaacaagtcttctggatcagactgttgttggttatctggtgtggagtaagtgcacttagcatgctgacttgctcatcagttttgcacagtggcaattttgggactgatttagaacagaaactccattggaaccccgaggacaaaggttatgtgcttcacatgatcactactgcagcagaatggtctatgtcattttccttctttgtttttttcctgacttacattcgtgattttcagaaaatttctttacgggtggaagccaatttacatggattaaccctctatgacactgcaccttgccctattaacaatgaacgaacacggctactttccagagatatttga

SEQ ID NO: 131 - DNA, 2136 bp, Homo sapiens
atggggtggctcccactcctgctgcttctgactcaatgcttaggggtccctgggcagcgctcgccattgaatgacttccaagtgctccggggcacagagctacagcacctgctacatgcggtggtgcccgggccttggcaggaggatgtggcagatgctgaagagtgtgctggtcgctgtgggcccttaatggactgccgggccttccactacaacgtgagcagccatggttgccaactgctgccatggactcaacactcgccccacacgaggctgcggcgttctgggcgctgtgacctcttccagaagaaagactacgtacggacctgcatcatgaacaatggggttgggtaccggggcaccatggccacgaccgtgggtggcctgccctgccaggcttggagccacaagttcccgaatgatcacaagtacacgcccactctccggaatggcctggaagagaacttctgccgtaaccctgatggcgaccccggaggtccttggtgctacacaacagaccctgctgtgcgcttccagagctgcggcatcaaatcctgccgggaggccgcgtgtgtctggtgcaatggcgaggaataccgcggcgcggtagaccgcacggagtcagggcgcgagtgccagcgctgggatcttcagcacccgcaccagcaccccttcgagccgggcaagttcctcgaccaaggtctggacgacaactattgccggaatcctgacggctccgagcggccatggtgctacactacggatccgcagatcgagcgagagttctgtgacctcccccgctgcgggtccgaggcacagccccgccaagaggccacaactgtcagctgcttccgcgggaagggtgagggctaccggggcacagccaataccaccactgcgggcgtaccttgccagcgttgggacgcgcaaatccctcatcagcaccgatttacgccagaaaaatacgcgtgcaaagaccttcgggagaacttctgccggaaccccgacggctcagaggcgccctggtgcttcacactgcggcccggcatgcgcgcggccttttgctaccagatccggcgttgtacagacgacgtgcggccccaggactgctaccacggcgcaggggagcagtaccgcggcacggtcagcaagacccgcaagggtgtccagtgccagcgctggtccgctgagacgccgcacaagccgcagttcacgtttacctccgaaccgcatgcacaactggaggagaacttctgccggaacccagatggggatagccatgggccctggtgctacacgatggacccaaggaccccattcgactactgtgccctgcgacgctgcgctgatgaccagccgccatcaatcctggaccccccagaccaggtgcagtttgagaagtgtggcaagagggtggatcggctggatcagcggcgttccaagctgcgcgtggttgggggccatccgggcaactcaccctggacagtcagcttgcggaatcggcagggccagcatttctgcggggggtctctagtgaaggagcagtggatactgactgcccggcagtgcttctcctcctgccatatgcctctcacgggctatgaggtatggttgggcaccctgttccagaacccacagcatggagagccaagcctacagcgggtcccagtagccaagatggtgtgtgggccctcaggctcccagcttgtcctgctcaagctggagagatctgtgaccctgaaccagcgtgtggccctgatctgcctgccccctgaatggtatgtggtgcctccagggaccaagtgtgagattgcaggctggggtgagaccaaaggtacgggtaatgacacagtcctaaatgtggccttgctgaatgtcatctctaaccaggagtgtaacatcaagcaccgaggacgtgtgcgggagagtgagatgtgcactgagggactgttggcccctgtgggggcctgtgagggtgactacgggggcccacttgcctgctttacccacaactgctgggtcctggaaggaattataatccccaaccgagtatgcgcaaggtcccgctggccagctgtcttcacgcgtgtctctgtgtttgtggactggattcacaaggtcatgagactgggttag

SEQ ID NO: 132 - DNA, 186 bp, Homo sapiens
atggaccccaactgctcttgcgccactggtggctcctgcacgtgcgccggctcctgcaagtgcaaagagtgcaaatgcacctcctgcaagaagagctgctgttcctgctgccccgtgggctgtgccaagtgtgcccagggctgcgtctgcaaaggggcatcggagaagtgcagctgctgtgcctga

SEQ ID NO: 133 - DNA, 816 bp, Homo sapiens
atggaggcgctgccgctgctagccgcgacaactccggaccacggccgccaccgaaggctgcttctgctgccgctactgctgttcctgctgccggctggagctgtgcagggctgggagacagaggagaggccccggactcgcgaagaggagtgccacttctacgcgggtggacaagtgtacccgggagaggcatcccgggtatcggtcgccgaccactccctgcacctaagcaaagcgaagatttccaagccagcgccctactgggaaggaacagctgtgatcgatggagaatttaaggagctgaagttaactgattatcgtgggaaatacttggttttcttcttctacccacttgatttcacatttgtgtgtccaactgaaattatcgcttttggcgacagacttgaagaattcagatctataaatactgaagtggtagcatgctctgttgattcacagtttacccatttggcctggattaatacccctcgaagacaaggaggacttgggccaataaggattccacttctttcagatttgacccatcagatctcaaaggactatggtgtatacctagaggactcaggccacactcttagaggtctcttcattattgatgacaaaggaatcctaagacaaattactctgaatgatcttcctgtgggtagatcagtggatgagacactacgtttggttcaagcattccagtacactgacaaacacggagaagtctgccctgctggctggaaacctggtagtgaaacaataatcccagatccagctggaaagctgaagtatttcgataaactgaattga

SEQ ID NO: 134 - DNA, 1446 bp, Homo sapiens
atgccccagacgtccgttgtcttctccagcatccttgggcccagctgtagcggacaggtgcagcctggcatgggggagcgtggaggcggggccggtggcggctccggggacctcatcttccaagatggacacctcatctctgggtccctggaggccctgatggagcaccttgttcccacggtggactattaccccgataggacgtacatcttcacctttctcctgagctcccgggtctttatgccccctcatgacctgctggcccgcgtggggcagatctgcgtggagcagaagcagcagctggaagccgggcctgaaaaggccaagctgaagtctttctcagccaagatcgtgcagctcctgaaggagtggaccgaggccttcccctatgacttccaggatgagaaggccatggccgagctgaaagccatcacacaccgtgtcacccagtgtgatgaggagaatggcacagtgaagaaggccattgcccagatgacacagagcctgttgctgtccttggctgcccggagccagctccaggaactgcgagagaagctccggccaccggctgtagacaaggggcccatcctcaagaccaagccaccagccgcccagaaggacatcctgggcgtgtgctgcgaccccctggtgctggcccagcagctgactcacattgagctggacagggtcagcagcatttaccctgaggacttgatgcagatcgtcagccacatggactccttggacaaccacaggtgccgaggggacctgaccaagacctacagcctggaggcctatgacaactggttcaactgcctgagcatgctggtggccactgaggtgtgccgggtggtgaagaagaaacaccggacccgcatgttggagttcttcattgatgtggcccgggagtgcttcaacatcgggaacttcaactccatgatggccatcatctctggcatgaacctcagtcctgtggcaaggctgaagaaaacttggtccaaggtcaagacagccaagtttgatgtcttggagcatcacatggacccgtccagcaacttctgcaactaccgtacagccctgcagggggccacgcagaggtcccagatggccaacagcagccgtgaaaagatcgtcatccctgtgttcaacctcttcgttaaggacatctacttcctgcacaaaatccataccaaccacctgcccaacgggcacattaactttaagaaattctgggagatctccagacagatccatgagttcatgacatggacacaggtagagtgtcctttcgagaaggacaagaagattcagagttacctgctcacggcgcccatctacagcgaggaagctctcttcgtcgcctcctttgaaagtgagggtcccgagaaccacatggaaaaagacagctggaagaccctcaggaccacccttctgaacagagcctga

SEQ ID NO: 135 - DNA, 648 bp, Homo sapiens
atggtgttcaggcgcttcgtggaggttggccgggtggcctatgtctcctttggacctcatgccggaaaattggtcgcgattgtagatgttattgatcagaacagggctttggtcgatggaccttgcactcaagtgaggagacaggccatgcctttcaagtgcatgcagctcactgatttcatcctcaagtttccgcacagtgcccaccagaagtatgtccgacaagcctggcagaaggcagacatcaatacaaaatgggcagccacacgatgggccaagaagattgaagccagagaaaggaaagccaagatgacagattttgatcgttttaaagttatgaaggcaaagaaaatgaggaacagaataatcaagaatgaagttaagaagcttcaaaaggcagctctcctgaaagcttctcccaaaaaagcacctggtactaagggtactgctgctgctgctgctgctgctgctgctgctaaagttccagcaaaaaagatcaccgccgcgagtaaaaaggctccagcccagaaggttcctgcccagaaagccacaggccagaaagcagcgcctgctccaaaagctcagaagggtcaaaaagctccagcccagaaagcacctgctccaaaggcatctggcaagaaagcataa

SEQ ID NO: 136 - DNA, 753 bp, Homo sapiens
atgaccctgtcgccacttctgctgttcctgccaccgctgctgctgctgctggacgtccccacggcggcggtgcaggcgtcccctctgcaagcgttagacttctttgggaatgggccaccagttaactacaagacaggcaatctatacctgcgggggcccctgaagaagtccaatgcaccgcttgtcaatgtgaccctctactatgaagcactgtgcggtggctgccgagccttcctgatccgggagctcttcccaacatggctgttggtcatggagatcctcaatgtcacgctggtgccctacggaaacgcacaggaacaaaatgtcagtggcaggtgggagttcaagtgccagcatggagaagaggagtgcaaattcaacaaggtggaggcctgcgtgttggatgaacttgacatggagctagccttcctgaccattgtctgcatggaagagtttgaggacatggagagaagtctgccactatgcctgcagctctacgccccagggctgtcgccagacactatcatggagtgtgcaatgggggaccgcggcatgcagctcatgcacgccaacgcccagcggacagatgctctccagccaccacacgagtatgtgccctgggtcaccgtcaatgggaaacccttggaagatcagacccagctccttacccttgtctgccagttgtaccagggcaagaagccggatgtctgcccttcctcaaccagctccctcaggagtgtttgcttcaagtga

SEQ ID NO: 137 - DNA, 816 bp, Homo sapiens
atggaagattcccacaagagtaccacgtcagagacagcacctcaacctggttcagcagttcagggagctcacatttctcatattgctcaacaggtatcatctttatcagaaagtgaggagtcccaggactcatccgacagcataggctcctcacagaaagcccacgggatcctagcacggcgcccatcttacagaaaaattttgaaagacttatcttctgaagatacacggggcagaaaaggagacggagaaaattctggagtttctgctgctgtcacttctatgtctgttccaactcccatctatcagactagcagcggacagtacattgccattgccccaaatggagccttacagttggcaagtccaggcacagatggagtacagggacttcagacattaaccatgacaaattcaggcagtactcagcaaggtacaactattcttcagtatgcacagacctctgatggacagcagatacttgtgcccagcaatcaggtggtcgtacaaactgcatcaggagatatgcaaacatatcagatccgaactacaccttcagctacttctctgccacaaactgtggtgatgacatctcctgtgactctcacctctcagacaactaagacagatgacccccaattgaaaagagaaataaggttaatgaaaaacagagaagctgctcgagaatgtcgcagaaagaagaaagaatatgtgaaatgcctggaaaaccgagttgcagtcctggaaaatcaaaataaaactctaatagaagagttaaaaactttgaaggatctttattccaataaaagtgtttga

SEQ ID NO: 138 - DNA, 1968 bp, Homo sapiens
atgcaggcggctcggatggccgcgagcttggggcggcagctgctgaggctcgggggcggaagctcgcggctcacggcgctcctggggcagccccggcccggccctgcccggcggccctatgccgggggtgccgctcagctggctctggacaagtcagattcccacccctctgacgctctgaccaggaaaaaaccggccaaggcggaatctaagtcctttgctgtgggaatgttcaaaggccagctcaccacagatcaggtgttcccatacccgtccgtgctcaacgaagagcagacacagtttcttaaagagctggtggagcctgtgtcccgtttcttcgaggaagtgaacgatcccgccaagaatgacgctctggagatggtggaggagaccacttggcagggcctcaaggagctgggggcctttggtctgcaagtgcccagtgagctgggtggtgtgggcctttgcaacacccagtacgcccgtttggtggagatcgtgggcatgcatgaccttggcgtgggcattaccctgggggcccatcagagcatcggtttcaaaggcatcctgctctttggcacaaaggcccagaaagaaaaatacctccccaagctggcatctggggagactgtggccgctttctgtctaaccgagccctcaagcgggtcagatgcagcctccatccgaacctctgctgtgcccagcccctgtggaaaatactataccctcaatggaagcaagctttggatcagtaatgggggcctagcagacatcttcacggtctttgccaagacaccagttacagatccagccacaggagccgtgaaggagaagatcacagcttttgtggtggagaggggcttcgggggcattacccatgggccccctgagaagaagatgggcatcaaggcttcaaacacagcagaggtgttctttgatggagtacgggtgccatcggagaacgtgctgggtgaggttgggagtggcttcaaggttgccatgcacatcctcaacaatggaaggtttggcatggctgcggccctggcaggtaccatgagaggcatcattgctaaggcggtagatcatgccactaatcgtacccagtttggggagaaaattcacaactttgggctgatccaggagaagctggcacggatggttatgctgcagtatgtaactgagtccatggcttacatggtgagtgctaacatggaccagggagccacggacttccagatagaggccgccatcagcaaaatctttggctcggaggcagcctggaaggtgacagatgaatgcatccaaatcatggggggtatgggcttcatgaaggaacctggagtagagcgtgtgctccgagatcttcgcatcttccggatctttgaggggacaaatgacattcttcggctgtttgtggctctgcagggctgtatggacaaaggaaaggagctctctgggcttggcagtgctctaaagaatccctttgggaatgctggcctcctgctaggagaggcaggcaaacagctgaggcggcgggcagggctgggcagcggcctgagtctcagcggacttgtccacccggagttgagtcggagtggcgagctggcagtacgggctctggagcagtttgccactgtggtggaggccaagctgataaaacacaagaaggggattgtcaatgaacagtttctgctgcagcggctggcagacggggccatcgacctctatgccatggtggtggttctctcgagggcctcaagatccctgagtgagggccaccccacggcccagcatgagaaaatgctctgtgacacctggtgtatcgaggctgcagctcggatccgagagggcatggccgccctgcagtctgacccctggcagcaagagctctaccgcaacttcaaaagcatctccaaggccttggtggagcggggtggtgtggtcaccagcaacccacttggcttctga

SEQ ID NO: 139 - DNA, 882 bp, Homo sapiens
atggatgctccccactccaaagcagccctggacagcattaacgagctgcccgagaacatcctgctggagctgttcacgcacgtgcccgcccgccagctgctgctgaactgccgcctggtctgcagcctctggcgggacctcatcgacctcatgaccctctggaaacgcaagtgcctgcgagagggcttcatcaccaaggactgggaccagcccgtggccgactggaaaatcttctacttcctacggagcctgcataggaacctcctgcgcaacccgtgtgctgaagaggatatgtttgcatggcaaattgatttcaatggtggggaccgctggaaggtggagagcctccctggagcccacgggacagattttcctgaccccaaagtcaagaagtattttgtcacatcctacgaaatgtgcctcaagtcccagctggtggaccttgtagccgagggctactgggaggagctactagacacattccggccggacatcgtggttaaggactggtttgctgccagagccgactgtggctgcacctaccaactcaaagtgcagctggcctcggctgactacttcgtgttggcctccttcgagcccccacctgtgaccatccaacagtggaacaatgccacatggacagaggtctcctacaccttctcagactacccccggggtgtccgctacatcctcttccagcatgggggcagggacacccagtactgggcaggctggtatgggccccgagtcaccaacagcagcattgtcgtcagccccaagatgaccaggaaccaggcctcctccgaggctcagcctgggcagaagcatggacaggaggaggctgcccaatcgccctaccgagctgttgtccagattttctga

SEQ ID NO: 140 - DNA, 696 bp, Homo sapiens
atggcaggtaagaaagtactcattgtctatgcacaccaggaacccaagtctttcaacggatccttgaagaatgtggctgtagatgaactgagcaggcagggctgcaccgtcacagtgtctgatttgtatgccatgaactttgagccgagggccacagacaaagatatcactggtactctttctaatcctgaggttttcaattatggagtggaaacccacgaagcctacaagcaaaggtctctggctagcgacatcactgatgagcagaaaaaggttcgggaggctgacctagtgatatttcagttcccgctgtactggttcagcgtgccggccatcctgaagggctggatggatagggtgctgtgccagggctttgcctttgacatcccaggattctacgattccggtttgctccagggtaaactagcgctcctttccgtaaccacgggaggcacggccgagatgtacacgaagacaggagtcaatggagattctcgatacttcctgtggccactccagcatggcacattacacttctgtggatttaaagtccttgcccctcagatcagctttgctcctgaaattgcatccgaagaagaaagaaaggggatggtggctgcgtggtcccagaggctgcagaccatctggaaggaagagcccatcccctgcacagcccactggcacttcgggcaataa

SEQ ID NO: 141 - DNA, 561 bp, Homo sapiens
atgggtctgatgatggtgggcgtcctcatcggcaccttcatcgcccatgtggtctgcaagcggctcctcaccgcctgggtggccgccaggatccagagcagcgagaagctgagcgcggttattcgcgtagtggagggaggaagcggcctgaaagtggtggcgctggccagactgacacccataccttttgggcttcagaatgcagtgttttcgattactgatctctcattacccaactatctgatggcatcttcggttggactgcttcctacccagcttctgaattcttacttgggtaccaccctgcggacaatggaagatgtcattgcagaacagagtgttagtggatattttgttttttgtttacagattattataagtataggcctcatgttttatgtagttcatcgagctcaagtggaattgaatgcagctattgtagcttgtgaaatggaactgaaatcttctctggttaaaggcaatcaaccaaataccagtggctcttcattctacaacaagaggaccctaacattttctggaggtggaatcaatgttgtatga

SEQ ID NO: 142 - DNA, 807 bp, Homo sapiens
atggcggagttggacatcgggcagcactgccaggtggagcattgccggcagcgagattttcttccatttgtgtgtgatgattgttcaggaatattttgccttgaacacagaagcagggagtctcatggttgtcctgaggtgactgtaatcaatgagagactgaagacagatcaacatacatcttacccatgctctttcaaagactgtgctgagagagaacttgtggcagttatatgtccttattgtgagaagaatttttgcctgagacaccgtcatcagtcagatcatgagtgtgaaaaactggaaatcccaaagcctcgaatggctgccactcagaaacttgttaaagacattattgattccaagacaggagaaacagcaagtaaacgatggaaaggtgccaaaaatagtgaaacagctgcaaaggttgcattgatgaaattaaagatgcatgctgatggcgataagtcattaccacagacagaaagaatttactttcaggttttcttacctaaagggagcaaagagaagagcaaaccaatgctcttttgccaccgatggagcattggaaaggccatagactttgccgcttctctagccaggcttaaaaatgacaataacaaatttacagctaagaaattaaggctgtgtcacattacttcaggagaagccttacccttggatcatactttggaaacctggattgctaaggaggattgtcctttatataatggtggaaatataatcttggaatatctcaatgatgaagaacaattctgtaaaaatgttgaatcttacttggaatag

SEQ ID NO: 143 - DNA, 690 bp, Homo sapiens
atgggcgacaagatctggctgcccttccccgtgctccttctggccgctctgcctccggtgctgctgcctggggcggccggcttcacaccttccctcgatagcgacttcacctttacccttcccgccggccagaaggagtgcttctaccagcccatgcccctgaaggcctcgctggagatcgagtaccaagttttagatggagcaggattagatattgatttccatcttgcctctccagaaggcaaaaccttagtttttgaacaaagaaaatcagatggagttcacactgtagagactgaagttggtgattacatgttctgctttgacaatacattcagcaccatttctgagaaggtgattttctttgaattaatcctggataatatgggagaacaggcacaagaacaagaagattggaagaaatatattactggcacagatatattggatatgaaactggaagacatcctggaatccatcaacagcatcaagtccagactaagcaaaagtgggcacatacaaattctgcttagagcatttgaagctcgtgatcgaaacatacaagaaagcaactttgatagagtcaatttctggtctatggttaatttagtggtcatggtggtggtgtcagccattcaagtttatatgctgaagagtctgtttgaagataagaggaaaagtagaacttaa

SEQ ID NO: 144 - DNA, 1560 bp, Homo sapiens
atggcgcgggccgggccggcgtggctgctgctggcaatctgggtggtcctgccatcatggctgtcctctgcaaaggtctcctcgctcattgagagaatctctgaccccaaggacttgaaaaaactgctcagaacccggaataatgtactggtgctttactccaaatctgaggtggcagctgaaaatcatctcaggttactgtccacagtggcccaggcggtgaaaggacaagggaccatctgctgggtggactgtggtgatgcagagagtagaaaattgtgcaagaagatgaaagttgacctgagcccgaaggacaaaaaggttgaattattccattaccaggatggtgcatttcatactgaatataaccgagctgtgacatttaagtccatagtggcctttttgaaggatccaaaagggcccccactgtgggaggaagatcctggagccaaagatgttgtccaccttgacagtgaaaaggacttcagacggctcctgaagaaggaagagaagccgctcctgatcatgttttatgccccctggtgcagcatgtgcaagaggatgatgccgcatttccagaaggctgcgactcagctgcgaggccacgccgtgctggccgggatgaatgtctactcctctgaatttgaaaacatcaaggaggagtacagcgtgcgcggcttccccaccatctgctattttgagaaaggacggttcttgttccagtatgacaactatgggtccacagctgaggacattgtggagtggctgaagaatccgcagccgccacagccccaggtccctgagactccctgggcagatgagggcggctccgtttatcacctgaccgatgaagactttgaccagtttgtgaaggaacactcctctgtcctcgtcatgttccacgccccatggtgtggccactgtaagaaaatgaagccggagtttgagaaggcagcagaagccctccatggagaagcggatagctctggtgtccttgcagctgtcgatgccactgtcaacaaggccctggcagaaagattccacatctcagagtttcctacgttgaagtattttaagaatggagagaaatacgcagtgcctgtgctcaggacaaagaagaagtttctcgagtggatgcaaaaccctgaggcccccccgcccccagagcccacgtgggaagagcagcagacaagcgtgttgcacctggtgggggacaacttccgggagaccctgaagaagaagaaacacaccttggtcatgttctacgccccttggtgcccacactgtaagaaggtcattccgcactttactgctactgctgatgccttcaaagatgaccgaaagattgcctgtgccgctgttgactgtgtcaaagacaagaaccaagacctgtgccagcaggaggcggtcaagggctaccccactttccactactaccactatgggaagttcgcagaaaagtatgacagcgaccgcacagaattgggatttaccaattatattcgagccctccgggagggagaccatgaaagactagggaaaaagaaggaagagttataa

SEQ ID NO: 145 - DNA, 3087 bp, Homo sapiens
atggagagtgcgctcaccgcccgtgaccgggtgggggtgcaggatttcgtgctgctggagaacttcaccagcgaggccgccttcatcgagaacctgcggcggcgatttcgggagaatctcatctacacctacattggccccgtcctggtctctgtcaatccctaccgggacctgcagatctacagccggcagcatatggagcgttaccgtggcgtcagcttctatgaagtgccccctcacctgtttgccgtggcggacactgtgtaccgagcactgcgcacggagcgtcgggaccaggctgtgatgatctctggggagagcggggcaggcaagaccgaggccaccaagaggctgctgcagttctatgcagagacctgcccagcccccgagcgcggaggtgccgtgcgggaccggctgctacagagcaacccggtgctggaggcctttggaaatgccaagaccctccggaacgataactccagcaggttcgggaagtacatggatgtgcagtttgacttcaagggtgcccccgtgggtggccacatcctcagttacctcctggaaaagtcacgagtggtgcaccagaatcatggggagcggaacttccacatcttctaccagctgctggaggggggcgaggaggagactcttcgcaggctgggcttggaacggaacccccagagctacctgtacctggtgaagggccagtgtgccaaagtctcctccatcaacgacaagagtgactggaaggtcgtcaggaaggctctgacagtcattgatttcaccgaggatgaagtggaggacctgctgagcatcgtggccagcgtccttcatttgggcaacatccactttgctgccaacgaggagagcaatgcccaggtcaccaccgagaaccagctcaagtatctgaccaggctcctcagcgtggaaggctcgacgctgcgagaagccctgacacacaggaagatcatcgccaagggggaggagctcctgagcccgctgaacctggagcaggccgcgtacgcacgagacgccctcgccaaggctgtgtacagccgcacttttacctggctcgtcgggaagatcaacaggtcgctggcctccaaggacgtggagagccccagctggcggagcaccacggttctcgggctcctggatatttatggctttgaagtgtttcagcataacagctttgagcagttctgcatcaattactgcaacgagaagctgcagcagctcttcatcgagctcacgctcaagtcggagcaggaggagtacgaggcagagggcatcgcgtgggagcccgtccagtatttcaacaacaaaatcatctgtgatctggtggaggagaagtttaagggcatcatctcgattttggatgaggagtgtctgcgccccggggaggccacagacctgaccttcctggagaagctggaggatactgtcaagcaccatccacacttcctgacgcacaagctggctgaccagcggaccaggaaatctctgggccgaggggaattccgccttctgcactatgcgggggaggtgacctacagcgtgaccgggtttctggacaaaaacaatgaccttctcttccggaaccttaaggagaccatgtgtagctcaaagaatcccattatgagccagtgctttgaccggagcgagctcagtgacaagaagcggccagagacggtcgccacccagttcaagatgagcctcctgcagctggtggagatcctgcagtctaaggagcccgcctacgtccgctgcatcaaacccaatgatgccaaacagcccggccgctttgacgaggtgctgatccgccaccaggtgaagtacctggggctgttggaaaacctgcgcgtgcgcagagccggctttgcctatcgccgcaaatacgaagctttcctgcaaaggtacaagtcactgtgcccagagacgtggcccacgtgggcaggacggccgcaggatggggtggctgtgctggtccgacacctgggctacaagccagaagagtacaagatgggcaggaccaagatcttcatccgcttccccaagaccctgtttgccacagaggatgccctggaggtccggcggcagagcctggccacaaagatccaagctgcctggaggggctttcactggcggcagaaattcctccgggtgaagagatcagccatctgcatccagtcgtggtggcgtggaacactgggccggaggaaggcagccaagaggaagtgggcggcacagaccatccggcggctcatccgaggcttcgtcctgcgccacgccccccgctgccccgagaacgccttcttcctggaccatgtgcgcacctcttttttgctaaacctgaggcggcagctgccccagaatgtcctggacacctcgtggcccacgcccccacctgccctgcgtgaggcctcagagcttctgcgggagttgtgcataaagaacatggtgtggaaatactgccggagtatcagccctgagtggaagcagcagctgcagcagaaggccgtggctagtgagatcttcaagggcaagaaggataattaccctcagagtgtacccaggctcttcatcagcactcggcttggtacagatgagatcagcccccgagtgctgcaggccttgggctctgagcccattcagtatgcggtgcctgttgtgaaatacgaccgcaagggctacaagcctcgctcccggcagctgctgctcacgcccaacgccgtcgtcatcgtggaggacgccaaagtcaagcagaggattgattacgccaacctgaccggaatctctgtcagcagcctgagcgacagtctttttgtgcttcatgtacagcgtgcggacaataagcaaaagggagatgtggtgctgcagagtgaccacgtgattgagacgctgaccaagacagccctcagtgccaaccgcgtgaacagcatcaacatcaaccagggcagcatcacgtttgcagggggccccggcagggatggcaccattgacttcacacccggctcggagctgctcatcaccaaggccaagaacgggcacctggctgtggtcgccccacggctgaattctcggtga

SEQ ID NO: 146 - DNA, 3771 bp, Homo sapiens
atgctgttcaagctcctgcagagacagacctatacctgcctgtcccacaggtatgggctctacgtgtgcttcttgggcgtcgttgtcaccatcgtctccgccttccagttcggagaggtggttctggaatggagccgagatcaataccatgttttgtttgattcctatagagacaatattgctggaaagtcctttcagaatcggctttgtctgcccatgccgattgacgttgtttacacctgggtgaatggcacagatcttgaactactgaaggaactacagcaggtcagagaacagatggaggaggagcagaaagcaatgagagaaatccttgggaaaaacacaacggaacctactaagaagagtgagaagcagttagagtgtttgctaacacactgcattaaggtgccaatgcttgtcctggacccagccctgccagccaacatcaccctgaaggacctgccatctctttatccttcttttcattctgccagtgacattttcaatgttgcaaaaccaaaaaacccttctaccaatgtctcagttgttgtttttgacagtactaaggatgttgaagatgcccactctggactgcttaaaggaaatagcagacagacagtatggaggggctacttgacaacagataaagaagtccctggattagtgctaatgcaagatttggctttcctgagtggatttccaccaacattcaaggaaacaaatcaactaaaaacaaaattgccagaaaatctttcctctaaagtcaaactgttgcagttgtattcagaggccagtgtagcgcttctaaaactgaataaccccaaggattttcaagaattgaataagcaaactaagaagaacatgaccattgatggaaaagaactgaccataagtcctgcatatttattatgggatctgagcgccatcagccagtctaagcaggatgaagacatctctgccagtcgttttgaagataacgaagaactgaggtactcattgcgatctatcgagaggcatgcaccatgggttcggaatattttcattgtcaccaacgggcagattccatcctggctgaaccttgacaatcctcgagtgacaatagtaacacaccaggatgtttttcgaaatttgagccacttgcctacctttagttcacctgctattgaaagtcacattcatcgcatcgaagggctgtcccagaagtttatttacctaaatgatgatgtcatgtttgggaaggatgtctggccagatgatttttacagtcactccaaaggccagaaggtttatttgacatggcctgtgccaaactgtgccgagggctgcccaggttcctggattaaggatggctattgtgacaaggcttgtaataattcagcctgcgattgggatggtggggattgctctggaaacagtggagggagtcgctatattgcaggaggtggaggtactgggagtattggagttggacagccctggcagtttggtggaggaataaacagtgtctcttactgtaatcagggatgtgcgaattcctggctcgctgataagttctgtgaccaagcatgcaatgtcttgtcctgtgggtttgatgctggcgactgtgggcaagatcattttcatgaattgtataaagtgatccttctcccaaaccagactcactatattattccaaaaggtgaatgcctgccttatttcagctttgcagaagtagccaaaagaggagttgaaggtgcctatagtgacaatccaataattcgacatgcttctattgccaacaagtggaaaaccatccacctcataatgcacagtggaatgaatgccaccacaatacattttaatctcacgtttcaaaatacaaacgatgaagagttcaaaatgcagataacagtggaggtggacacaagggagggaccaaaactgaattctacagcccagaagggttacgaaaatttagttagtcccataacacttcttccagaggcggaaatcctttttgaggatattcccaaagaaaaacgcttcccgaagtttaagagacatgatgttaactcaacaaggagagcccaggaagaggtgaaaattcccctggtaaatatttcactccttccaaaagacgcccagttgagtctcaataccttggatttgcaactggaacatggagacatcactttgaaaggatacaatttgtccaagtcagccttgctgagatcatttctgatgaactcacagcatgctaaaataaaaaatcaagctataataacagatgaaacaaatgacagtttggtggctccacaggaaaaacaggttcataaaagcatcttgccaaacagcttaggagtgtctgaaagattgcagaggttgacttttcctgcagtgagtgtaaaagtgaatggtcatgaccagggtcagaatccacccctggacttggagaccacagcaagatttagagtggaaactcacacccaaaaaaccataggcggaaatgtgacaaaagaaaagcccccatctctgattgttccactggaaagccagatgacaaaagaaaagaaaatcacagggaaagaaaaagagaacagtagaatggaggaaaatgctgaaaatcacataggcgttactgaagtgttacttggaagaaagctgcagcattacacagatagttacttgggctttttgccatgggagaaaaaaaagtatttccaagatcttctcgacgaagaagagtcattgaagacacaattggcatacttcactgatagcaaaaatactgggaggcaactaaaagatacatttgcagattccctcagatatgtaaataaaattctaaatagcaagtttggattcacatcgcggaaagtccctgctcacatgcctcacatgattgaccggattgttatgcaagaactgcaagatatgttccctgaagaatttgacaagacgtcatttcacaaagtgcgccattctgaggatatgcagtttgccttctcttatttttattatctcatgagtgcagtgcagccactgaatatatctcaagtctttgatgaagttgatacagatcaatctggtgtcttgtctgacagagaaatccgaacactggctaccagaattcacgaactgccgttaagtttgcaggatttgacaggtctggaacacatgctaataaattgctcaaaaatgcttcctgctgatatcacgcagctaaataatattccaccaactcaggaatcctactatgatcccaacctgccaccggtcactaaaagtctagtaacaaactgtaaaccagtaactgacaaaatccacaaagcatataaggacaaaaacaaatataggtttgaaatcatgggagaagaagaaatcgcttttaaaatgattcgtaccaacgtttctcatgtggttggccagttggatgacataagaaaaaaccctaggaagtttgtttgcctgaatgacaacattgaccacaatcataaagatgctcagacagtgaaggctgttctcagggacttctatgaatccatgttccccataccttcccaatttgaactgccaagagagtatcgaaaccgtttccttcatatgcatgagctgcaggaatggagggcttatcgagacaaattgaagttttggacccattgtgtactagcaacattgattatgtttactatattctcattttttgctgagcagttaattgcacttaagcggaagatatttcccagaaggaggatacacaaagaagctagtcccaatcgaatcagagtatag

SEQ ID NO: 147 - DNA, 867 bp, Homo sapiens
atggctgctgtactgcagcgcgtcgagcggctgtccaatcgagtcgtgcgtgtgttgggctgtaacccgggtcccatgaccctccaaggcaccaacacctacctagtggggaccggccccaggagaatcctcattgacactggagaaccagcaattccagaatacatcagctgtttaaagcaggctctaactgaatttaacacagcaatccaggaaattgtagtgactcactggcaccgagatcattctggaggcataggagatatttgtaaaagcatcaataatgacactacctattgcattaaaaaactcccacggaatcctcagagagaagaaattataggaaatggagagcaacaatatgtttatctgaaagatggagatgtgattaagactgagggagccactctaagagttctatatacccctggccacactgatgatcacatggctctactcttagaagaggaaaatgctatcttttctggagattgcatcctaggggaaggaacaacggtatttgaagacctctatgattatatgaactctttaaaagagttattgaaaatcaaagctgatattatatatccaggacatggcccagtaattcataatgctgaagctaaaattcaacaatacatttctcacagaaatattcgagagcagcaaattcttacattatttcgtgagaactttgagaaatcatttacagtaatggagcttgtaaaaattatttacaagaatactcctgagaatttacatgaaatggctaaacataatctcttacttcatttgaaaaaactagaaaaagaaggaaaaatatttagcaacacagatcctgacaagaaatggaaagctcatctttag

SEQ ID NO: 148 - DNA, 387 bp, Homo sapiens
atggctcctgtgaaaaagcttgtggtgaaggggggcaaaaaaaagaagcaagttctgaagttcactcttgattgcacccaccctgtagaagatggaatcatggatgctgccaattttgagcagtttttgcaagaaaggatcaaagtgaacggaaaagctgggaaccttggtggaggggtggtgaccatcgaaaggagcaagagcaagatcaccgtgacatccgaggtgcctttctccaaaaggtatttgaaatatctcaccaaaaaatatttgaagaagaataatctacgtgactggttgcgcgtagttgctaacagcaaagagagttacgaattacgttacttccagattaaccaggacgaagaagaggaggaagacgaggattaa

SEQ ID NO: 149 - DNA, 717 bp, Homo sapiens
atggcgcgcgcctgcctccaggccgtcaagtacctcatgttcgccttcaacctgctcttctggctgggaggctgtggcgtgctgggtgtcggcatctggctggccgccacacaggggagcttcgccacgctgtcctcttccttcccgtccctgtcggctgccaacttgctcatcatcaccggcgcctttgtcatggccatcggcttcgtgggctgcctgggtgccatcaaggagaacaagtgcctcctgctcactttcttcctgctgctgctgctggtgttcctgctggaggccaccatcgccatcctcttcttcgcctacacggacaagattgacaggtatgcccagcaagacctgaagaaaggcttgcacctgtacggcacgcagggcaacgtgggcctcaccaacgcctggagcatcatccagaccgacttccgctgctgtggcgtctccaactacactgactggttcgaggtgtacaacgccacgcgggtacctgactcctgctgcttggagttcagtgagagctgtgggctgcacgcccccggcacctggtggaaggcgccgtgctacgagacggtgaaggtgtggcttcaggagaacctgctggctgtgggcatctttgggctgtgcacggcgctggtgcagatcctgggcctgaccttcgccatgaccatgtactgccaagtggtcaaggcagacacctactgcgcgtag

SEQ ID NO: 150 - DNA, 615 bp, Homo sapiens
atgggtgcatacaagtacatccaggagctatggagaaagaagcagtctgatgtcatgcgctttcttctgagggtccgctgctggcagtaccgccagctctctgctctccacagggctccccgccccacccggcctgataaagcgcgccgactgggctacaaggccaagcaaggttacgttatatataggattcgtgttcgccgtggtggccgaaaacgcccagttcctaagggtgcaacttacggcaagcctgtccatcatggtgttaaccagctaaagtttgctcgaagccttcagtccgttgcagaggagcgagctggacgccactgtggggctctgagagtcctgaattcttactgggttggtgaagattccacatacaaattttttgaggttatcctcattgatccattccataaagctatcagaagaaatcctgacacccagtggatcaccaaaccagtccacaagcacagggagatgcgtgggctgacatctgcaggccgaaagagccgtggccttggaaagggccacaagttccaccacactattggtggctctcgccgggcagcttggagaaggcgcaatactctccagctccaccgttaccgctaa

SEQ ID NO: 151 - DNA, 1620 bp, Homo sapiens
atggcggcggcattacgggtggcggcggtcggggcaaggctcagcgttctggcgagcggtctccgcgccgcggtccgcagcctttgcagccaggccacctctgttaacgaacgcatcgaaaacaagcgccggaccgcgctgctgggagggggccaacgccgtattgacgcgcagcacaagcgaggaaagctaacagccagggagaggatcagtctcttgctggaccctggcagctttgttgagagcgacatgtttgtggaacacagatgtgcagattttggaatggctgctgataagaataagtttcctggagacagcgtggtcactggacgaggccgaatcaatggaagattggtttatgtcttcagtcaggattttacagtttttggaggcagtctgtcaggagcacatgcccaaaagatctgcaaaatcatggaccaggccataacggtgggggctccagtgattgggctgaatgactctgggggagcacggatccaagaaggagtggagtctttggctggctatgcagacatctttctgaggaatgttacggcatccggagtcatccctcagatttctctgatcatgggcccatgtgctggtggggccgtctactccccagccctaacagacttcacgttcatggtaaaggacacctcctacctgttcatcactggccctgatgttgtgaagtctgtcaccaatgaggatgttacccaggaggagctcggtggtgccaagacccacaccaccatgtcaggtgtggcccacagagcttttgaaaatgatgttgatgccttgtgtaatctccgggatttcttcaactacctgcccctgagcagtcaggacccggctcccgtccgtgagtgccacgatcccagtgaccgtctggttcctgagcttgacacaattgtccctttggaatcaaccaaagcctacaacatggtggacatcatacactctgttgttgatgagcgtgaattttttgagatcatgcccaattatgccaagaacatcattgttggttttgcaagaatgaatgggaggactgttggaattgttggcaaccaacctaaggtggcctcaggatgcttggatattaattcatctgtgaaaggggctcgttttgtcagattctgtgatgcattcaatattccactcatcacttttgttgatgtccctggctttctacctggcacagcacaggaatacgggggcatcatccggcatggtgccaagcttctctacgcatttgctgaggcaactgtacccaaagtcacagtcatcaccaggaaggcctatggaggtgcctatgatgtcatgagctctaagcacctttgtggtgataccaactatgcctggcccaccgcagagattgcagtcatgggagcaaagggcgctgtggagatcatcttcaaagggcatgagaatgtggaagctgctcaggcagagtacatcgagaagtttgccaaccctttccctgcagcagtgcgagggtttgtggatgacatcatccaaccttcttccacacgtgcccgaatctgctgtgacctggatgtcttggccagcaagaaggtacaacgtccttggagaaaacatgcaaatattccattgtaa

SEQ ID NO: 152 - DNA, 990 bp, Homo sapiens
atggcgactggacagaagttgatgagagctgttagagtttttgaatttggtgggccagaagtcctgaaattgcgatcagatattgcagtaccgattccaaaagaccatcaggttctaatcaaggtccatgcatgtggtgtcaaccccgtggagacatacattcgctctggtacttatagtagaaaaccactcttaccctatactcctggctcagatgtggctggggtgatagaagctgttggagataatgcatctgctttcaagaaaggtgacagagttttcactagcagcacgatctctgggggttatgcagagtatgctcttgcagcagaccacactgtttacaaactacctgaaaaactggactttaaacaaggagctgccatcggcattccatattttactgcttatcgagctctgatccacagtgcctgtgtgaaagctggagagagtgttctggttcatggggcaagtggaggagttggattagcagcatgccaaattgctagagcttatggcttaaagattttgggcactgctggtactgaggaaggacaaaagattgttttgcaaaatggagcccatgaagtgttcaatcacagagaagtgaattacattgataaaattaagaagtatgttggtgagaaaggaattgatataattattgaaatgttagctaatgtaaatcttagtaaagacttgagtcttctgtcacatggaggacgagtgatagttgttggcagcagaggtactattgaaataaacccacgagacaccatggcaaaggagtcgagtataattggagttactctcttttcctcaaccaaggaggaatttcagcaatatgcagcagcccttcaagctggaatggaaattggctggttgaaacctgtgataggttctcaatatccattggagaaggtggccgaggctcatgaaaatatcattcatggtagtggggctactggaaaaatgattcttctcttatga

SEQ ID NO: 153 - DNA, 2382 bp, Homo sapiens
atgggagtctggttaaataaagatgactatatcagagacttgaaaaggatcattctctgttttctgatagtgtatatggccattttagtgggcacagatcaggatttttacagtttacttggagtgtccaaaactgcaagcagtagagaaataagacaagctttcaagaaattggcattgaagttacatcctgataaaaacccgaataacccaaatgcacatggcgattttttaaaaataaatagagcatatgaagtactcaaagatgaagatctacggaaaaagtatgacaaatatggagaaaagggacttgaggataatcaaggtggccagtatgaaagctggaactattatcgttatgattttggtatttatgatgatgatcctgaaatcataacattggaaagaagagaatttgatgctgctgttaattctggagaactgtggtttgtaaatttttactccccaggctgttcacactgccatgatttagctcccacatggagagactttgctaaagaagtggatgggttacttcgaattggagctgttaactgtggtgatgatagaatgctttgccgaatgaaaggagtcaacagctatcccagcctcttcatttttcggtctggaatggccccagtgaaatatcatggagacagatcaaaggagagtttagtgagttttgcaatgcagcatgttagaagtacagtgacagaactttggacaggaaattttgtcaactccatacaaactgcctttgctgctggtattggctggctgatcactttttgttcaaaaggaggagattgtttgacttcacagacacgactcaggcttagtggcatgttggatggtcttgttaatgtaggatggatggactgtgccacccaggataacctttgtaaaagcttagatattacaacaagtactactgcttattttcctcctggagccactttaaataacaaagagaaaaacagtattttgtttctcaactcattggatgctaaagaaatatatttggaagtaatacataatcttccagattttgaactactttcggcaaacacactagaggatcgtttggctcatcatcggtggctgttattttttcattttggaaaaaatgaaaattcaaatgatcctgagctgaaaaaactaaaaactctacttaaaaatgatcatattcaagttggcaggtttgactgttcctctgcaccagacatctgtagtaatctgtatgtttttcagccgtctctagcagtatttaaaggacaaggaaccaaagaatatgaaattcatcatggaaagaagattctatatgatatacttgcctttgccaaagaaagtgtgaattctcatgttaccacgcttggacctcaaaattttcctgccaatgacaaagaaccatggcttgttgatttctttgccccctggtgtccaccatgtcgagctttactaccagagttacgaagagcatcaaatcttctttatggtcagcttaagtttggtacactagattgtacagttcatgagggactctgtaacatgtataacattcaggcttatccaacgacagtggtattcaaccagtccaacattcatgagtatgaaggacatcactctgctgaacaaatcttggagttcatagaggatcttatgaatccttcagtggtctcccttacacccaccaccttcaacgaactagttacacaaagaaaacacaacgaagtctggatggttgatttctattctccgtggtgtcatccttgccaagtcttaatgccagaatggaaaagaatggcccggacattaactggactgatcaacgtgggcagtatagattgccaacagtatcattctttttgtgcccaggaaaacgttcaaagataccctgagataagattttttcccccaaaatcaaataaagcttatcattatcacagttacaatggttggaatagggatgcttattccctgagaatctggggtctaggatttttacctcaagtatccacagatctaacacctcagactttcagtgaaaaagttctacaagggaaaaatcattgggtgattgatttctatgctccttggtgtggaccttgccagaattttgctccagaatttgagctcttggctaggatgattaaaggaaaagtgaaagctggaaaagtagactgtcaggcttatgctcagacatgccagaaagctgggatcagggcctatccaactgttaaattttatttctacgaaagagcaaagagaaattttcaagaagagcagataaataccagagatgcaaaagcaatcgctgccttaataagtgaaaaattggaaactctccgaaatcaaggcaagaggaataaggatgaactttga

SEQ ID NO: 154 - DNA, 642 bp, Homo sapiens
atggcaggtactctcctgtcgccccccagtggcgtccccctggagagactcatacgggtggccacggaaagaggctacacggcccagggagagatgttctcagtggccgatatgggcaggctggcccaggaggtgctgggctgccaggccaagctgctctctggtggcctgggcggtcccaacagagacctcgtcctgcagcacctggtcactggacatcccctgctcatcccctacgacgaggacttcaaccatgagccgtgtcagaggaagggccacaaggcacactgggcggtgagtgcaggggtcctgctgggtgttcgggctgtgcccagtctcggctacactgaggaccctgagctgccgggcctgttccacccagtgctgggcacgccctgccaaccaccatccctgccagaggagggctccccgggagctgtctacctgctgtccaagcagggcaagagttggcactatcagctgtgggactacgaccaggtccgggagagcaacctgcagctgacggacttctcgccctcacgggccactgacggccgggtgtacgtggtgcccgtgggtggggtacgggctggcctctgtggccaggccctgctcctcacaccacaggactgcagccattag

SEQ ID NO: 155 - DNA, 309 bp, Homo sapiens
atggcaggacaagcgtttagaaagtttcttccactctttgaccgagtattggttgaaaggagtgctgctgaaactgtaaccaaaggaggcattatgcttccagaaaaatctcaaggaaaagtattgcaagcaacagtagtcgctgttggatcgggttctaaaggaaagggtggagagattcaaccagttagcgtgaaagttggagataaagttcttctcccagaatatggaggcaccaaagtagttctagatgacaaggattatttcctatttagagatggtgacattcttggaaagtacgtagactga

SEQ ID NO: 156 - DNA, 369 bp, Homo sapiens
atgttgccagtattgccaaagcctgggatgcccttggcggcactggtgacggggctgtcaggactgttatggccctgttgtgctgagttagttggaacagaattcaagctccctgcactagtccacctgccccactgcttcttcgcttctctcttggaaagtccagtctctcctcggcttgcaatggaccccaactgctcctgcgccgctggtgtctcctgcacctgcgctggttcctgcaagtgcaaagagtgcaaatgcacctcctgcaagaagagctgctgctcctgctgccccgtgggctgtagcaagtgtgcccagggctgtgtttgcaaaggggcgtcagagaagtgcagctgctgcgactga

SEQ ID NO: 157 - protein, 11 aa, Homo sapiens
RASQSIGSSLH

SEQ ID NO: 158 - protein, 7 aa, Homo sapiens
YASQSLS

SEQ ID NO: 159 - protein, 9 aa, Homo sapiens
HQSSRLPHT

SEQ ID NO: 160 - protein, 5 aa, Homo sapiens
SFAMH

SEQ ID NO: 161 - protein, 16 aa, Homo sapiens
VIDTRGATYYADSVKG

SEQ ID NO: 162 - protein, 10 aa, Homo sapiens
LGNFYYGMDV

SEQ ID NO: 163 - DNA, 522 bp, Homo sapiens
atggctcgaatgaaccgcccagctcctgtggaagtcacatacaagaacatgagatttcttattacacacaatccaaccaatgcgaccttaaacaaatttatagaggaacttaagaagtatggagttaccacaatagtaagagtatgtgaagcaacttatgacactactcttgtggagaaagaaggtatccatgttcttgattggccttttgatgatggtgcaccaccatccaaccagattgttgatgactggttaagtcttgtgaaaattaagtttcgtgaagaacctggttgttgtattgctgttcattgcgttgcaggccttgggagagctccagtacttgttgccctagcattaattgaaggtggaatgaaatacgaagatgcagtacaattcataagacaaaagcggcgtggagcttttaacagcaagcaacttctgtatttggagaagtatcgtcctaaaatgcggctgcgtttcaaagattccaacggtcatagaaacaactgttgcattcaataa

SEQ ID NO: 164 - DNA, 624 bp, Homo sapiens
atggccccctttgagcccctggcttctggcatcctgttgttgctgtggctgatagcccccagcagggcctgcacctgtgtcccaccccacccacagacggccttctgcaattccgacctcgtcatcagggccaagttcgtggggacaccagaagtcaaccagaccaccttataccagcgttatgagatcaagatgaccaagatgtataaagggttccaagccttaggggatgccgctgacatccggttcgtctacacccccgccatggagagtgtctgcggatacttccacaggtcccacaaccgcagcgaggagtttctcattgctggaaaactgcaggatggactcttgcacatcactacctgcagttttgtggctccctggaacagcctgagcttagctcagcgccggggcttcaccaagacctacactgttggctgtgaggaatgcacagtgtttccctgtttatccatcccctgcaaactgcagagtggcactcattgcttgtggacggaccagctcctccaaggctctgaaaagggcttccagtcccgtcaccttgcctgcctgcctcgggagccagggctgtgcacctggcagtccctgcggtcccagatagcctga

SEQ ID NO: 165 - DNA, 1506 bp, Homo sapiens
atgcaggtttgcagccagccccaaagggggtgtgtgcgcgagcagagcgctataaatacggcgcctcccagtgcccacaacgcggcgtcgccaggaggagcgcgcgggcacagggtgccgctgaccgaggcgtgcaaagactccagaattggaggcatgatgaagactctgctgctgtttgtggggctgctgctgacctgggagagtgggcaggtcctgggggaccagacggtctcagacaatgagctccaggaaatgtccaatcagggaagtaagtacgtcaataaggaaattcaaaatgctgtcaacggggtgaaacagataaagactctcatagaaaaaacaaacgaagagcgcaagacactgctcagcaacctagaagaagccaagaagaagaaagaggatgccctaaatgagaccagggaatcagagacaaagctgaaggagctcccaggagtgtgcaatgagaccatgatggccctctgggaagagtgtaagccctgcctgaaacagacctgcatgaagttctacgcacgcgtctgcagaagtggctcaggcctggttggccgccagcttgaggagttcctgaaccagagctcgcccttctacttctggatgaatggtgaccgcatcgactccctgctggagaacgaccggcagcagacgcacatgctggatgtcatgcaggaccacttcagccgcgcgtccagcatcatagacgagctcttccaggacaggttcttcacccgggagccccaggatacctaccactacctgcccttcagcctgccccaccggaggcctcacttcttctttcccaagtcccgcatcgtccgcagcttgatgcccttctctccgtacgagcccctgaacttccacgccatgttccagcccttccttgagatgatacacgaggctcagcaggccatggacatccacttccatagcccggccttccagcacccgccaacagaattcatacgagaaggcgacgatgaccggactgtgtgccgggagatccgccacaactccacgggctgcctgcggatgaaggaccagtgtgacaagtgccgggagatcttgtctgtggactgttccaccaacaacccctcccaggctaagctgcggcgggagctcgacgaatccctccaggtcgctgagaggttgaccaggaaatacaacgagctgctaaagtcctaccagtggaagatgctcaacacctcctccttgctggagcagctgaacgagcagtttaactgggtgtcccggctggcaaacctcacgcaaggcgaagaccagtactatctgcgggtcaccacggtggcttcccacacttctgactcggacgttccttccggtgtcactgaggtggtcgtgaagctctttgactctgatcccatcactgtgacggtccctgtagaagtctccaggaagaaccctaaatttatggagaccgtggcggagaaagcgctgcaggaataccgcaaaaagcaccgggaggagtga